(19) United States
(12) Patent Application Publication
(10) Pub. No.: US 2021/0401987 A1
(43) Pub. Date: Dec. 30, 2021

(54) METHODS AND COMPOSITIONS FOR TREATING INFLAMMATORY AND FIBROTIC PULMONARY DISORDERS

(71) Applicants: University of Houston System, Houston, TX (US); The Board of Regents of The University of Texas System, Austin, TX (US); Tract Pharmaceuticals, Inc., West Hartford, CT (US)

(72) Inventors: Frank McKEON, Houston, TX (US); Wei RAO, Houston, TX (US); Jingzhong XIE, Houston, TX (US); Shan WANG, Houston, TX (US); Wa XIAN, Houston, TX (US); Matthew VINCENT, West Hartford, CT (US); Suchan NIROULA, Houston, TX (US)

(73) Assignees: University of Houston System, Houston, TX (US); The Board of Regents of The University of Texas System, Austin, TX (US); Tract Pharmaceuticals, Inc., West Hartford, CT (US)

(21) Appl. No.: 17/475,545

(22) Filed: Sep. 15, 2021

Related U.S. Application Data
(63) Continuation-in-part of application No. PCT/US2021/023060, filed on Mar. 19, 2021.
(60) Provisional application No. 62/992,282, filed on Mar. 20, 2020.

(51) Int. Cl. A61K 45/00

(52) U.S. Cl. CPC A61K 45/00

(57) ABSTRACT

The present disclosure addresses inflammatory lung diseases, such as COPD, and fibrotic lung diseases such as idiopathic pulmonary fibrosis, from the standpoint of inhibiting or ablating pathogenic epithelial stem cells found in the pulmonary tract.

### PRIORITY CLAIM

This application is a continuation-in-part of PCT Application Serial No. PCT/US2021/023060, filed Mar. 19, 2021, which claims benefit of priority to U.S. Provisional Application Ser. No. 62/992,282, filed Mar. 20, 2020, the entire contents of each of which are hereby incorporated by reference.

### BACKGROUND

Chronic obstructive pulmonary disease (COPD) is an inflammatory condition of the lung marked by chronic bronchitis, small airway occlusion, inflammation, fibrosis, and emphysematous destruction of alveoli (Hogg et al., 2004; McDonough et al., 2011; Decramer et al., 2012; Barnes et al., 2015). The global impact of COPD is enormous as evidenced by the 250 million patients with this condition and the 50%, 5-year survival of those with GOLD stage 3 and 4 disease (FEV1<50%; Quanderi and Hurst, 2018; Hogg et al., 2004; McDonough et al., 2011; Decramer et al., 2012; Barnes et al., 2015, Singh et al., 2019). Risk factors include chronic exposure to cigarette smoke and environmental pollutants, early-life respiratory illnesses such as asthma, viral pneumonia, and perinatal bronchopulmonary dysplasia, as well as genetics (Fletcher and Peto, 1977; McGeachie et al., 2016; Martinez, 2016; Busch et al., 2017; Martinez et al., 2018).

Given that 80% of COPD cases are associated with long-term chronic smoking, absolute abstinence will limit risk for this disease. However, the success of smoking cessation programs on patients with established COPD has been disappointing (Fletcher and Peto, 1977; Scanlon et al., 2000; Gamble et al., 2007; Hogg and Timens, 2009; Wen et al., 2010; Miller et al., 2011; Barnes, 2016), and most people with moderate COPD progress to severe disease (FEV1<30) in a 5- to 20-year interval despite tobacco cessation. The mechanistic basis for this dogged progression of COPD is unclear, but multiple studies have shown that lung inflammatory pathways remain activated years after smoking cessation (Scanlon et al., 2000; Gamble et al., 2007; Wen et al., 2010; Miller et al., 2011; Barnes, 2016).

The predisposing influences of early-life pulmonary disease, coupled with the general irreversibility of this condition in previous smokers, suggest that inertial processes may drive COPD. Consistent with this notion, squamous and goblet cell metaplasia typically observed in COPD likewise are not fully abolished by smoking cessation (Hogg and Timens, 2009; Lapperre et al., 2007; Raju et al., 2016; Zhou-Suckow et al., 2017). This persistence was surprising as metaplasia has been tied to acute effects of cigarette smoke and inflammatory cytokines on normal airway progenitors (Wills-Karp et al., 1998; Araya et al., 2007; Schamberger et al., 2015). Removal of these toxicants was expected to enable distal airway progenitors to resume their roles in restoring tissue homeostasis in small airways and terminal bronchioles (Kumar et al., 2011; Zuo et al., 2015; Vaughan et al., 2015; Ray et al., 2016; Yang et al., 2018; Tanaka et al., 2018). In this context, the inventors speculated that permanent alteration of distal airway progenitors or their clonal composition could underlie their failure to restore airways homeostasis post-smoking cessation.

### SUMMARY

Chronic obstructive pulmonary disease is a progressive, potentially life-threatening disease characterized by airflow limitation and for which no curative treatment is available. COPD is a prevalent condition, affecting between 10 and 20% of the population, depending on the definition being used and countries being investigated. It is currently the fourth leading mortality cause worldwide and is expected to become third in 2020. COPD encompasses two main conditions, chronic bronchitis and emphysema, characterized by persistent inflammation of the airways and widespread destruction of the alveolar walls, respectively.

In making the present disclosure, the inventors leveraged robust technologies that enable the cloning of distal airway epithelial cells (Kumar et al., 2011; Zuo et al., 2015) to perform a comparative analysis of clonogenic cells in patients with and without COPD. The data revealed that the COPD distal airways are populated by a highly stereotyped triad of clonogenic cells (referred to as “Cluster 2”, “Cluster 3” and “Cluster 4” stem cells, and collectively to as “pathogenic lung epithelial stem cells” or “PLESCs”) in addition to the normal clonogenic cell type (referred to as “Cluster 1” stem cells, or “normal regenerative stem cells”) that predominates control lungs. The inventors demonstrate that the Cluster 2, 3, and 4 clonogenic cells are stably committed to the mucous and squamous metaplastic fates and autonomously drive mucus hypersecretion, pro-inflammatory and pro-fibrotic programs akin to those implicated in COPD pathology. In addition, the data shows that a similar triad of clonogenic cells in COPD also exists in control adult and 13-, 14-, and 17-week fetal lung, albeit at low ratios to the predominant distal airway clone type seen in normal lung. Thus the variant clones predate any disease state and yet have features that could contribute to the emergence, pathology, or progression of COPD as a function of their numbers.

As described in greater detail in the appended examples, figures and tables, despite this clonal heterogeneity in COPD, all clones from Clusters 1-4 shared the expression of established markers of distal airway progenitors (p63, KRT5), displayed a clonogenicity of >50%, and showed a long-term proliferative potential of at least 25 passages (>8 mos) while maintaining a clonogenicity of >50%.

In vitro differentiation of Cluster 1 clones from both COPD and control libraries yielded normal epithelia (NM) marked by p63+ basal cells and suprabasal, differentiated cells expressing SCGB1A1, SFTPB, and AQP4, similar to human epithelia of small airways, terminal bronchioles and aveoli. In contrast, clones of Cluster 2 differentiated to a goblet cell metaplasia (GCM) marked by p63+ basal cells and differentiated goblet cells co-expressing SCGB1A1, MUC5AC, and MUC5B. Clones from Clusters 3 and 4 gave rise to squamous cell metaplasia (SCM) marked by immature p63 cells and differentiated, keratin 10 (Krt10)- and involucrin (IVL)-expressing cells.

Normal control clones, as well as those from Cluster 1 of COPD patients, assembled into a polarized epithelium in vivo comprised of cells positive for p63, Krt5, SCGB1A1, AQP4, AQP5, SFTPB, and SFTPC like that of normal human small airway, terminal bronchioles and alveoli or that produced by in vitro differentiation of Cluster 1 clones.

Also mirroring the in vitro ALI cultures, the xenografts of Cluster 2 clones from COPD libraries formed an epithelium dominated by large goblet cells expressing MUC5AC and MUC5B, and both Cluster 3 and 4 clone xenografts yielded squamous cell metaplasia expressing IVL and Krt10.

In addition, Cluster 4 clones in particular constitutively express a broad array of genes related to inflammation and are denoted herein as ‘inflammatory SCM’ or ‘iSCM’ versus ‘SCM’ for Cluster 3 clones.

Lastly, whole exome DNA sequencing of Cluster 2, 3, and 4 clones derived from these COPD patients showed an absence of copy number variation events greater than 10 Kb. Moreover, the 127-157 single nucleotide variation (synonymous, nonsynonymous, indels) events in these clones were consistent in number with other somatic cells and did not impact known tumor suppressor or oncogenes such as p16 or p53, arguing against the possibility these clones are related to neoplastic lesions.

In addition, antibody binding (such as by flow cytometric analyses) provides distinction between the normal regerenative and pathogenic stem cells, with antibodies to AQP5 bing specific to Cluster 1 cells, CXCL8 for Cluster 4 cells, and TRPC6 antibodies for both Clusters 2 and 3.

Endothelial cell activation can also be used to identify and distinguish between normal regenerative stem cells (Cluster 1) and the pathogenic lung stem cells (Cluster 2, 3 and 4). To illustrate, as described below, endothelial cell activation was assessed by the expression of Vcam1 (CD106), a vascular adhesion protein that binds VLA-1 (alpha4beta1 integrin) on monocytes and lymphocytes, and it was found that Cluster 1 clones from COPD patients showed no induction, whereas IL-13 challenge yielded a 30-fold induction of Vcam1. In this same assay media conditioned by Cluster 2 (GCM) or Cluster 3 (SCM) clones, respectively, yielded 4- and 10-fold inductions of VCAM1, and while media from Cluster 4 (iSCM) clones induced Vcam1 by 60-fold. In addition to identifying the pathogenic clone clusters, this data also provides functional support for the notion that each of the pathogenic clone clusters can promote an early step in inflammatory responses.

In one aspect, the disclosure provides a composition comprising a clonal population of a pathogenic lung stem cell, such as may be isolated from the lungs of a subject suffering from COPD, idiopathic pulmonary fibrosis, cystic fibrosis, Asthma, bronchopulmonary dysplasia (BPD), chronic bronchitis or emphysema, wherein the stem cells differentiate into inflammatory lung epithelium. Preferably the composition, with respect to the cellular component, is at least 50 percent pathogenic lung epithelial stem cell, more preferably at least 75, 80, 85, 90, 95 or even 99 percent pathogenic lung epithelial stem cell.

In certain preferred embodiments, the composition comprises an enriched population of pathogenic lung epithelial stem cells of Cluster 2 (GCM), such as may be characterized as having an mRNA profile with upregulated expression of B4GALT1, SPC24, APOBEC3C and EPPK1 (i.e., relative to normal regenerative lung epithelial stem cells), and more preferably 3, 4, 5, 6 or more (including all) of B4GALT1, TOR1AIP2, SPC24, APOBEC3C, EPPK1, RIN2, TRA2A, ANP32E, GTF3A, TP53, HMGN2, TNRC6B, CDK4, DTYMK, PHF19, DEK, ZDHHC12, COPRS, CD47, BIRC5, LSM4, LSM2, YIF1A, CISD2, OAZ1, TCEA1, CLSPN, CDKN3, R3HDM2, CTDNEP1, NABP2, PDGFA, DCTPP1, SAC3D1, H2AFV, FAM96A, MYBL2, CACYBP, TYMS, SSRP1 and/or ICMT. The cells making up the composition can be at least 50%, 60%, 70%, 80%, 90%, 95% or even 98% Cluster 2 cells.

In certain preferred embodiments, the composition comprises an enriched population of pathogenic lung epithelial stem cells of Cluster 3 (SCM), such as may be characterized as having an mRNA profile with upregulated expression of SCD (i.e., relative to normal regenerative lung epithelial stem cells), and more preferably 3, 4, 5, 6 or more (including all) of SCD, SQLE, INSIG1, FDPS, FDFT1, FASN, MVD, HMGCS1, ACAT2, EBP, DHCR7, HMGCR, ACLY, PGP, MVK, SLC25A1, PCYT2, RDH11 and/or TMEM97. The cells making up the composition can be at least 50%, 60%, 70%, 80%, 90%, 95% or even 98% Cluster 3 cells.

In certain preferred embodiments, the composition comprises an enriched population of pathogenic lung epithelial stem cells of Cluster 4 (iSCM), such as may be characterized as having an mRNA profile with upregulated expression of CXCL8 (i.e., relative to normal regenerative lung epithelial stem cells), and more preferably 3, 4, 5, 6 or more (including all) of CXCL8, CCL20, PLAU, CXCL1, NFKBIA, BIRC3, PLAUR, TNFAIP3, TNIP1, HBEGF, PTP4A2, BID, HS3ST1, CDCl42EP1, PIM3, TINAGLI, TRAF4, SPATS2L, IFNGR2, CLDN1, MAP2K3, TPM1, CEBPB, CXCL16, SNX3, PIM1, UBE2F, RAC1, MAST4, CCND3, TRAM1 and/or CSTB. The cells making up the composition can be at least 50%, 60%, 70%, 80%, 90%, 95% or even 98% Cluster 4 cells.

In certain embodiments, the composition of cells can be at least 50%, 60%, 70%, 80%, 90%, 95% or even 98% Cluster 2, Cluster 3, or Cluster 4 cells, or a combination thereof.

In certain embodiments, the composition of cells can be a mix of normal regenerative lung epithelial stem cells and one or more of Cluster 2, Cluster 3 and/or Cluster 4 cells.

In certain embodiments, the disclosure provides an array of cells, e.g., in the form of a multiwell plate of other means for providing the cells (preferably in culture media) as discrete populations of normal regenerative lung epithelial stem cells and one or more of Cluster 2, Cluster 3 and/or Cluster 4 cells.

The disclosure further provides a composition comprising a population of cells enriched in a clonal subpopulation of pathogenic lung epithelial stem cells from the lung of a subject, wherein the clonal subpopulation of cells differentiates into inflammatory lung epithelium (i.e., having a microarchitecture resembling lung epithelia from a patient having COPD, bronchopulmonary dysplasia, cystic fibrosis, Asthma, chronic bronchitis, emphysema, idiopathic pulmonary fibrosis, chronic rhinosinusitis or a combination thereof).

The disclosure further provides a method of screening for an agent effective in the treatment or prevention of an inflammatory lung disorder, such as COPD, bronchopulmonary dysplasia, chronic bronchitis, emphysema, idiopathic pulmonary fibrosis, Asthma, chronic rhinosinusitis or a combination thereof, including the steps of providing a population of pathogenic lung epithelial stem cells, wherein the pathogenic lung epithelial stem cells are able to differentiate into inflammatory lung epithelium; providing a test agent; and exposing the pathogenic lung epithelial stem cells to the test agent; wherein if the test agent is cytotoxic, cytostatic and/or able to inhibit the differentiation of the pathogenic lung epithelial stem cells to inflammatory lung epithelium, the test agent is an agent effective in the treatment or prevention of inflammatory lung diseases such as COPD, bronchopulmonary dysplasia (BPD), chronic bronchitis, emphysema, idiopathic pulmonary fibrosis, Asthma, chronic rhinosinusitis or a combination thereof.

In certain embodiments, the pathogenic lung epithelial stem cells are mammalian pathogenic lung epithelial stem cells, such as human pathogenic lung epithelial stem cells.

In certain embodiments, candidate therapeutic agents reduce the viability, growth or ability to differentiation by 70, 80, 90, 95, 96, 97, 98, 99 or even 100%.

The disclosure further provides a method of screening for an agent effective in the detection of an inflammatory lung disorder, such as COPD, bronchopulmonary dysplasia, chronic bronchitis, emphysema, idiopathic pulmonary fibrosis, Asthma, chronic rhinosinusitis or a combination thereof, including the steps of providing pathogenic lung epithelial stem cells; providing a test agent; and exposing the pathogenic lung epithelial stem cells to the test agent; wherein if the test agent specifically binds to the pathogenic lung epithelial stem cells, i.e., relative to normal regenerative lung stem cells, the test agent is an agent effective in the detection of stem cells giving rise to an inflammatory lung disorder.

In certain embodiments, the pathogenic lung epithelial stem cells are mammalian, and more preferably are human.

The disclosure further provides a method of detecting the presence of an inflammatory lung disorder, such as COPD, bronchopulmonary dysplasia, chronic bronchitis, emphysema, idiopathic pulmonary fibrosis, Asthma, chronic rhinosinusitis or a combination thereof, in a subject including the steps of providing a detection agent that specifically binds to pathogenic lung epithelial stem cells and cells differentiated therefrom; administering the detection agent to a subject; and detecting whether the detection agent specifically binds to PLESCs or tissue derived therefrom in the lungs of the subject, wherein, if the detection agent specifically binds to a cell in the lung of the subject to a higher degree than the average normal lung, the subject is diagnosed with and inflammatory lung disorder or as having a risk of developing an inflammatory lung disorder.

Moreover, the inventors have demonstrated the ability to target the pathogenic stem cells with drugs that selectively inhibit growth or differentiation, or which are lethal to those cells, relative to normal regenerative lung stem cells.

One aspect of the present disclosure provides a method for treating a patient suffering from a chronic inflammatory disorder, metaplasia, dysplasia or even certain cancers of pulmonary tissue, which method comprises administering to the patient an agent that selectively kills or inhibits the proliferation or differentiation of pathogenic lung epithelial stem cells (PLESCs) in the pulmonary tissue relative to normal epithelial stem cells in pulmonary tissue in which the PLESC is found. Representative pulmonary diseases and disorder that can be treated include COPD, bronchopulmonary dysplasia (BPD), chronic bronchitis, emphysema, idiopathic pulmonary fibrosis, Asthma, chronic rhinosinusitis or a combination thereof, and patients suffering from combinations of these conditions as well.

Another aspect of the disclosure provides a method of reducing proliferation, survival, migration, or colony formation ability of PLESCs in a subject in need thereof comprising contacting the PLESC with a therapeutically effective amount of an anti-PLESC agent that selectively kills or inhibits the proliferation or differentiation of a PLESC population relative to normal epithelial stem cells in the tissue in which the PLESCs is found.

Another aspect of the disclosure provides a pharmaceutical preparation for treating one or more of chronic inflammatory injury, metaplasia, dysplasia or cancer of pulmonary epithelial tissue, which preparation comprises an anti-PLESC agent that selectively kills or inhibits the proliferation or differentiation of PLESCs relative to normal epithelial stem cells. Suitable anti-PLESC agents may include, for example, small molecule drugs, peptides and polypeptides (including antibodies and antibody mimetics), carbohydrates and nucleic acids. Exemplary anti-PLESC agents are described herein, and those skilled in the art will be able to identify other suitable agents using the cells and drug screening assays of the disclosure.

Another aspect of the disclosure provides a pharmaceutical preparation for treating one or more of COPD, BPD, chronic bronchitis, emphysema, idiopathic pulmonary fibrosis, Asthma, chronic rhinosinusitis or a combination thereof, which preparation comprises an anti-PLESC agent that selectively kills or inhibits the proliferation or differentiation of PLESCs relative to normal pulmonary stem cells.

In certain embodiments, the disclosure provides inhaled formulations for pulmonary delivery of an anti-PLESC agent. For inflammatory lung diseases such as COPD and idiopathic pulmonary fibrosis, the pulmonary delivery formulation is one that delivers the anti-PLESC to the deep lung tissues (i.e., preferentially relative to the upper airways). Minimally invasive drug delivery through the lung can be achieved using environment friendly propellants, non-aqueous inhalers, user-friendly dry powder inhalers, and jet or ultrasonic nebulizers. These include metered dose inhalers, nebulizers, and dry powder inhalers.

Yet another aspect of the disclosure provides a drug eluting device, such as an airway stent or nanofibers, for sustained delivery of an anti-PLESC agent, which device comprises drug release means including an anti-PLESC agent that selectively kills or inhibits the proliferation or differentiation of PLESCs relative to normal epithelial stem cells, which device when deployed in a patient positions the drug release means proximal to the surface of the lung (such as bronchial or bronchiole placement) and releases the agent in an amount sufficient to achieve a therapeutically effective exposure of the luminal surface to the agent. Examples of drug eluting devices are drug eluting stents, drug eluting collars and drug eluting ballons.

In other embodiments, there are provided drug eluting devices that can be implanted proximal to the diseased portion of the luminal surface of the pulmonary tract, such as implanted extraluminally (i.e., submucosally or in or on the circular muscle or longitudinal muscle) rather than intraluminally.

In certain embodiments, the anti-PLESC agent has an IC₅₀ for selectively killing PLESCs that is ⅕^{th} or less the IC₅₀ for killing normal epithelial stem cells in the tissue in which the PLESCs are found, more preferably 1/10^{th}, 1/20^{th}, 1/50^{th}, 1/100^{th}, 1/250th, 1/500^{th} or even 1/1000^{th} or less the IC₅₀ for killing normal epithelial stem cells.

In certain embodiments, the anti-PLESC agent has an IC₅₀ for selectively killing PLESCs that is ⅕^{th} or less the IC₅₀ for killing normal pulmonary stem cells, more preferably 1/10^{th}, 1/20^{th}, 1/50^{th}, 1/100^{th}, 1/250^{th}, 1/500^{th} or even 1/1000^{th} or less the IC₅₀ for killing normal pulmonary stem cells.

In certain embodiments, the anti-PLESC agent has an IC₅₀ for selectively killing COPD PLESCs that is ⅕^{th} or less the IC₅₀ for killing normal lung stem cells, more preferably 1/10^{th}, 1/20^{th}, 1/50^{th}, 1/100^{th}, 1/250^{th}, 1/500^{th} or even 1/1000^{th} or less the IC₅₀ for killing normal lung stem cells.

In certain embodiments, the anti-PLESC agent has an IC₅₀ for selectively inhibiting the proliferation of PLESCs that is ⅕^{th} or less the IC₅₀ for inhibiting normal epithelial stem cells in the pulmonary tissue in which the PLESCs are found, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal epithelial stem cells.

In certain embodiments, the anti-PLESC agent has an IC₅₀ for selectively inhibiting the proliferation of PLESCs that is ⅕^{th} or less the IC₅₀ for inhibiting the proliferation of normal pulmonary stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal pulmonary stem cells.

In certain embodiments, the anti-PLESC agent has an IC₅₀ for selectively inhibiting the proliferation of COPD PLESCs that is ⅕^{th} or less the IC₅₀ for inhibiting the proliferation of normal lung stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the proliferation of normal lung stem cells.

In certain embodiments, the anti-PLESC agent has an IC₅₀ for selectively inhibiting the differentiation of PLESCs that is ⅕^{th} or less the IC₅₀ for inhibiting the differentiation of normal pulmonary stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal pulmonary stem cells.

In certain embodiments, the anti-PLESC agent has an IC₅₀ for selectively inhibiting the differentiation of COPD PLESCs that is ⅕^{th} or less the IC₅₀ for inhibiting the differentiation of normal lung stem cells, more preferably 1/10^{th}, 1/20^{th}, 1/50^{th}, 1/100^{th}, 1/250^{th}, 1/500^{th} or even 1/1000^{th} or less the IC₅₀ for inhibiting the differentiation of normal lung stem cells.

In certain embodiments, the anti-PLESC agent has a therapeutic index (TI) for treating inflammatory diseases and disorders of the pulmonary tract, or other metaplasia, dysplasia and cancers of the the pulmonary tract, of at least 2, and more preferably has a therapeutic index of at least 5, 10, 20, 50, 100, 250, 500 or 1000.

In certain embodiments, the anti-PLESC agent has a therapeutic index (TI) for treating COPD of at least 2, and more preferably has a therapeutic index of at least 5, 10, 20, 50, 100, 250, 500 or 1000.

In certain embodiments, the anti-PLESC agent has a therapeutic index (TI) for treating bronchopulmonary dysplasia (BPD) of at least 2, and more preferably has a therapeutic index of at least 5, 10, 20, 50, 100, 250, 500 or 1000.

In certain embodiments, the anti-PLESC agent has a therapeutic index (TI) for treating chronic bronchitis of at least 2, and more preferably has a therapeutic index of at least 5, 10, 20, 50, 100, 250, 500 or 1000.

In certain embodiments, the anti-PLESC agent has a therapeutic index (TI) for treating one or more of emphysema, idiopathic pulmonary fibrosis or chronic rhinosinusitis of at least 2, and more preferably has a therapeutic index of at least 5, 10, 20, 50, 100, 250, 500 or 1000.

In certain embodiments, the anti-PLESC agent inhibits the proliferation or differentiation of PLESCs, or kills PLESCs, with an IC₅₀ of 10⁻⁶ M or less, more preferably 10⁻⁷ M or less, 10⁻⁸ M or less or 10⁻⁹ M or less.

In certain embodiments, the anti-PLESC agent inhibits the proliferation or differentiation of COPD PLESCs, or kills COPD PLESCs, with an IC₅₀ of 10⁻⁶ M or less, more preferably 10⁻⁷ M or less, 10⁻⁸ M or less or 10⁻⁹ M or less.

In certain embodiments, the anti-PLESC agent inhibits the proliferation or differentiation of PLESCs, or kills PLESCs, with an IC₅₀ of 10⁻⁶ M or less, more preferably 10⁻⁷ M or less, 10⁻⁸ M or less or 10⁻⁹ M or less.

In certain embodiments, the anti-PLESC agent is administered by topical application, such as inhalation.

In certain embodiments, the anti-PLESC agent is administered by submucosal injection.

In certain embodiments, the anti-PLESC agent is formulated as part of a bioadhesive formulation.

In certain embodiments, the anti-PLESC agent is formulated as part of a drug-eluting particle, drug eluting matrix or drug-eluting gel.

In certain embodiments, the anti-PLESC agent is formulated as part of a bioerodible drug-eluting particle, bioerodible drug eluting matrix or bioerodible drug-eluting gel.

In certain embodiments, the anti-PLESC agent is formulated as a single oral dose.

In certain embodiments, the anti-PLESC agent is delivered by a drug eluting device, such as a drug eluting stent or fiber.

In certain embodiments, the anti-PLESC agent is cell permeable, such as characterized by a permeability coefficient of 10⁻⁹ or greater, more preferably 10⁻⁸ or greater or 10⁻⁷ or greater.

In certain embodiments, the anti-PLESC agent is an mTOR inhibitor. In certain embodiments, the mTOR inhibitor is a PI3K/mTOR inhibitor.

In certain embodiments, the anti-PLESC agent is a CDK inhibitor. In certain embodiments, the CDK inhibitor is a selective inhibitor of CDK2, or a selective inhibitor of CDK2 and CDK7 and/or CDK9.

In certain embodiments, the anti-PLESC agent is a histone deacetylase (HDAC) inhibitor (“HDACi”).

In certain embodiments, the anti-PLESC agent is a proteasome inhibitor, preferably an immunoproteasome inhibitor.

In certain embodiments, the anti-PLESC agent is an HSP90 inhibitor, a HSP70 inhibitor or a dual HSP90/HSP70 inhibitor.

In certain embodiments, the anti-PLESC agent is an AKT inhibitor.

In certain embodiments, the anti-PLESC agent is an RAR antagonist.

In certain embodiments, the anti-PLESC agent is an Aryl Hydrocarbon Receptor antagonist.

In certain embodiments, the anti-PLESC agent is a multiple ion channel blocker.

In certain embodiments of the methods, preparations and devices of the present disclosure the anti-PLESC agent is administered with a second drug agent that selectively promotes proliferation or other regenerative and wound healing activities of normal pulmonary stem cells (a “Pulmonary Regenerative agent”) with an EC₅₀ at least 5 times more potent than for PLESCs, more preferably with an EC₅₀ 10 times, 50 times, 100 times or even 1000 times more potent for normal pulmonary stem cells relative to for PLESCs.

In certain embodiments of the methods, preparations and devices of the present disclosure the anti-PLESC agent is administered with a Pulmonary Regenerative agent that selectively promotes proliferation or other regenerative and wound healing activities of normal epithelial stem cells with an EC₅₀ at least 5 times more potent than for PLESCs, more preferably with an EC₅₀ 10 times, 50 times, 100 times or even 1000 times more potent for normal epithelial stem cells relative to for PLESCs.

In certain embodiments of the methods, preparations and devices of the present disclosure the anti-PLESC agent is administered with a Pulmonary Regenerative agent selectively promotes proliferation of normal pulmonary stem cells with an EC₅₀ of 10⁻⁶ M or less, more preferably 10⁻⁷ M or less, 10⁻⁸ M or less or 10⁻⁹ M or less.

In certain embodiments of the methods, preparations and devices of the present disclosure the anti-PLESC agent is administered with a Pulmonary Regenerative agent selectively promotes proliferation of normal epithelial stem cells with an EC₅₀ of 10⁻⁶ M or less, more preferably 10⁻⁷ M or less, 10⁻⁸ M or less or 10⁻⁹ M or less.

In certain embodiments, the combined administration of the anti-PLESC agent and the Pulmonary Regenerative agent has a therapeutic index (TI) for treating COPD, bronchopulmonary dysplasia (BPD), chronic bronchitis, emphysema, idiopathic pulmonary fibrosis, Asthma, chronic rhinosinusitis or a combination thereof, of at least 2, and more preferably has a therapeutic index of at least 5, 10. 20. 50. 100, 250, 500 or 1000.

In certain embodiments, the anti-PLESC agent and the Pulmonary Regenerative agent are administered to the patient as separate formulations.

In certain embodiments, the anti-PLESC agent and the Pulmonary Regenerative agent are co-formulated together. For instance, in certain embodiments, the anti-PLESC agent and the Pulmonary Regenerative agent are co-formulated together for oral delivery (i.e., as a single dose oral formulation); in certain embodiments, the anti-PLESC agent and the Pulmonary Regenerative agent are co-formulated together for inhalation delivery (i.e., an inhalation formulation); in certain embodiments, the anti-PLESC agent is an EGFR inhibitor, and preferably an ErbB1 inhibitor, and the Pulmonary Regenerative agent is a BCR-ABL Kinase Inhibitor or FLT3 Inhibitor; and in certain embodiments, the anti-PLESC agent is an EGFR inhibitor is PD168393 and the Pulmonary Regenerative agent is Ponatinib.

Moreover, these pathogenic stem cells are in fact present in normal tissue from infancy, and activation of one or more of the PLESC clusters may contribute to pulmonary function decline with age or pulmonary distress if activated from pathogens or environmental factors even in the absence of presenting with COPD like conditions. For instance, angiotensin-converting enzyme 2 (ACE2) and TMPRSS2—enzymes believed to be necessary for human SARS coronavirus (SARS-CoV) infection—are upregulated in certain of the PLESC clusters and the tissue produced by those stem cells and not by normal regenerative lung stem cells. This may help to explain the higher mortality rates, and general pulmonary distress, dispropriately caused in older patients and patients with COPD by SARS-COV such as COVID19.

In certain embodiments, the present disclosure provides for therapies which include the use of anti-PLESC agents in order to reduce the suscpetiblity of pathogen infection and/or to reduce the severity of infection with respect to pulmonary tissue.

In certain embodiments, the present disclosure provides for anti-aging therapies which include the use of anti-PLESC agents in order to reduce the role of PLESCs in degrading pulmonary function with agent, and (optionally) the use of a Pulmonary Regenerative agent or a administration of normal regenerative lung stem cells in order to promote normal lung function.

The disclosure further provides a method of detecting the presence of an inflammatory lung disorder, such as COPD, bronchopulmonary dysplasia, chronic bronchitis, emphysema, idiopathic pulmonary fibrosis, chronic rhinosinusitis or a combination thereof, in a subject including the steps of providing a detection agent that specifically binds to pathogenic lung epithelial stem cells; administering the detection agent to a subject; and detecting whether the detection agent specifically binds to a pathogenic lung epithelial stem cell in the lung of the subject, wherein, if the detection agent specifically binds to a cell in the lung of the subject to a higher degree than the average normal lung, the subject is diagnosed with an inflammatory lung disorder, such as COPD, bronchopulmonary dysplasia, chronic bronchitis, emphysema, idiopathic pulmonary fibrosis, Asthma, chronic rhinosinusitis or a combination thereof or as having a risk of developing Barrett's esophagus.

In one embodiment, the patient is a human. In another embodiment, the detection step is performed in vitro on a biopsy sample. Specifically, the detection step can be performed in vivo. The detection agent can be an antibody. More specifically, the detection agent is a monoclonal antibody.

In other embodiments, the detection agent is a Positron Emission Tomography (PET) imaging agent or a magnetic resonance imaging (MRI) contrast agent. The detection agent can be a radioisotope or contrast enhancing isotope, such as 3H, 11C, 177Lu, 111 Indium, 67Cu, 99mTc, 124I, 125I, 131 I and 89Zr. The detection agent can also be detected in the patient by Single Photon Emission Computed Tomography (SPECT), Positron Emission Tomography (PET), Magnetic Resonance Imaging (MRI), Fluorescent Imaging, or Near-infrared (NIR) Emission Spectroscopy.

OCT detection/contrast agents are can include near-infrared dyes, polypyrrole nanoparticles, optical detection/contrast agents and engineered microsphere contrast agents. PET detection/contrast can include ¹⁸F-fluoride, 3′-deoxy-3′-[¹⁸F]fluorothymidine, ¹⁸F-fluoromisonidazole, gallium, technetium-99m, thallium, oxygen, nitrogen, iron, carbon, 43K, ⁵²Fe, ⁵⁷Co, ⁶⁷Cu, ⁶⁷Ga, ⁶⁸Ga, ¹²³I, ¹²⁵I, ¹³¹I, ¹³²I, and ³⁹Tc. MRI detection/contrast agents can include ferro, antiferro. ferrimagnetic or superparamagnetic material, ferrite with spinel structure, ferrite with a magnetoplumbite structure other hexagonal ferrite structures, paramagnetic ions, comprise a paramagnetic contrast agent, a super paramagnetic contrast agent, a diamagnetic agent and combinations thereof. Ultrasound detection/contrast agents can include shell encapsulated gas bubbles; shell encapsulated droplets; and nanoparticles. X-ray detection/contrast agents can include lodinated contrast-enhancing units; barium sulfate-based contrast-enhancing units; metal ion chelates; boron clusters with a high proportion of iodine; lodinated polysaccharides, polymeric triiodobenzenes; particles from lodinated compounds displaying low water solubility; liposomes containing lodinated compounds: and lodinated. SPECT detection/contrast agents can include ⁹⁹mTc, ¹²³I, ¹³¹I, ⁶⁷Cu, ¹¹¹In, and ²⁰¹TI.

Binding agent can include antibodies, aptamers, peptides, cell surface receptor ligands, and small molecules.

Still another aspect of the disclosure provides animal models of inflammatory lung tissues. In certain embodiments, the animal is an immunocompromised rodent (such as a mouse or rat) able to tolerate human xenografted tissue, having one or more human lung tissue xenografts formed from injected human PLESCs or a mix of human PLESCs with normal regenerative lung stem cells. In certain embodiments, the cells are injected subcutaneously. In certain embodiments, the cells are injected subcutaneously in a Matrigel or other biodegradable matrix. In certain embodiments, the injected cells form a cyst having a luminal center (a “luminal cyst”) with an epithelial lining having microarchitecture resembling lung tissue (particularly inflammatory lung tissue).

### BRIEF DESCRIPTION OF THE DRAWINGS AND TABLES

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Epithelial clone heterogeneity in COPD. FIG. 1A. Schematic depicting workflow of generating p63+ epithelial colony pools from resected normal and COPD lung tissue. Individual colonies are captured and expanded individually to generate clonal cell lines. Scale bar, 500 μm. FIG. 1B. Principal component analysis of RNAseq differential expression genes (DEGs) (FDR<0.05) derived from multiple clones from control (SPN-12, -14) and COPD (SPN-13, -07) lungs. FIG. 1C. RNAseq expression heatmap of the clones depicted in the PCA plot is shown highlighting Cluster 1 (control) vs Clusters 1-4 in COPD. FIG. 1D. Top, Individual colonies from cloned representatives of Clusters 1-4 stained with antibody to p63 showing uniform nuclear staining. Scale bar, 100 μm. Bottom, Rhodamine redstained colonies arising on lawns of irradiated 3T3-J2 fibroblasts from 500 cells from each of cloned representatives of Clusters 1-4. Right, Histogram of clonogenicity based on percentage of plated cells that formed colonies using representative clones from Clusters 1-4 at passage 5 (P5) and 25 (P25).

FIGS. 2A-F. Commitment of variant clones to metaplastic fates. FIG. 2A. From left, Schematic of clonal expansion and differentiation in air-liquid interface (ALI) cultures assessed by immunofluorescence on histological sections. Cluster 1 clones differentiated to an epithelium characterized by expression of SCGB1A1, SFTPB, α-TUB and AQP4 and the absence of MUC5AC. Cluster 2 clones differentiated into an epithelium characterized by SCGB1A1+, MUC5AC+, AQP4-goblet cells. Cluster 3 and Cluster 4 clones differentiated into squamous epithelia that expressed involucrin (IVL) but not SCGB1A1, AQP4, or SFTPB. Scale bar, 100 μm. FIG. 2B. Schematic of subcutaneous transplants of immature cells from clones or library of clones into immunodeficient mice. Nodules formed at 4 weeks were processed for histology and immunostaining and showed polarized epithelia that reacted with antibodies to the humanspecific marker STEM121. Scale bar, 100 μm. FIGS. 2C-D. In vivo differentiation following subcutaneous transplantation of cloned representatives of Cluster 1 (SCGB1A1+, SFTPB+, AQP4+), Cluster 2 (MUC5AC+GCM), and Clusters 3 and 4 (IVL+; SCM). Scale bar, 100 μm. FIG. 2E. Xenografts of Cluster 1-4 clones previously grown in vitro to passage 5 (P5) and passage 25 (P25) showing stability of fate differentiation and expression of Cluster-specific markers. Scale bar, 100 μm. FIG. 2F. Copy number and single nucleotide variation analysis derived from whole exome sequencing of representative clones of Clusters 1-4 from SPN-13 relative to patient blood. CNV events of larger than 10-20 Kb were not detected in any of the clones. Venn diagram of all detected exonic SNVs (synonymous, non-synonymous, indels) in each clone relative to all others.

FIGS. 3A-E. p63+ cells in COPD metaplasia and clone libraries. FIG. 3A. Top, p63 immunohistochemistry of distal lung of GOLD Stage 4 COPD showing contiguous regions of squamous and goblet cell metaplasia (SCM and GCM) subtended by p63+ basal cells (brown). Scale bar, 200 μm. Bottom, Immunofluorescence micrographs of expanded regions of SCM and GCM stained with antibodies to p63 (green), IVL (red) and MUC5AC (red). Scale bar, 100 μm. FIG. 3B. Box-Whisker plots for the linear occupancy of metaplastic lesions (GCM of P=4.1e−06, SCM of P=1.2e−07, and inflammatory SCM of P=4.9e−07, Student's t-test) across distal airways ten Stage 4 COPD lungs compared with five normal lungs without disease. FIG. 3C. Top, p63 immunohistochemistry micrographs of regions of COPD distal airways showing, from left, examples of terminal bronchiole, goblet cell metaplasia, and squamous cell metaplasia. Scale bar, 200 am. Bottom, immunofluorescence micrographs of expanded regions of terminal bronchiole (p63+, Aqp5+), GCM (p63+, TRPC6+), and inflammatory SCM (p63+, CXCL8+). Scale bar, 100 μm. FIG. 3D. Box-Whisker plots of distribution of CXCL8+(P=2.5e−06, Student's t-test) and TRPC6+ basal cells (P=1.2e−08, Student's t-test) in 10 cases of Stage 4 COPD distal lung compared with five normal lungs. FIG. 3E. Single and aggregate tSNE profiles of single cell RNAseq data of three COPD and three control clone libraries. Pie charts indicate the fractional contributions of clones of Clusters 1-4 to patient-specific clone libraries (NM, blue; GCM, green, SCM, orange, iSCM, red).

FIGS. 4A-E. Library composition and pro-inflammatory response in xenografts. FIG. 4A. FACS profiles of COPD and control clone libraries using markers established from library scRNAseq and clonal RNAseq including anti-AQP5 (Cluster 1), anti-TRPC6 (Clusters 2+3), and anti-CXCL8 (Cluster 4). FIG. 4B. Histogram compiling FACS quantification data on the relative clone composition of each patient library. FIG. 4C. Histological sections of four-week xenograft of clone libraries from control (SPN-21) showing epithelial cysts devoid of luminal cells. FIG. 4D. Histological sections of four-week xenograft of clone libraries from COPD case (SPN-04) showing epithelial cysts marked by abundant infiltration of CD45+/Ly6G+ leukocytes (insets). FIG. 4E. Histogram depicting the quantification of leukocyte infiltration in xenografts of clone libraries from 11 control and 19 COPD patients based on degree of CD45+ cells in cysts (right). Scale bar, 100 μm.

FIGS. 5A-F. Cluster 4 COPD clones are constitutively hyperinflammatory. FIG. 5A. Histogram depicting most significant (P<1.0e−8) pathways determined by Ingenuity Pathway Analysis of RNAseq differentially expressed genes (FDR<0.05) of patient-matched clones representative of Clusters 1-4. FIG. 5B. Differential expression heatmaps of chemokine, interleukin, and interferon genes among RNAseq DEGs (FDR<0.05) of patient-matched clones representative of Clusters 1-4 (SPN-13). FIG. 5C. H&E on sections through four-week xenografts of patient-matched clones of Clusters 1-4 showing that only Cluster 4 clones are accompanied by abundant intraluminal leukocytes. Scale bar, 100 μm. FIG. 5D. Immunofluorescence micrographs of Cluster 4 xenografts revealing high expression in epithelia of inflammatory mediators including IL33, CXCL8, and IL1B. Scale bar, 50 μm. FIG. 5E. CD45 immunohistochemistry of xenografts derived from Cluster 4 clone grown in vitro to passage 5 and to passage 25. Scale bar, 200 μm. FIG. 5F. Histogram of CXCL8, CCL20, and CXCL10 gene expression in clonal representatives of Clusters 1-4 at in vitro passage 5 and passage 25.

FIGS. 6A-F. Cluster 3 and 4 clones drive host myofibroblast activation. FIG. 6A. Immunofluorescence micrographs of xenografts derived from control case (SPN-12; left) and COPD case (SPN-02; right) clone libraries stained with antibodies to the myofibroblast marker alpha-smooth muscle actin (SMA). Scale bar, 200 μm. FIG. 6B. Quantification of myofibroblast submucosal accumulation in xenografts based on general scale applied to cysts within 11 control and 19 COPD clone library transplants. FIG. 6C. Box-Whisker plot representation of fibrosis accumulation about cysts in each of 19 COPD and 11 control library xenografts (P=7.0e−15, Student's t-test). FIG. 6D. Immunofluorescence micrographs of xenografts derived from patient-matched clones of Clusters 1-4 using antibodies to E-cadherin (ECAD, red) and alpha-smooth muscle actin (SMA, green). Scale bar, 100 μm. FIG. 6E. Differential expression heatmap of fibrosis-related genes (1.5-fold, p<0.05) of xenografts derived from patient-matched clones representative of Clusters 1-4. FIG. 6F. Schematic TGF-β pathway including genes differentially expressed in clones of Clusters 3 and 4.

FIGS. 7A-G. Identification of variant clone types in normal and fetal lung. FIG. 7A. Schematic of clone library generation from pseudoglandular fetal lung and analysis by scRNAseq and xenografting. FIG. 7B. Single cell RNA sequencing of clone library from 13-wk fetal lung yielding tSNE profile (left), integration with three adult control and three COPD libraries (middle), and the fetal subset profile based on the integrated profile (right). FIG. 7C. Histology and indicated marker analysis of xenografts of 13-wk fetal clones corresponding to Clusters 1-4. Cluster 4 xenografts are further assessed by immunohistochemistry with antibodies to CD34 and Ly6G. FIG. 7D. Ratio of qPCR-determined marker expression across Cluster 1-4 clones from COPD, Control, and 13-wk fetal lung. Cluster 1 markers, AQP5 and NDRG1; Clusters 2 and 3, TRPC6 and ANLN; Cluster 4, CXCL8 and CCL20. FIG. 7E. Percentage composition of Cluster 4 clones across 11 Control and 19 COPD clone libraries. FIG. 7F. Schematic for generating xenografts from defined ratios of Cluster 4 and Cluster 1 cells. PANEL G. Histogram of quantification of host inflammatory response to co-xenografts of Cluster 4 and Cluster 1 clones based on CD45 and Ly6G monitoring of cystic infiltration by leukocytes.

FIGS. 8A-G. In vitro and in vivo differentiation of Cluster 1-4 clones; Related to FIGS. 2A-F. FIG. 8A. H&E staining of histological sections of air-liquid interface (ALI)-differentiated Cluster 1-4 clones. Scale bar, 100 μm. FIG. 8B. Left, Immunohistochemistry of p63 antibody staining (brown) of the distal airway of normal human lung. Scale bar, 500 μm. Right from top, Magnification of indicated terminal bronchiole epithelium region of p63 immunohistochemistry; immunofluorescence micrograph of terminal bronchiolar epithelium stained with DAPI, anti-p63, anti-SCGB1A1, and anti-SFTPB, and immunofluorescence micrograph of terminal bronchiolar epithelium showing anti-p63 and anti-AQP4 staining. Alveo, alveolar epithelia. Scale bar, 100 μm. FIGS. 8C-F. Immunofluorescence micrographs of xenograft sections derived from transplants of clones of Clusters 1-4 probed with the indicated antibodies. Scale bar, 100 μm. FIG. 8G. Venn diagrams showing overlap between nonsynonymous SNV events detected from whole genome exome sequencing of clones of Clusters 1-4 from COPD (SNP-13) lung and 202 oncogenes and 231 tumor suppressor genes.

FIGS. 9A-B. Distribution of metaplasia in Stage 4 COPD lung; Related to FIGS. 3A-E. FIG. 9A. Immunohistochemistry micrograph of section of distal lung of normal donor showing distribution of p63 staining (brown) in bronchioles and terminal bronchioles. Scale bar, 1,000 μm. Boxed region indicates region presented as inset right top) and corresponding region of serial section examined by p63 and AQP5 immunofluorescence (lower right). Scale bar of insets, 100 μm. FIG. 9B. Immunohistochemistry micrograph of Stage 4 COPD distal lung stained with p63 antibodies (brown). (*) denote regions of submucosal inflammation. Scale bar, 1,000 μm. Boxed regions corresponding to inflammatory SCM, GCM, and SCM are shown at higher magnification below along with immunofluorescence micrographs of similar regions from serial sections stained with antibodies to p63/CXLC8 (iSCM), p63/TRPC6 (GCM), and p63/TRPC6 (SCM). Inset region scale bars, 100 μm.

FIGS. 10A-C. Clone variant analysis by single cell RNA-seq; Related to FIGS. 3A-E. FIG. 10A. Differential gene expression determined by scRNAseq of three COPD and three control clone libraries and the corresponding differential gene expression determined by RNAseq of discrete clones of Clusters 1-4 of SPN-13 (detailed in Tables S6, S7). FIG. 10B. tSNE profiles of single cell RNAseq data derived from clone libraries from the individual COPD and control cases. FIG. 10C. Mapping of cluster-specific gene expression markers onto an integrated tSNE profile assembled from three Control and three COPD cases.

FIGS. 11A-D. Leukocyte transepithelial migration in xenografts; Related to FIGS. 4A-E. FIG. 11A. Box-Whisker plot representation of Cluster 2-4 FACS proportions in each of 19 COPD and 11 control libraries (P<2.2e−16, Student's t-test). FIGS. 11B-C. Histological sections through nodules formed four weeks after transplantation of clonogenic cell pools of six representative non-COPD cases (upper) and COPD cases (lower) to immunodeficient mice. Scale bar, 200 μm. FIG. 11D. Box-Whisker plot representation of severe inflammation fraction in each of 19 COPD and 11 control library xenografts (P=1.2e−09, Student's t-test).

FIGS. 12A-C. Inflammatory gene expression by clones of Clusters 1 to 4; Related to FIGS. 5A-F. FIG. 12A. Top 10 pathways of differentially expressed genes (FDR<0.05) of patient-matched clones representative of Clusters 1-4 using Ingenuity Pathway Analysis (IPA) software. FIG. 12B. Immunofluorescence of Vcam1 (red) in human lung endothelial cells following exposure to media conditioned by clones of Clusters 1-4 compared to IL-13 challenge. Scale bar, 100 μm. FIG. 12C. Expression heatmaps (FDR<0.05) depicting pair-wise gene expression analysis of Clusters 1-4 focused on chemokine, interleukin, and interferon genes.

FIGS. 13A-C. Fibrosis in xenografts; Related to FIGS. 6A-F. FIGS. 13A-B. Immunofluorescence micrographs of sections of nodules formed four weeks after transplantation of clone libraries from six representative control cases (upper) and COPD cases (lower). Green, E-cad, Ecadherin; red, SMA, anti-alpha smooth muscle actin. Scale bar, 200 μm. FIG. 13C. Immunofluorescence micrographs of E-cadherin (Ecad) and alpha smooth muscle actin (SMA) expression in xenografts of clones of Cluster 3 (left) and Cluster 4 (right) from in vitro passage 5 and passage 25. Scale bar, 100 μm.

FIGS. 14A-E. Variant clones in normal adult and fetal lung; Related to FIGS. 7A-G. FIG. 14A. Histology and marker analysis of xenografts of cloned representatives of Clusters 1-4 from adult Control lung (SPN-14). FIG. 14B. Left, Principal component analysis of RNAseq profiles of Clusters 1-4 clones from control lung (SPN-14) and those of Clusters 1-4 of two COPD cases (SPN-7 and SPN-13). Right, Expression heatmap comparing RNAseq profiles of Clusters 1-4 clones of control lung (SPN-14) with those of clones of COPD cases SPN-7 and SPN-13. FIG. 14C. Histology of pseudoglandular epithelia of 13-wk fetal lung and marker immunofluorescence including antibodies to p63, AQP5, AQP4, α-TUB, and SFTPB. FIG. 14D. Left, Distribution of cluster-specific expression markers on scRNAseq tSNE profile of clone library from 13-wk fetal lung. Right, Pie chart showing distribution of variants in 13-wk fetal library. FIG. 14E. Dot plots of expression for cluster-specific marker genes in the integrated tSNE profiles of lung clones from three adult Controls, three COPD cases, and the 13-wk fetal lung. The size of the dot corresponds to the percentage of cells expressing the feature in each cluster. The color represents the average expression level.

FIG. 15. Biopsies from Indiopathic Pulmonary Fibrosis (IPF) Patients Reveal to Stem Cell Types. While COPD patients were revealed to have four stem cell phenotypes (one normal regenerative and three pathogenic stem cell types), biopsies from a range of IPF patients indicating that those patients have two discrete stem cell types (one normal regenerative and one pathogenic stem cell types). This figure shows an expression heatmap comparing RNAseq profiles of normal (CLST1) clones from a control lung with those of pathogenic (IPF CLST2) clones from an IPF patient.

FIG. 16. Pathogenic stem cells (IPF CLST2) from IPF Patients Have Distinct Regulatory Pathways Upregualted relative to Normal. Top 30 pathways of differentially expressed genes of patient-matched clones representative ofCLST1 and IPF CLST2 Ingenuity PathwayAnalysis (IPA) software.

FIG. 17. Pathogenic stem cells (IPF CLST2) from IPF Patients Promote Fibrocyte Infiltration but Not Immune Cell Infiltration. Mixed cell cultures of human lung fibroblasts (SMA^{neg}) with clonogenic cell pools of representative non-IPF cases (Control Library) and IPF cases (IPF Library) demonstrates that the IPF pathogenc stem cell is able to induce differentiation of the fibroblasts to a myeofibroblast phenotype, consistent with IPF disease.

FIG. 18. Use of normal and pathogenic lung stem cells for Drug Discovery. Overview of process used to identify the compounds described herein as selective inhibitors of the pathogenic stem cells or promoters of the normal regenerative stem cells. Stem cell clones or pools were adapted for growth and display in high throughput multiwell plates. The effect on cell growth after treatment with a test compound was measured, and those which either selectively killed the COPD or IPF pathogenic stem cells were identified, as were compounds that selectively promoted the proliferation of the normal regenerative stem cells. The right hand panel demonstrates the ability of one of the compounds of the present invention, identified to selectively kill the IPF CLST2 stem cell, to inhibit fibrocyte infiltration into the luminal cyst formed by Matrigel including normal and IPF CLST2 cells being injected subcutaneously.

FIGS. 19A-B. HTS Screening Output. Differential screening can be run in high throughput formats, with the goal being to find drugs that are more toxic to the pathogenic stem cells (Y axis) than to normal regenerative lung stem cells (X-axis). In this case, the pathogenic stem cells are IPF CLST2 cells. FIG. 19A—The darker dots represent compounds that were selected and further evaluated, FIG. 19B, in dose dependent experiments in which the dose response of normal and pathogenic stem cells is evaluated to establish differential effects on the pathogenic stem cells. This assay system was used to determine compounds described herein as anti-PLESC agents (selectively lethal to COPD or IPF pathogenic stem cells) or Stem Cell Promoters.

FIGS. 20A-C. IPF Lung Stem Cell Libraries Promote Fibrosis in Immunodeficient Mice. FIG. 20A depicts the generation of libraries of clonogenic epithelial stem cells expressing p63 from control and IPF lungs. FIG. 20B shows immunofluorescence micrographs of subcutaneous nodules formed by the transplantation of five million stem cells of control (left) and IPF (right) libraries two weeks prior. The stem cells form polarized epithelial cysts marked by ECAD staining (red) which in the case of the IPF cysts are surrounded by αSMA+ myofibroblasts (green). FIG. 20C shows the morphometric quantification of the percentage of epithelial cystic surfaces occupied by submucosal myofibroblasts in xenografts of 10 control and 16 IPF libraries.

FIGS. 21A-C. Stem Cell Heterogeneity of IPF Lung Reveals a Major Pro-Fibrotic Variant. FIG. 21A depicts single cell RNA sequencing profiling of two IPF stem cell libraries showing normal lung stem cells (Cluster 1; blue) and a variant denoted as “Cluster 2” that is marked by the differential expression of CXCL17, CEACAM6, IL1RN, and CLDN4. FIG. 21B depicts the use of the Cluster 2 cell surface marker CEACAM6 to quantify by fluorescence-activated cell sorting (FACS) the percentage of Cluster 2 stem cells in control and IPF libraries and to generate single cell-derived clones of both Cluster 1 and Cluster 2 cells for functional and molecular analysis. From left, FACS profiles of CEACAM6+ cells in control and IPF libraries, a histogram of percentages of CEACAM6+ cells across 10 control libraries and those from 16 IPF cases, the use of FACS to sort single CEACAM6- and CEACAM6+ cells to 384-well plates for generation of clonal lines of each, and lists of differentially expressed genes in the Lung Fibrosis gene set (NCATS BioPlanet) in Cluster 1 and Cluster 2 clones. Underlined genes in red indicate those previously linked to the risk of IPF by GWAS, those associated with familial IPF via rare mutations (in orange), or GDF15, an indicator of IPF severity (in green). FIG. 21C depicts experiments that demonstrate the ability of Cluster 2 cells to promote the conversion of human lung fibroblasts (HLF) to fibrosis-associated, αSMA-expressing myofibroblasts in vitro, and how this conversion is dependent on a threshold ratio of Cluster 2 to Cluster 1 cells. From left, schematic of HLF co-culture experiments with either Cluster 1 or Cluster 2 cells from IPF lung, immunofluorescence micrographs of co-cultures of HLF with Cluster 1 (top) and Cluster 2 (bottom) cells stained with antibodies to the epithelial marker ECAD (red) and the myofibroblast marker αSMA (green); FACS profiles of αSMA+ cells from the corresponding co-cultures, and a graphical representation of HLF conversion to αSMA+ myofibroblasts determined by quantitative FACS profiling as a function of discrete ratios of Cluster 2 to Cluster 1 cells.

FIG. 22. Cluster 2 Cells in Regions of Low and High UIP Histopathology. From left, Drawing of left lung portraying the stereotypical bias of UIP histopathology in lower lobes including peripheral reticulation and honeycombing, patient-matched histological sections of upper (top) and lower (bottom) lung stained with hematoxylin and eosin (H&E), FACS profiling of CEACAM6+ cells from stem cell libraries generated from corresponding regions of upper and lower lung, and whisker plot of CEACAM6+ cells in libraries derived from upper and lower lung from three IPF cases and from 10 control libraries.

FIGS. 23A-C. IPF Cluster 2 Variant is Distinct from Major COPD Variants. FIG. 23A shows the differential gene expression in in vitro differentiated Cluster 2 stem cells relative to Cluster 1 cells in a volcano plot and as a histogram of significant gene sets (NCATS BioPlanet). FIG. 23B compares the gene expression profiles of in vitro differentiated IPF Cluster 2 stem cells with those of the two pro-fibrotic COPD stem cell variants SCM (squamous cell metaplasia) and iSCM (inflammatory squamous cell metaplasia) in a Venn diagram, and the gene sets (NCATS BioPlanet) represented by the genes in common among Cluster 2, SCM, and iSCM cells. FIG. 23C shows tSNE profiles of stem cell libraries from control, COPD, and IPF cases showing the respective clusters of normal lung stem cells (Cluster 1, blue), IPF Cluster 2 (gray), squamous cell metaplasia (orange), inflammatory squamous cell metaplasia (red), and goblet cell metaplasia (green).

FIGS. 24A-B. Vulnerability of Cluster 2 Cells to Bioactive Small Molecules. As proof-of-concept experiments to assess differential sensitively to small molecule inhibitors, cloned Cluster 1 and Cluster 2 stem cells from IPF libraries were screened in parallel against libraries of established and experimental drugs. FIG. 24A shows, from left, the survival of the Cluster 2 and Cluster 1 three days after exposure to these compounds at 1 uM, with those annotated to be inhibitors of EGFR labelled red, a pie chart highlighting the most abundant inhibitor classes differentially affecting Cluster 2 cells, and a heatmap of differentially expressed genes of the EGFR gene set (NCATS Bioplanet) in Cluster 1 and Cluster 2 cells that had undergone in vitro differentiation. FIG. 24B shows, from left, dose-response curves of one EGFR inhibitor (PD168393), against Cluster 1 and Cluster 2 stem cells, CEACAM6 FACS profiles of an IPF stem cell library and following exposure to PD168393, and a whisker plot of CEACAM6+ cells in all 16 IPF libraries as a function of exposure to the EGFR inhibitor.

FIG. 25. Comparison of small molecule screens across two IPF cases Survival profiles of small molecule screens of Cluster 2 versus Cluster 1 cells from two cases of IPF. Red dots in circles indicate common small molecules that differentially impact Cluster 2 cells across the two IPF cases. The pie chart shows the distribution of common hits with specific pathways including EGFR, mTOR, and MEK/ERK.

### DESCRIPTION OF ILLUSTRATIVE EMBODIMENTS

### I. Overview

Chronic obstructive pulmonary disease (COPD) is a progressive condition of chronic bronchitis, small airway obstruction, emphysema, idiopathic pulmonary fibrosis and chronic rhinosinusitis, and represents a leading cause of death worldwide. While inflammation, fibrosis, and metaplastic epithelial lesions are hallmarks of this disease, their origins and relationships remain unclear and they generally persist despite smoking cessation. Here, the inventors apply single-cell cloning technologies to lung tissue of patients with and without COPD. Unlike control lungs, which were dominated by normal distal airway progenitor cells, COPD lungs were inundated by three variant progenitors epigenetically committed to distinct metaplastic lesions. When transplanted to immunodeficient mice, these variant clones induced pathology akin to the mucous and squamous metaplasia, neutrophilic inflammation, and fibrosis seen in COPD. Remarkably, similar variants pre-exist as minor constituents of control and fetal lung and conceivably act in normal processes of immune surveillance. However, these same variants likely catalyze the pathologic and progressive features of COPD when expanded to high numbers.

Most approaches to the treatment of COPD, and other forms of inflammatory pulmonary diseases focus on reducing or inhibiting the inflammatory components of these diseases, or the consequence thereof such as the use of bronchodilators. However, as described here, the inflammatory symptoms of COPD are a consequence of an altered epithelial lining generated by an epigenetically shifted stem cell in the tissue—with inflammation and other physiological and structural changes in the lung being caused by the altered epithelial linings/structures. As described in greater detail below and the attached figures, in the case of COPD, the inventors have found an epigenetic shift in the stem cells of the pulmonarytract—where the inflammatory and fibrotic aspects that characterize this disease occur. In this case, the stem cells that give rise to the lining of the lung are altered in a way that cause the resulting epithelial lining to have genes encoding proteins which attract immune cells and fibrosis causing cells to be upregulated, such as a signals and activators of the innate and/or adaptive immune systems and signals which attract fibrocytes.

Moreover, these pathogenic stem cells are in fact present in normal tissue from infancy, and activation of one or more of the PLESC clusters may contribute to pulmonary function decline with age or pulmonary distress if activated from pathogens or environmental factors even in the absence of presenting with COPD like conditions. For instance, angiotensin-converting enzyme 2 (ACE2) and TMPRSS2-enzymes believed to be necessary for human SARS coronavirus (SARS-CoV) infection—are upregulated in certain of the PLESC clusters and the tissue produced by those stem cells and not by normal regenerative lung stem cells. This may help to explain the higher mortality rates, and general pulmonary distress, dispropriately caused in older patients and patients with COPD by SARS-COV such as COVID19.

The present disclosure derives from correlations between the abundance of pathogenic stem cell clones and COPD stage as well as their correlation with the known regional, intrapulmonary variations in COPD pathology.

If indeed these clones contribute to COPD, multiple opportunities become available to limit their impact on disease progression. These include neutralizing one or more of the pathogenically relevant chemokines or cytokines secreted by individual variants, targeting particular variants with cell surface-directed antibodies, or through the discovery of small molecules that selectively affect one or more of these clone types. This latter strategy could be predicated on the observation that COPD patients retain normal clones that would potentially compensate for the loss of their pathogenic counterparts.

### II. Definitions

The term “airway”, as used herein, means part of or the whole respiratory system of a subject which exposes to air. The airway includes, but not exclusively, throat, windpipes, nasal passages, sinuses, a respiratory tract, lungs, and lung lining, among others. The airway also includes trachea, bronchi, bronchioles, terminal bronchioles, respiratory bronchioles, alveolar ducts, and alveolar sacs.

The terms “inflammatory lung disorder”, “airway inflammation” and the like, as used herein, means a disease or condition related to inflammation on airway of subject. The airway inflammation may be caused or accompanied by allergy(ies), asthma, impeded respiration, cystic fibrosis (CF), Chronic Obstructive Pulmonary Diseases (COPD), allergic rhinitis (AR), Acute Respiratory Distress Syndrome (ARDS), microbial or viral infections, pulmonary hypertension, lung inflammation, bronchitis, airway obstruction, and bronchoconstriction.

The term “an aberrant expression”, as applied to a nucleic acid of the present disclosure, refers to level of expression of that nucleic acid which differs from the level of expression of that nucleic acid in healthy gastroinstestinal tissue, or which differs from the activity of the polypeptide present in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in the activity; for example, an aberrant polypeptide can interact with a different target peptide. A cell can have an aberrant expression level of a gene due to overexpression or underexpression of that gene.

“Amino acid sequence” as used herein refers to an oligopeptide, peptide, polypeptide, or protein sequence, and fragment thereof, and to naturally occurring or synthetic molecules. Fragments of an expression product of a COPD gene sequence (an “COPD gene product”) are preferably about 5 to about 15 amino acids in length and retain the biological activity or the immunological activity of an COPD gene product. Where “amino acid sequence” is recited herein to refer to an amino acid sequence of a naturally occurring protein molecule, amino acid sequence, and like terms, are not meant to limit the amino acid sequence to the complete, native amino acid sequence associated with the recited protein molecule.

The term “antibody” broadly refers to any immunoglobulin (Ig) molecule and immunologically active portions of immunoglobulin molecules (i.e., molecules that contain an antigen binding site that immunospecifically bind an antigen) comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference) and/or antigen-binding fragments of any of the above (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Antibodies also refer to immunoglobulin molecules and immunologically active portions of immunoglobulin molecules, i.e., molecules that contain antigen or target binding sites or “antigen-binding fragments.” The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain.

The term “specific affinity binder” refers to an antibody as well as to a non-antibody protein scaffold i.e., smaller proteins that are capable of achieving comparable affinity and specificity using molecular structures that can be for example one-fifth to one-tenth the size of full antibodies, and also to nucleic acid aptamers. In some embodiments, the specific affinity binder of the present disclosure is a non-antibody polypeptide. In some embodiments, the non-antibody polypeptide can include but is not limited to peptibodies, DARPins, avimers, adnectins, anticalins, affibodies, affilins, atrimers, bicyclic peptides, centryins, Cys-knots, Fynomers, Kunitz domains, Obodies, pronectins, Tn3, maxibodies, or other protein structural scaffold, or a combination thereof.

A disease, disorder, or condition “associated with” or “characterized by” an aberrant expression of an COPD gene sequence refers to a disease, disorder, or condition in a subject which is caused by, contributed to by, or causative of an aberrant level of expression of a nucleic acid.

“Biological activity” or “bioactivity” or “activity” or “biological function”, which are used interchangeably, herein mean an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands and in the design and use of PNA molecules.

A “composition comprising a given polynucleotide sequence” as used herein refers broadly to any composition containing the given polynucleotide sequence. The composition may comprise a dry formulation or an aqueous solution. Compositions comprising polynucleotide sequences encoding an COPD gene product or fragments thereof may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

The term “correlates with expression of a polynucleotide”, as used herein, indicates that the detection of the presence of ribonucleic acid that is similar to one of COPD genes by northern analysis is indicative of the presence of mRNA encoding an COPD gene product in a sample and thereby correlates with expression of the transcript from the polynucleotide encoding the protein.

A “deletion”, as used herein, refers to a change in the amino acid or nucleotide sequence and results in the absence of one or more amino acid residues or nucleotides.

As is well known, genes or a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an COPD polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

As used herein, the terms “gene”, “recombinant gene”, and “gene construct” refer to a nucleic acid of the present disclosure associated with an open reading frame, including both exon and (optionally) intron sequences.

A “recombinant gene” refers to nucleic acid encoding a polypeptide and comprising exon sequences, though it may optionally include intron sequences which are derived from, for example, a related or unrelated chromosomal gene. The term “intron” refers to a DNA sequence present in a given gene which is not translated into protein and isgenerallyfound between exons.

The term “growth” or “growth state” of a cell refers to the proliferative state of a cell as well as to its differentiative state. Accordingly, the term refers to the phase of the cell cycle in which the cell is, e.g., G0, G1, G2, prophase, metaphase, or telophase, as well as to its state of differentiation, e.g., undifferentiated, partially differentiated, or fully differentiated. Without wanting to be limited, differentiation of a cell is usually accompanied by a decrease in the proliferative rate of a cell.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. An “unrelated” or “non-homologous” sequence shares less than 40% identity, though preferably less than 25% identity, with one of the sequences of the present disclosure.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Centerfor Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

The term “hybridization”, as used herein, refers to any process by which a strand of nucleic acid binds with a complementary strand through base pairing.

An “insertion” or “addition”, as used herein, refers to a change in an amino acid or nucleotide sequence resulting in the addition of one or more amino acid residues or nucleotides, respectively, as compared to the naturally occurring molecule.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Microarray” refers to an array of distinct polynucleotides or oligonucleotides synthesized on a substrate, such as paper, nylon or other type of membrane, filter, chip, glass slide, or any other suitable solid support.

The terms “modulated” and “differentially regulated” as used herein refer to both upregulation (i.e., activation or stimulation (e.g., by agonizing or potentiating)) and downregulation (i.e., inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, “gene silencing” or “gene silenced” in reference to an activity of an RNAi molecule, for example a siRNA or miRNA refers to a decrease in the mRNA level in a cell for a target gene (i.e., an COPD gene sequence) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of downstream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to alter the expression of, such as in particular inhibit the expression of an COPD gene sequence.

As used herein, a “siRNA” refers to a nucleic acid that forms a double stranded RNA, which double stranded RNA has the ability to reduce or inhibit expression of a target COPD gene sequence when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim et al., Genes & Development, 17, p. 991-1008 (2003), Lim et al., Science 299, 1540 (2003), Lee and Ambros Science, 294, 862 (2001), Lau et al., Science 294, 858-861 (2001), Lagos-Quintana et al., Current Biology, 12, 735-739 (2002), Lagos Quintana et al., Science 294, 853-857 (2001), and Lagos-Quintana et al., RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al., 2004. Cell 116:281-297), comprises a dsRNA molecule.

As used herein, the term “promoter” means a DNA sequence that regulates expression of a selected DNA sequence operably linked to the promoter, and which effects expression of the selected DNA sequence in cells. The term encompasses “tissue specific” promoters, i.e., promoters which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively expressed or that are inducible (i.e., expression levels can be controlled).

The terms “protein”, “polypeptide”, and “peptide” are used interchangeably herein when referring to a gene product.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the disclosure to identify compounds that modulate a bioactivity.

A “substitution”, as used herein, refers to the replacement of one or more amino acids or nucleotides by different amino acids or nucleotides, respectively.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of the polypeptide.

As used herein, the term “transgene” means a nucleic acid sequence (or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g., either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” as used herein refers to any animal, such as a mammal, for example a human. The methods and compositions described herein can be useful in both human therapeutics and veterinary applications. In some embodiments, the patient is a mammal, and in some embodiments, the patient is human. For veterinary purposes, the terms “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

As used herein, “pharmaceutically acceptable salt thereof” includes an acid addition salt or a base salt.

As used herein, “pharmaceutically acceptable carrier” includes any material which, when combined with a compound of the disclosure, allows the compound to retain biological activity, such as the ability to induce apoptosis of leukemia or breast tumor cells, and is non-reactive with the subject's immune system. Examples include, but are not limited to, any of the standard pharmaceutical carriers such as a phosphate buffered saline solution, water, emulsions such as oil/water emulsions, and various types of wetting agents. Compositions comprising such carriers are formulated by well known conventional methods (see, for example, Remington's Pharmaceutical Sciences, Chapter 43, 14^{th} Ed., Mack Publishing Co., Easton, Pa.).

### III. Lung Pathogenic Stem Cell Inhibitors

The inventors have observed that certain of these drug agents are able to selectively kill pathogenic stem cells isolated from pulmonary biopsies.

HSP90, HSP70 and dual HSP90/70 Inhibitors

For example, one aspect of the disclosure relates to the use of an HSP90 inhibitor, an HSP70 inhibitor or a combination thereof including in the form of a single molecule dual HSP90/70 inhibitor, as part of a treatment for COPD.

Examples of Hsp90 inhibitors include, but are not limited to, geldanamycin, radicicol, 17-N-allylamino-17-demethoxygeldanamycin (also known as tanespicmycin or 17-AAG) (BMS), herbimycin A, novobiocin sodium (U-6591), 17-GMB-APA-GA, 17-AAG-nab, 17-AEP, macbecin I, CCT 018159, gedunin, PU24FC1, PU-H71, PU-DZ8, PU3, NVP-AUY922 (Novartis), NVP-HSP990 (Novartis), retaspimycin hydrochloride/IPI-504 (Infinity), BIlB021/CNF2024 (Biogen Idec), ganetespib (STA-9090, Synta), STA-1474, SNX-5422/mesylate (Pfizer), B11B028 (Biogen Idec), KW-2478 (Kyowa Hakko Kirin), AT13387 (Astex), XL888 (Exelixis), MPC-3100 (Myriad), ABI-010/nab (nanoparticle, albumin bound)-17AAG (Abraxis), 17-aminodemethoxygeldanamycin (IPI-493), 17-dimethylaminoethylamino-17-demethoxygeldanamycin (17-DMAG), SNX-2112, SNX-7081, Debio0932, B11B021, MPC-3100, MPC-0767, PU3, PU-H58, DS-2248, CCT018159, CCT0129397, BJ-B11, elesclomol (STA-4783), G3130, a herbimycin (such as Herbimycin A; Herbimycin B; Herbimycin C), radester, KNK437, HSP990, NVP-BEP800, Celastrol, Alvespimycin, Autolytimycin, AUY13387, BX-2819, CUDC-305, Curvularin, Flavopiridol, Lebstatin, L-783,277, LL-Z1640-2, Maytansine, MPC-6827, Mycograb, NCS-683664, NXD30001, PF-04929113, Pochonin D, Reblastatin, Redicicol, Rifabutin, VER49009, Xestodccalactone, and Zearalenone.

In certain embodiments, the HSP90 inhibitor is selected from the group consisting of 17-AAG, 17-AEP, 17-DMAG, B11B021, CCT018159, Celastrol, Gedunin, NVP-AUY922 (aka AUY922), PU-H71, and Radicicol.

In certain embodiments, the HSP90 Inhibitor is a benzoquinone class of compounds known as ansamycins (e.g., herbimycin A, geldanamycin, 17-AAG, macbecin, and ansatrienins). These include:

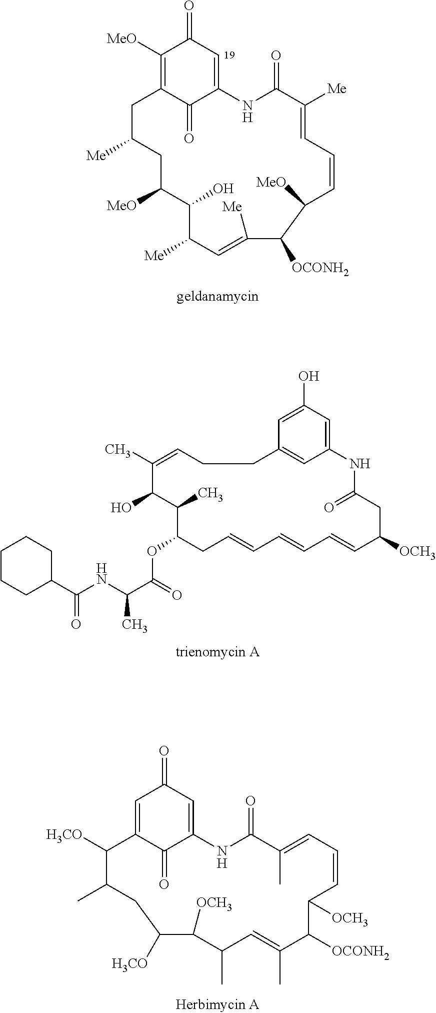

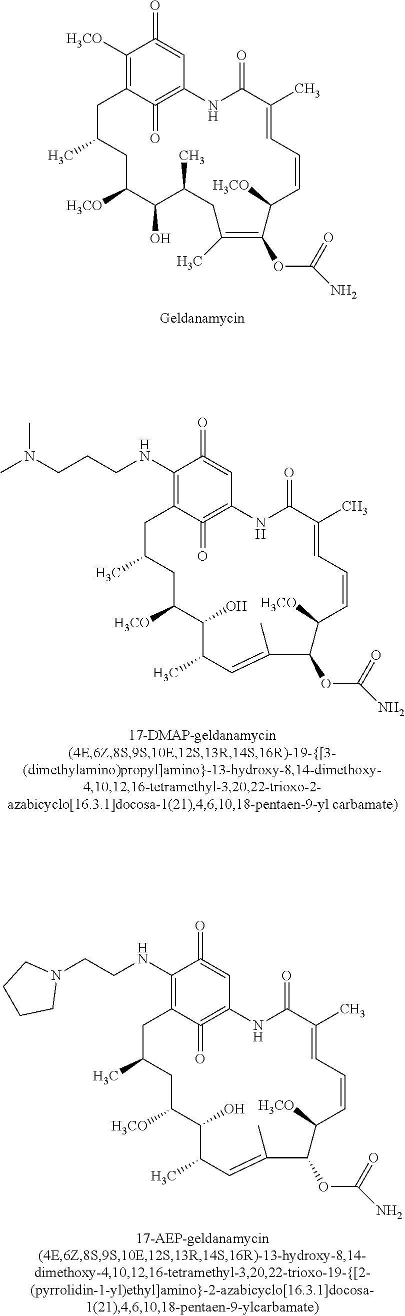

In certain embodiments, the HSP90 Inhibitor is a benzoyl compound represented by formula:

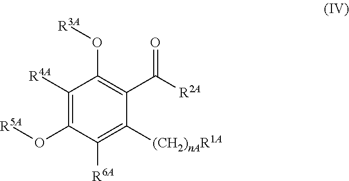

wherein
nA represents an integer of 1 to 5;
R1A represents substituted or unsubstituted lower alkyl, substituted or unsubstituted lower alkoxy, substituted or unsubstituted cycloalkyl, substituted or unsubstituted lower alkoxycarbonyl, substituted or unsubstituted heterocycle-alkyl, substituted or unsubstituted aryl, —C(═O)N(R7A)(R8A) (wherein R7A and R8A may be the same or different, and each represents a hydrogen atom, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, or substituted or unsubstituted aroyl, or R7A and R8A are combined together with the adjacent nitrogen atom thereto to form a substituted or unsubstituted heterocyclic group), or —N(R9A)(R10A) (wherein R9A and R10A have the same meanings as the above R7A and R8A, respectively);
R2A represents substituted or unsubstituted aryl or a substituted or unsubstituted aromatic heterocyclic group;
R3A and R5A may be the same or different, and each represents a hydrogen atom, substituted or unsubstituted lower alkyl, substituted or unsubstituted lower alkenyl, substituted or unsubstituted lower alkanoyl, substituted or unsubstituted cycloalkyl, substituted or unsubstituted aralkyl, or substituted or unsubstituted aroyl;
R4A represents a hydrogen atom, hydroxy, or halogen; and
R6A represents a hydrogen atom, halogen, cyano, nitro, substituted or unsubstituted lower alkyl, substituted or unsubstituted lower alkenyl, substituted or unsubstituted lower alkynyl, substituted or unsubstituted lower alkoxy, substituted or unsubstituted cycloalkyl, amino, lower alkylamino, di(lower alkyl)amino, carboxy, substituted or unsubstituted lower alkoxycarbonyl, substituted or unsubstituted lower alkanoyl, substituted or unsubstituted aryloxy, substituted or unsubstituted aryl, a substituted or unsubstituted heterocyclic group, substituted or unsubstituted aralkyl, or substituted or unsubstituted heterocycle-alkyl;
   or is a prodrug thereof; or a pharmaceutically acceptable salt thereof, and the like.

In certain embodiments, the HSP90 Inhibitor is a benzene derivative represented by formula:

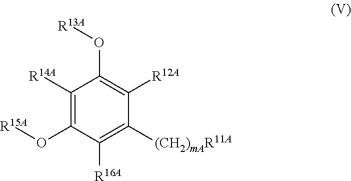

wherein
mA represents an integer of 0 to 10;
R11A represents a hydrogen atom, hydroxy, cyano, carboxy, nitro, halogen, substituted or unsubstituted lower alkyl, substituted or unsubstituted lower alkenyl, substituted or unsubstituted lower alkynyl, substituted or unsubstituted cycloalkyl, substituted or unsubstituted lower alkoxycarbonyl, substituted or unsubstituted aroyl, substituted or unsubstituted lower alkanoyl, substituted or unsubstituted heterocycle-alkyl, substituted or unsubstituted aryl, substituted or unsubstituted aralkyl, substituted or unsubstituted arylsulfonyl, a substituted or unsubstituted heterocyclic group, —C(═O)N(R17A)(R18A) (wherein R17A and R18A may be the same or different, and each represents a hydrogen atom, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, or substituted or unsubstituted aroyl, or R17A and R18A are combined together with the adjacent nitrogen atom thereto to form a substituted or unsubstituted heterocyclic group), or —N(R19A)(R20A) (wherein R19A and R20A may be the same or different, and each represents a hydrogen atom, substituted or unsubstituted lower alkylsulfonyl, substituted or unsubstituted lower alkyl, substituted or unsubstituted cycloalkyl, substituted or unsubstituted lower alkanoyl, substituted or unsubstituted aryl, a substituted or unsubstituted heterocyclic group, substituted or unsubstituted aralkyl, substituted or unsubstituted heterocycle-alkyl, substituted or unsubstituted aroyl, or R19A and R20A are combined together with the adjacent nitrogen atom thereto to form a substituted or unsubstituted heterocyclic group), or —C(═O)N(R21A)(R22A) (wherein R21A and R22A have the same meanings as R17 and R18 defined above, respectively, or R21A and R22A are combined together with the adjacent nitrogen atom thereto to form a substituted or unsubstituted heterocyclic group) or —OR23A (wherein R23A represents substituted or unsubstituted lower alkyl, substituted or unsubstituted lower alkenyl, substituted or unsubstituted lower alkanoyl, substituted or unsubstituted aryl, a substituted or unsubstituted heterocyclic group, substituted or unsubstituted aralkyl, or substituted or unsubstituted heterocycle-alkyl);
R12A represents substituted or unsubstituted lower alkyl, substituted or unsubstituted lower alkenyl, substituted or unsubstituted lower alkynyl, substituted or unsubstituted aryl or a substituted or unsubstituted heterocyclic group;
R13A and R15A may be the same or different, and each represents a hydrogen atom, substituted or unsubstituted lower alkyl, substituted or unsubstituted lower alkenyl, substituted or unsubstituted lower alkanoyl, substituted or unsubstituted cycloalkyl, substituted or unsubstituted lower alkylsulfonyl, substituted or unsubstituted arylsulfonyl, carbamoyl, sulfamoyl, substituted or unsubstituted lower alkylaminocarbonyl, substituted or unsubstituted di(lower alkyl)aminocarbonyl, substituted or unsubstituted lower alkoxycarbonyl, substituted or unsubstituted heterocycle-carbonyl, substituted or unsubstituted aralkyl, or substituted or unsubstituted aroyl;
R14A and R16A may be the same or different, and each represents a hydrogen atom, hydroxy, halogen, cyano, nitro, substituted or unsubstituted lower alkyl, substituted or unsubstituted lower alkenyl, substituted or unsubstituted lower alkynyl, substituted or unsubstituted lower alkoxy, substituted or unsubstituted cycloalkyl, amino, lower alkylamino, di(lower alkyl)amino, carboxy, substituted or unsubstituted lower alkoxycarbonyl, substituted or unsubstituted aryloxy, substituted or unsubstituted aryl, a substituted or unsubstituted heterocyclic group, substituted or unsubstituted lower alkanoyl, substituted or unsubstituted aralkyl, or substituted or unsubstituted heterocycle-alkyl),
   or is a prodrug thereof; or a pharmaceutically acceptable salt thereof, and the like.

Radicicol, a macrocyclic lactone antibiotic, has been shown to inhibit the function of HSP90. To further investigate the biological mechanism of radicicol and its analogs in regulating HSP90 and establish the fundamental structure-activity relationship, a number of radicicol analogs have been synthesized and studied. The term “radicicol analogs” or “radicicol derivatives” as used herein denotes macrocyclic lactone compounds that are structurally similar to radicicol. Specifically, the “radicicol analogs” or “radicicol derivatives” refer to compounds of fused bicyclic ring structure wherein a six-membered aromatic ring shares two carbon atoms with a 12- to 16-membered non-aromatic ring containing a lactone group and at least one olefin group in the core of the 12- to 16-membered ring. The radicicol analogs/derivatives may have one or more substituents on the six-membered aromatic ring or the 12- to 16-membered non-aromatic ring. It is noted that the terms “analog” and “derivative” are used interchangeably in the present application. A number of radicicol analogs have been disclosed in patent publications including WO 96/33989, WO 98/18780, WO 99/55689, U.S. Pat. Nos. 7,115,651, 5,731,343, and 5,077,165, all of which are herein incorporated by reference in their entirety.

It has been reported that certain purine scaffold-based compounds are HSP90 inhibitors. (See, for example, WO 02/36705, WO 03/037860, and WO 2006/084030, all of which are herein incorporated by reference in their entirety) These purine scaffold-based HSP90 inhibitors typically have a structure wherein an adenine ring and a six-membered aryl or heteroaryl ring are linked through a linker which can be methylene, fluorinated methylene, sulfur, oxygen, nitrogen, carbonyl, imine, sulfinyl, or sulfonyl. PU3 and PU24FCI are examples of two compounds exemplifying the purine scaffold-based HSP90 inhibitors.

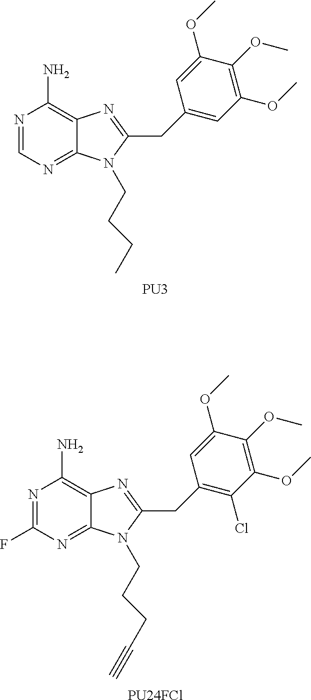

Some pyrazole or imidazole scaffold-based compounds are known to inhibit HSP90. These pyrazole or imidazole scaffold-based HSP90 inhibitors are typically non-fused tricyclic compounds wherein two aryl or heteroaryl rings are attached to two adjacent positions (carbon or nitrogen atom) of a pyrazole or imidazole ring, respectively. (See, for example, WO 2007/021877, which is herein incorporated by reference in its entirety, or Vernalis Ltd, Bioorg Med Chem Lett, 2006, 16, 2543-2548, or Sharp et al., Molecular Cancer Therapeutics, 2007, 6, 1 198-1211). Examples of pyrazole or imidazole scaffold-based HSP90 inhibitors include:

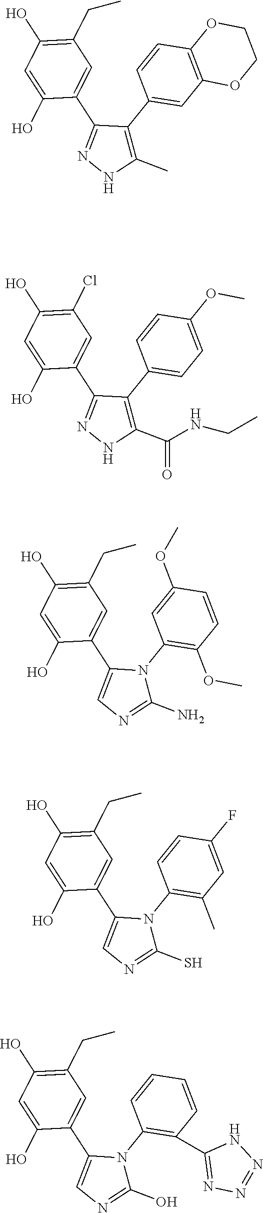 [SMILES: CCc1cc(-c2n[nH]c(C)c2-c2ccc3c(c2)OCCO3)c(O)cc1O ; CCNC(=O)c1[nH]nc(-c2cc(Cl)c(O)cc2O)c1-c1ccc(OC)cc1 ; CCc1cc(-c2cnc(N)n2-c2cc(OC)ccc2OC)c(O)cc1O ; CCc1cc(-c2cnc(S)n2-c2ccc(F)cc2C)c(O)cc1O ; CCc1cc(-c2cnc(O)n2-c2ccccc2-c2nnn[nH]2)c(O)cc1O]

Another class of HSP90 inhibitors are tetrahydroindolone and tetrahydroindazolone derivatives reported in WO 2006/091963, the disclosure of which is herein incorporated by reference in its entirety. These tetrahydroindolone or tetrahydroindazolone based HSP90 inhibitors generally have a structure wherein a substituted aryl group is directly attached to the nitrogen atom of a tetrahydroindolone or tetrahydroindazolone. Examples in WO 2006/091963 include:

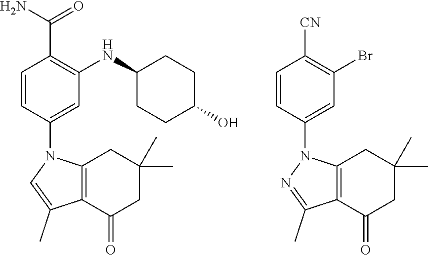 [SMILES: Cc1cn(-c2ccc(C(N)=O)c(N[C@H]3CC[C@H](O)CC3)c2)c2c1C(=O)CC(C)(C)C2 ; Cc1nn(-c2ccc(C#N)c(Br)c2)c2c1C(=O)CC(C)(C)C2]

A number of HSP90 inhibitors in various compound classes have been developed as potential agents for cancer treatment. These include purine-based compounds (PCT publications WO/2006/084030; WO/2002/036075; U.S. Pat. No. 7,138,401; US20050049263; Biamonte et al., 2006, J. Med. Chem. 49:817-828; Chiosis, 2006, Curr. Top. Med. Chem. 6:1183-1191; He et al., 2006, J. Med. Chem. 49:381-390), pyrazole-based compounds (Rowlands et al., 2004, Anal. Biochem. 327:176-183; Dymock et al., 2005, J. Med. Chem. 48:4212-4215; PCT publication WO/2007/021966; WO/2006/039977; WO/2004/096212; WO/2004/056782; WO/2004/050087; WO/2003/055860; U.S. Pat. No. 7,148,228), peptidomimetic shepherdin (Plescia et al., 2005, Cancer Cell 7:457-468; US publication 20060035837), and HSP90 inhibitors in other compound classes (PCT publications WO/2006/123165; WO/2006/109085; WO/2005/028434; U.S. Pat. Nos. 7,160,885; 7,138,402; 7,129,244; US20050256183; US20060167070; US20060223797; WO2006091963).

In certain embodiments, the anti-PLESC agent is an HSP90 inhibitor is a compound selected from the group:
geldamycin;
17-AAG (17-allyl-17-demethoxygeldanamycin);
17-DMAG (17-desmethoxy-17-N,N-dimethylaminoethylaminogel danamycin); IPI-504 (17-allylamino-I 7-demethoxygeldanamycin hydroquinone hydrochloride); IP 1-493 (17-desmethoxy-17-amino geldanamycin);
BIIB021 ([6-Chloro-9-(4-methoxy-3,5-dimethylpyridin-2-ylmethyl)-9H-purin-2-yl]amine);
MPC-3100 ((S)-1-(4-(2-(6-amino-8-((6-bromobenzo[d][I,3]dioxol-5-yl)thio)-9H-purin-9-yl)ethyl)piperidin-1-yl)-2-hydro xypropan-1-one);
Debio 0932 (2-((6-(dimethylamino)benzo [d] [1,3]dioxol-5-yl)thio)-1-(2-(neopentylamino)ethyl)-IH-imidazo[4,5-c]pyridin-4-amine);
PU-H71 (6-Amino-8-[(6-iodo-I,3-benzodioxol-5-yl)thio]-N-(I-methylethyl)-9H-purine-9-propanamine);
STA-9090 (5-[2,4-dihydroxy-5-(I-methylethyl)phenyl]-4-(I-methyl-IH-indol-5-yl)-2,4-dihydro-3H-1,2,4-triazol-3-one);
VER52296 (5-(2,4-Dihydroxy-5-isopropylphenyl)-N-ethyl-4-(4-(morpholinomethyl)phenyl)isoxazole-3-carboxamide);
KW-2478 (2-(2-ethyl-3,5-dihydroxy-6-(3-methoxy-4-(2-morpholinoethoxy)benzoyl)phenyl)-N,N-bis(2-methoxyethyl)acetamide); AT-13387 ((2,4-dihydroxy-5-isopropylphenyl)(5-((4-methylpiperazin-1-yl)methyl) iso indolin-2-yl)methanone);
Radicicol ((1 aR,2Z,4E, 14R, 15aR)-8-Chloro-Ia, 14, 15, 15a-tetrahydro-9, 11-dihydroxy-14-methyl-6H-oxireno[e][2]benzoxacyclotetradecin-6, 12(7H)-dione);
and
Celastrol ((9 β, 13 a, 14β,20α)-3-Hydroxy-9, 13-dimethyl-2-oxo-24,25,26-trinoroleana-I(10),3,5,7-tetraen-29-oic acid);
   or is a combination thereof, or a pharmaceutically acceptable salt thereof.

Exemplary HSP70 inhibitors include, but are not limited to, MKT-077 (1-Ethyl-2-[[3-ethyl-5-(3-methyl-2(3H)-benzothiazolylidene)-4-oxo-2-thiazolidinylidene]methyl]-pyridinium chloride), Omeprazole (5-Methoxy-2-[[(4-methoxy-3,5-dimethyl-2-pyridinyl)methyl]sulfinyl]-1H-benzimidazole), 5-(N,N-Dimethyl)amiloride hydrochloride (DMA), 2-phenylethynesulfonamide (PES), JG-98 (Li et al., ACS Med. Chem. Lett., (2013)4: 1042-1047), VER-155008, 2-phenylethynesulfonamide (PES), JG-98, 115-7c, apoptozole, JG-13, JG-48, MAL3-101, pifithrin-mu, spergualin, YM-01, YM-08, VER15508 (5′-O-[(4-Cyanophenyl)methyl]-8-[[(3,4-dichlorophenyl)methyl]amino]-adenosine), Apoptozole (4-((2-(3,5-bis(trifluoromethyl)phenyl)-4,5-bis(4-methoxyphenyl)-1H-imidazol-1-yl)methyl)benzamide), HSP70-IN-1, JG2-38 ((2Z,5E)-5-(3,5-Dimethylbenzo[d]thiazol-2(3H)-ylidene)-3-ethyl-2-((3-((2-fluorophenyl)amino)pyridin-4-yl)methylene)thiazolidin-4-one). Additional HSP70 inhibitors are described in U.S. Pat. No. 9,642,843 and U.S. Patent Publication Nos. 2012/0252818, 2017/0014434, and 2018/0002325. See also, Taldone T, et al., Heat shock protein 70 inhibitors. 2,5′-thiodipyrimidines, 5-(phenylthio)pyrimidines, 2-(pyridin-3-ylthio)pyrimidines, and 3-(phenylthio)pyridines as reversible binders to an allosteric site on heat shock protein 70. J Med Chem. 2014 Feb. 27; 57(4):1208-24.

Exemplary HSP70 inhibitor structures include:

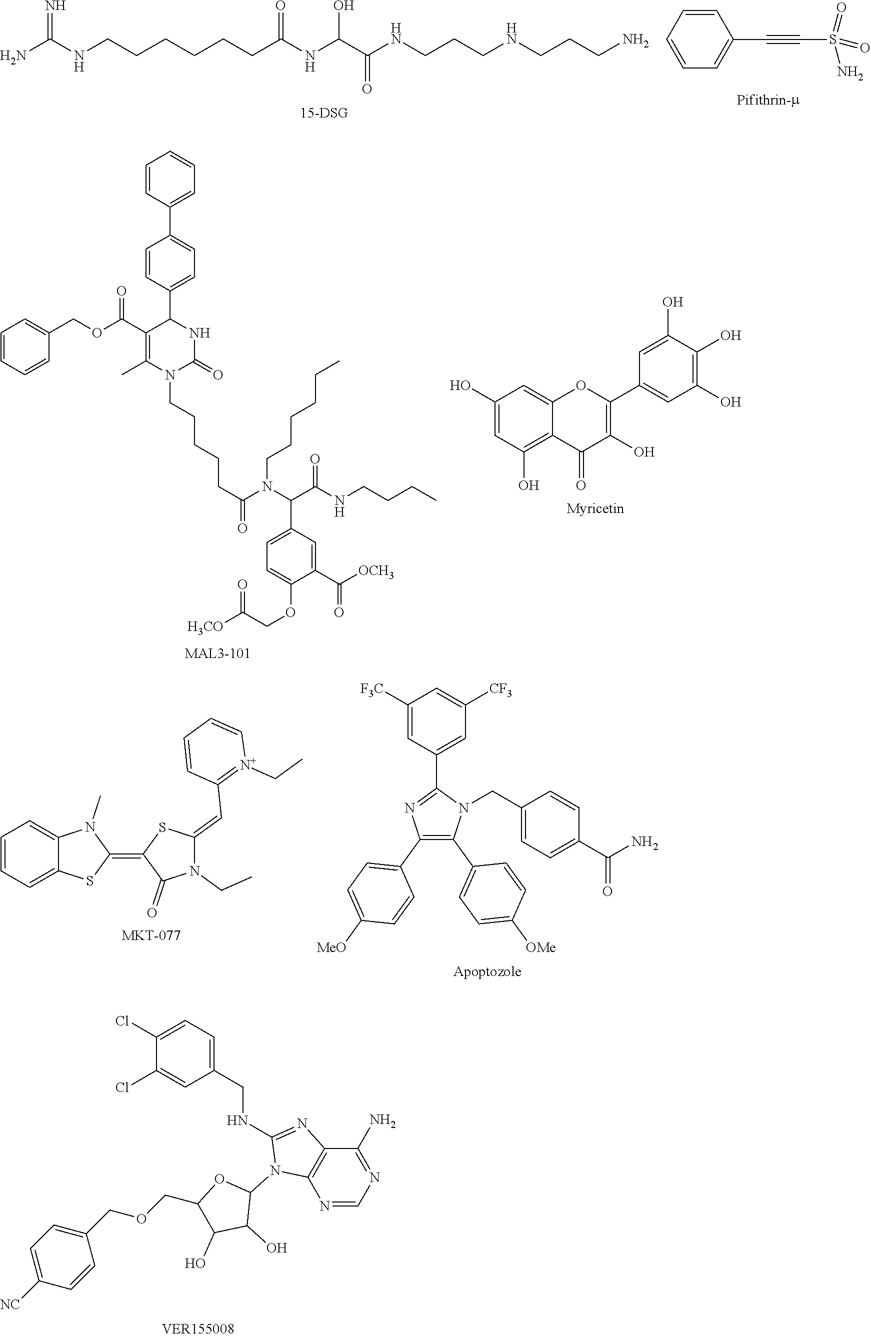

In certain embodiments the present disclosure provides compounds of formula I:

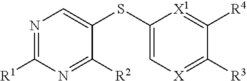

or a pharmaceutically acceptable salt thereof, wherein:
X is —N═ or —CH═;
X¹ is —N═ or —C(R⁵)═;
R¹ is

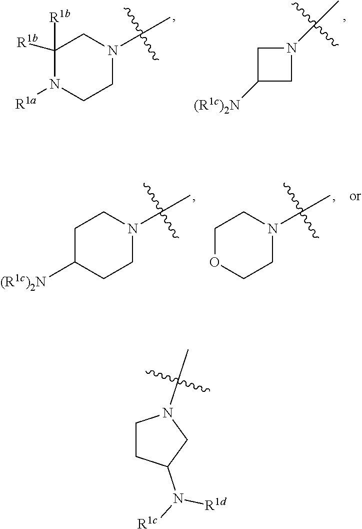

R^{1a} is

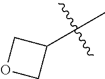 [SMILES: *C1COC1]

or C1-6 aliphatic optionally substituted with one or more groups independently selected from —OH, cyclopropyl, or 5-membered heteroaryl having 1-2 heteroatoms independently selected from nitrogen, oxygen or sulfur;
each R^{1b} is independently hydrogen, C1-4 alkyl, or two R^{1b} groups are optionally taken together to form an oxo group;
each of R^{1c} and R^{1d} is independently hydrogen or C1-4 alkyl;
R² is —O—CH₂—Ring A, —NH—CH₂—Ring A, or —O—CH₂CH₂—Ring A;
   Ring A is unsubstituted phenyl, unsubstituted furanyl,

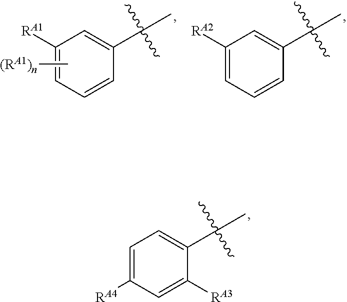

or pyridinyl optionally substituted with R^{A5};
each of R^{A1} is independently halogen, —CN, —C(═O)N(R)₂, —N(R)₂, —OR, —C(═O)R, —N₃, an optionally substituted 5- or 6-membered heterocyclyl or heteroaryl having one or two heteroatoms independently selected from nitrogen, oxygen, or sulfur, or C1-4 alkyl optionally substituted with one or more halogen;
each R is independently hydrogen or C1-4 alkyl optionally substituted with one or more halogen;
R^{A2} is —Cl, —Br, —I, —CN, —C(═O)N(R)₂, —N(R)₂, —OR, —C(═O)R, —N₃, an optionally substituted 5- or 6-membered heterocyclyl or heteroaryl having one or two heteroatoms independently selected from nitrogen, oxygen or sulfur, or C1-4 alkyl optionally substituted with one or more halogen;
n is 1 to 4;
R^{A3} is —H or —F;
R^{A4} is —F or —OR;
R^{A5} is —OR or —N(R)₂;
R³ is —C(O)N(R^{3a})₂, —OR^{3b}, —C(O)H, —C(O)OR, or —N(R^{3c})₂;
   each R³, is independently hydrogen or Ci alkyl optionally substituted with one or more groups independently selected from halogen or 1-pyrrolidinyl;
   R^{3b} is hydrogen or C1-4 alkyl optionally substituted with one or more groups independently selected from halogen, C1-4 alkyl, C1-4 haloalkyl, oxo, or —N(R)₂;
   each R^{3c} is independently hydrogen or C1-4 alkyl optionally substituted with one or more groups independently selected from halogen, C1-4 alkyl, C1-4 haloalkyl, oxo, or —N(R)₂;
R⁴ is R, halogen, or —N(R)₂; and
R⁵ is hydrogen, methyl or —N(R)₂.

In certain embodiments, the anti-PLESC agent is an HSP70 inhibitor is a compound selected from the group:
2-phenylethynesulfonamide (Pifithrin-μ);
MKT-077 (I-Ethyl-2-[[3-ethyl-5-(3-methyl-2(3H)-benzothiazolylidene)-4-oxo-2-thiazolidinylidene]methyl]-pyridinium chloride);
methylene blue;
VER155088 (5′-0-[(4-Cyanophenyl)methyl]-8-[[(3,4-dichlorophenyl)methyl]amino]-adenosine);
   or is a combination thereof, or a pharmaceutically acceptable salt thereof.

In certain embodiments, the anti-PLESC agent is a combination of each of an HSP70 inhibitor and HSP90 inhibitor, i.e., the combination inhibits both HSP70 and HSP90.

In certain embodiments, the anti-PLESC agent is a dual HSP70/HSP90 inhibitor, i.e., inhibits both HSP70 and HSP90. In certain embodiments, the dual inhibitor inhibits each of HSP70 and HSP90 with EC₅₀'s preferably within at least 100× of each other, more preferable within 10×, 5× or even 2× of each other. In addition to certain agents described above that can be used as dual HSP70/90 inhibitors,

mTOR Inhibitors

In certain embodiments, the anti-PLESC agent is an mTor inhibitor.

Non-limiting examples of mTOR inhibitors include rapamycin (sirolimus), everolimus, ridaforolimus, temsirolimus, zotarolimus, rapamycin prodrug AP-23573 (deforolimus), AP-23675, AP-23481, torin-I, torin-2, WYE-354, dactolisib, voxtalisib, omipalisib, apitolisib, vistusertib, gedatolisib, WYE-125132, BGT226, palomid 529, GDC-0349, XL388, CZ415, CC-223, SF1 126, INK128, biolimus-7, biolimus-9 (umirolimus), GSK2126458, OS1027, PP121, Torkinib (PP242), RTB 101, TAM-01, TAM-03, LY294002, CCI-779 (rapamycin 42-ester with 3-hydroxy-2-(hydroxymethyl)-2-methylpropionic acid), AZD8055 ((5-(2,4-bis((S)-3-methylmorpholino)pyrido[2,3-d]pyrimidin-7-yl)-2-methox-yphenyl)methanol); PKI-587 (1-(4-(4-(dimethylamino)piperidine-1-carbonyl)phenyl)-3-(4-(4,6-dimorphol-ino-1,3,5-triazin-2-yl)phenyl)urea), NVP-BEZ235 (2-methyl-2-{4-[3-methyl-2-oxo-8-(quinolin-3-yl)-2,3-dihydro-1H-imidazo[4-,5-c]quinolin-1-yl]phenyl}propanenitrile), LY294002 ((2-(4-morpholinyl)-8-phenyl-4H-1-benzopyran-4-one), 40-O-(2-hydroxyethyl)-rapamycin; ABT578 (zotarolimus), TAFA-93, 42-O-(methyl-D-glucosylcarbonyl)rapamycin, 42-O-[2-(methyl-D-glucosylcarbonyloxy)ethyl]rapamycin, 31-O-(methyl-D-glucosylcarbonyl)rapamycin, 42-O-(2-hydroxyethyl)-31-O-(methyl-D-glucosylcarbonyl)rapamycin, 42-O-(2-O-methyl-D-fructosylcarbonyl)rapamycin, 42-O-[2-(2-O-methyl-D-fructosylcarbonyloxy)ethyl]rapamycin, 42-O-(2-O-methyl-L-fructosylcarbonyl)rapamycin, 42-O-[2-(2-O-methyl-L-fructosylcarbonyloxy)ethyl]rapamycin, 31-O-(2-O-methyl-D-fructosylcarbonyl)rapamycin, 42-O-(2-hydroxyethyl)-31-O-(2-O-methyl-D-fructosylcarbonyl)rapamycin, 31-O-(2-O-methyl-L-fructosylcarbonyl)rapamycin, 42-O-(2-hydroxyethyl)-31-O-(2-O-methyl-L-fructosylcarbonyl)rapamycin, 42-O-(D-allosylcarbonyl)rapamycin, 42-O-[2-(D-allosylcarbonyloxy)ethyl]rapamycin, 42-O-(L-allosylcarbonyl)rapamycin, 42-O-[2-(L-allosylcarbonyloxy)ethyl]rapamycin, 31-O-(D-allosylcarbonyl)rapamycin, 42-O-(2-hydroxyethyl)-31-O-(D-allosylcarbonyl)rapamycin, 31-O-(L-allosylcarbonyl)rapamycin, 42-O-(2-hydroxyethyl)-31-O-(L-allosylcarbonyl)rapamycin, 42-O-(D-fructosylcarbonyl)rapamycin, 42-O-[2-(D-fructosylcarbonyloxy)ethyl]rapamycin, 42-O-(L-fructosylcarbonyl)rapamycin, 42-O-[2-(L-fructosylcarbonyloxy)ethyl]rapamycin, 31-O-(D-fructosylcarbonyl)rapamycin, 42-O-(2-hydroxyethyl)-31-O-(D-fructosylcarbonyl)rapamycin, 31-O-(L-fructosylcarbonyl)rapamycin, 42-O-(2-hydroxyethyl)-31-O-(L-fructosylcarbonyl)rapamycin, 42-O-(D-fucitolylcarbonyl)rapamycin, 42-O-[2-(D-fucitolylcarbonyloxy)ethyl]rapamycin, 42-O-(L-fucitolylcarbonyl)rapamycin, 42-O-[2-(L-fucitolylcarbonyloxy)ethyl]rapamycin, 31-O-(D-fucitolylcarbonyl)rapamycin, 42-O-(2-hydroxyethyl)-31-O-(D-fucitolylcarbonyl)rapamycin, 31-O-(L-fucitolylcarbonyl)rapamycin, 42-O-(2-hydroxyethyl)-31-O-(L-fucitolylcarbonyl)rapamycin, 42-O-(D-glucalylcarbonyl)rapamycin, 42-O-[2-(D-glucalylcarbonyloxy)ethyl]rapamycin, 42-O-(D-glucosylcarbonyl)rapamycin, 42-O-[2-(D-glucosylcarbonyloxy)ethyl]rapamycin, 42-O-(L-glucosylcarbonyl)rapamycin, 42-O-[2-(L-glucosylcarbonyloxy)ethyl]rapamycin, 31-O-(D-glucalylcarbonyl)rapamycin, 42-O-(2-hydroxyethyl)-31-O-(D-glucalylcarbonyl)rapamycin, 31-O-(D-glucosylcarbonyl)rapamycin, 42-O-(2-hydroxyethyl)-31-O-(D-glucosylcarbonyl)rapamycin, 31-O-(L-glucosylcarbonyl)rapamycin, 42-O-(2-hydroxyethyl)-31-O-(L-glucosylcarbonyl)rapamycin, 42-O-(L-sorbosylcarbonyl)rapamycin, 42-O-(D-sorbosylcarbonyl)rapamycin, 31-O-(L-sorbosylcarbonyl)rapamycin, 31-O-(D-sorbosylcarbonyl)rapamycin, 42-O-[2-(L-sorbosylcarbonyloxy)ethyl]rapamycin, 42-O-[2-(D-sorbosylcarbonyloxy)ethyl]rapamycin, 42-O-(2-hydroxyethyl)-31-O-(D-sorbosylcarbonyl)rapamycin, 42-O-(2-hydroxyethyl)-31-O-(L-sorbosylcarbonyl)rapamycin, 42-O-(D-lactalylcarbonyl)rapamycin, 42-O-[2-(D-lactalylcarbonyloxy)ethyl]rapamycin, 31-O-(D-lactalylcarbonyl)rapamycin, 42-O-(2-hydroxyethyl)-31-O-(D-lactalylcarbonyl)rapamycin, 42-O-(D-sucrosylcarbonyl)rapamycin, 42-O-[2-(D-sucrosylcarbonyloxy)ethyl]rapamycin, 31-O-(D-sucrosylcarbonyl)rapamycin, 42-O-(2-hydroxyethyl)-31-O-(D-sucrosylcarbonyl)rapamycin, 42-O-(D-gentobiosylcarbonyl)rapamycin 42-O-[2-(D-gentobiosylcarbonyloxy)ethyl]rapamycin, 31-O-(D-gentobiosylcarbonyl)rapamycin 42-O-(2-hydroxyethyl)-31-O-(D-gentobiosylcarbonyl)rapamycin 42-O-(D-cellobiosylcarbonyl)rapamycin, 42-O-[2-(D-cellobiosylcarbonyloxy)ethyl]rapamycin, 31-O-(D-cellobiosylcarbonyl)rapamycin, 42-O-(2-hydroxyethyl)-31-O-(D-cellobiosylcarbonyl)rapamycin, 42-O-(D-turanosylcarbonyl)rapamycin, 42-O-[2-(D-turanosylcarbonyloxy)ethyl]rapamycin, 31-O-(D-turanosylcarbonyl)rapamycin, 42-O-(2-hydroxyethyl)-31-O-(D-turanosylcarbonyl)rapamycin, 42-O-(D-palatinosylcarbonyl)rapamycin, 42-O-[2-(D-palatinosylcarbonyloxy)ethyl]rapamycin, 31-O-(D-palatinosylcarbonyl)rapamycin, 42-O-(2-hydroxyethyl)-31-O-(D-palatinosylcarbonyl)rapamycin, 42-O-(D-isomaltosylcarbonyl)rapamycin, 42-O-[2-(D-isomaltosylcarbonyloxy)ethyl]rapamycin, 31-O-(D-isomaltosylcarbonyl)rapamycin, 42-O-(2-hydroxyethyl)-31-O-(D-isomaltosylcarbonyl)rapamycin, 42-O-(D-maltulosylcarbonyl)rapamycin, 42-O-[2-(D-maltulosylcarbonyloxy)ethyl]rapamycin, 42-O-(D-maltosylcarbonyl)rapamycin, 42-O-[2-(D-maltosylcarbonyloxy)ethyl]rapamycin, 31-O-(D-maltulosylcarbonyl)rapamycin, 42-O-(2-hydroxyethyl)-31-O-(D-maltulosylcarbonyl)rapamycin, 31-O-(D-maltosylcarbonyl)rapamycin, 42-0-(2-hydroxyethyl)-31-O-(D-maltosylcarbonyl)rapamycin, 42-O-(D-lactosylcarbonyl)rapamycin, 42-O-[2-(D-lactosylcarbonyloxy)ethyl]rapamycin, 31-O-(methyl-D-lactosylcarbonyl)rapamycin, 42-O-(2-hydroxyethyl)-31-O-(methyl-D-lactosylcarbonyl)rapamycin, 42-O-(D-melibiosylcarbonyl)rapamycin, 31-O-(D-melibiosylcarbonyl)rapamycin, 42-O-(2-hydroxyethyl)-31-O-(D-melibiosylcarbonyl)rapamycin, 42-O-(D-leucrosylcarbonyl)rapamycin, 42-O-[2-(D-leucrosylcarbonyloxy)ethyl]rapamycin, 31-O-(D-leucrosylcarbonyl)rapamycin, 42-O-(2-hydroxyethyl)-31-O-(D-leucrosylcarbonyl)rapamycin, 42-O-(D-raffi nosylcarbonyl)rapamycin, 42-O-[2-(D-raffinosylcarbonyloxy)ethyl]rapamycin, 31-O-(D-raffinosylcarbonyl)rapamycin, 42-O-(2-hydroxyethyl)-31-O-(D-raffinosylcarbonyl)rapamycin, 42-O-(D-isomaltotriosylcarbonyl)rapamycin, 42-O-[2-(D-isomaltosylcarbonyloxy)ethyl]rapamycin, 31-O-(D-isomaltotriosylcarbonyl)rapamycin, 42-O-(2-hydroxyethyl)-31-O-(D-isomaltotriosylcarbonyl)rapamycin, 42-O-(D-cellotetraosylcarbonyl)rapamycin, 42-O-[2-(D-cellotetraosylcarbonyloxy)ethyl]rapamycin, 31-O-(D-cellotetraosylcarbonyl)rapamycin, 42-O-(2-hydroxyethyl)-31-O-(D-cellotetraosylcarbonyl)rapamycin, 42-O-(valiolylcarbonyl)rapamycin, 42-O-[2-(D-valiolylcarbonyloxy)ethyl]rapamycin, 31-O-(valiolylcarbonyl)rapamycin, 42-O-(2-hydroxyethyl)-31-O-(valiolylcarbonyl)rapamycin, 42-O-(valiolonylcarbonyl)rapamycin, 42-O-[2-(D-valiolonylcarbonyloxy)ethyl]rapamycin, 31-O-(valiolonylcarbonyl)rapamycin, 42-O-(2-hydroxyethyl)-31-O-(valiolonylcarbonyl)rapamycin, 42-O-(valienolylcarbonyl)rapamycin 42-O-[2-(D-valienolylcarbonyloxy)ethyl]rapamycin, 31-O-(valienolylcarbonyl)rapamycin, 42-O-(2-hydroxyethyl)-31-O-(valienolylcarbonyl)rapamycin, 42-O-(valienoneylcarbonyl)rapamycin, 42-O-[2-(D-valienoneylcarbonyloxy)ethyl]rapamycin, 31-O-(valienoneylcarbonyl)rapamycin, 42-O-(2-hydroxyethyl)-31-O-(valienoneylcarbonyl)rapamycin, PI-103 (3-[4-(4-morpholinyl)pyrido[3′,2′:4,5]furo[3,2-d]pyrimidin-2-yl]-phenol), KU-0063794 ((5-(2-((2R,6S)-2,6-dimethylmorpholino)-4-morpholinopyrido[2,3-d]pyrimidi-n-7-yl)-2-methoxyphenyl)methanol), PF-04691502 (2-amino-8-((1r,4r)-4-(2-hydroxyethoxy)cyclohexyl)-6-(6-methoxypyridin-3-yl)-4-methylpyrido[2,3-d]pyrimidin-7(8H)-one), CH132799, RG7422 ((S)-1-(4-((2-(2-aminopyrimidin-5-yl)-7-methyl-4-morpholinothieno[3,2-d]p-yrimidin-6-yl)methyl)piperazin-1-yl)-2-hydroxypropan-1-one), Palomid 529 (3-(4-methoxybenzyloxy)-8-(1-hydroxyethyl)-2-methoxy-6H-benzo[c]chromen-6-one), PP242 (2-(4-amino-1-isopropyl-1H-pyrazolo[3,4-d]pyrimidin-3-yl)-1H-indol-5-ol), XL765 (N-[4-[[[3-[(3,5-dimethoxyphenyl)amino]-2-quinoxalinyl]amino]sulfon-yl]phenyl]-3-methoxy-4-methyl-benzamide), GSK1059615 ((Z)-5-((4-(pyridin-4-yl)quinolin-6-yl)methylene)thiazolidine-2,4-dione), PKI-587 (1-(4-(4-(dimethylamino)piperidine-1-carbonyl)phenyl)-3-(4-(4,6-d-imorpholino-1,3,5-triazin-2-yl)phenyl)urea), WAY-600 (6-(1H-indol-5-yl)-4-morpholino-1-(1-(pyridin-3-ylmethyl)piperidin-4-yl)-1H-pyrazolo[3,4-d]pyrimidine), WYE-687 (methyl 4-(4-morpholino-1-(1-(pyridin-3-ylmethyl)piperidin-4-yl)-1H-pyrazolo[3,4-d]pyrimidin-6-yl)phenylcarbamate), WYE-125132 (N-[4-[1-(1,4-dioxaspiro[4.5]dec-8-yl)-4-(8-oxa-3-azabicyclo[3.2.1]oct-3-yl)-1H-pyrazolo[3,4-d]pyrimidin-6-yl]phenyl]-N′-methyl-urea), and WYE-354; as well as pharmaceutically acceptable salts, hydrates, solvates, or amorphous solid thereof, and combinations thereof.

Additional inhibitors of mTOR are described in the following United States patents and patent applications, all of which are incorporated herein by this reference: U.S. Pat. No. 8,461,157 to Cai et al.; U.S. Pat. No. 8,440,662 to Smith et al.; U.S. Pat. No. 8,436,012 to Ohtsuka et al.; U.S. Pat. No. 8,394,818 to Gray et al.; U.S. Pat. No. 8,362,241 to D'Angelo et al.; U.S. Pat. No. 8,314,111 to Chen et al.; U.S. Pat. No. 8,309,546 to Nakayama et al., (including 6-morpholinopurine derivatives); U.S. Pat. No. 8,268,819 to Jin et al.; U.S. Pat. No. 8,211,669 to Reed et al.; U.S. Pat. No. 8,163,755 Jin et al.; U.S. Pat. No. 8,129,371 Zask et al.; U.S. Pat. No. 8,097,622 to Nakayama et al.; U.S. Pat. No. 8,093,050 to Cho et al.; U.S. Pat. No. 8,008,318 to Beckmann et al.; U.S. Pat. No. 7,943,767 to Chen et al.; U.S. Pat. No. 7,923,555 to Chen et al.; U.S. Pat. No. 7,897,608 to Wilkinson et al.; U.S. Pat. No. 7,700,594 to Chen et al.; U.S. Pat. No. 7,659,274 to Crew et al.; U.S. Pat. No. 7,655,673 to Zhang et al., (39-desmethoxyrapamycin); U.S. Pat. No. 7,648,996 to 20 Beckman et al.; U.S. Pat. No. 7,504,397 to Hummersone et al.; U.S. Pat. No. 7,169,817 to Pan et al.; U.S. Pat. No. 7,160,867 to Abel et al., (carbohydrate derivatives of rapamycin); U.S. Pat. No. 7,091,213 to Metcalf III et al., (“rapalogs”); United States Patent Application Publication No. 2013/0079303 by Andrews et al.; and United States Patent Application Publication No. 2013/0040973 by Vannuchi et al.

The structures of certain mTOR inhibitors are disclosed below:

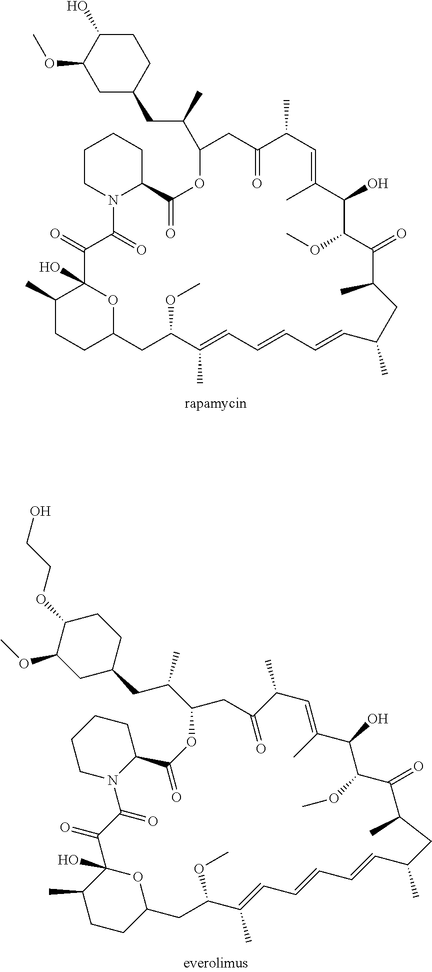

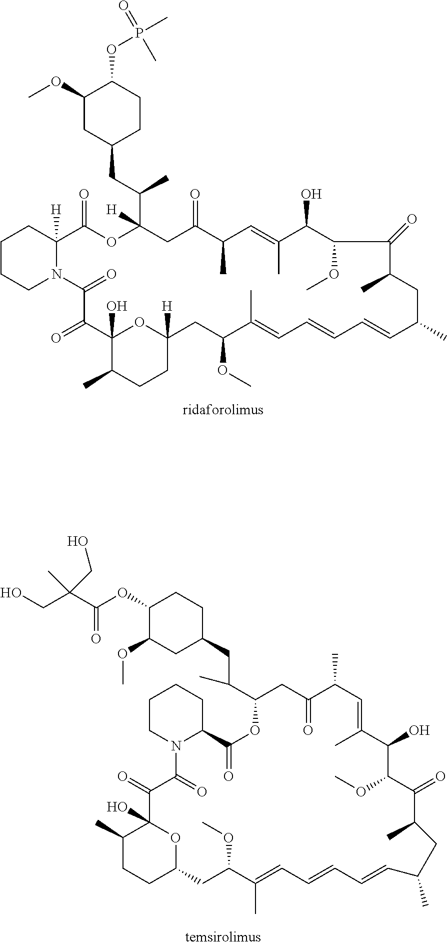 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H](C(C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

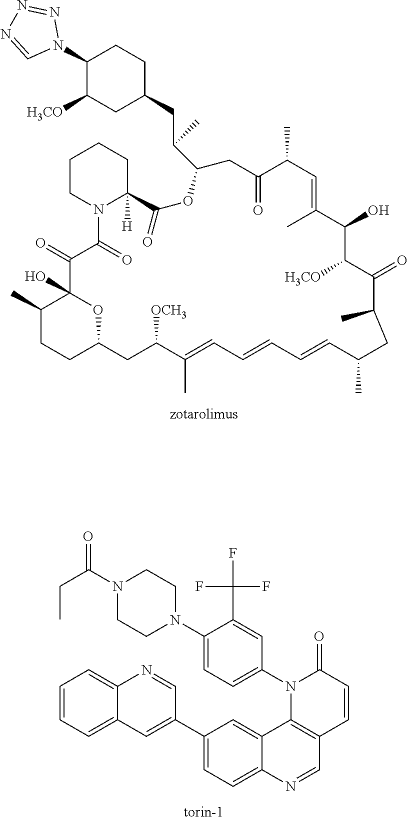 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C ; CCC(=O)N1CCN(c2ccc(-n3c(=O)ccc4cnc5ccc(-c6cnc7ccccc7c6)cc5c43)cc2C(F)(F)F)CC1]

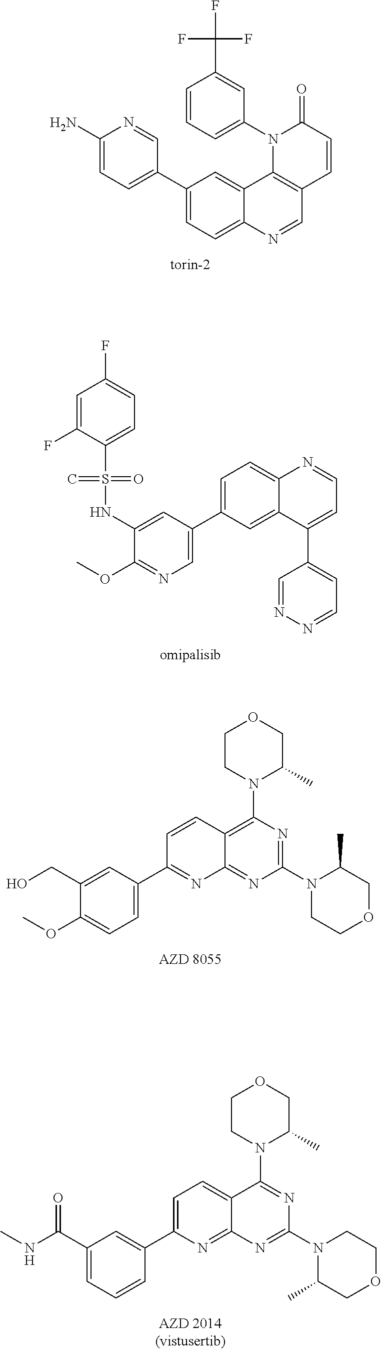 [SMILES: Nc1ccc(-c2ccc3ncc4ccc(=O)n(-c5cccc(C(F)(F)F)c5)c4c3c2)cn1 ; C=S(=O)(Nc1cc(-c2ccc3nccc(-c4ccnnc4)c3c2)cnc1OC)c1ccc(F)cc1F ; COc1ccc(-c2ccc3c(N4CCOC[C@@H]4C)nc(N4CCOC[C@@H]4C)nc3n2)cc1CO ; CNC(=O)c1cccc(-c2ccc3c(N4CCOC[C@@H]4C)nc(N4CCOC[C@@H]4C)nc3n2)c1]

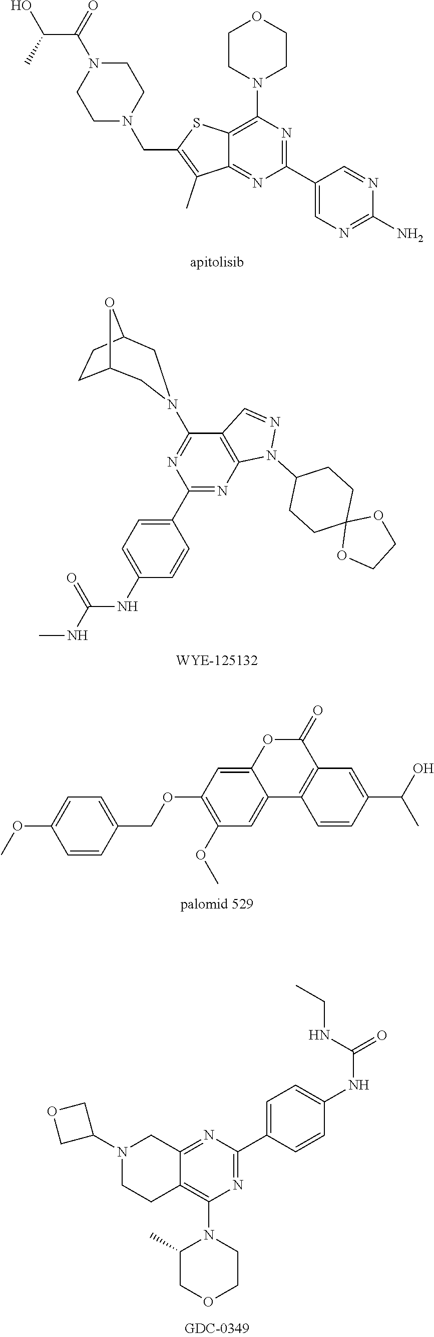 [SMILES: Cc1c(CN2CCN(C(=O)[C@H](C)O)CC2)sc2c(N3CCOCC3)nc(-c3cnc(N)nc3)nc12 ; CNC(=O)Nc1ccc(-c2nc(N3CC4CCC(C3)O4)c3cnn(C4CCC5(CC4)OCCO5)c3n2)cc1 ; COc1ccc(COc2cc3oc(=O)c4cc(C(C)O)ccc4c3cc2OC)cc1 ; CCNC(=O)Nc1ccc(-c2nc3c(c(N4CCOC[C@@H]4C)n2)CCN(C2COC2)C3)cc1]

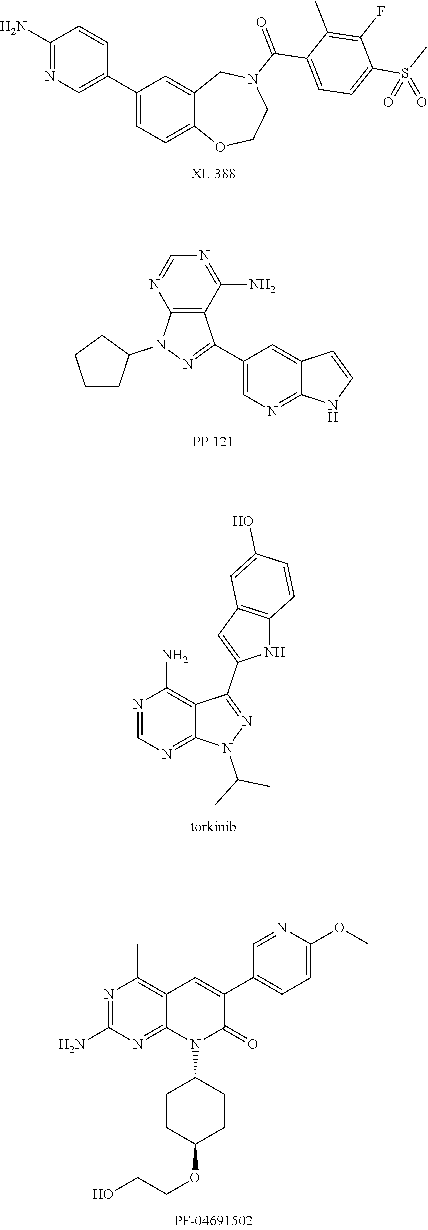 [SMILES: Cc1c(C(=O)N2CCOc3ccc(-c4ccc(N)nc4)cc3C2)ccc(S(C)(=O)=O)c1F ; Nc1ncnc2c1c(-c1cnc3[nH]ccc3c1)nn2C1CCCC1 ; CC(C)n1nc(-c2cc3cc(O)ccc3[nH]2)c2c(N)ncnc21 ; COc1ccc(-c2cc3c(C)nc(N)nc3n([C@H]3CC[C@H](OCCO)CC3)c2=O)cn1]

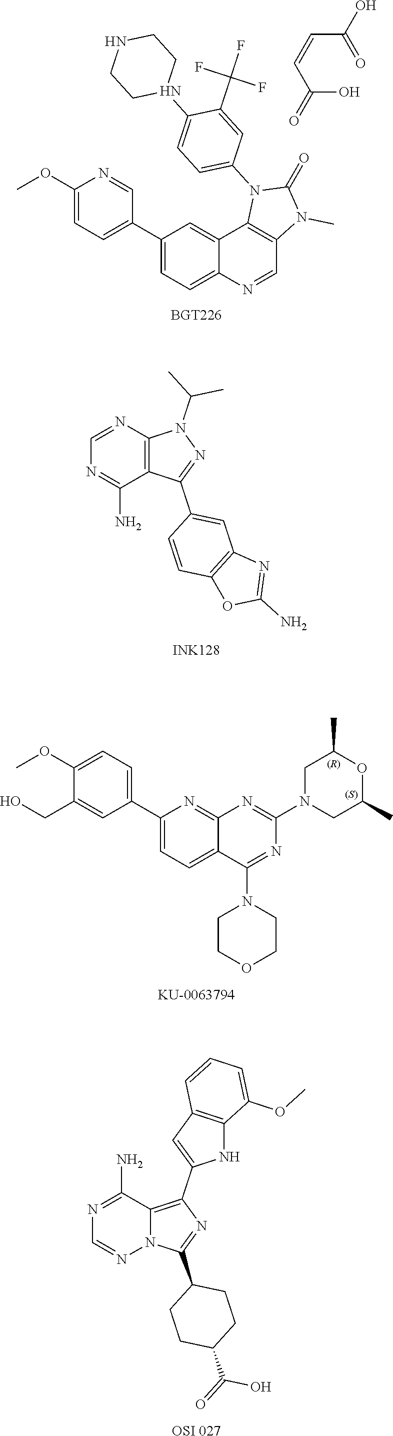

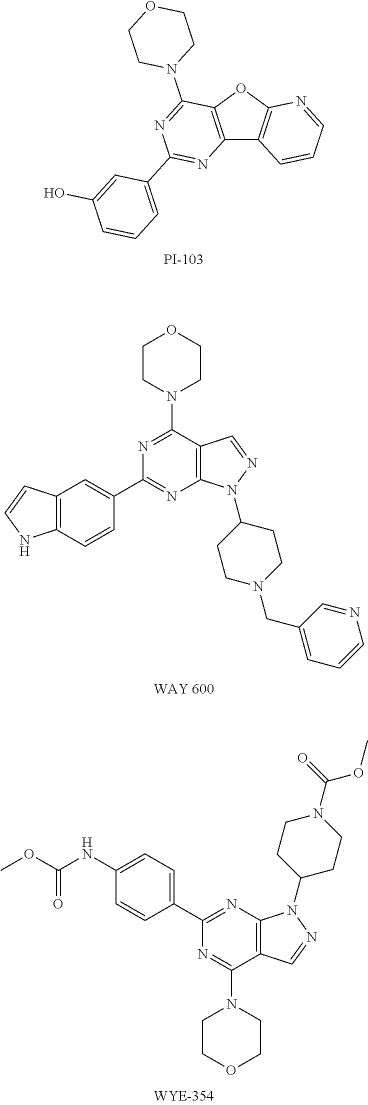

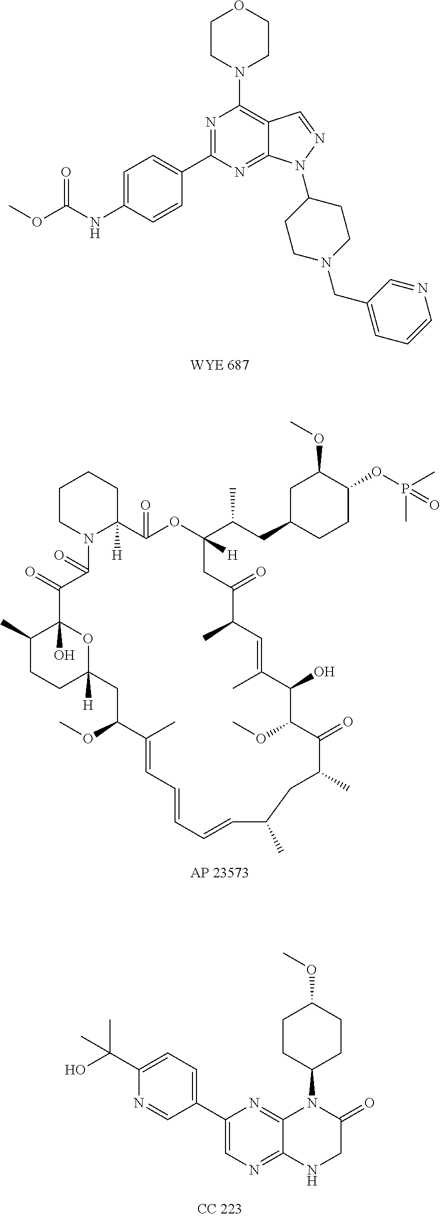 [SMILES: COC(=O)Nc1ccc(-c2nc(N3CCOCC3)c3cnn(C4CCN(Cc5cccnc5)CC4)c3n2)cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1CC[C@H](N2C(=O)CNc3ncc(-c4ccc(C(C)(C)O)nc4)nc32)CC1]

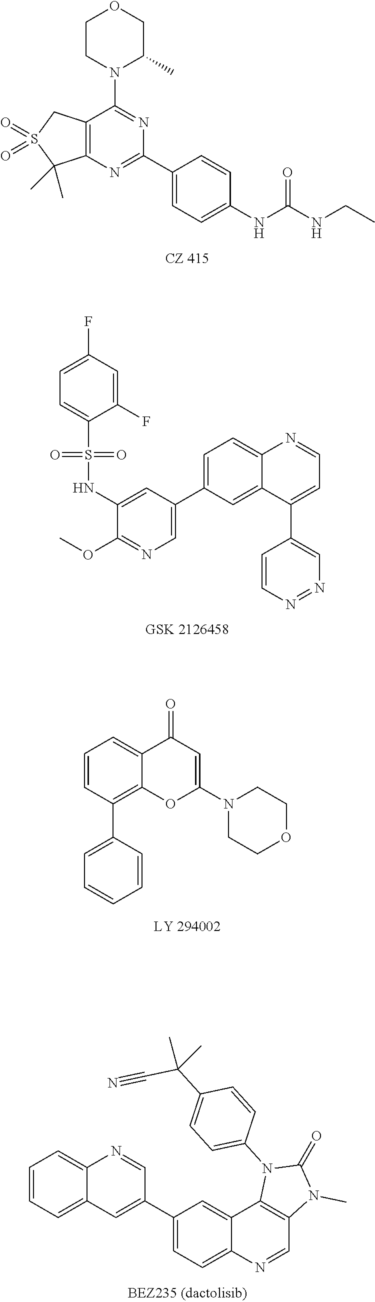 [SMILES: CCNC(=O)Nc1ccc(-c2nc(N3CCOC[C@@H]3C)c3c(n2)C(C)(C)S(=O)(=O)C3)cc1 ; COc1ncc(-c2ccc3nccc(-c4ccnnc4)c3c2)cc1NS(=O)(=O)c1ccc(F)cc1F ; O=c1cc(N2CCOCC2)oc2c(-c3ccccc3)cccc12 ; Cn1c(=O)n(-c2ccc(C(C)(C)C#N)cc2)c2c3cc(-c4cnc5ccccc5c4)ccc3ncc21]

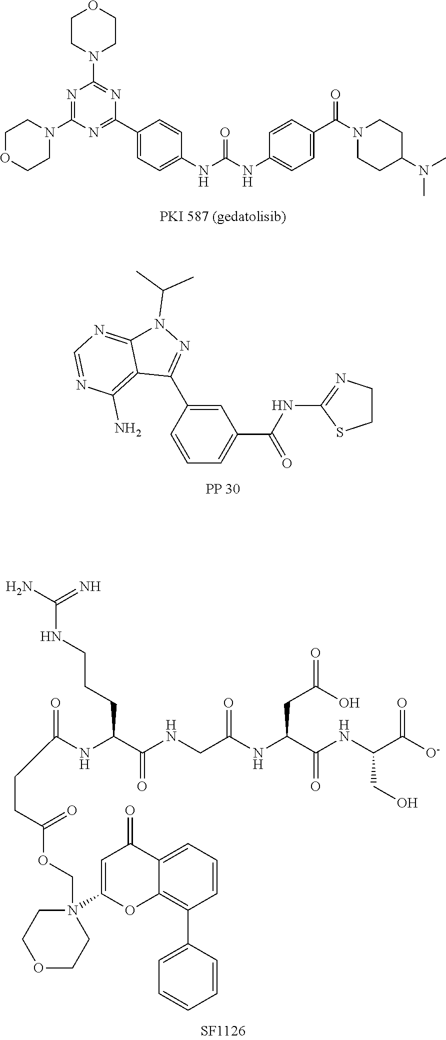

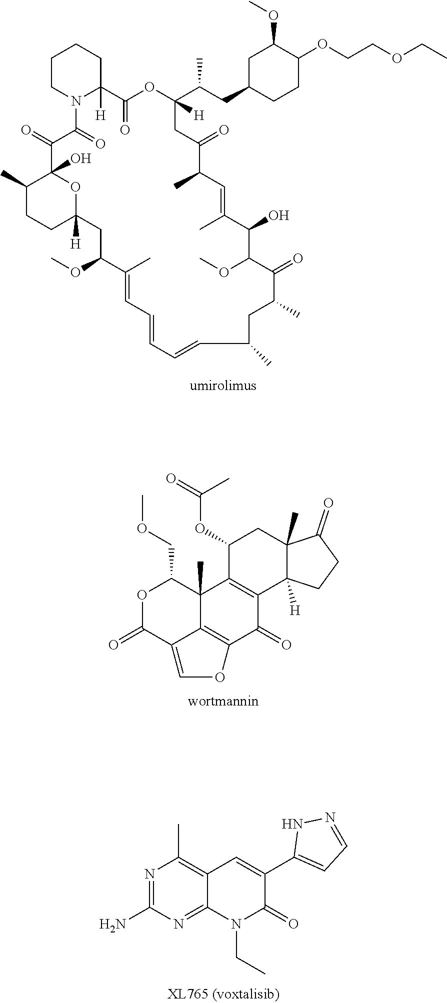 [SMILES: CCOCCOC1CC[C@@H](C[C@@H](C)[C@@H]2CC(=O)[C@H](C)/C=C(\C)[C@@H](O)C(OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCCC3C(=O)O2)C[C@H]1OC ; COC[C@H]1OC(=O)c2coc3c2[C@@]1(C)C1=C(C3=O)[C@@H]2CCC(=O)[C@@]2(C)C[C@H]1OC(C)=O ; CCn1c(=O)c(-c2ccn[nH]2)cc2c(C)nc(N)nc21]

In some embodiments, mTOR inhibitors also include specific inhibitors of mTOR complex 1, specific inhibitors of mTOR complex 2, and the like. In one embodiment, agents that can be used to inhibit mTOR complex 2 include but are not limited to small molecules, nucleic acids, proteins, and antibodies. Small molecules include but are not limited to pyridinonequinolines, pyrazolopyrimidines, and pyridopyrimidines. In a further embodiment, small molecules that inhibit mTOR complexes 1 and 2 include Torin 1, Torin 2, torkinib (PP242), PP30, KU-0063794, WAY-600, WYE-687, WYE-354, AZD8055, INK128, OS1027, AZD2014, omipalisib, wortmannin, LY294002, PI-103, BGT226, XL765, NVP-BEZ235, RTB IOI(RestorBio), and TAM-01 and TAM-03 (Mount Tam Biotechnologies). In a further embodiment, the inhibitors include but is not limited to antisense oligonucleotide, siRNA, shRNA, and combinations thereof. In a further embodiment, the agent that inhibits mTOR complex 2 would not inhibit mTOR complex 1.

In certain embodiments the mTOR inhibitors also inhibit other mTOR-mediated signaling pathways, and may serve also as inhibitors of, e.g., phosphoinositide 3-kinase (PI3K). Exemplary PI3K/mTOR inhibitors include BTG226, gedatolisib, apitolisib, omipalisib, dactolisib, duvelisib, and idelalisib can be used in lieu of or in addition to mTOR inhibitors. Inhibitors of Akt (Protein Kinase B) such as 8-[4-(1-aminocyclobutyl)phenyl]-9-phenyl-2H-[1,2,4]triazolo[3,4-f][1,6]na-phthyridin-3-one; dihydrochloride (MK-2206) also can be used in lieu of or in addition to mTOR inhibitors.

CDK Inhibitors

In certain embodiments, the anti-PLESC agent is a CDK inhibitor.

Non-limiting examples of CDK inhibitors include SNS-032 (BMS-387032). Other CDK inhibitors include SB1317, AUZ 454, NU6300, CDK2-IN-4, BGG463, Desmethylglycitein, Dinaciclib, Abemaciclib, Seliciclib, Flavopiridol, Ro-3306, PF-06873600, CYC065, AT7519, CT7001 hydrochloride, AZD-5438, JNJ-7706621, GSK 3 Inhibitor IX, PHA-767491 hydrochloride, CVT-313, Milciclib, THZ2, Purvalanol A, LDC000067, BMS-265246, Flavopiridol Hydrochloride, Atuveciclib, R547, PHA-793887, SU9516, CDKI-73, AT7519 Hydrochloride, MC180295, FN-1501, Roniciclib, Lerociclib dihydrochloride, Purvalanol B, AMG 925, JSH-150, FMF-04-159-2, Bisindolylmaleimide×hydrochloride, Riviciclib hydrochloride, (R)—CR8, NU6140, Alsterpaullone, AMG 925 HCl, NU2058, (R)—CR8 trihydrochloride, AT7519 trifluoroacetate, PHA-767491, BS-181 hydrochloride, NVP-LCQ195, CDK9-IN-9, IIIM-290, Riviciclib, PROTAC CDK2/9 Degrader-1, CDK12-IN-E9, Lerociclib and Indirubin-5-sulfonate In one embodiment, the CDK2 inhibitor is selected from the group consisting of: Milciclib, Dinaciclib, AG-024322, 2-Hydroxybohemine, Aloisine A, AZD 5438, BMS-265246, Butyrolactone I, CDK2 Inhibitor II (CAS #222035-13-4), CDK2 Inhibitor IV, NU6140 (CAS #444723-13-1), CYC-065, NU2058 (CAS #161058-83-9), NU6102 (CAS #444722-95-6), Olomoucine, PHA-793887 (CAS #718630-59-2), Roscovitine (seliciclib), SCH 900776 (CAS #891494-63-6), SNS-032 (BMS-387032), SU9516 (CAS #377090-84-1), WHI-P180 (CAS #21 1555-08-7), or any pharmaceutically acceptable salt thereof, and any combination thereof.

In one embodiment, the CDK2 inhibitor is milciclib or a pharmaceutically acceptable salt thereof.

HDAC Inhibitors (HDACi)

In one embodiment, the HDACi is suberoylanilide hydroxamic acid (SAHA), or a derivative thereof. In this embodiment, SAHA inhibits fibroblast migration and activation. In another aspect of this embodiment, myofibroblast formation is inhibited. In a particular embodiment, myofibroblast formation is inhibited while preserving cell viability.

In other embodiments, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (C1994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

In certain embodiments, the HDAC inhibitor is selected from vorinostat, panobinostat, valproic acid, phenylbutyrate, entinostat, CI-994, mocetinostat, dacinostat, givinostat, belinostat, pivanex or SB93.

RAR Agonists

In another aspect, the agent is an agonist of a retinoic acid receptor (RAR), and preferably a pan-RAR agonist. Known RAR agonists include but are not limited to, TTNPB (4-[(E)-2-(5,6,7,8-Tetrahydro-5,5,8,8-tetramethyl-2-naphthalenyl)-1-propenyl]benzoic acid), tamibarotene, 9-cis-retinoic acid (alitretinoin), all-trans-retinoic acid (tretinoin), AGN193836, Ro 40-6055, CD666, and BMS753.

isotretinoin, AC261066, AC55649, adapalene, AM580, AM80, BMS961, CD1530, CD2314, CD437, tazarotene, Tazarotenic acid, bexarotene, MDI 301, R667, 9-cis UAB30, LG100268, LGD1069, BMS 270394, BMS 189961, CH 55, LE 135, AM 580, 9CDHRA, Acitretin, AM-580, BMS-453, BMS-493, BMS-753, BMS-961, CD-1530, CD-2314, CD-437, Ch-55, EC 19, EC 23, Etretinate, Fenretinide, Isotretinoin, Palovarotene, and Retinol (vitamin A).

In certain embodiments, the RAR agonist also includes RXR agonist activity.

To further illustrate, the RAR agonist can be

| Name | Specificity | Structure |
|---|---|---|
| Tretinoin | Pan-RAR agonist | 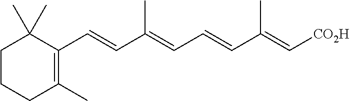 [SMILES: CC1=C(/C=C/C(C)=C/C=C/C(C)=C/C(=O)O)C(C)(C)CCC1] |
| 9-cis RA | Pan-RAR and RXR agonist | 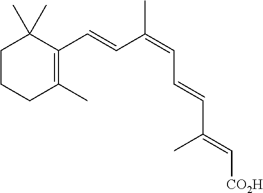 [SMILES: CC1=C(/C=C/C(C)=C\C=C\C(C)=C\C(=O)O)C(C)(C)CCC1] |
| 13-cis-RA | Pan-RAR agonist | 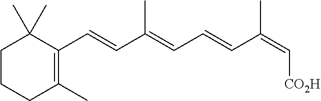 [SMILES: CC1=C(/C=C/C(C)=C/C=C/C(C)=C\C(=O)O)C(C)(C)CCC1] |
| Fenretinide | RAR agonist | 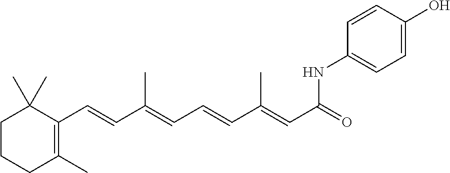 [SMILES: CC1=C(/C=C/C(C)=C/C=C/C(C)=C/C(=O)Nc2ccc(O)cc2)C(C)(C)CCC1] |
| EC 23 | Pan-RAR agonist | 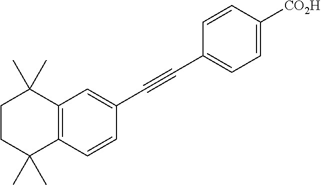 [SMILES: CC1(C)CCC(C)(C)c2cc(C#Cc3ccc(C(=O)O)cc3)ccc21] |
| TTNPB | Pan-RAR agonist | 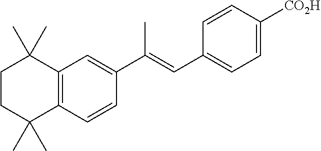 [SMILES: C/C(=C\c1ccc(C(=O)O)cc1)c1ccc2c(c1)C(C)(C)CCC2(C)C] |
| Ch 55 | Pan-RAR agonist | 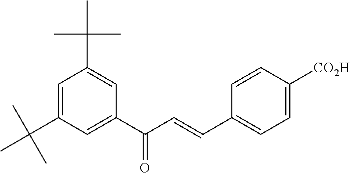 [SMILES: CC(C)(C)c1cc(C(=O)/C=C/c2ccc(C(=O)O)cc2)cc(C(C)(C)C)c1] |
| Tazarotene | RARB/γ agonist | 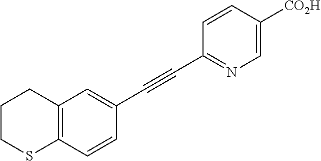 [SMILES: O=C(O)c1ccc(C#Cc2ccc3c(c2)CCCS3)nc1] |
| BMS 753 | RARα agonist | 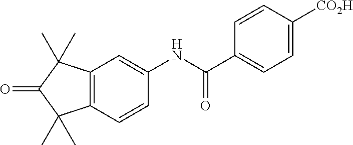 [SMILES: CC1(C)C(=O)C(C)(C)c2cc(NC(=O)c3ccc(C(=O)O)cc3)ccc21] |
| AM80 | RARα agonist | 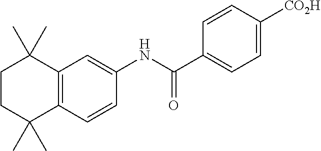 [SMILES: CC1(C)CCC(C)(C)c2cc(NC(=O)c3ccc(C(=O)O)cc3)ccc21] |
| AM580 | RARα agonist | 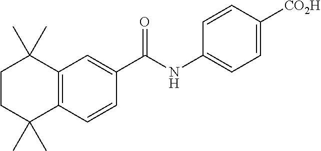 [SMILES: CC1(C)CCC(C)(C)c2cc(C(=O)Nc3ccc(C(=O)O)cc3)ccc21] |
| AC55649 | RARβ2 agonist | 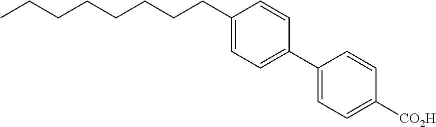 [SMILES: CCCCCCCCc1ccc(-c2ccc(C(=O)O)cc2)cc1] |
| AC261066 | RARβ2 agonist | 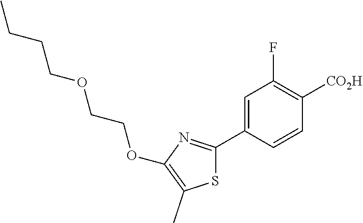 [SMILES: CCCCOCCOc1nc(-c2ccc(C(=O)O)c(F)c2)sc1C] |
| Adapalene | RARβ and γ agonist | 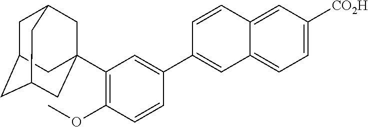 [SMILES: COc1ccc(-c2ccc3cc(C(=O)O)ccc3c2)cc1C12CC3CC(CC(C3)C1)C2] |
| CD437 | RARγ agonist | 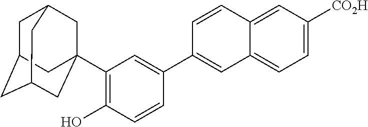 [SMILES: O=C(O)c1ccc2cc(-c3ccc(O)c(C45CC6CC(CC(C6)C4)C5)c3)ccc2c1] |
| CD1530 | RARγ agonist | 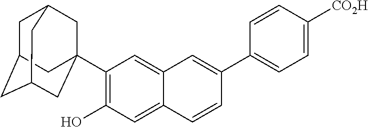 [SMILES: O=C(O)c1ccc(-c2ccc3cc(O)c(C45CC6CC(CC(C6)C4)C5)cc3c2)cc1] |
| CD2665 | RARγ agonist | 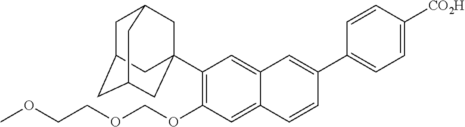 [SMILES: COCCOCOc1cc2ccc(-c3ccc(C(=O)O)cc3)cc2cc1C12CC3CC(CC(C3)C1)C2] |
| MM11253 | RAR agonist | 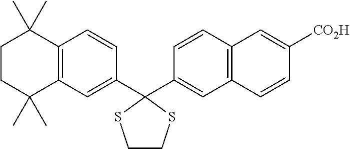 [SMILES: CC1(C)CCC(C)(C)c2cc(C3(c4ccc5cc(C(=O)O)ccc5c4)SCCS3)ccc21] |
| LE135 | RARβ agonist | 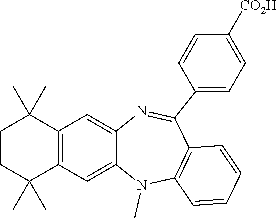 [SMILES: CN1c2cc3c(cc2N=C(c2ccc(C(=O)O)cc2)c2ccccc21)C(C)(C)CCC3(C)C] |

Proteasome Inhibitors

In still another aspect, the agent is a proteasome inhibitor, preferably an immunoproteasome inhibitor.

The proteasome inhibitor may be any proteasome inhibitor known in the art. In particular, it is one of the proteasome inhibitors described in more detail in the following paragraphs.

Exemplary proteasome inhibitors include bortezomib, carfilzomib, ixazomib, oprozomib, marizomib, CEP-18770, disulfiram, epigallocatechin-3-gallate, epoxomicin, lactacystin, MG132, MLN9708, ONX 0912, PR-924, PR-957, KZR-504, LMP7-IN-1, salinosporamide A, epoxomycine, eponemycine, aclacinomycine A (aclarubicine), celastrol, withaferin A, Gliotoxin, epipolythiodioxo-piperazines, green tea polyphenolic catechins (−)-epigallocatechin-3-gallate, Disulfuram, acridine derivatives, tetra-acridine derivatives with betulinic acid, as 3′,3′-dimethylsuccinyl betulinic acid, dihydroeponemycin analogs, PR39, PR11, argyrin A, Tyropeptin A, TMC-86, TMC-89 calpain inhibitor I, Mal-β-Ala-Val-Arg-al, fellutamide B, syringolin A, glidobactin A, syrbactins, TMC-95 family of cyclic tripeptides, TMC-95A, TMC-95A endocyclic oxindole-phenyl clamp (BIA-la) derivatives, TMC-95A endocyclic biphenyl-ether clamp (BIA-2a) derivatives, lactacystine, Omuralide, Homobelactosin C, Salinosporamide A, NEOSH-101, CEP-18770, IPS1001, IPS1007, MLN2238, MLN9708, ONX 0914, AA-102, 26 S PI, AVR-147, 4E12, N-carbobenzoxy-L-leucinyl-L-leucinyl-1-leucinal and its boronic acid derivative, N-carbobenzoxy-Leu-Leu-Nva-H, N-acetyl-L-leuzinyl-L-leuzinyl-L-norleuzinal, N-carbobenzoxy-Ile-Glu(Obut)-Ala-Leu-H, Ac-Leu-Leu-Nle-H, Ac-Arg-Val-Arg-H, carbobenzoxy-L-leucinyl-L-leucinyl-L-leucin-vinyl sulfone, 4-hydroxy-5-iodo-3-nitrophenylacetyl-L-leucinyl-L-leucinyl-L-leucin-vinyl-sulfone, Ac-Pro-Arg-Leu-Asn-vinyl-sulfone, pyrazyl-CONH(CHPhe)CONH(CHisobutyl)B(OH)2, pyrazyl-2,5-bis-CONH(CHPhe)CONH(CHisobutyl)-B(OH)2, Benzoyl(Bz)-Phe-boroLeu, Ph-acetyl-Leu-Leu-boroLeu, Cbz-Phe-boroLeu, benzyloxycarbonyl(CbZ)-Leu-Leu-boroLeu-pinacol-ester, (1R-[1S, 4R,5S]]-1-(1-hydroxy-2-methylpropyl)-4-propyl-6-oxa-2-azabicyclo[3.2.0]heptanes-3,7-dione, (Morpholin-CONH—(CH-napthyl)-CONH—(CH-isobutyl)-B(OH)2 and its enantiomer PS-293, 8-quinolyl-sulfonyl-CONH—(CH-napthyl)-CONH(—CH-isobutyl)-B(OH)2, NH2(CH-Napthyl)-CONH—(CH-isobutyl)-B(OH)2, morpholino-CONH—(CH-napthyl)-CONH—(CH-phenylalanine)-B(OH)2, CH3-NH—(CH-napthyl-CONH—(CH-isobutyl)-B(OH)2, 2-quinole-CONH—(CH-homo-phenylalanin)-CONH—(CH-isobutyl)-B(OH)2, Phenyalanine-CH2-CH2-CONH—(CH-phenylalanine)-CONH—(CH-isobutyl)1-B(OH)2, “PS-383” (pyridyl-CONH—(CHpF-phenylalanine)-CONH—(CH-isobutyl)-B(OH)2, (PEG)19-25-Leu-Leu-Nle-H, (PEG)19-25-Arg-Val-Arg-H, H-Nle-Leu-Leu-(PEG)19-25-Leu-Leu-Nle-H, H-Arg-Val-Arg-(PEG)19-25-Arg-Val-Arg-H ZLLL-vs), ZLLVS, YLVS, MG-262, ALLnL, ALLnM, LLnV, DFLB Ada-(Ahx)3-(Leu)3-vs, YU101 (Ac-hFLFL-ex), MLN519 and S-2209.

To further illustrate, in certain embodiments suitable proteasome inhibitors for use in combinations described herein include (a) peptide boronates, such as bortezomib (also known as Velcade™ and PS341), delanzomib (also known as CEP-18770), ixazomib(also known as MLN9708) or ixazomib citrate; (b) peptide aldehydes, such as MG132 (Z-Leu-Leu-Leu-H), MG115 (Z-Leu-Leu-Nva-H), IPSI 001, fellutamide B, ALLN (Ac-Leu-Leu-N1e-H, also referred to as calpain inhibitor I), and leupeptin (Ac-Leu-Leu-Arg-al); (c) peptide vinyl sulfones, (d) epoxyketones, such as epoxomicin, oprozomib (also referred to as PR-047 or ONX 0912), PR-957 (also known as ONX 0914), and carfilzomib (also referred to as PR-171); and (e) β-lactones, such as lactacystin, omuralide, salinosporamide A (also known as NPI-0052 and marizomib), salinosporamide B, belactosines, cinnabaramides, polyphenols, TMC-95, and PS-519.

In certain preferred embodiments, the proteasome inhibitor is a boronic acid class inhibitor, i.e., such as a peptide borinic acid, such as a dipeptide or tripeptide boronic acid.

In certain embodiments, the proteasome inhibitor is bortezomib, also known as VELCADE and PS341. In a preferred embodiment, the proteasome inhibitor is [(1R)-3-methyl-1-[[(2S)-3-phenyl-2-(pyrazine-2-carbonylamino)propanoyl]amino]butyl]boronic acid. In a preferred embodiment, the proteasome inhibitor is the compound of Formula:

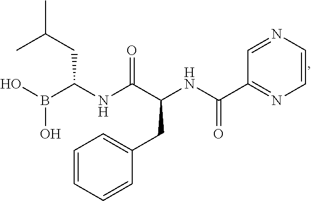 [SMILES: CC(C)C[C@H](NC(=O)[C@H](Cc1ccccc1)NC(=O)c1cnccn1)B(O)O]

or a pharmaceutically acceptable salt, solvate, hydrate, cocrystal, or prodrug thereof.

In certain embodiments, the proteasome inhibitor is delanzomib, also known as CEP-18770 or [(1R)-1-[[(2S,3R)-3-hydroxy-2-[(6-phenylpyridine-2-carbonyl)amino]butanoyl]amino]-3-methylbutyl]boronic acid. In a preferred embodiment, the proteasome inhibitor is the compound of Formula:

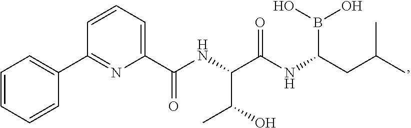 [SMILES: CC(C)C[C@H](NC(=O)[C@@H](NC(=O)c1cccc(-c2ccccc2)n1)[C@@H](C)O)B(O)O]

or a pharmaceutically acceptable salt, solvate, hydrate, cocrystal, or prodrug thereof.

In certain embodiments, the proteasome inhibitor is ixazomib, also known as MLN-9708 or ixazomib citrate or 4-(carboxymethyl)-24(R)-1-(2-(2,5-dichlorobenzamido)acetamido)-3-methylbutyl)-6-oxo-1,3,2-dioxaborinane-4-carboxylic acid, or a pharmaceutically acceptable salt, solvate, hydrate, cocrystal, or prodrug thereof.

In certain embodiment, the proteasome inhibitor is 1,3,2-dioxaborolane-4,4-diacetic acid, 2-[(1R)-1-[[2-[(2,5-dichlorobenzoyl)amino] acetyl] amino]-3-methylbutyl]-5-oxo-, or a pharmaceutically acceptable salt, solvate, hydrate, cocrystal, or prodrug thereof.

In a preferred embodiment, the proteasome inhibitor is 2,2′-{2-[(1R)-1-{[N-(2,5-dichlorobenzoyl)glycyl]amino}-3-methylbutyl]-5-oxo-1,3,2-dioxaborolane-4,4-diyl}diacetic acid, or a pharmaceutically acceptable salt, solvate, hydrate, cocrystal, or prodrug thereof. In a preferred embodiment, the proteasome inhibitor is the compound of Formula:

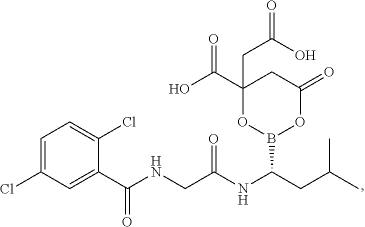 [SMILES: CC(C)C[C@H](NC(=O)CNC(=O)c1cc(Cl)ccc1Cl)B1OC(=O)CC(CC(=O)O)(C(=O)O)O1]

or a pharmaceutically acceptable salt, solvate, hydrate, cocrystal, or prodrug thereof.

In certain embodiments, the proteasome inhibitor is 1B-{(1R)-1-[2-(2,5-dichlorobenzamido)acetamido]-3-methylbutyl}boronic acid, or a pharmaceutically acceptable salt, solvate, hydrate, cocrystal, or prodrug thereof. In a preferred embodiment, the proteasome inhibitor is the compound of Formula:

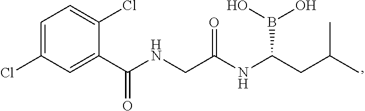 [SMILES: CC(C)C[C@H](NC(=O)CNC(=O)c1cc(Cl)ccc1Cl)B(O)O]

or a pharmaceutically acceptable salt, solvate, hydrate, cocrystal, or prodrug thereof.

In certain embodiments, the proteasome inhibitor is marizomib, also known as NPI-0052 and Salinosporamide A or (4R,5S)-4-(2-chloroethyl)-1-((1S)-cyclohex-2-enyl(hydroxy)methyl)-5-methyl-6-oxa-2-azabicyclo[3.2.0]heptane-3,7-dione, or a pharmaceutically acceptable salt, solvate, hydrate, cocrystal, or prodrug thereof. In a preferred embodiment, the proteasome inhibitor is the compound of Formula:

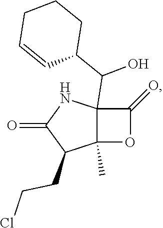 [SMILES: C[C@@]12OC(=O)C1(C(O)[C@@H]1C=CCCC1)NC(=O)[C@@H]2CCCl]

or a pharmaceutically acceptable salt, solvate, hydrate, cocrystal, or prodrug thereof.

In certain preferred embodiments, the proteasome inhibitor is an epoxyketone class inhibitor, i.e., such as an peptide epoxyketone, such as a tetrapeptide epoxyketone or tripeptide epoxyketone, and may be an an analog of epoxomicin.

In one embodiment, the protoeasome inhibitor is carfilzomib, also known as PX-171-007, or (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4 phenylbutanamido)-4-methylpentanamide, or a pharmaceutically acceptable salt, solvate, hydrate, cocrystal, or prodrug thereof. In certain embodiments, the proteasome inhibitor is the compound of Formula:

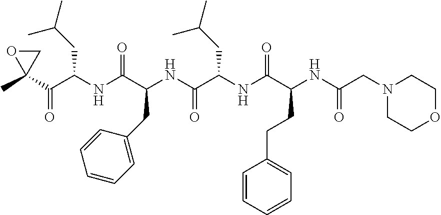 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

In certain embodiments, the proteasome inhibitor is oprozimib, also known as PR-047 or ONX 0912, or N-[(2S)-3-methoxy-1-[[(2S)-3-methoxy-1-[[(2S)-1-[(2R)-2-methyloxiran-2-yl]-1-oxo-3-phenylpropan-2-yl]amino]-1-oxopropan-2-yl]amino]-1-oxopropan-2-yl]-2-methyl-1,3-thiazole-5-carboxamide, or a pharmaceutically acceptable salt, solvate, hydrate, cocrystal, or prodrug thereof. In certain embodiments, the proteasome inhibitor is the compound of Formula:

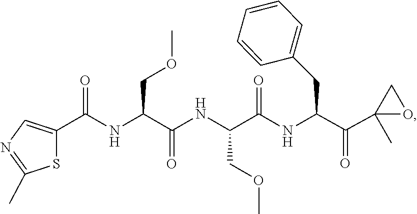 [SMILES: COC[C@H](NC(=O)c1cnc(C)s1)C(=O)N[C@@H](COC)C(=O)N[C@@H](Cc1ccccc1)C(=O)C1(C)CO1]

In certain preferred embodiments, the epoxyketone is an immunoproteasome inhibitor, i.e., is inhibitor of βSi/LMP7, and even more preferably is a selective inhibitor of βSi/LMP7.

In certain embodiments, the proteasome inhibitor is oprozimib, also known as PR-957 or ONX 0914, or (2S)-3-(4-methoxyphenyl)-N-[(2S)-1-(2-methyloxiran-2-yl)-1-oxo-3-phenylpropan-2-yl]-2-[[(2S)-2-[(2-morpholin-4-ylacetyl)amino]propanoyl]amino]propanamide, or is a pharmaceutically acceptable salt, solvate, hydrate, cocrystal, or prodrug thereof. In certain embodiments, the proteasome inhibitor is the compound of Formula:

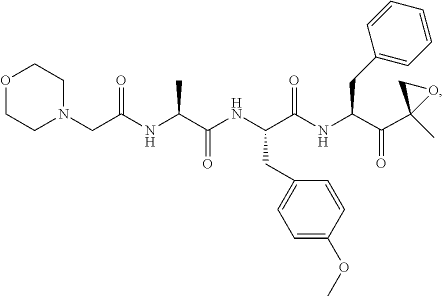 [SMILES: COc1ccc(C[C@H](NC(=O)[C@H](C)NC(=O)CN2CCOCC2)C(=O)N[C@@H](Cc2ccccc2)C(=O)[C@]2(C)CO2)cc1]

Other exemplary immunoproteasome inhibitors include:

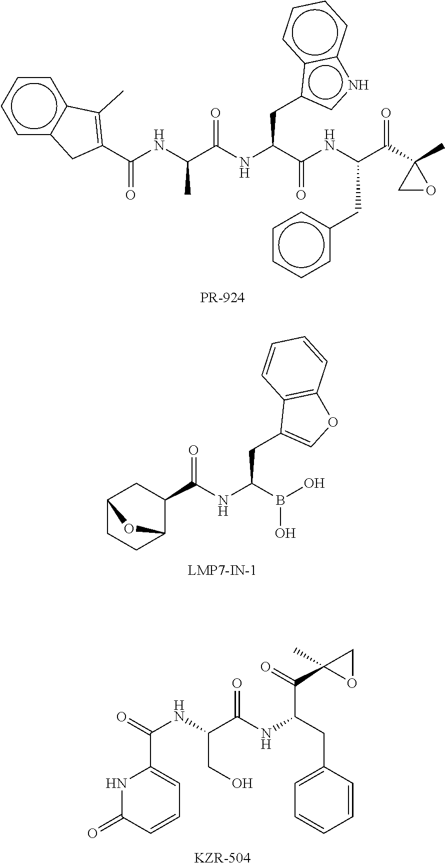

Aryl Hydrocarbon Receptor Antagonists

In still another aspect, the agent is an aryl hydrocarbon receptor (AHR) antagonist.

For instance, the aryl hydrocarbon receptor antagonist can be StemRegenin-1, CH-223191, PD98059, GNF351, BAY-2416964, PDM-11, PDM2, Perillaldehyde, BAY-218, retusin-7-methylether, UM0125464, 3-chloro-N-(2,3-dihydro-1,4-benzodioxin-6-yl)-1-benzithiophene-2-carboxamide, chrysin, kaempferide, 5-methoxyflavone, or N-methyl-p-carboline-3-carboxamide.

In certain aspects, the aryl hydrocarbon receptor antagonist is StemRegenin-1 (SR-1) (4-(2-(2-(benzo[b]thiophen-3-yl)-9-isopropyl-9H-purin-6-ylamino)ethyl)phenol).

In certain aspects, the aryl hydrocarbon receptor antagonist is the compound CH223191 (1-Methyl-N-[2-methyl-4-[2-(2-methylphenyl)diazenyl]phenyl-1H-pyrazole-5-carboxamide].

Multiple Ion Channel Blockers

In still another aspect, the agent is a multiple ion channel blocker, and preferably is a Class III agent, and more preferably is a potassium channel blocker. Exemplary multiple ion channel blockers include amiodarone, dronedarone, budiodarone, vernakalant, celivarone, and AZD1305.

For instance, the xxx can be Amiodarone a structural analog thereof, such as dronedarone, i.e., N-(2-Butyl-3-(p-(3-(dibutylamino)propoxy)benzoyl)-5-benzofuranyl)methane-sulfonamide.

EGFR Inhibitor

In certain embodiments, the agent is a receptor tyrosine kinase inhibitor, and is preferably an EGFR inhibitor, or an ErbB kinase inhibitor (such as an ErbB1, ErbB2, ErbB3 or ErbB4 inhibitor) or a dual EGFR/ErbB inhibitor.

Exemplary EGFR inhibitors/antagonists include, inter alia, small-molecule EGFR inhibitors/antagonists, such as gefitinib, erlotinib, lapatinib, afatinib (also referred to as BIBW2992), neratinib, ABT-414, dacomitinib (also referred to as PF-00299804), AV-412, PD 153035, vandetanib, PKI-166, pelitinib (also referred to as EKB-569), canertinib (also referred to as CI-1033), icotinib, poziotinib (also referred to as NOV120101), BMS-690514, CUDC-101, AP26113, XL647, AZD9291, CO-1686 (rotsiletinib), WZ4002, PF 00299804,BDTX-189, mavelertinib, JBJ-04-125-02, AG-490, tucatinib, genistein, pyrotinib, sapitinib, mobocertinib, AZ-5104, mubritinib, zorifertinib, rociletinib, lazertinib, lifirafenib, butein, PD168393, PD153035, daphnetin, tarloxtinib, and icotinib.

WZ8040 is a novel mutant-selective irreversible EGFRT790M inhibitor, does not inhibit ERBB2 phosphorylation (T7981).

In certain embodiments, the agent is an EGFR tyrosine kinase inhibitor (EGFR-TKI). Exemplary EGFR-TKI include afatinib, erlotinib, gefitinib, icotinib, neratinib, dacomitinib and osimertinib.

In certain embodiments, the EGFR tyrosine kinase inhibitor is erlotinib.

In certain embodiments, the agent is a selective ErbB1 (Her1) inhibitor, e.g., is at least 2, 5, 10, 50, 100 or even 1000 more potent an inhibitor of ErbB1 relative to ErbB2 (HER2), ErbB3 (HER3) or ErbB4 (HER4). Exemplary selective ErbB1 inhibitors include erlotinib, gefitinib, saracatinib, WZ4002, AG-1478 (Tyrphostin AG-1478), PD153035, OSI-420, WZ3146, Rociletinib (CO-1686), WHI-P154, JND3229, PD153035, AZD3759, Erlotinib (OSI-774), Osimertinib (AZD9291) or PD168393.

In certain preferred embodiments, the EGFR Inhibitor is PD168393, e.g., having the structure

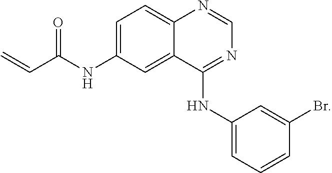 [SMILES: C=CC(=O)Nc1ccc2ncnc(Nc3cccc(Br)c3)c2c1]

IAP Inhibitor

IAP (Inhibitor of apoptosis) proteins, a family of anti-apoptotic proteins, have an important role in evasion of apoptosis, as they can both block apoptosis-signaling pathways and promote survival. Eight members of this family have been described in humans (BIRC1/NAIP, BIRC2/cIAP1, BIRC3/cIAP2, BIRC4/XIAP, BIRC5/Survivin, BIRC6/Apollon, BIRC7/ML-IAP and BIRC8/ILP2). In certain embodiments, the agent is an IAP Inhibitor (i.e., an IAP Antagonist). Exemplary IAP Inhibitors include XIAP inhibitors, CIAP inhibitors, and agents acting as dual XIAP and CIAP inhibitors.

Exemplary IAP inhibitors and antagonists include Birinapant (a bivalent Smac mimetic, which is a potent antagonist for XIAP and cIAP1 with Kds of 45 nM and less than 1 nM, respectively), LCL161
Inhibitor (an IAP inhibitor which inhibits XIAP and cIAP1 with IC₅₀s of 35 and 0.4 nM), AZD5582 (AZD5582 an IAP antagonist which binds to the BIR3 domains cIAP1, cIAP2, and XIAP), SM-164 (a cell-permeable Smac mimetic compound that binds to XIAP protein containing both the BIR2 and BIR3 domains with an IC₅₀ value of 1.39 nM and functions as an extremely potent antagonist of XIAP), BV6 (an antagonist of cIAP1 and XIAP), Xevinapant (or AT-406, is a potent and orally bioavailable Smac mimetic and an antagonist of IAPs, and it binds to XIAP, cIAP1, and cIAP2 proteins), GDC-0152 (a potent IAPs inhibitor, and binds to the BIR3 domains of XIAP, cIAP1, cIAP2 and the BIR domain of ML-IAP), ASTX660 (an orally bioavailable dual antagonist of cIAPs and XIAPs), CUDC-427 (a potent second-generation pan-selective IAP antagonist), Embelin (or Embelic acid, a potent, nonpeptidic XIAP inhibitor). APG-1387 (a bivalent SMAC mimetic and an IAP antagonist, blocks the activity of IAPs family proteins (XIAP, cIAP-1, cIAP-2, and ML-IAP), MX69 (an inhibitor of MDM2/XIAP), MV1, Polygalacin D, UC-112, AZD5582 dihydrochloride, HY-125378m Tolinapant (ASTX660) and SBP-0636457.

In certain embodiments, the IAP inhibitor is a selective XIAP inhibitor (having an IC₅₀ for XIAP inhibition at least 10-fold less than the IC₅₀ for CIAP inhibition, and more preferably at least 20. 50 or 100-fold less), such as SM-164.

Inhibiting Expression of Anti-PLESC Targets

In addition to using small molecule inhibitors of the anti-PLESC targets above, another aspect of the disclosure relates to the use of the nucleic acid therapeutics to reduce or inhibit the expression of the target of the anti-PLESC drug (“anti-PLESC Gene Target”), such as to inhibit expression of HSP90, HSP70, mTOR, RAR, proteaseome or immunoprotease subunits, or a combination thereof. Eemplary nucleci acid therapeutics can include antisense therapy or RNA intereference therapy (such as small interfering RNA (siRNA), micro RNA (miRNA) or short-hairpin RNA (shRNA)), a sequence-directed ribozyme or gene inactivating CRISPR RNA (crRNA).

As used herein, antisense therapy refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. The binding may be by conventional base pair complementarity, or, for example, in the case of binding to DNA duplexes, through specific interactions in the major groove of the double helix. In general, antisense therapy refers to the range of techniques generally employed in the art and includes any therapy which relies on specific binding to oligonucleotide sequences.

An antisense construct of the present disclosure can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell, causes inhibition of expression by hybridizing with the mRNA and/or genomic sequences of a subject nucleic acid. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6:958-976 (1988); and Stein et al., Cancer Res. 48:2659-2668 (1988). With respect to antisense DNA, oligodeoxyribonucleotides derived from the translation initiation site, e.g., between the −10 and +10 regions of the nucleotide sequence of interest, are preferred.

Antisense approaches involve the design of oligonucleotides (either DNA or RNA) that are complementary to mRNA. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, Nature 372:333 (1994)). Therefore, oligonucleotides complementary to either the 5′ or 3′ untranslated, non-coding regions of a gene could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are typically less efficient inhibitors of translation but could also be used in accordance with the disclosure. Whether designed to hybridize to the 5, 3, or coding region of subject mRNA, antisense nucleic acids should be at least six nucleotides in length, and are preferably less that about 100 and more preferably less than about 50, 25, 17 or 10 nucleotides in length.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al., Proc. Natl. Acad. Sci. U.S.A. 93:14670 (1996) and in Eglom et al., Nature 365:566 (1993). One advantage of PNA oligomers is their capability to bind to complementary DNA essentially independently from the ionic strength of the medium due to the neutral backbone of the DNA.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an—anomeric oligonucleotide. An—anomeric oligonucleotide forms specific double-stranded hybrids with complementary RNA in which, contrary to the usual—units, the strands run parallel to each other (Gautier et al., Nucl. Acids Res. 15:6625-6641 (1987)). The oligonucleotide is a 2-O-methylribonucleotide (Inoue et al., Nucl. Acids Res. 15:6131-12148 (1987)), or a chimeric RNA-DNA analogue (Inoue et al., FEBS Lett. 215:327-330 (1987)).

The antisense molecules can be delivered to cells which express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

In another aspect of the disclosure, ribozyme molecules designed to catalytically cleave target mRNA transcripts corresponding to one or more anti-PLESC Gene Target can be used to prevent translation of target mRNA and expression of a target protein by the COPD or IPF stem cell or its progeny (See, e.g., PCT International Publication WO90/11364; Sarver et al., Science 247:1222-1225 (1990) and U.S. Pat. No. 5,093,246). While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5-UG-3. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target mRNA; i.e., to increase efficiency and minimize the intracellular accumulation of non-functional mRNA transcripts.

The ribozymes of the present disclosure also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., Science, 224:574-578 (1984); Zaug and Cech, Science, 231:470-475 (1986); Zaug, et al., Nature, 324:429-433 (1986); published International patent application No. WO88/04300; Been and Cech, Cell, 47:207-216 (1986)). The Cech-type ribozymes have an eight base pair active site which hybridizes to a target RNA sequence whereafter cleavage of the target RNA takes place. The disclosure encompasses those Cech-type ribozymes which target eight base-pair active site sequences that are present in a target anti-PLESC Gene Target.

As in the antisense approach, the ribozymes can be composed of modified oligonucleotides (e.g., for improved stability, targeting, etc.) and should be delivered to cells which express the target anti-PLESC Gene Target in vivo. A preferred method of delivery involves using a DNA construct “encoding” the ribozyme under the control of a strong constitutive pol III or pol II promoter, so that transfected cells will produce sufficient quantities of the ribozyme to destroy endogenous messages and inhibit translation. Because ribozymes, unlike antisense molecules, are catalytic, a lower intracellular concentration is required for efficiency.

Antisense RNA, DNA, RNA Interference constructs and ribozyme molecules of the disclosure may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In other embodiments, the nucleic acid is a a “decoy” nucleic acid which corresponds to a transciptional regulatory sequence and binds to a transcription factor that is involved in upregulated expression of one or more genes in an COPD Stem Cell population or IPF Stem Cell Population. The decoy nucleic acid therefore competes with natural binding target for the binding of the transcription factor and acts an antagonist to reduce the expression of those genes under the transcriptional control of the targeted transcription factor.

Increased efficiency can also be gained through other techniques, such as in which delivery of the therapeutic nucleic acid is improved by use of chemical carriers-cationic polymers or lipids- or via a physical approach-gene gun delivery or electroporation. See Tranchant et al., (2004) “Physicochemical optimisation of plasmid delivery by cationic lipids” J. Gene Med., 6 (Suppl. 1):S24-S35; and Niidome et al., (2002) “Gene therapy progress and prospects: nonviral vectors” Gene Ther., 9:1647-1652. Electroporation is especially regarded as an interesting technique for nonviral gene delivery. Somiari, et al., (2000) “Theory and in vivo application of electroporative gene delivery” Mol. Ther. 2:178-187; and Jaroszeski et al., (1999) “In vivo gene delivery by electroporation” Adv. Drug Delivery Rev., 35:131-137. With electroporation, pulsed electrical currents are applied to a local tissue area to enhance cell permeability, resulting in gene transfer across the membrane. Research has shown that in vivo gene delivery can be at least 10-100 times more efficient with electroporation than without. See, for example, Aihara et al., (1998) “Gene transfer into muscle by electroporation in vivo” Nat. Biotechnol. 16:867-870; Mir, et al., (1999) “High-efficiency gene transfer into skeletal muscle mediated by electric pulses” PNAS 96:4262-4267; Rizzuto, et al., (1999) “Efficient and regulated erythropoietin production by naked DNA injection and muscle electroporation” PNAS 96: 6417-6422; and Mathiesen (1999) “Electropermeabilization of skeletal muscle enhances gene transfer in vivo” Gene Ther., 6:508-514.

The therapeutic nucleic acids of the present disclosure can be delivered by a wide range of gene delivery system commonly used for gene therapy including viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ.

### IV. Normal Pulmonary Stem Cell Promoters

The inventors have also observed that certain of the drug agents they screened were able to selectively promote the proliferation and regenerative capabilities of normal pulmonary stem cells, relative to COPD or IPF stem cells, i.e., are Pulmonary Regenerative Agents.

### BCR-ABL Kinase Inhibitor

In certain embodiments, the Pulmonary Regenerative Agent is a tyrosine kinase inhibitor, preferably an ABL1 Kinase Inhibitor, and more preferably is a BCR-ABL Kinase inhibitor. Examples of BCR-ABL tyrosine kinase inhibitors include imatinib, dasatinib, nilotinib, bosutinib, ponatinib, bafetinib, saracatinib, tozasertib and rebastinib.

| Drug | Structure |
|---|---|
| Imatinib (STI571) | 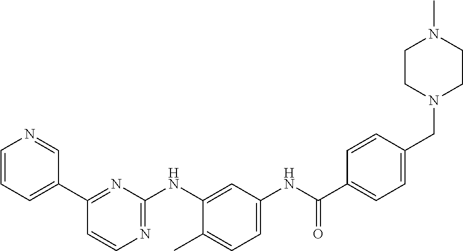 [SMILES: Cc1ccc(NC(=O)c2ccc(CN3CCN(C)CC3)cc2)cc1Nc1nccc(-c2cccnc2)n1] |
| Nilotinib (AMN107) | 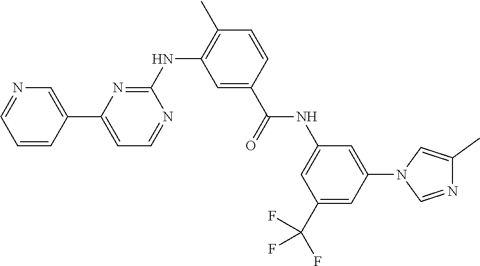 [SMILES: Cc1cn(-c2cc(NC(=O)c3ccc(C)c(Nc4nccc(-c5cccnc5)n4)c3)cc(C(F)(F)F)c2)cn1] |
| Dasatinib (BMS-345825) | 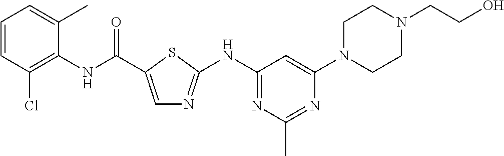 [SMILES: Cc1nc(Nc2ncc(C(=O)Nc3c(C)cccc3Cl)s2)cc(N2CCN(CCO)CC2)n1] |
| Bosutinib (SKI-606) | 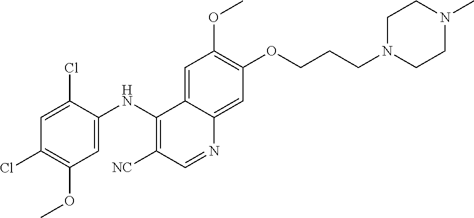 [SMILES: COc1cc(Nc2c(C#N)cnc3cc(OCCCN4CCN(C)CC4)c(OC)cc23)c(Cl)cc1Cl] |
| Ponatinib (AP-24534) | 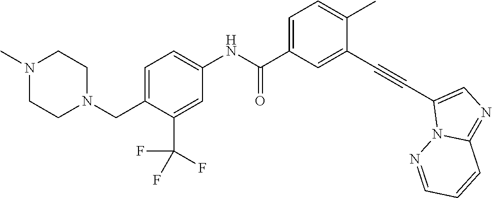 [SMILES: Cc1ccc(C(=O)Nc2ccc(CN3CCN(C)CC3)c(C(F)(F)F)c2)cc1C#Cc1cnc2cccnn12] |
| Bafetinib (INNO-406) | 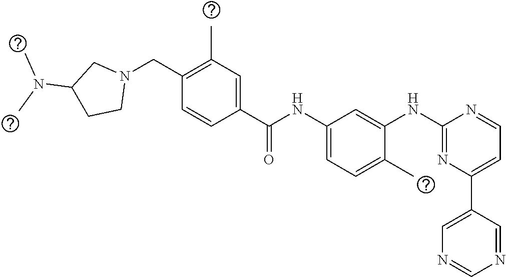 [SMILES: Cc1cc(C(=O)Nc2ccc(C)c(Nc3nccc(-c4cncnc4)n3)c2)ccc1CN1CCC(N(C)C)C1] |

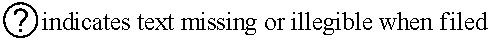

FLT3 Inhibitors

In certain embodiments, the ESO Regenerative agent is a FLT3 inhibitor. Exemplary FLT3 inhibitors to be used herein are quizartinib (AC220), crenolanib (CP-868596), midostaurin (PKC-412), lestaurtinib (CEP-701), 4SC-203, TTT-3002, sorafenib (Bay-43-0006), Ponatinib (AP-24534), sunitinib (SU-11248), and/or tandutinib (MLN-0518), or (a) pharmaceutically acceptable salt(s), solvate(s), and/or hydrate(s) thereof. Preferably, the FMS-like tyrosine kinase 3 (FLT3) inhibitor is quizartinib (AC220) or pharmaceutically acceptable salt(s), solvate(s), and/or hydrate(s) thereof.

These and further exemplary inhibitors to be used herein are described in more detail below.

Brand Name: Quizartinib

Structure:

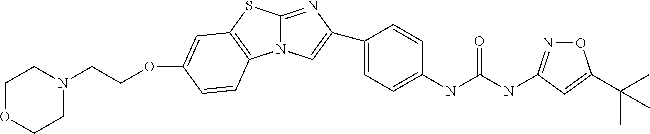 [SMILES: CC(C)(C)c1cc(NC(=O)Nc2ccc(-c3cn4c(n3)sc3cc(OCCN5CCOCC5)ccc34)cc2)no1]

Affinities: FLT3 (1.6 nM), KIT (4.8 nM), PDGFRB (7.7 nM), RET (9.9 nM)ₛ PDGFRA (1 InM),CSF1R (12 nM)

Brand Name: Crenolanib

Structure:

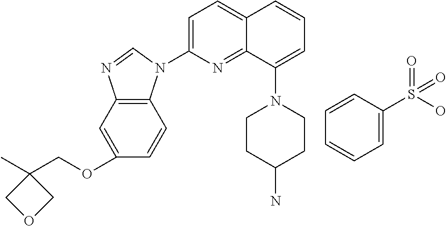

Affinities: FLT3, PDGFRb

Brand Name: Midostaurin

Structure:

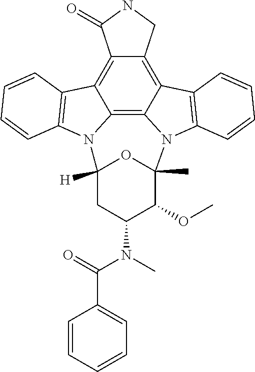 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

Affinities: PKNI (93 nM), TBI (9 3 nM), FT (1 lnM), JAK3 (1.2M), MLKI (1 znM), and 30 targets in the range 15-110 nM

Brand Name: Lestaurtinib

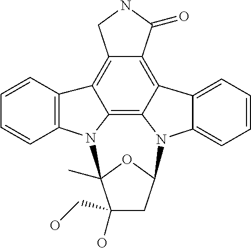 [SMILES: C[C@]12O[C@H](C[C@]1(O)CO)n1c3ccccc3c3c4c(c5c6ccccc6n2c5c31)CNC4=O]

Affinities: FLT3, TRKA, TRKB, TRKC

Brand Name: 45C-203

Structure:

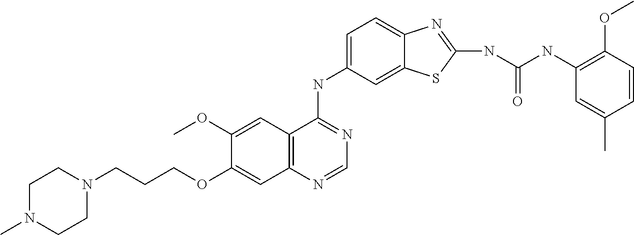

Affinities: FLT3, VEGFR

Structure:

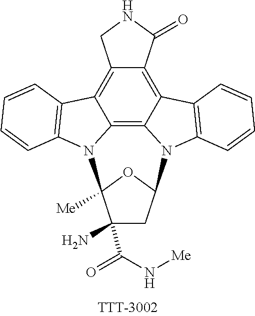 [SMILES: CNC(=O)[C@@]1(N)C[C@H]2O[C@]1(C)n1c3ccccc3c3c4c(c5c6ccccc6n2c5c31)C(=O)NC4]

Affinities: FLT3 (Wall, Blood (ASH Annual Meeting Abstracts). 2012; 1.20:866);

LRRK2 (Yao, Human molecular genetics. 2013; 22(2):328-44).

Clinical Phase: Preclinical

Developer: Tautatis (originator)

Brand Name: Sorafenib

Code Name: Bay-43-0006

Structure:

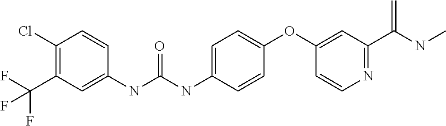 [SMILES: C=C(NC)c1cc(Oc2ccc(NC(=O)Nc3ccc(Cl)c(C(F)(F)F)c3)cc2)ccn1]

IUPAC Name: 4-[4-[3-4-Chloro-3-(trifluoromethyl)phenyl]ureido)phenoxy]-N-methylpyridine-2-carboxamide

Affinities: DDRI (1.5 nM), HIPK4 (3 nM)h ZAK (6 nM), DDR2 (7 nM), FLT3 (13 nM), and 15 targets in the range 13-130 nM (Zarrinkar, Gunawardane et al. 2009, loc, cit.) Clinical Phase: Launched (renal and heptacellular carcinoma), Phase I/O (blood cancer) Developer: Bayer

Brand Name: Ponatinib

Code Name: AP-24534 Structure:

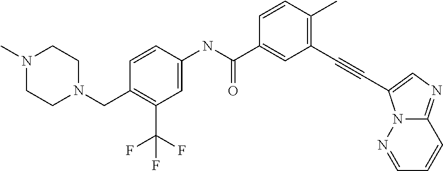 [SMILES: Cc1ccc(C(=O)Nc2ccc(CN3CCN(C)CC3)c(C(F)(F)F)c2)cc1C#Cc1cnc2cccnn12]

IUPAC Name: 3-[2-(Imidazo[1,2-b]pyridazin-3-yl)ethynyl]-4-methyl-N-[4(4-methylpiperazin-1-ylmethyl)-3-(trifluoromethyl)phenyl]benzamide

Affinities: BCR-ABL, FLT3, KIT, FGFR1, PDGFRa (Gozgit, Mol Cancer Ther

2011; 10(6):1028-35).

Clinical Phase: Phase II (AML)

Developer: Ariad Pharmaceuticals (originator)

Brand Name: Sunitinib

Code Name: SU-11248

Structure:

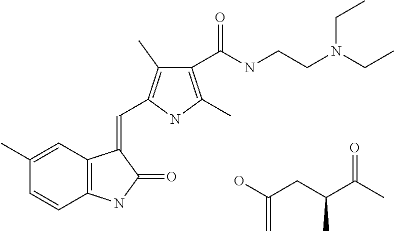

IUPAC Name: (Z)—N-[2-(Diethylarino)ethyl]-5-(5-fluoro-2-oxo-2,3-dihydro-IH-indol-3-ylidenemethyl)-2,4-dimethyl-1H-pyrrole-3-carboxamide 2(S)˜hydroxybutanedioic acid (1:1)N-[2-(Diethylamino)ethyl]-5-[(Z)-(5-fluoro-2-oxo-1,2-dihydro-3H-indol-3-ylidene)methyl]-2,4-dimethyl-1 H-pyrrole-3-carboxamide L-malate

Affinities: PDGFRB (0.075 nM), KIT (0.37 nM), FLT3 (0.47 nM), PDGFRA (0.79 nM),

DRAK1 (I.O nM), VEGFR2 (1.5 nM), FLT1 (1.8 nM), CSF1R (2.0 nM) (Zarrinkar, Gunawardane et al. 2009, loc. cit.)

Clinical Phase: Launched (renal cell carcinoma, gastrointestinal stromal cancer, neuroendocrine pancreas), phase I (AML)

Developer: Pfizer (Originator)

Brand Name: Tandutinib

Code Name: MLN-0518

Structure:

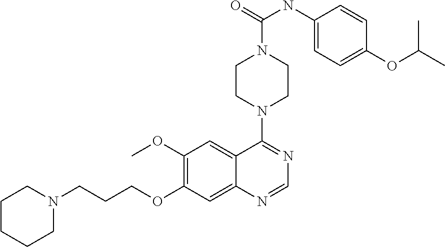 [SMILES: COc1cc2c(N3CCN(C(=O)Nc4ccc(OC(C)C)cc4)CC3)ncnc2cc1OCCCN1CCCCC1]

IUP AC Name: N-(4-Isopropoxyphenyl)-4-[6-methoxy-7-[3-(I-piperidinyl)propoxy]quinazolin-4-yl]piperazine-1-carboxamide

Affinities: PDGFRA (2.4 nM), KIT (2.7 nM), FLT3 (3 nM), PDGFRB (4.5 nM), CSF1R (4.9 nM) (Zarrinkar, Gunawardane et al. 2009, loc, cit.)

Clinical Phase: discontinued

Developer: Kyowa Hakko Kirin (Originator), Millennium Pharmaceuticals (Originator),

Code Name: FF-101.01

Structure:

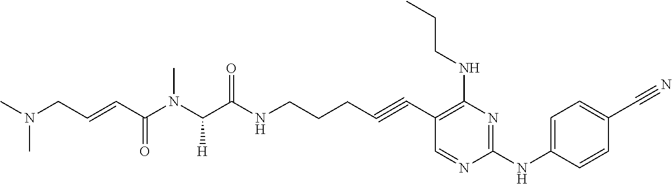

National Cancer Institute, Takeda (Originator) FLT3 inhibitors to be used in accordance with the present invention are not limited to the herein described or further known exemplary inhibitors. Accordingly, also further inhibitors or even yet unknown inhibitors may be used in accordance with the present invention. Such inliibitors may be identified by the methods described and provided herein and methods known in the art, like high-throughput screening using biochemical assays for inhibition of FLT3.

Assays for screening potential FLT3 inhibitors and, in particular, for identifying FLT3 inhibitors as defined herein, comprise, for example, in vitro competition binding assays to quantitatively measure interactions between test compounds and recombinantly expressed kinases (Fabian et al., Nat Biotechnol. 2005 23(3):329-36). Hereby, competition with immobilized capture compounds and free test compounds is performed. Test compounds that bind the kinase active site will reduce the amount of kinase captured on solid support, whereas test molecules that do not bind the kinase have no effect on the amount of kinase captured on the solid support. Furthermore, inhibitor selectivity can also be assessed in parallel enzymatic assays for a set of recombinant protein kinases.^{2,3} (Davies et al., Biochem. J. 2000 351:95-105; Bain et of, Biochem. J. 2003 371:199-204). These assays are based on the measurement of the inhibitory effect of a kinase inhibitor and determine the concentration of compound required for 50% inhibition of the protein kinases of interest. Proteomics methods are also an efficient tool to identify cellular targets of kinase inliibitors. Kinases are enriched from cellular lysates by immobilized capture compounds, so the native target spectrum of a kinase inhibitor can be determined.⁴ (Godl et al., Proc Natl Acad Sci USA. 2003 100(26):5434-9).

Assays for screening of potential inhibitors and, in particular, for identifying inhibitors as defined herein, are, for example, described in the following papers:
Fabian et al., Nat Biotechnol. 2005 23(3):329-36
Davies at al., Biochem. J. 2000 351:95-105.
Bain et al., Biochem. J. 2003 371:199-204.
Godl et al., Proc Natl Acad Sci USA. 2003 100(26): 15434-9.

The above papers are incorporated herein in their entirety by reference.

BACE Inhibitors

In certain embodiments, the Pulmonary Regenerative Agent is a β-secretase (BACE) inhibitor, and more preferably a selective BACE1 inhibitor.

A number of BACE1 inhibitors are known in the art, including small molecules and inhibitory antibodies. BACE1 inhibitors include LY2886721 and LY2811376 (Lilly); MBI-1, MBI-3, MBI-5, and MK-8931 (Merck); E2609 (Eisai); RG7129 (Roche); TAK-070 (Takeda); CTS-21166 (CoMentis); AZ3971, AZ4800, AZD-3289, AZD-3293 and AZ4217 (AstraZeneca); HPP854 (High Point Pharmaceuticals); Ginsenoside Rg1 (CID 441923); Hispidin (CID310013); TDC (CID 5811533); Monacolin K (CID 53232); SCH 1359113; Spirocyclic inhibitors (e.g., as described in Hunt et al., J Med Chem. 2013 Apr. 25; 56(8):3379-403, such as compound (R)-50); fluorine-substituted 1,3-oxazines (e.g., as described in Hilpert et al., J Med Chem. 2013 May 23; 56(10):3980-95, such as the CF3 substituted oxazine 89). Inhibitory antibodies include bispecific antibodies with one arm targeting BACE and the other recognizing transferrin receptor to boost brain penetrance (see, e.g., Yu et al., Sci Transl Med. 2011 May 25; 3(84):84ra44; Atwal et al., Sci Transl Med. 2011 May 25; 3(84):84ra43, and U.S. Pat. No. 8,772,457) and camelid antibodies that bind and inhibit BACE1 encoded by virus (see e.g., U.S. Pat. No. 8,568,717 and US20110091446).

Other exemplary BACE1 inhibitors include AM-6494; AMG-8718; Anisomycin; Atabecestat; Aurapten; C000000956; CL82198; Corynoline; Donepezil; EBI-2511; Elenbecestat; Felbinac; Ginsenoside Re; L 651580; L 655240; L 8412; Laciniatoside V; Lanabecestat (i.e., such as free base or camsylate); Lanabecestat (also known as AZD3293 and LY3314814); LDN-57444; Loganin; Methylguanidine hydrochloride; NB-360 (particularly the free base form); PF-05297909; PF-06663195; PF-06751979 (particularly the free base form); PH-002; R05508887 (particularly the free base form); Sinensetin; Taxifolin; Tolfenamic acid; Trientine-2HCI (also known as Triethylenetetramine, abbreviated TETA and trien); Umibecestat (particularly the free base form or HCl salt); Verubecestat (particularly the free base and TFA forms).

These and other BACE1 inhibitors useful in the present methods are described in the following US Pre-Grant Publications: 20140286963; 20140275165; 20140235626; 20140228356; 20140228277; 20140186357; 20140179690; 20140112867; 20140057927; 20140051691; 20140011802; 20130289050; 20130217705; 20130210839; 20130108645; 20130105386; 20120258961; 20120245157; 20120245155; 20120245154; 20120238557; 20120237526; 20120232064; 20120214186; 20120202828; 20120202804; 20120190672; 20120172355; 20120171120; 20120148599; 20120094984; 20120093916; 20120064099; 20120015961; 20110288083; 20110237576; 20110207723; 20110158947; 20110152341; 20110152253; 20110091446; 20110071124; 20110033463; 20100317850; 20100285597; 20100273671; 20100221760; 20100144790; 20100132060; 20100093999; 20100075957; 20100063134; 20090258925; 20090209755; 20090176836; 20090162878; 20090136977; 20090081731; 20090060987; 20090042993; 20080124379; 20070224656; 20070185042; 20060216292; 20060182736; 20060178328; 20060052327; 20050196398; 20050048641; 20040248231; 20040220132; 20040162255; 20040132680; 20040063161; 20030194745; 20020159991; and 20020157122, and U.S. Pat. Nos. 8,772,457; 8,703,785; 8,568,717; 8,415,319; 8,288,354; 8,198,269; 8,183,219; 8,058,251; 7,829,694; 7,816,378; 7,618,948; 7,273,743; and 6,713,276.

FAK Inhibitors

Focal adhesion kinase (FAK), also known as cytoplasmic protein-tyrosine kinase (PTK2), is a cytosolic protein tyrosine kinase concentrated in the focal adhesions that form among cells attaching to extracellular matrix constituents.

In certain embodiments, the Pulmonary Regenerative Agent is an inhibitor of focal adhesion kinase (FAK), i.e., is a FAK Inhibitor. Exemplary FAK inhibitors include PF-562271, PF-00562271, PND-1186, GSK2256098, PF-431396, PF-4618433, TAE226, CEP-37440, PF-03814735, PF-573228, BI-4464, NVP-TAE 226, PND-1186 and Defactinib. In certain embodiments, the FAK inhibitor is a Dual FAK/PYK2 inhibitor such as PF-431396. In other embodiments, the FAK inhibitor is a selective FAK inhibitor, such as FAK Inhibitor 14, PF-573228 or Y-11.

The structures of exemplary inhibitors of FAK are provided in the table below.

| Inhibitor | |
|---|---|
| PF-562271 | 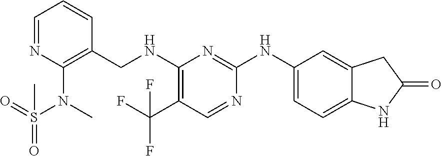 [SMILES: CN(c1ncccc1CNc1nc(Nc2ccc3c(c2)CC(=O)N3)ncc1C(F)(F)F)S(C)(=O)=O] |
| PF-573228 | 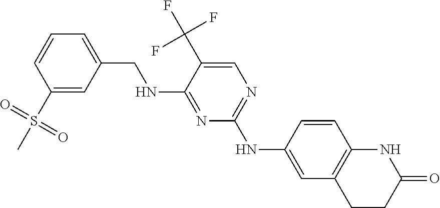 [SMILES: CS(=O)(=O)c1cccc(CNc2nc(Nc3ccc4c(c3)CCC(=O)N4)ncc2C(F)(F)F)c1] |
| TAE226 (NVP- TAE226) | 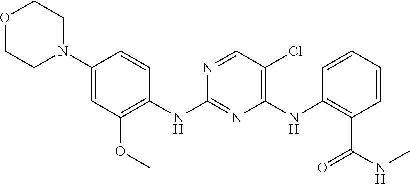 [SMILES: CNC(=O)c1ccccc1Nc1nc(Nc2ccc(N3CCOCC3)cc2OC)ncc1Cl] |
| PF-03814735 | 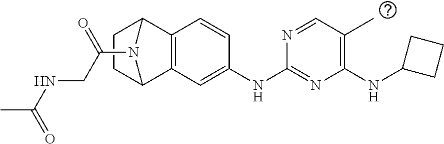 [SMILES: CC(=O)NCC(=O)N1C2CCC1c1cc(Nc3ncc(C)c(NC4CCC4)n3)ccc12] |
| PF-562271 HCl | 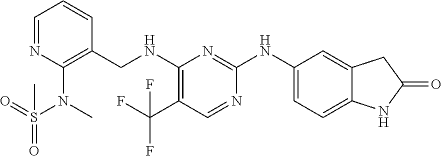 [SMILES: CN(c1ncccc1CNc1nc(Nc2ccc3c(c2)CC(=O)N3)ncc1C(F)(F)F)S(C)(=O)=O] |
| GSK2256098 | 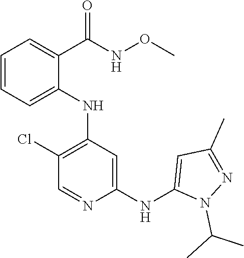 [SMILES: CONC(=O)c1ccccc1Nc1cc(Nc2cc(C)nn2C(C)C)ncc1Cl] |
| PF-431396 | 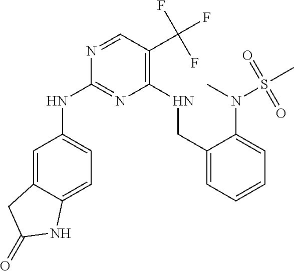 [SMILES: CN(c1ccccc1CNc1nc(Nc2ccc3c(c2)CC(=O)N3)ncc1C(F)(F)F)S(C)(=O)=O] |
| PND-1186 (VS- 4718) | 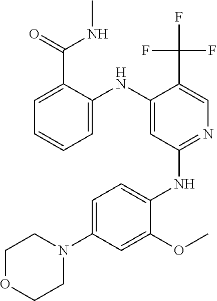 [SMILES: CNC(=O)c1ccccc1Nc1cc(Nc2ccc(N3CCOCC3)cc2OC)ncc1C(F)(F)F] |
| Defactinib (VS- 6063, PF-04554876) | 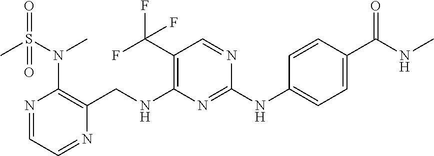 |
| Solanesol (Nonaisoprenol) | 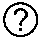 |

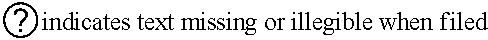

VEGFR Inhibitors

In certain embodiments, the Pulmonary Regenerative Agent is a VEGF receptor pathway inhibitor, preferably a VEGF receptor tyrosine kinase inhibitor. Exemplary VEGF receptor pathway inhibitors include vatalanib succinate (or other compounds disclosed in EP 296122), bevacizumab (AVASTIN®), axitinib (INLYTA®), brivanib alaninate (BMS-582664, (S)—((R)-1-(4-(4-Fluoro-2-methyl-1H-indol-5-yloxy)-5-methylpyrrolo[2,1-f-][1,2,4]triazin-6-yloxy)propan-2-yl)2-aminopropanoate), sorafenib (NEXAVAR®), pazopanib (VOTRIENT®), sunitinib malate (SUTENT®), cediranib (AZD2171, CAS 288383-20-1), vargatef (BIBF1120, CAS 928326-83-4), Foretinib (GSK1363089), telatinib (BAY57-9352, CAS 332012-40-5), apatinib (YN968D1, CAS 811803-05-1), imatinib (GLEEVEC®), ponatinib (AP24534, CAS 943319-70-8), tivozanib (AV951, CAS 475108-18-0), regorafenib (BAY73-4506, CAS 755037-03-7), vatalanib dihydrochloride (PTK787, CAS 212141-51-0), brivanib (BMS-540215, CAS 649735-46-6), vandetanib (CAPRELSA® or AZD6474), motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470), dovitinib dilactic acid (TK1258, CAS 852433-84-2), linfanib (ABT869, CAS 796967-16-3), cabozantinib (XL184, CAS 849217-68-1), lestaurtinib (CAS 111358-88-4), N-[5-[[[5-(1,1-dimethylethyl)-2-oxazolyl]methyl]thio]-2-thiazolyl]-4-pipe-ridinecarboxamide (BMS38703, CAS 345627-80-7), (3R,4R)-4-amino-1-((4-((3-methoxyphenyl)amino)pyrrolo[2,1-f][1,2,4]triazi-n-5-yl)methyl)piperidin-3-ol (BMS690514), N-(3,4-Dichloro-2-fluorophenyl)-6-methoxy-7-[[(3α,5ρ,6aα-)-octahydro-2-methylcyclopenta[c]pyrrol-5-yl]methoxy]-4-quinazolinamine (XL647, CAS 781613-23-8), 4-methyl-3-[[1-methyl-6-(3-pyridinyl)-1H-pyrazolo[3,4-d]pyrimidin-4-yl]am-ino]-N-[3-(trifluoromethyl)phenyl]-benzamide (BHG712, CAS 940310-85-0), aflibercept (EYLEA®), and endostatin (ENDOSTAR®).

In some embodiment, the VEGFR inhibitor is an inhibitor of one or more of VEGFR-2, PDGFR [symbol] , KIT or Raf kinase C, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine (Compound A37) or a compound disclosed in PCT Publication No. WO 2007/030377.

AKT Inhibitors

In certain embodiments, the Pulmonary Regenerative Agent is an AKT Inhibitor such as GDC0068 (also known as GDC-0068, ipatasertib and RG7440), MK-2206, perifosine (also known as KRX-0401), GSK690693, AT7867, triciribine, CCT128930, A-674563, PHT-427, Akti-1/2, afuresertib (also known as GSK2110183), AT13148, GSK2141795, BAY1125976, uprosertib (aka GSK2141795), Akt Inhibitor VIII (1,3-dihydro-1-[1-[[4-(6-phenyl-1H-imidazo[4,5-g]quinoxalin-7-yl)phenyl]m-ethyl]-4-piperidinyl]-2H-benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][-1,6]naphthyridin-3(2H)-one), uprosertib (N—((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-c-yclopenta[d]pyrimidin-4-yl)piperazin-1-yl)-3-(isopropylamino)propan-1-one)-, AZD 5363 (4-Piperidinecarboxamide, 4-amino-N-[(1S)-1-(4-chlorophenyl)-3-hydroxypropyl]-1-(7H-pyrrolo[2,3-d]p-yrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6).

Additional Akt inhibitors include: ATP-competitive inhibitors, e.g., isoquinoline-5-sulfonamides (e.g., H-8, H-89, NL-71-101), azepane derivatives (e.g., (−)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3(2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl)carbonyl]-N′-(3-bromophenyl)-thiourea), purine derivatives (e.g., Triciribine (TCN, NSC 154020), triciribine mono-phosphate active analogue (TCN-P),4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3.alpha.- and 3.beta.-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives

### V. Local Delivery

The disclosure provides for use of these drug agents, systemically of by localized delivery to the pulmonary tract of patients, in order to more effectively treat COPD and other inflammatory diseases/conditions of the lung, as well as forms of metaplasia, neoplasia and cancers of the pulmonary tract. In certain embodiments, one or both of the inhibitor and promoter are formulated, together or separately, for local delivery to pulmonary tract.

Merely to illustrate an embodiment, the present disclosure provides a colon targeted bioadhesive modified release formulation, comprising a promoter and/or inhibitor as described above, or a pharmaceutically acceptable salt. For instance, the formulation can comprise a bioadhesive coating that is disposed over all or a portion of the surface of a core containing one or more of the subject drug agents, which core may optionally be coated with a rate-controlling membrane system, thus yielding a monolithic system that releases the agent in a regulated manner. Representative synthetic polymers for use in bioadhesive coatings include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers suitable for use in the disclosure include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers for use in bioadhesive coatings include polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Polyanhydrides are particularly suitable for use in bioadhesive delivery systems because, as hydrolysis proceeds, causing surface erosion, more and more carboxylic groups are exposed to the external surface. However, polylactides erode more slowly by bulk erosion, which is advantageous in applications where it is desirable to retain the bioadhesive coating for longer durations. In designing bioadhesive polymeric systems based on polylactides, polymers that have high concentrations of carboxylic acid are preferred. The high concentrations of carboxylic acids can be attained by using low molecular weight polymers (MW of 2000 or less), because low molecular weight polymers contain a high concentration of carboxylic acids at the end groups.

When the bioadhesive polymeric coating is a synthetic polymer coating, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers of thereof. In an exemplary embodiment, the synthetic polymer is poly(fumaric-co-sebacic) anhydride.

Another group of polymers suitable for use as bioadhesive polymeric coatings are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymeric coatings are polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines. Preferably, the hydrophilic groups are selected to not interfere and instead to enhance the bioadhesiveness of the polymers. The hydrophilic groups can be either directly attached to a hydrophobic polymer backbone or attached through a spacer group. Typically, a spacer group is an alkylene group, particularly a C1-C8 alkyl group such as a C2-C6 alkyl group. Preferred compounds containing one or more hydrophilic groups include amino acids (e.g., phenyalanine, tyrosine and derivatives thereof) and amine-containing carbohydrates (sugars) such as glucosamine.

Formulation Approaches for Pulmonary Delivery to the Lung

The drug moieties of the disclosure can be administered by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the disclosure comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

In the case of dry powder inhalers and aerosols, the dosage unit is determined by means of a valve which delivers a metered amount. Units in accordance with the disclosure are typically arranged to administer a metered dose or “puff” containing from 0.001 mg to 10 mg of the compound of the drug moiety. The overall daily dose will typically be in the range 0.001 mg to 40 mg which may be administered in a single dose or, more usually, as divided doses throughout the day.

Many conventional techniques have been reported to produce dry powder inhalers (DPI) formulations. However, these methods have number of limitations, such as particle size, size distribution, shape and poor control over powder crystallinity. These problems can be rectified by specialized milling techniques. Jet-milling of drug under nitrogen gas with new nanojet milling instrument is the most suitable method for creating nanoparticles meant for pulmonary drug delivery. Below are some of the illustrative techniques.

The powdered formulation may be prepared starting from a dry product comprising an anti-PLESC agent and/or a Pulmonary Regenerative agent, its salt or mixtures thereof, by altering the particle size of the agent to form a dry formulation of particle size about 0.01 μm to about 500 μm in diameter; and selecting particles of the formulation comprising at least or greater than about 80%, about 85%, about 90%, about 95%, or about 100% particles of about 0.01 μm, 0.1 am or 0.5 am to about 100 am or 200 am in diameter. The particle size is desirably less than about 200 μm, preferably in the range about 0.05 μm, about 0.1 μm, about 1 μm, about 2 am to about 5 μm, about 6 μm, about 8 μm, about 10 μm, about 20 μm, about 50 μm, about 100 μm. Preferably, the selected particles of the formulation of about 0.1 to about 200 μm in diameter. More preferably, the selected particles of the formulation of about 0.1 to about 100 μm in diameter. Even more preferably, the selected particles of the formulation of about 0.1 to about 10 μm in diameter. Even much more preferably, the selected particles of the formulation of about 0.1 to about 8 μm in diameter. Even further much more preferably, the selected particles of the formulation of about 0.1 to about 5 μm in diameter.

The particle size of the dry agent may be then altered so as to permit the absorption of a substantial amount of the agent into the lungs upon inhalation of the formulation. The particle size of the medicament may be reduced by any known means, for example by milling or micronization. Typically, the particle size for the agent is altered by milling the dry agent either alone or in combination with a formulation ingredient to a suitable average particle size, preferably in the about 0.05 μm, about 5 am range (inhalation) or about 10 μm, to about 50 am (nasal delivery or lung instillation). Jet milling, also known as fluid energy milling, may be employed and are preferred among the procedures to give the particle size of interest using known devices. Jet milling is the preferred process. It should be understood that although a large percentage of the particles will be in the narrow range desired, this will not generally be true for all particles. Thus, it is expected that the overall particle range may be broader than the preferred range as stated above. The proportion of particles within the preferred range may be greater than about 80%, about 85%, about 90%, about 95%, and so on, depending on the needs of a specific formulation.

The particle size may be also altered by sieving, homogenization, and/or granulation, amongst others. These techniques are used either separately or in combination with one another. Typically, milling, homogenization and granulation are applied, followed by sieving to obtain the dry altered particle size formulation. These procedures may be applied separately to each ingredient, or the ingredients added together and then formulated.

Examples of the formulation ingredients that may be employed are not limited to, but include, an excipient, preservatives, stabilizers, powder flowability improving agents, a cohesiveness improving agent, a surfactant, other bioactive agents, a coloring agent, an aromatic agent, anti-oxidants, fillers, volatile oils, dispersants, flavoring agents, buffering agents, bulking agents, propellants or preservatives. One preferred formulation comprises the active agent and an excipient(s) and/or a propellant(s).

The particle size may be altered not only in a dry atmosphere but also by placing the active agent in solution, suspension or emulsion in inter-mediate steps. The active agent may be placed in solution, suspension, or emulsion, either prior to, or after, altering the particle size of the agent. An example of this embodiment that may be performed by dissolving the agent in a suitable solvent solution, and heating to an appropriate temperature. The temperature may be maintained in the vicinity of the appropriate temperature for a predetermined period of time to allow for crystals to form. The solution and the fledgling crystals then are cooled to a second lower temperature to grow the crystals by maintaining them at the second temperature for a period of time as is known in the art. The crystals are then allowed to reach room temperature when recrystalization is completed and the crystals of the agent have grown sufficiently. The particle size of the agent may also be altered by sample precipitation, which is conducted from solution, suspension or emulsion in an adequate solvent(s).

Spray drying is useful in altering the particle size, as well. By “spray dried or spray drying” what is meant is that the agent or composition is prepared by a process in which a homogeneous mixture of the agent in a solvent or composition termed herein the “pre-spray formulation”, is introduced via an atomizer, e.g., a two-fluid nozzle, spinning disk or an equivalent device into a heated atmosphere or a cold fluid as fine droplets. The solution may be an aqueous solution, suspension, emulsion, slurry or the like, as long as it is homogeneous to ensure uniform distribution of the material in the solution and, ultimately, in the powdered formulation. When sprayed into a stream of heated gas or air, the each droplet dries into a solid particle. Spraying of the agent into the cold fluid results in a rapid formation of atomized droplets that form particles upon evaporation of the solvent. The particles are collected, and then any remaining solvent may be removed, generally through sublimation (lyophilization), in a vacuum. As discussed below, the particles may be grown, e.g., by raising the temperature prior to drying. This produces a fine dry powder with particles of a specified size and characteristics, that are more fully discussed below. Suitable spray drying methodologies are also described below. See, for example U.S. Pat. Nos. 3,963,559; 6,451,349; and, 6,458,738, the relevant portions of which are incorporated herein by reference.

As used herein, the term “powder” means a composition that consists of finely dispersed solid particles that are relatively free flowing and capable of being readily dispersed in an inhalation or dry powder device and subsequently inhaled by a patient so that the particles can reach the intended region of the lung. Thus, the powder is “respirable” and suitable for pulmonary delivery. When the particle size of the next agent or the formulation is above about 10 μm, the particles are of such size that a good proportion of them will deposit in the nasal cavities, and will be absorbed there through.

The term “dispersibility” means the degree to which a dry powder formulation may be dispersed, i.e. suspended, in a current of air so that the dispersed particles may be respired or inhaled into the lungs or absorbed through the walls of the nasal cavities of a subject. Thus, a powder that is only 20% dispersible means that only 20% of the mass of particles may be suspended for inhalation into the lungs. The present formulation preferably has a dispersibility of about 1 to 99%, although others are also suitable.

The dry powder formulation may be characterized on the basis of a number of parameters, including, but not limited to, the average particle size, the range of particle size, the fine powder fraction (FPF), the average particle density, and the mass median aerodynamic diameter (MMAD), as is known in the art.

In a preferred embodiment, the agent is DHEA-S in a dihydrate crystalline form. The DHEA-S is first crystallized into the dihydrate crystalline form. The crystals are then put through the jet mill to produce it into a powder form. The preparation can further comprise lactose that is separately sieved or milled and mixed with the powdered crystalline dihydrate DHEA-S.

In a preferred embodiment, the dry powder formulation of this disclosure is characterized on the basis of their average particle size that was described above. The average particle size of the powdered agent or formulation may be measured as the mass mean diameter (MMD) by conventional techniques. The term, “about” means the numerical values could have an error in the range of about 10% of the numerical value. The dry powdered formulation of this disclosure may also be characterized on the basis of its fine particle fraction (FPF). The FPF is a measure of the aerosol performance of a powder, where the higher the fraction value, the better. The FPF is defined as a powder with an aerodynamic mass median diameter of less than 6.8 μm as determined using a multiple-stage liquid impinger with a glass throat (MLSI, Astra, Copley Instrument, Nottingham, UK) through a dry powder inhaler (Dryhalter™, Dura Pharmaceuticals). Accordingly, the dry powder formulation of the disclosure preferably has a FPF of at least about 10%, with at least about 20% being preferred, and at least about 30% being especially preferred. Some systems may enable very high FPFs, of the order of 40 to 50%.

The dry powdered formulation may be characterized also on the basis of the density of the particles containing the agent of the disclosure. In a preferred embodiment, the particles have a tap density of less than about 0.8 g/cm3, with tap densities of less than about 0.4 g/cm3 being preferred, and a tap density of less than about 0.1 g/cm3 being especially preferred. The tap density of dry powder particles may be measured using a GeoPyc™ (Micrometrics Instruments Corp), as is known in the art. Tap density is a standard measure of the envelope mass density, which is defined generally as the mass of the particle divided by the minimum sphere envelope volume within which it may be enclosed.

In another preferred embodiment, the aerodynamic particle size of the dry powdered formulation may be characterized as is generally outlined in the Examples. Similarly, the mass median aerodynamic diameter (MMAD) of the particles may be evaluated, using techniques well known in the art. The particles may be characterized on the basis of their general morphology as well.

The term “dry” means that the formulation has a moisture content such that the particles are readily dispersible in an inhalation device to form an aerosol. The dry powdered formulation in the disclosure comprises preferably substantially active compound, although some aggregation may occur, particularly upon long storage periods. As is known for many dry powder formulation, some percentage of the material in a powder formulation may aggregate, this resulting in some loss of activity. Accordingly, the dry powdered formulation has at least about 70% w/w active compound, i.e. % of total compound present, with at least about 80% w/w active compound being preferred, and at least about 90% w/w active compound being especially preferred. More highly active compound or agent is also contemplated, and may be prepared by the present method, i.e., an activity greater than about 95% and higher. The measurement of the total compound present will depend on the compound and, generally, will be done as is known in the art, on the basis of activity assays, etc. The measurement of the activity of the agent will be dependent on the compound and will be done on suitable bioactivity assays as will be appreciated by those in the art.

In spray drying, an individual stress event may arise due to atomization (shear stress and air-liquid interfacial stress), cold or heat denaturation, optionally freezing (ice-water interfacial stress and shear stress), and/or dehydration. Cryoprotectants and lyoprotectants have been used during lyophilization to counter freezing destabilization, and dehydration and long-term storage destabilization, respectively. Cryoprotectant molecules, e.g., sugars, amino acids, polyols, etc., have been widely used to stabilize active compounds in highly concentrated unfrozen liquids associated with ice crystallization. These are not required in the formulation.

The dry powdered formulations comprising an active compound may or not contain an excipient. “Excipients” or “protectants” including cryoprotectants and lyoprotectants generally refers to compounds or materials that are added as diluents or to ensure or increase flowability and aerosol dispersibility of the active compounds during the spray drying step and afterwards, and for long-term flowability of the powdered product. Suitable excipients are generally relatively free flowing particulate solids, do not thicken or polymerize upon contact with water, are basically innocuous when placed in the respiratory tract of a patient and do not substantially interact with the active compound in a manner that alters its biological activity.

Suitable excipients include, but are not limited to, proteins such as human and bovine serum albumin, gelatin, immunoglobulins, carbohydrates including monosaccharides (galactose, D-mannose, sorbose, etc.), disaccharides (lactose, trehalose, sucrose, etc.), cyclodextrins, and polysaccharides (raffinose, maltodextrins, dextrans, etc.); an amino acid such as monosodium glutamate, glycine, alanine, arginine or histidine, as well as hydrophobic amino acids (tryptophan, tyrosine, leucine, phenylalanine, etc.); a lubricant such as magnesium stearate; a methylamine such as betaine; an excipient salt such as magnesium sulfate; a polyol such as trihydric or higher sugar alcohols, e.g., glycerin, erythritol, glycerol, arabitol, xylitol, sorbitol, and mannitol; propylene glycol; polyethylene glycol; pluronics; surfactants; (lipid and non-lipid.surfactants) and combinations thereof. Preferred excipients are trehalose, sucrose, sorbitol, and lactose: as well as mixtures thereof. When excipients are used, they are used generally in amounts ranging from about 0.1, about 1, about 2, about 5, about 10 to about 15, about 10, about 15, about 20, about 40, about 60, about 99% w/w composition. Preferred are formulations containing lactose, or low amounts of excipient or other ingredients.

In another preferred embodiment, the dry powdered formulation of this disclosure is substantially free of excipients. “Substantially free” in this case generally means that the formulation contains less than about 10%, w/w preferably less than about 5%, w/w more preferably less than about 2-3% w/w, still more preferably less than about 1% w/w of any components other than the agent. Generally, for the purposes of this disclosure, the formulation may include a propellant and a co-solvent, buffers or salts, and residual water. In one preferred embodiment the dry powdered formulation (prior to the addition of bulking agent, discussed below) consists of the agent and protein as a major component, with small amounts of buffer(s), salt(s) and residual water. Generally, in this embodiment, the spray drying process comprises a temperature raising step prior to drying, as is more fully outlined below.

In another preferred embodiment, the pre-spray dried formulation, i.e. the solution formulation used in the spray drying process comprises the active agent in solution, e.g., aqueous solution, with only negligible amounts of buffers or other compounds. The pre-spray dried formulation containing little or no excipient may not be highly stable over a long period of time. It is, thus, desirable to perform the spray drying process within a reasonable short time after the pre-spray dried formulation is produced. Although, the pre-spray dried formulation utilizing little or no excipient may not be highly stable, the dry powder made from it may, and generally is both surprisingly stable and highly dispersible, as shown in the Examples.

The agents that are spray dried to form the formulations of the disclosure comprise the agent and optionally a buffer, and may or may not contain additional salts. The suitable range of the pH of the buffer in solution can be readily ascertained by those in the art. Generally, this will be in the range of physiological pH, although the agent of the disclosure may flowable at a wider range of pHs, for example acidic pH. Thus, preferred pH ranges of the pre-spray dry formulation are about 1, about 3, about 5, about 6 to about 7, about 8, about 10, and a pH about 7 being especially preferred. As will be appreciated by those in the art, there are a large number of suitable buffers that may be used. Suitable buffers include, but are not limited to, sodium acetate, sodium citrate, sodium succinate, sodium phosphate, ammonium bicarbonate and carbonate. Generally, buffers are used at molarities from about 1 mM, about 2 mM to about 200 mM about 10 mM, about 0.5 M, about 1 M, about 2 M, about 50 M being particularly preferred.

When water, buffers or solvents are used during the preparation process, they may additionally contain salts as already indicated.

In addition, the dry powdered formulation of the disclosure is generally substantially free of “stabilizers”. The formulation may contain, however, an additional surfactant that has its own prophylactic or therapeutic effect on the respiratory system on the lungs. These active agents may compensate for loss of lung surfactant or generally act by other mechanisms. The dry powdered formulations of the disclosure is also generally substantially free of microsphere-forming polymers. See, e.g., WO 97/44013; U.S. Pat. No. 5,019,400. That is, the powders of the disclosure generally comprise the active agent(s) and excipient, and do not require the use of polymers for structural or other purposes. The dry powdered formulations of the disclosure is also preferably stable. “Stability” may mean one of two things, retention of biological activity and retention of dispersibility over time, with preferred embodiments showing stability in both areas.

The dry powdered formulation of the disclosure generally retains biological activity over time, e.g., physical and chemical stability and integrity upon storage. Losses of biological activity are generally due to aggregation, and/or oxidation of agent's particles. However, when the agent is agglomerate around particles of excipient, the resulting agglomerates are highly stable and active. As will be appreciated by those in the art, there may be an initial loss of biological activity as a result of spray drying, due to the extreme temperatures used in the process. Once this has occurred, however, further loss of activity will be negligible, as measured from the time the powder is made. Moreover, the dry powdered formulation of the disclosure have been found to retain dispersibility over time, as quantified by the retention of a high FPF over time, the minimally aggregation, caking or clumping observed over time.

The agent(s) of the disclosure is (are) made by methods known in the art. See, for example, U.S. Pat. Nos. 6,087,351; 5,175,154; and, 6,284,750. The pre-spray drying composition may be formulated for stability as a liquid or solid formulation. For spray drying, the liquid formulations are subjected generally to diafiltration and/or ultrafiltration, as required, for buffer exchange (or removal) and/or concentration, as is known in the art. The pre-spray dry formulations comprise from about 1 mg/ml, about 5 mg/ml, about 10 mg/ml, about 20 mg/ml to about 60 mg/ml, about 75 mg/ml of the agent. Buffers and excipients, if present, are present at concentrations discussed above. The pre-spray drying formulation is then spray dried by dispersing the agent into hot air or gas, or by spraying it into a cold or freezing fluid, e.g., a liquid or gas. The pre-spray dry formulation may be atomized as is known in the art, for example via a two-fluid or ultrasonic nozzle using filtered pressurized air, into, for example, a fluid. Spray drying equipment may be used (Buchi; Niro Yamato; Okawara; Kakoki). It is generally preferable to slightly heat the nozzle, for example by wrapping the nozzle with heating tape to prevent the nozzle head from freezing when a cold fluid is used. The pre-spray dry formulation may be atomized into a cold fluid at a temperature of about −200° C. to about −100° C., about −80° C. The fluid may be a liquid such as liquid nitrogen or other inert fluids, or a gas such as air that is cooled. Dry ice in ethanol may be used as well as super-critical fluids. In one embodiment it is preferred to stir the liquid as the atomization process occurs, although this may not be required.

Micronization techniques involve placing bulk drug into a suitable mill. Such mills are commercially available from, for example, DT Industries, Bristol, Pa., under the tradename STOKES™. Briefly, the bulk drug is placed in an enclosed cavity and subjected to mechanical forces from moving internal parts, e.g., plates, blades, hammers, balls, pebbles, and so forth. Alternatively, or in addition to parts striking the bulk drug, the housing enclosing the cavity may turn or rotate such that the bulk drug is forced against the moving parts. Some mills, e.g., fluid energy or airjet mills, include a high-pressure air stream that forces the bulk powder into the air within the enclosed cavity for contact against internal parts. Once the size and shape of the drug is achieved, the process may be stopped and drug having the appropriate size and shape is recovered. Generally, however, particles having the desired particle size range are recovered on a continuous basis by elutriation.

There are many different types of size reduction techniques that can be used to reduce to size of the particles. There is the cutting method employing the use of a cutter mill that can reduce the size of particles to about 100 μm. There is the compression method employing the use of an end-runner mill that can reduce the size of particles to less than about 50 μm. There is the impact method employing the use of a vibration mill that can reduce the size of particles to about 1 μm or a hammer mill that can reduce the size of particles to about 8 μm. There is the attrition method employing the use of a roller mill that can reduce the size of particles to about 1 μm. There is the combined impact and attrition method employing the use of a pin mill that can reduce the size of particles to about 10 μm, a ball mill that can reduce the size of particles to about 1 μm, a fluid energy mill (or jet mill) that can reduce the size of particles to about 1 μm. One of ordinary skill in the art is able through routine experimentation determine the particle size reduction method and means to produce the desired particle size of the composition.

Supercritical fluid processes may be used for altering the particle size of the agent. Supercritical fluid processes involve precipitation by rapid expansion of supercritical solvents, gas anti-solvent processes, and precipitation from gas-saturated solvents. A supercritical fluid is applied at a temperature and pressure that are greater than its critical temperature (Tc) and critical pressure (Pc), or compressed fluids in a liquid state. It is known that at near-critical temperatures, large variations in fluid density and transport properties from gas-like to liquid-like can result from relatively moderate pressure changes around the critical pressure (0.9-1.5 Pc). While liquids are nearly incompressible and have low diffusivity, gases have higher diffusivity and low solvent power. Supercritical fluids can be made to possess an optimum combination of these properties. The high compressibility of supercritical fluids (implying that large changes in fluid density can be brought about by relatively small changes in pressure, making solvent power highly controllable) coupled with their liquid-like solvent power and better-than-liquid transport properties (higher diffusivity, lower viscosity and lower surface tension compared with liquids), provide a means for controlling mass transfer (mixing) between the solvent containing the solutes (such as a drug) and the supercritical fluid.

The two processes that use supercritical fluids for particle formation and that have received attention in the recent past are: (1) Rapid Expansion of Supercritical Solutions (RESS) (Tom, J. W. Debenedetti, P. G., 1991, The formation of bioerodible polymeric microspheres and microparticles by rapid expansion of supercritical solutions. BioTechnol. Prog. 7:403-411), and (2) Gas Anti-Solvent (GAS) Recrystallization (Gallagher, P. M., Coffey, M. P., Krukonis, V. J., and Klasutis, N., 1989, GAS antisolvent recrystallization: new process to recrystallize compounds in soluble and supercritical fluids. Am. Chem. Sypm. Ser., No. 406; Yeo et al., (1993); U.S. Pat. No. 5,360,478 to Krukonis et al.; U.S. Pat. No. 5,389,263 to Gallagher et al.). In the RESS process, a solute (from which the particles are formed) is first solubilized in supercritical CO₂ to form a solution. The solution is then, for example, sprayed through a nozzle into a lower pressure gaseous medium. Expansion of the solution across this nozzle at supersonic velocities causes rapid depressurization of the solution. This rapid expansion and reduction in CO₂ density and solvent power leads to supersaturation of the solution and subsequent recrystallization of virtually contaminant-free particles. The RESS process, however, may not be suited for particle formation from polar compounds because such compounds, which include drugs, exhibit little solubility in supercritical CO₂ Cosolvents (e.g., methanol) may be added to CO₂ to enhance solubility of polar compounds; this, however, affects product purity and the otherwise environmentally benign nature of the RESS process. The RESS process also suffers from operational and scale-up problems associated with nozzle plugging due to particle accumulation in the nozzle and to freezing of CO₂ caused by the Joule-Thompson effect accompanying the large pressure drop.

In the GAS process, a solute of interest (typically a drug) that is in solution or is dissolved in a conventional solvent to form a solution is sprayed, typically through conventional spray nozzles, such as an orifice or capillary tube, into supercritical CO₂ which diffuses into the spray droplets causing expansion of the solvent. Because the CO₂-expanded solvent has a lower solubilizing capacity than pure solvent, the mixture can become highly supersaturated and the solute is forced to precipitate or crystallize. The GAS process enjoys many advantages over the RESS process. The advantages include higher solute loading (throughput), flexibility of solvent choice, and fewer operational problems in comparison to the RESS process. In comparison to other conventional techniques, the GAS technique is more flexible in the setting of its process parameters, and has the potential to recycle many components, and is therefore more environmentally acceptable. Moreover, the high pressure used in this process (up to 2,500 psig) can also potentially provide a sterilizing medium for processed drug particles; however, for this process to be viable, the selected supercritical fluid should be at least partially miscible with the organic solvent, and the solute should be preferably insoluble in the supercritical fluid.

Gallagher et al., (1989) teach the use of supercritical CO₂ to expand a batch volume of a solution of nitroguanadine and recrystallize particles of the dissolved solute. Subsequent studies disclosed by Yeo et al., (1993) used laser-drilled, 25-30 μm capillary nozzles for spraying an organic solution into CO₂. Use of 100 μm and 151 μm capillary nozzles also has been reported (Dixon, D. J. and Johnston, K. P., 1993, Formation of microporous polymer fibers and oriented fibrils by precipitation with a compressed fluid antisolvent. J. App. Polymer Sci. 50:1929-1942; Dixon, D. G., Luna-Barcenas, G., and Johnson K. P., 1994, Microcellular microspheres and microballoons by precipitation with a vapor-liquid compressed fluid antisolvent. Polymer 35:3998-4005).

Examples of solvents are selected from carbon dioxide (CO₂), nitrogen (N₂), Helium (He), oxygen (O₂), ethane, ethylene, ethylene, ethane, methanol, ethanol, trifluoromethane, nitrous oxide, nitrogen dioxide, fluoroform (CHF₃), dimethyl ether, propane, butane, isobutanes, propylene, chlorotrifluormethane (CClF₃), sulfur hexafluoride (SF₆), bromotrifluoromethane (CBrF₃), chlorodifluoromethane (CHClF₂), hexafluoroethane, carbon tetrafluoride carbon dioxide, 1,1,1,2-tetrafluoroethane, 1,1,1,2,3,3,3-heptafluoropropane, xenon, acetonitrile, dimethylsulfoxide (DMSO), dimethylformamide (DMF), and mixtures of two or more thereof.

The atomization conditions, including atomization gas flow rate, atomization gas pressure, liquid flow rate, etc., are generally controlled to produce liquid droplets having an average diameter of from about 0.5 μm, about 1 μm, about 5 am to about 10 μm, about 30 am, about 50 μm, about 100 μm, with droplets of average size about 10 μm and about 5 μm being preferred. Conventional spray drying equipment is generally used. (Buchi, Niro Yamato, Okawara, Kakoki, and the like). Once the droplets are produced, they are dried by removing the water and leaving the active agent, any excipient(s), and residual buffer(s), solvent(s) or salt(s). This may be done in a variety of ways, such as by lyophilization, as is known in the art. i.e. freezing as a cake rather than as droplets. Generally, and preferably, vacuum is applied, e.g., at about the same temperature as freezing occurred. However, it is possible to relieve some of the freezing stress on the agent by raising the temperature of the frozen particles slightly prior to or during the application of vacuum. This process, termed “armealing”, reduces agent inactivation, and may be done in one or more steps, e.g., the temperature may be increased one or more times either before or during the drying step of the vacuum with a preferred mode utilizing at least two thermal increases. The particles may be incubated for a period of time, generally sufficient time for thermal equilibrium to be reached, i.e. depending on sample size and efficiency of heat exchange 1 to several hours, prior to the application of the vacuum, then vacuum is applied, and another annealing step is done. The particles may be lyophilized for a period of time sufficient to remove the majority of the water not associated with crystalline structure, the actual period of time depending on the temperature, vacuum strength, sample size, etc.

Spheronization involves the formation of substantially spherical particles and is well known in the art. Commercially available machines for spheronizing drugs are known and include, for example, Marumerizer™ from LCI Corp. (Charlotte, N.C.) and CF-Granulator from Vector Corp. (Marion, Iowa). Such machines include an enclosed cavity with a discharge port, a circular plate and a means to turn the plate, e.g., a motor. Bulk drug or moist granules of drug from a mixer/granulator are fed onto the spinning plate, which forces them against the inside wall of the enclosed cavity. The process results in particles with spherical shape. An alternative approach to spheronization that may be used includes the use of spray drying under controlled conditions. The conditions necessary to spheronize particles using spray-drying techniques are known to those skilled in the art and described in the relevant references and texts, e.g., Remington: The Science and Practice of Pharmacy, Twentieth Edition (Easton, Pa.: Mack Publishing Co., 2000).

In a preferred embodiment, a secondary lyophilization drying step is conducted to remove additional water at temperatures about 0° C., about 10° C., to about 20° C., to about 25° C., with about 20° C. being preferred. The powder is collected then by using conventional techniques, and bulking agents, if desirable, may be added although not required. Once made, the dry powder formulation of the disclosure may be being readily dispersed by a dry powder inhalation device and subsequently inhaled by a patient so that the particles penetrate into the target regions of the lungs. The powder of the disclosure may be formulated into unit dosages comprising therapeutically effective amounts of the active agent and used for delivery to a patient, for example, for the prevention and treatment of respiratory and lung disorders.

The dry powder formulation of this disclosure is formulated and dosed in a fashion consistent with good medical practice, taking into account, for example, the type of disorder being treated, the clinical condition of the individual patient, whether the active agent is administered for preventative or therapeutic purposes, its concentration in the dosage, previous therapy, the patient's clinical history and his/her response to the active agent, the method of administration, the scheduling of administration, the discretion of the attending physician, and other factors known to practitioners. The “effective amount” or “therapeutically effective amount” of the active compound for purposes of this patent include preventative and therapeutic administration, and will depend on the identity of the active agent and is, thus, determined by such considerations and is an amount that increases and maintains the relevant, favorable biological response of the subject being treated. The active agent is suitably administered to a patient at one time or over a series of treatments, preferably once a day, and may be administered to the patient at any time from diagnosis onwards. A “unit dosage” means herein a unit dosage receptacle containing a therapeutically effective amount of a micronized active agent. The dosage receptacle is one that fits within a suitable inhalation device to allow for the aerosolization of the dry powdered formulation by dispersion into a gas stream to form an aerosol. These can be capsules, foil pouches, blisters, vials, etc. The container may be formed from any number of different materials, including plastic, glass, foil, etc, and may be disposable or rechargeable by insertion of a filled capsule, pouch, blister etc. The container generally holds the dry powder formulation, and includes directions for use. The unit dosage containers may be associated with inhalers that will deliver the powder to the patient. These inhalers may optionally have chambers into which the powder is dispersed, suitable for inhalation by a patient.

The dry powdered formulations of the disclosure may be further formulated in other ways, e.g., as a sustained release composition, for example, for implants, patches, etc. Suitable examples of sustained-release compositions include semi-permeable polymer matrices in the form of shaped articles, e.g., films or microcapsules. Sustained-release matrices include for example polylactides. See for example, U.S. Pat. No. 3,773,919; EP 58,481. Copolymers of L-glutamic acid and gamma-ethyl-L-glutamate are also suitable. See, e.g., Sidman et al., Biopolymers 22: 547-556 (1983) as poly(2-hydroxyethyl methacrylate). See Langer et al., J. Biomed. Mater. Res. 15: 167-277 (1981); Langer, Chem. Tech., 12: 98-105 (1982). Also suitable are ethylene vinyl acetate and poly-D-(−)-3-hydroxybutyric acid. See, Langer et al., supra; (EP 133,988). Sustained-release compositions also include liposomally entrapped agent, that may be prepared by known methods. See, for example, DE 3,218,121; Epstein et al., Proc. Natl. Acad. Sci. USA 82: 3688-3692 (1985); Hwang et al., Proc. Natl. Acad. Sci. USA 77: 4030-4034 (1980); EP 52,322; EP 36,676; EP 88,046; EP 143,949; EP 142,641; Japanese Pat. Application 83-118008; U.S. Pat. Nos. 4,485,045 and 4,544,545; EP 102,324. The relevant sections of all referenced techniques are hereby incorporated by reference. Ordinarily, the liposomes are of the small unilamellar liposomes in about 200 to 800 Angstroms which the lipid content is greater than about 30 mol. % cholesterol, the selected proportion being adjusted for optimal therapy.

In a preferred embodiment, the dry powdered formulation in the disclosure may not be inhaled but rather injected as a dry powder, using relatively new injection devices and methodologies for injecting powders. In this embodiment, the dispersibility and respirability of the powder is not important, and the particle size may be larger, for example in about 10 μm, about 20 μm to about 40 μm, about 50 μm to about 70 am, about 100 μm. The dry powdered formulations in the disclosure may be reconstituted for injection as well. As the powder of the disclosure shows good stability, it may be reconstituted into liquid form using a diluent and then used in non-pulmonary routes of administration, e.g., by injection, subcutaneously, intravenously, etc. Known diluents may be used, including physiological saline, other buffers, salts, as well as non-aqueous liquids etc. It is also possible to reconstitute the dry powder of the disclosure and use it to form liquid aerosols for pulmonary delivery, either for nasal or intrapulmonary administration or for inhalation. As used herein, the term “treating” refers to therapeutic and maintenance treatment as well as prophylactic and preventative measures. Those in need of treatment include those already diagnosed with the disorder as well as those prone to having the disorder and those where the disorder is to be prevented. Consecutive treatment or administration refers to treatment on at least a daily basis without interruption in treatment for one or more days. Intermittent treatment or administration, or treatment or administration in an intermittent fashion, refers to treatment that is not consecutive, but rather cyclic in nature. The treatment regime herein may be consecutive or intermittent or of any other suitable mode. The dry powdered formulation may be obtained, for example, by sieving, lyophilization, spray-lyophilization, spray drying, and freeze drying, etc. These methods may be combined for improved effect. Filters may be employed for sieving, as will be known to a skilled artisan. The alteration and selection of the agent's particle size may be conducted in a single step, preferably, by micronizing under conditions effective to attain the desired particle size as previously described.

The dry powdered formulation may be then stored under controlled conditions of temperature, humidity, light, pressure etc., so long as the flowability of the agent is preserved. The agent's stability upon the storing may be measured at a selected temperature for a selected time period and for rapid screening a matrix of conditions are run, e.g., at 2-8° C., 30° C. and sometimes 40° C., for periods of 2, 4 and 24 weeks. The length of time and conditions under which a formulation should be stable will depend on a number of factors, including the above, amount made per batch, storage conditions, turnover of the product, etc. These tests are usually done at 38% (rh) relative humidity. Under these conditions, the agent generally loses less than about 30% biological activity over 18 months, sometimes less than about 20%, or less than about 10%. The dry powder of the disclosure loses less than about 50% FPF, in some cases less than about 30%, and in others less than about 20%.

The dry powder formulation of the disclosure may be combined with formulation ingredients, such as bulking agents or carriers, which are used to reduce the concentration of the agent in the dry powder being delivered to a patient. The addition of these ingredients to the formulation is not required, however, in some cases it may be desirable to have larger volumes of material per unit dose. Bulking agents may also be used to improve the flowability and dispersibility of the powder within a dispersion device, or to improve the handling characteristics of the powder. This is distinguishable from the use of bulking agents or carriers during certain particle size reduction processes (e.g., spraying drying). Suitable bulking agents or excipients are generally crystalline (to avoid water absorption) and include, but are not limited to, lactose and mannitol. If lactose, is added, for example, in amounts of about 99: about 1: about 5: active agent to bulking agent to about 1:99 being preferred, and from about 5 to about 5: and from about 1:10 to about 1:20.

The dry powder formulations of the disclosure may contain other drugs, e.g., combinations of therapeutic agents may be processed together, e.g., spray dried, or they may be processed separately and then combined, or one component may be spray dried and the other may not, while it is processed in one of the other manners enabled herein. The combination of drugs will depend on the disorder for which the drugs are given, as will be appreciated by those in the art. The dry powder formulation of the disclosure may also comprise, as formulation ingredients, excipients, preservatives, detergents, surfactants, antioxidants, etc, and may be administered by any means that transports the agent to the airways by any suitable means, but are preferably administered through the respiratory system as a respirable formulation, more preferably in the form of an aerosol or spray comprising the agent's particles, and optionally, other therapeutic agents and formulation ingredients.

In another embodiment, the dry powdered formulations may comprise the dry pharmaceutical agent of this disclosure and one or more surfactants. Suitable surfactants or surfactant components for enhancing the uptake of the active compounds used in the disclosure include synthetic and natural as well as full and truncated forms of surfactant protein A, surfactant protein B, surfactant protein C, surfactant protein D and surfactant protein E, disaturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine, phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamelar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomeric- and polymeric-, poly(vinylamine) with dextran and/or alkanoyl side chains, Brij 35©, Triton X-100©, and synthetic surfactants ALEC®, Exosurt®, Survan®, and Atovaquone®, among others. These surfactants may be used either as single or part of a multiple component surfactant in a formulation, or as covalently bound additions to the active compounds.

Examples of other therapeutic agents for use in the present formulation are analgesics such as Acetaminophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx.

Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti-anxiety agents associated with mental depression, such as Chlordiazepoxide, Amitriptyline, Loxapine Maprotiline and Perphenazine, among others. Anti-inflammatory agents such as non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, treatments such as Tricyclic Antidepressants, including Amitriptyline HCl (Elavil), Amitriptyline HCl, Perphenazine (Triavil) and Doxepin HCl (Sinequan). Examples of tranquilizers Estazolam, Flurazepam, Halazepam, Ketazolam, Lorazepam, Nitrazepam, Prazepam Quazepam, Temazepam, Triazolam, Zolpidem and Sopiclone, among others. Sedatives including Diphenhydramine, Hydroxyzine, Methotrimeprazine, Promethazine, Propofol, Melatonin, Trimeprazine, and the like.

Sedatives and agents used for treatment of petit mal and tremors, among other conditions, such as Amitriptyline HCl; Chlordiazepoxide, Amobarbital; Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g., for treatment of severe head injury; orphan status, Warner Lambert), cytoprotective agents, and agents for the treatment of menopause, menopausal symptoms (treatment), e.g., Ergotamine, Belladonna Alkaloids and Phenobarbital, for the treatment of menopausal vasomotor symptoms, e.g., Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre-menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications.

Such combination therapeutic formulations can be manufactured using many conventional techniques. It may be necessary to micronize the active compounds and if appropriate (i.e. where an ordered mixture is not intended) any carrier in a suitable mill, for example in a jet mill at some point in the process, in order to produce primary particles in a size range appropriate for maximal deposition in the lower respiratory tract (i.e., from about 0.1 μm to about 10 μm). For example, one can dry mix DHEA and carrier, where appropriate, and then micronize the substances together; alternatively, the substances can be micronized separately, and then mixed. Where the compounds to be mixed have different physical properties such as hardness and brittleness, resistance to micronization varies and they may require different pressures to be broken down to suitable particle sizes. When micronized together, therefore, the obtained particle size of one of the components may be unsatisfactory. In such case it would be advantageous to first micronize the different components separately and then mix them.

It is also possible first to dissolve the active component including, where an ordered mixture is not intended, any carrier in a suitable solvent, e.g., water, to obtain mixing on the molecular level. This procedure also makes it possible to adjust the pH-value to a desired level. The pharmaceutically accepted limits of pH 3.0 to 8.5 for inhalation products must be taken into account, since products with a pH outside these limits may induce irritation and constriction of the airways. To obtain a powder, the solvent must be removed by a process which retains the biological activity of DHEA. Suitable drying methods include vacuum concentration, open drying, spray drying, freeze drying and use of supercritical fluids. Temperatures over 50° C. for more than a few minutes should generally be avoided, as some degradation of the DHEA may occur. After drying step the solid material can, if necessary, be ground to obtain a coarse powder, and then, if necessary, micronized.

If desired, the micronized powder can be processed to improve the flow properties, e.g., by dry granulation to form spherical agglomerates with superior handling characteristics, before it is incorporated into the intended inhaler device. In such a case, the device would be configured to ensure that the agglomerates are substantially deagglomerated prior to exiting the device, so that the particles entering the respiratory tract of the patient are largely within the desired size range. Where an ordered mixture is desired, the active compound may be processed, for example by micronization, in order to obtain, if desired, particles within a particular size range. The carrier may also be processed, for example to obtain a desired size and desirable surface properties, such as a particular surface to weight ratio, or a certain texture, and to ensure optimal adhesion forces in the ordered mixture. Such physical requirements of an ordered mixture are well known, as are the various means of obtaining an ordered mixture which fulfils the said requirements, and may be determined easily by one skilled in the art.

The dry powder formulation of this disclosure may be administered into the respiratory tract as a formulation of respirable size particles i.e. particles of a size sufficiently small to pass through the nose, mouth, larynx or lungs upon inhalation, nasal administration or lung instillation, to the bronchi and alveoli of the lungs. In general, respirable particles range from about 0.1 μm to about 100 μm, and inhalable particles are about 0.1 μm to about 10 μm, to about 5 μm in size. Mostly, when inhaled, particles of non-respirable size that are included in the aerosol tend to deposit in the throat and be swallowed, which reduces the quantity of nonrespirable particles in the aerosol. For nasal administration, a particle size in the range of about 10 μm to about 20 μm, about 50 μm, about 60 μm, or about 100 μm, is preferred to ensure retention in the nasal cavity.

The size and shape of the particles may be analyzed using known techniques for determine and ensure proper particle morphology. For example, one skilled in the art can visually inspect the particles under a microscope and/or determine particle size by passing them through a mesh screen. Preferred techniques for visualization of particles include scanning electron microscopy (SEM) and transmission electron microscopy (TEM). Particle size analysis may take place using laser diffraction methods. Commercially available systems for carrying out particle size analysis by laser diffraction are available from Clausthal-Zellerfeld, Germany (HELOS H1006).

The dry powdered formulation of the disclosure may be delivered with any device that generates solid particulate aerosols, such as aerosol or spray generators. These devices produce respirable particles, as explained above, and generate a volume of aerosol or spray containing a predetermined metered dose of a medicament at a rate suitable for human or animal administration. One illustrative type of solid particulate aerosol or spray generator is an insufflator, which are suitable for administration of finely comminuted powders. The latter may be taken also into the nasal cavity in the manner of a snuff. In the insufflator, the powder, e.g., a metered dose of the agent effective to carry out the treatments described herein, is contained in a capsule or a cartridge. These capsules or cartridges are typically made of gelatin, foil or plastic, and may be pierced or opened in situ, and the powder delivered by air drawn through the device upon inhalation or by means of a manually-operated pump. The dry powder formulation employed in the insufflator may consist either solely of the agent or of a powder blend comprising the agent, and the agent typically comprises from 0.01 to 100% w/w of the formulation. The dry powdered formulation generally contains the active compound in an amount of about 0.01% w/w, about 1% w/w/, about 5% w/w, to about 20%, w/w, about 40% w/w, about 99.99% w/w. Other ingredients, and other amounts of the agent, however, are also suitable within the confines of this disclosure.

In a preferred embodiment, the dry powdered formulation is delivered by a nebulizer. This is means is especially useful for patients or subjects who are unable to inhale or respire the powder pharmaceutical composition under their own efforts. In serious cases, the patients or subjects are kept alive through artificial respirator. The nebulizer can use any pharmaceutically or veterinarily acceptable carrier, such as a weak saline solution. Preferably, the weak saline solution is less than about 1.0 or 0.5% NaCl. More preferably, the weak saline solution is less than about 0.2% or 0.15% NaCl. Even more preferably, the weak saline solution is less than about 0.12% NaCl. The nebulizer is the means by which the powder pharmaceutical composition is delivered to the target of the patients or subjects in the airways. The stability of anhydrous compounds, such as anhydrous DHEA-S, can be maintained or increased by eliminating or reducing the water content within the sealed container, e.g., vial, containing the compound. Preferably, besides the compound, it is a vacuum within the sealed container.

The formulation of the disclosure is also provided in various forms that are tailored for different methods of administration and routes of delivery. The formulations that are contemplated are, for example, a transdermal formulation also containing an excipient and other agents suitable for delivery through the skin, mouth, nose, vagina, anus, eyes, and other body cavities, intradermally, as a sustained release formulation, intrathecally, intravascularly, by inhalation, nasally, intrapulmonarily, into an organ, by implantation, by suppositories, as cremes, gels, and the like, all known in the art. In one embodiment, the dry powdered formulation comprises a respirable formulation, such as an aerosol or spray. The dry powder formulation of the disclosure is provided in bulk, and in unit form, as well as in the form of an implant, a capsule, blister or cartridge, which may be openable or piercable as is known in the art. A kit is also provided, that comprises a delivery device, and in separate containers, the dry powdered formulation of the disclosure, and optionally other excipient and therapeutic agents, and instructions for the use of the kit components.

In one preferred embodiment, the agent is delivered using suspension metered dose inhalation (MDI) formulation. Such a MDI formulation can be delivered using a delivery device using a propellant such as hydrofluroalkane (HFA). Preferably, the HFA propellants contain 100 parts per million (PPM) or less of water. N. C. Miller (In: Respiratory Drug Delivery, P. R. Bryon (ed.), CRC Press, Boca Raton, 1990, pp. 249-257) reviewed the effect of water content on crystal growth in MDI suspensions. When exposed to water, anhydrous DHEA-S will hydrate and eventually form large particles. This hydration process can happen in a suspension of the anhydrous DHEA-S in an HFA propellant which has a water content. This hydration process would accelerate the crystal growth due to the formation of strong interparticle bonds and cause the formation of large particles. In contrast, the dihydrate form is already hydrated thus more stable, and thus more preferred, than the anyhydrous form in a MDI, as the dihydrate form will not further form larger particles. If DHEA-S forms a solvate with a HFA propellant that has a lower energy than the dihydrate form, then this DHEA-S solvate would be the most stable, and hence more preferred, form for an MDI.

In one preferred embodiment, the delivery device comprises a dry powder inhalator (DPI) that delivers single or multiple doses of the formulation. The single dose inhalator may be provided as a disposable kit which is sterilely preloaded with enough formulation for one application. The inhalator may be provided as a pressurized inhalator, and the formulation in a piercable or openable capsule or cartridge. The kit may optionally also comprise in a separate container an agent such as other therapeutic compounds, excipients, surfactants (intended as therapeutic agents as well as formulation ingredients), antioxidants, flavoring and coloring agents, fillers, volatile oils, buffering agents, dispersants, surfactants, antioxidants, flavoring agents, bulking agents, propellants and preservatives, among other suitable additives for the different formulations. The dry powdered formulation of this disclosure may be utilized by itself or in the form of a composition or various formulations in the treatment and/or prevention of a disease or condition associated with bronchoconstriction, allergy(ies), lung cancer and/or inflammation. Examples of diseases are airway inflammation, allergy(ies), asthma, impeded respiration, CF, COPD, AR,ARDS, pulmonary hypertension, lung inflammation, bronchitis, airway obstruction, bronchoconstriction, microbial infection, viral infection (such as SARS), among others. Clearly the present formulation may be administered for treating any disease that afflicts a subject, with the above just being examples. Typically, the dry powdered formulation may be administered in an amount effective for the agent to reduce or improve the symptom of the disease or condition.

The dry powdered formulation may be administered directly to the lung(s), preferably as a respirable powder, aerosol or spray. Although an artisan will know how to titrate the amount of dry powdered formulation to be administered by the weight of the subject being treated in accordance with the teachings of this patent, the agent is preferably administered in an amount effective to attain an intracellular concentration of about 0.05 to about 10 μM agent, and more preferably up to about 5 μM. Propellants may be employed under pressure, and they may also carry co-solvents. The dry powdered formulation of the disclosure may be delivered in one of many ways, including a transdermal or systemic route, orally, intracavitarily, intranasally, intraanally, intravaginally, transdermally, intrabucally, intravenously, subcutaneously, intramuscularly, intratumorously, into a gland, by implantation, intradermally, and many others, including as an implant, slow release, transdermal release, sustained release formulation and coated with one or more macromolecules to avoid destruction of the agent prior to reaching the target tissue. Subject that may be treated by this agent include humans and other animals in general, and in particular vertebrates, and amongst these mammals, and more specifically and small and large, wild and domesticated, marine and farm animals, and preferably humans and domesticated and farm animals and pets.

### VI. Combination Therapies—Other Agents

In certain embodiments, the anti-PLESC agent can be administered conjointly with one or more agents that have other beneficial local activities in pulmonary tract.

In certain embodiments, the anti-PLESC agent is conjointly administered with an anti-inflammatory agent selected from an IL-1 inhibitor, an IL-1 receptor (IL-1R) inhibitor, an IL-6 inhibitor, an IL-6 receptor (IL-6R) inhibitor, a NLRP3 inhibitor, a TNF inhibitor, an IL-8 inhibitor, an IL-18 inhibitor, an inhibitor of natural killer cells, or combinations thereof. In some embodiments, the anti-inflammatory agent is a nucleic acid, an aptamer, an antibody or antibody fragment, an inhibitory peptide, or a small molecule.

In certain embodiments, the anti-PLESC agent is conjointly administered with an an NLRP3 inhibitor. In some embodiments, the NLPR3 inhibitor is an antisense oligonucleotide against NLPR3, colchicine, MCC950, CY-09, ketone metabolite beta-hydroxubutyrate (BHB), a type I interferon, resveratrol, arglabin, CB2R, Glybenclamide, Isoliquiritigenin, Z-VAD-FMK, or microRNA-223.

In certain embodiments, the anti-PLESC agent is conjointly administered with a TNF inhibitor. In some embodiments, the TNF inhibitor is an antisense oligonucleotide against TNF, infliximab, adalimumab, certolizumab pegol, golimumab, etanercept (Enbrel), thalidomide, lenalidomide, pomalidomide, a xanthine derivative, bupropion, 5-HT2A agonist or a hallucinogen.

In certain embodiments, the anti-PLESC agent is conjointly administered with an IL-18 inhibitor. In some embodiments, the IL-18 inhibitor is selected from the group consisting of: antisense oligonucleotides against IL-18, IL-18 binding protein, IL-18 antibody, NSC201631, NSC61610, and NSC80734.

In certain embodiments, the anti-PLESC agent is conjointly administered with an inhibitor of natural killer cells. In some embodiments, the inhibitor of natural killer cells is an antibody targeting natural killer cells.

In certain embodiments, the anti-PLESC agent is conjointly administered with methotrexate.

In certain embodiments, the anti-PLESC agent is conjointly administered with arhalofenate.

In certain embodiments, the anti-PLESC agent is conjointly administered with an IL-10 inhibitor.

STAT3 Inhibitors

In certain embodiments, the anti-PLESC agent is conjointly administered with a STAT3 inhibitor.

In one embodiment, the STAT3 inhibitor is Stattic. Stattic is nonpeptidic small molecule that potently inhibits STAT3 activation and nuclear translocation with IC₅₀ of 5.1 μM in cell-free assays, highly selectivity over STAT1.

Non-limiting examples of STAT3 inhibitors include BP-1-102, S31-M2001, STA-21, S3I-201, Galiellalactone, a polypeptide having the sequence PY*LKTK (where Y* represents phosphotyrosine), and a polypeptide having the sequence Y*LPQTV (where Y* represents phosphotyrosine). Additional non-limiting examples of STAT3 inhibitors are described in Yue and Turkson Expert Opin Investig Drugs. 2009 January; 18(1): 45-56, the entire content of which is incorporated herein by reference.

Other STAT3 inhibitors include: E1: 4_-Bromo-phenyl-2-N-aminoacyl-1 1-dioxide-benzo [b]thiophene; E2: 4_-bromo-2-N-(4-fluorophenyl) alanyl-1,1-dioxide, benzo [b] thiophene; E3: 4-bromo-benzo 2-N-(4-methoxyphenyl) alanyl-1,1-dioxide [b] thiophene; E4: 4_-bromo-2-N-aminoacyl-p-tolyl-1,1-oxidation benzo [b] thiophene; E5: 4_-bromo-2-N-(4-chlorophenyl) alanyl-1,1-dioxide, benzo [b] thiophene; E6: 4_-bromo-2-N-benzo (3-chlorophenyl) alanyl-1,1-dioxide [b] thiophene; E7: 4_-bromo-2-N-(2-chlorophenyl) alanyl-1,1-dioxide benzo [b]thiophene; E8: 4_-bromo-2-N-(3-chloro-4-fluorophenyl) alanyl-1,1-dioxide, benzo [b]thiophene; E9: 4_-chloro-2-N-aminoacyl-phenyl-1,1-dioxide, benzo [b] thiophene; E10: 5-bromo-phenyl-2-N-aminoacyl-1,1-dioxide, benzo [b] thiophene; EII: 6_bromo-phenyl-2-N-aminoacyl-1,1-dioxide, benzo [b] thiophene; E12: 2-N-aminoacyl-phenyl-1,1-dioxide, benzo [b]thiophene; E13: 5_-nitro-phenyl-2-N-Acyl-1,1-dioxide, benzo [b] thiophene; E14: 5_-bromo-n-butyl-2-N-aminoacyl-1,1-dioxide, benzo [b] thiophene; E15: 5_bromo-2-N-aminoacyl-t-butyl-1,1-dioxide, benzo [b] thiophene; E16: 5_-bromo-2-N-isopropyl-alanyl-1,1-benzo [b] dioxide thiophene; E17: 5_-bromo-2-N-cyclohexyl-alanyl-1,1-benzo [b] thiophene dioxide; E18: 5-bromo-2-N-[(3s, 5s, 7s)-1-adamantyl]-1,1-aminoacyl dioxide benzo [b] thiophene; E19: 4-bromo-benzo-2-N-benzyl-aminoacyl-1,1-dioxide [b] thiophene; E20: 4_-bromo-2-N-(4-bromophenethyl) benzo-1,1-dioxide aminoacyl [b] thiophene; E21: 5_-bromo-2-N-(4-phenoxy-phenyl) amino-benzo-1,1-dioxide group [b] thiophene; E22: 5_-bromo-2-N-[4-(I-piperidinyl-carbonyl) phenyl]-1,1-aminoacyl dioxide benzo [b] thiophene; E23: 5_-bromo-2-N-[4-(4-morpholin-ylcarbonyl) phenyl] carboxamido-1,1-dioxide, benzo [b] thiophene; E24: 5_-bromo-2-N-[4-(N-methyl-N-phenyl) carbamoyl] phenyl-carboxamido-1,1-dioxide, benzo [b]thiophene; E25: 4_bromo-2-p-tolyl-carboxy-1,1-dioxide [b] thiophene; E26: 5_bromo-2-N, N-diethyl-1,1-dioxide aminoacyl benzothienyl; E27: 5_-bromo-2-(I-pyrrolyl) carbonyl-1,1-dioxide, benzo [b] thiophene; E28: 5_-bromo-2-(1-piperidyl) carbonyl-1,1-dioxide, benzo [b] thiophene; E29: 5_-bromo-2-(2-methyl-piperidine yl) carbonyl-1,1-dioxide, benzo [b] thiophene; E30: benzo 5_-bromo-2-(3-methyl-1-piperidinyl) carbonyl-1,1-dioxide [b] thiophene; E31: 5_-bromo-2-morpholino-carbonyl-1,1-dioxide, benzo [b] thiophene; E32: 5_-bromo-2-(4-ethyl-1-piperazinyl) carbonyl-1, 1-dioxide-benzo [b] thiophene; E33: 5_-bromo-2-(N-methyl-N-phenyl) benzo-1,1-dioxide aminoacyl [b] thiophene; E34: 4_bromo-2-(I-piperidinyl) carbonyl-1,1-dioxide, benzo [b] thiophene; E35: 5_trifluoromethyl-2-(I-piperidinyl) carbonyl-1,1-dioxide benzo [b]thiophene; E36: 4_-bromo-2-methoxycarbonyl-1,1-dioxide, benzo [b] thiophene; E37: 2_methoxycarbonyl-1,1-oxidation benzo [b] thiophene; E38: benzo 5_acetamido-2-N-phenyl-1,1-dioxide aminoacyl [b] thiophene; E39: 5_benzoylamino-2-N-aminoacyl phenyl-1,1-dioxide, benzo [b] thiophene; E40: 5_of Methylbenzamido-2-N-aminoacyl-phenyl-1,1-dioxide, benzo [b]thiophene; E41: 5_Trifluoromethyl-benzoyl-phenylcarbamoyl group-2-N-acyl-1,1-dioxide, benzo [b] thiophene; E42: 5_p-chlorobenzoyl-N-phenylcarbamoyl group an acyl-2-1,1-benzo [b]thiophene dioxide; E43: 5_-cyclohexyl-carboxamido-2-N-phenyl-aminoacyl-1,1-dioxide, benzo [b] thiophene; or E44: 5_benzamido-2-(I-piperidinyl) carbonyl 1,1-dioxide, benzo [b] thiophene.

IL-6 Inhibitors

In certain embodiments, the anti-PLESC agent is conjointly administered with an IL-6 inhibitor, such agent that binds to IL-6 or the IL-6 receptor and prevents the interaction of those two molecules, or which inhibits signal transduction resulting from IL-6 binding to IL-6R containing receptor complexes. These include anti-IL-6 antibodies and antibody mimetic, anti-IL-6 receptor antibodies and antibody mimetics and small molecules, as well as nucleic acids which down-reulate IL-6 mediated signal transduction.

Exemplary agents targeting IL-6 or the IL-6 receptor include such as tocilizumab (Actemra), siltuximab (Sylvant), sarilumab, ALX-0061, sirukumab, MED15117, clazakizumab, and olokizumab. Tocilizumab is an example of an antibody directed against the IL6-receptor, siltuximab is directed against IL-6 itself.

In some embodiments, the anti-inflammatory agent comprises an IL-6 inhibitor. In some embodiments, the IL-6 inhibitor is an antisense oligonucleotide against IL-6, siltuximab, sirukumab, clazakizumab, olokizumab, elsilimomab, IG61, BE-8, CNT0328 PGE1 and its derivatives, Ppulmonary2 and its derivatives, or cyclophosphamide.

In some embodiments, the anti-inflammatory agent comprises an IL-6R inhibitor. In some embodiments, the IL-6R inhibitor is an IL-6R antagonist. In some embodiments, the IL-6R inhibitor is an antisense oligonucleotide against IL-6R, tocilizumab, sarilumab, PM1, AUK 12-20, AUK64-7, AUK146-15, MRA, or AB-227-NA.

IL-8 Inhibitors

In certain embodiments, the anti-PLESC agent is conjointly administered with an IL-8 inhibitor. In some embodiments, the IL-8 inhibitor is an antisense oligonucleotide against IL-8, HuMab-10F8, repertaxin, Curcumin, Antileukinate, Macrolide or a trifluoroacetate salt.

IL-1 Inhibitors

In some embodiments, the anti-inflammatory agent comprises an IL-1 inhibitor. In some embodiments, the IL-1 inhibitor is an IL-1a inhibitor. In some embodiments, the IL-1a inhibitor is an antisense oligonucleotide against IL-1a, MABpI, or sIL-1RI. In some embodiments, the IL-1 inhibitor is an IL-1b inhibitor. In some embodiments, the IL-1b inhibitor is an antisense oligonucleotide against IL-1b, canakinumab, diacerein, gevokizumab, LY2189102, CYT013, sIL-IRII, VX-740, or VX-765. In some embodiments, the IL-1 inhibitor is suramin sodium, methotrexate-methyl-d3, methotrexate-methyl-d3 dimethyl ester, or diacerein.

In some embodiments, the anti-inflammatory agent comprises an IL-1R inhibitor. In some embodiments, the IL-1R inhibitor is an IL-1R antagonist. In some embodiments, the IL-1R inhibitor is an anti-sense oligonucleotide against IL-1R, anakinra, Rilonacept, MEDI-8968, sIL-IRI, EBI-005, interleukin-I receptor antagonist (IL-1RA), or AMG108.

### VII. Certain Examples—Chronic Obstructive Pulmonary Fibrosis

### A. Results

In the study described below, the inventors leveraged robust technologies that enable the cloning of distal airway epithelial cells (Kumar et al., 2011; Zuo et al., 2015) to perform a comparative analysis of clonogenic cells in patients with and without COPD. The data revealed that the COPD distal airways are populated by a highly stereotyped triad of clonogenic cells in addition to the normal clonogenic cell type that predominates control lungs. The inventors show here these variants are stably committed to metaplastic fates and autonomously drive proinflammatory and pro-fibrotic programs akin to those implicated in COPD pathology. Lastly, they show that a similar triad of clonogenic cells in COPD also exists in control adult and 13-, 14- and 17-week fetal lung, albeit at low ratios to the predominant distal airway clone type seen in normal lung. Thus the variant clones predate any disease state and yet have features that could contribute to the emergence, pathology, or progression of COPD as a function of their numbers.

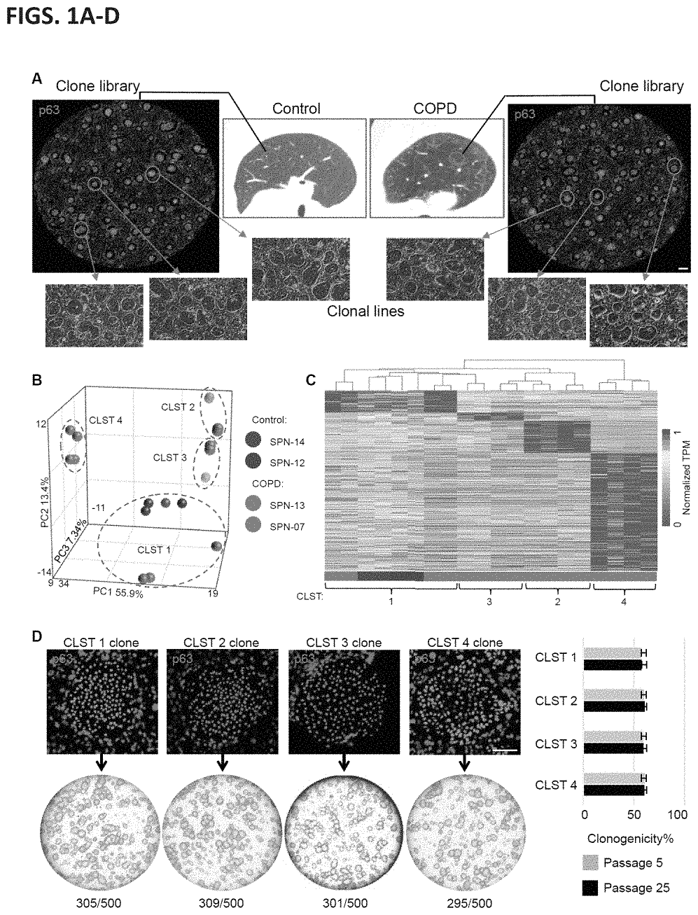

Heterogeneity of clonogenic cells in COPD. Libraries of clonogenic epithelial cells from resected lung tissue of 19 COPD and 11 control patients were generated by selective colony growth on lawns of irradiated, 3T3-J2 feeder cells (Kumar et al., 2011; Zuo et al., 2015; detailed in Methods). In brief, approximately 0.85+/−0.04% of all enzymatically digested lung cells yielded colonies from COPD patients and 0.15+/−0.02% from controls. These libraries have a complexity of 2,000 to 20,000 independent colonies per cubic centimeter of lung tissue and consist exclusively of cells that co-express E-cadherin and p63 (Ecad+/p63+). Single cells from these patient-specific libraries have a clonogenicity of greater than 50% as judged by single cell-sorting to 384-well plates and give rise to discrete lines of highly immature, epithelial cells characterized by a long-term (>1 yr) proliferative potential. Randomly sampled clones from libraries of control patients (SPN-12, -14) displayed whole genome expression profiles consistent with previously characterized clonogenic cells of distal airways (Kumar et al., 2011; Zuo et al., 2015) (FIG. 1B, C). In contrast, sampled clones from libraries derived from COPD patients (SPN-7, -13) yielded a more complex spectrum of gene expression profiles. In addition to the normal distal airway clones (hereafter “Cluster 1”), the majority of sampled clones showed whole genome expression profiles that diverged from Cluster 1 and readily segregated into three additional distinct clusters (Clusters 2, 3, and 4) (FIG. 1B,C). Despite this clonal heterogeneity in COPD, all clones from Clusters 1-4 shared the expression of established markers of distal airway progenitors (p63, KRT5), displayed a clonogenicity of 60+/−1.4%, and showed a long-term proliferative potential of at least 25 passages (>8 mos) while maintaining a clonogenicity of 59.9+/−1.6% (FIG. 1D). Herein the inventors leverage the regenerative properties of these patientspecific libraries of clonogenic cells and the single cell-derived clones from them to assess the properties of epithelial progenitors in COPD and control cases.

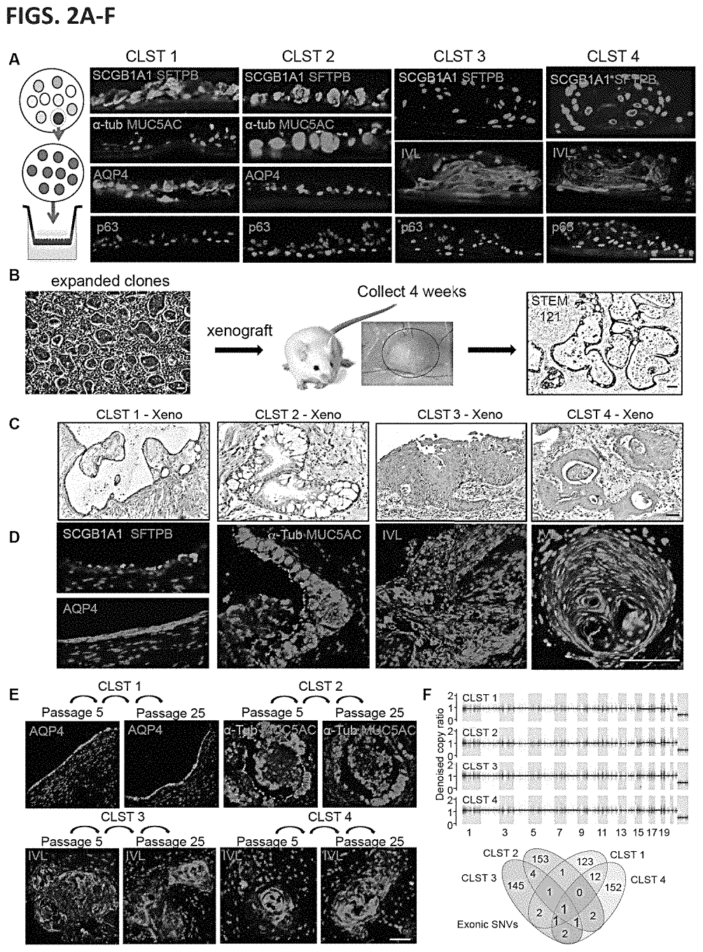

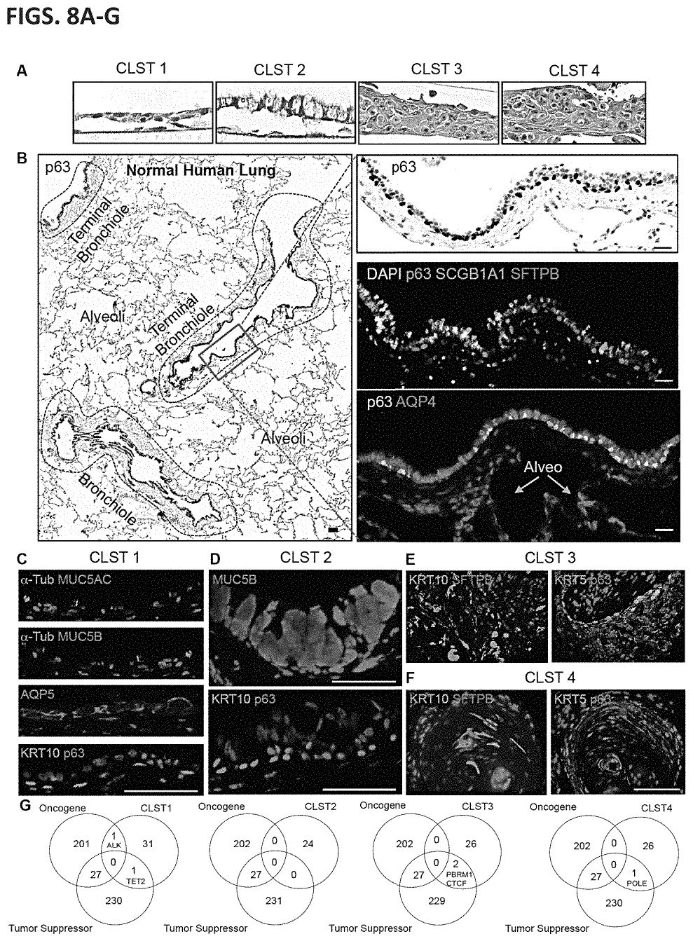

Metaplastic fate commitment of COPD clones. In vitro differentiation of Cluster 1 clones from both COPD and control libraries yielded normal epithelia (NM) marked by p63+ basal cells and suprabasal, differentiated cells expressing SCGB1A1, SFTPB, and AQP4, similar to human epithelia of small airways and terminal bronchioles (FIG. 2A; FIGS. 8A,B). In contrast, clones of Cluster 2 differentiated to a goblet cell metaplasia (GCM) marked by p63+ basal cells and differentiated goblet cells co-expressing SCGB1A1, MUC5AC, and MUC5B, whereas clones from Clusters 3 and 4 gave rise to squamous cell metaplasia (SCM) marked by immature p63 cells and differentiated, keratin 10 (Krt10)- and involucrin (IVL)-expressing cells (FIG. 2A; FIG. 8A).

We further tested the fates of cluster-specific clones in vivo by transplanting discrete clones into highly immunodeficient NSG (NODscidIL2ranull; Shultz et al., 1995) mice. Briefly, 1 million cells of a designated clone were mixed with 50% Matrigel, injected subcutaneously, and the resulting xenograft nodule examined four weeks later (FIG. 2B). Significantly, normal control clones, as well as those from Cluster 1 of COPD patients, assembled into a polarized epithelium in vivo comprised of cells positive for p63, Krt5, SCGB1A1, AQP4, AQP5, SFTPB, and SFTPC (FIG. 2C,D) like that of normal human terminal bronchioles (FIG. 8B) or that produced by in vitro differentiation of Cluster 1 clones (FIG. 2A FIG. 8A). Also mirroring the in vitro ALI cultures, the xenografts of Cluster 2 clones from COPD libraries formed an epithelium dominated by large goblet cells (FIG. 2C,D; FIG. 8D) expressing MUC5AC and MUC5B (Fahy and Dickey, 2010; Kesimer et al., 2017; Okuda et al., 2019), and both Cluster 3 and 4 clone xenografts yielded squamous cell metaplasia expressing IVL and Krt10 (FIG. 2C,D; FIGS. 8E,F). Despite their commitment to histologically similar squamous cell metaplasia, Cluster 3 and 4 clones exhibit distinct gene expression profiles (FIGS. 1B,C). Cluster 4 clones in particular constitutively express a broad array of genes related to inflammation and are denoted herein as ‘inflammatory SCM’ or ‘iSCM’ versus ‘SCM’ for Cluster 3 clones. Importantly, the respective differentiation fates of these clone types in xenografts proved to be remarkably stable to 250 days of continuous propagation in vitro, suggesting that these metaplasia are the consequences of highly stable, cell-autonomous fate programs (FIG. 2E). Lastly, whole exome DNA sequencing of Cluster 2, 3, and 4 clones derived from these COPD patients showed an absence of copy number variation events greater than 10 Kb (FIG. 2F). Moreover, the 127-157 single nucleotide variation (synonymous, nonsynonymous, indels) events in these clones were consistent in number with other somatic cells (Lee-Six et al., 2018) and did not impact known tumor suppressor or oncogenes such as p16 or p53 (FIG. 2G; FIG. 8G, arguing against the possibility these clones are related to neoplastic lesions.

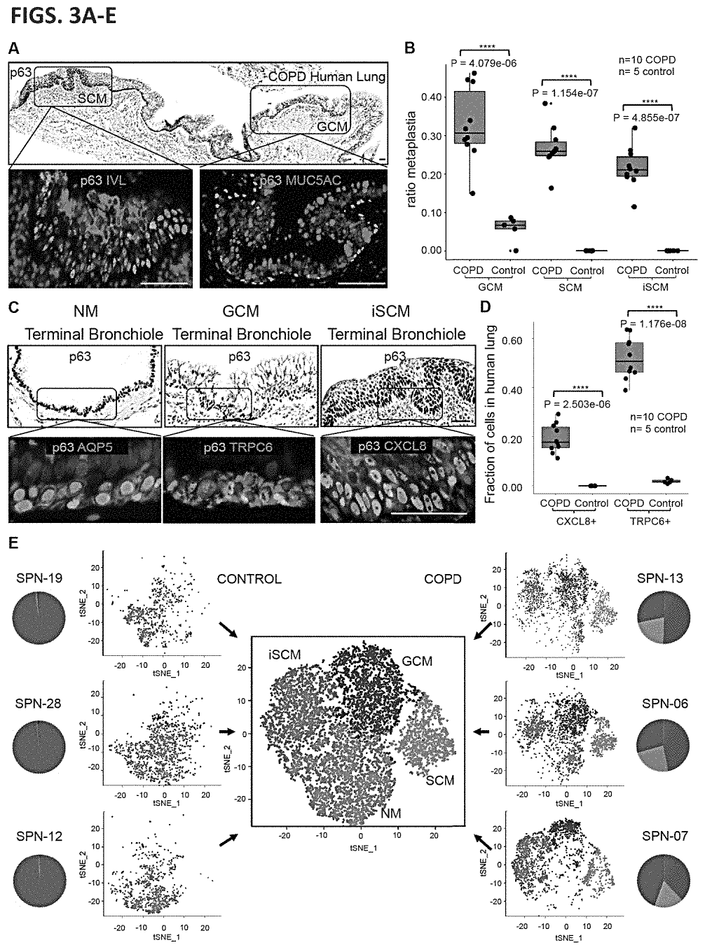

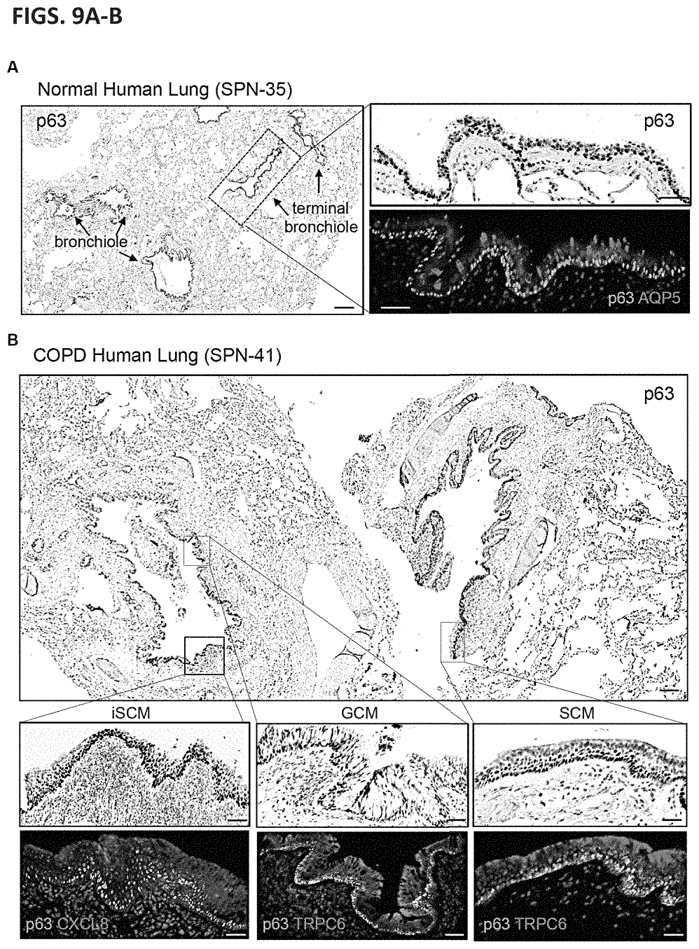

Metaplastic lesions dominate end-stage COPD lung. Given the observation that the Clusters 1-4 clones expressed p63 and yet differentiated to cluster specific metaplasia in vitro and in vivo, the inventors asked if metaplasia in the distal airways of COPD lung exhibited a similar association with p63+ basal cells. To address this question, they morphometrically quantified the distribution of normal distal airway epithelia and metaplasia in histological sections of distal lung from five additional donors lacking lung or systemic disease as well as from an additional 10 lungs of Stage 4 COPD transplant recipients. Whereas metaplastic lesions in the normal lungs were rare, the end-stage COPD distal airways were replete with goblet cell (MUC5AC+) and squamous cell (IVL+) metaplasia that, like normal distal airway epithelia, were subtended by basally-positioned p63+ cells (FIG. 3A). In quantitative terms, GCM occupied a mean of 32.7% of the distal airway epithelia (defined as that subtended by p63+ basal cells) of COPD lung versus 5.7% of the normal lungs, SCM occupied 26.8% of the COPD airways epithelia compared to less than 1% in normal lungs, and SCM associated with inflammation (iSCM) constituted approximately 21.6% of COPD airways and was not detected in normal lungs (FIG. 3B). The inventors also examined the distribution of marker genes associated with the p63+ basal cells of normal (AQP5+), iSCM (CXCL8+), and SCM/GCM (TRPC6+) airway epithelia in the same normal and Stage 4 COPD lungs (FIG. 3C). Consistent with our morphometric analyses of lung metaplasia in COPD and control lung, CXCL8+ basal cells of iSCM 7 comprised 19.3% of all p63+ distal airway basal cells of COPD lung while these were not observed in normal lungs. Fully 52% of the basal cells in COPD lung expressed TRPC6, a marker of both GCM and SCM, while fewer than 2% of basal cells showed TRPC6 expression in the normal lungs (FIG. 3D; FIGS. 9A,B). The close association of p63+ cells with the major forms of metaplasia in COPD is consistent with our finding that COPD lung possesses an array of p63+ clonogenic cells with absolute commitments to normal airway epithelia, goblet cell metaplasia, or squamous cell metaplasia. These findings are also in agreement with earlier studies that showed metaplasia in COPD and idiopathic pulmonary fibrosis were subtended by p63+ cells (Chilosi et al., 2002; Araya et al., 2007; Plantier et al., 2011; Seibold et al., 2013; Smirnova et al., 2016).

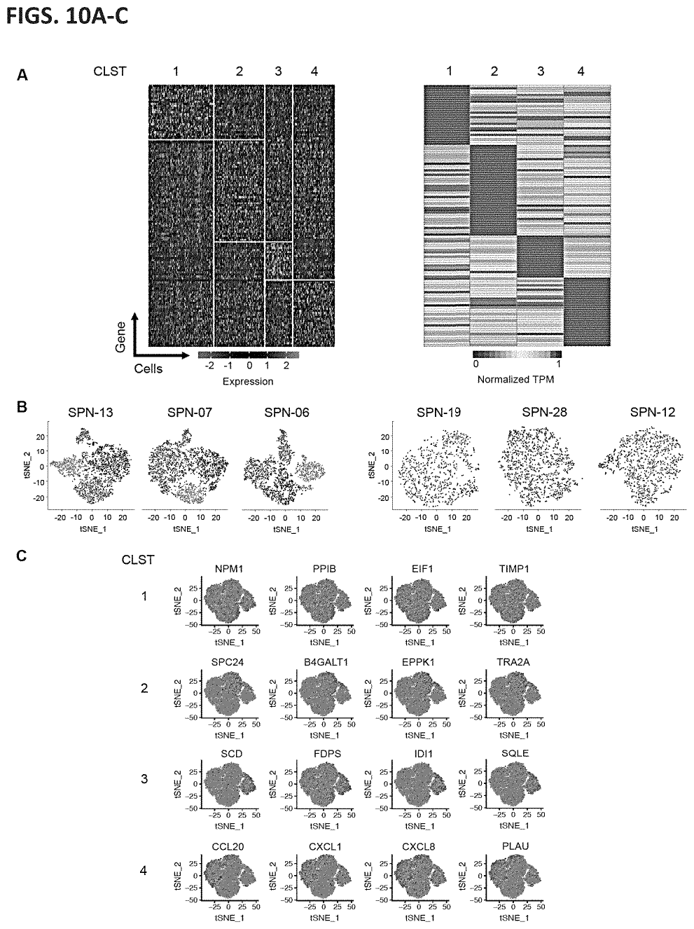

Clonal architecture of COPD and control libraries. The inventors next asked if widespread distribution of metaplastic lesions in the distal airways of end-stage COPD lung was reflected in the properties of the clonogenic cell libraries generated from our 19 COPD and 11 non-COPD cases. Single-cell RNA sequencing (scRNAseq; Satija et al., 2015) analyses of three COPD and three control libraries revealed four major clusters with gene expression profiles similar to those identified by single colony sampling as Clusters 1-4 (FIG. 3E; FIGS. 10A-C). In particular, the three COPD lungs showed major contributions to the clonal architecture of these libraries by clones committed to GCM, SCM, and iSCM represented by Clusters 2, 3, and 4, respectively. The coherence between markers identified by scRNAseq and by RNAseq of representative clones of each cluster was high, suggesting that our sampling approach likely captured all major clone variants in the COPD patients (FIG. 3E; FIG. 10A). This analysis also indicated that while control libraries were dominated by Cluster 1 clones committed to distal airway epithelia, these libraries also harbored small percentages (6-12%) of metaplastic variants (FIG. 3E; FIG. 10B).

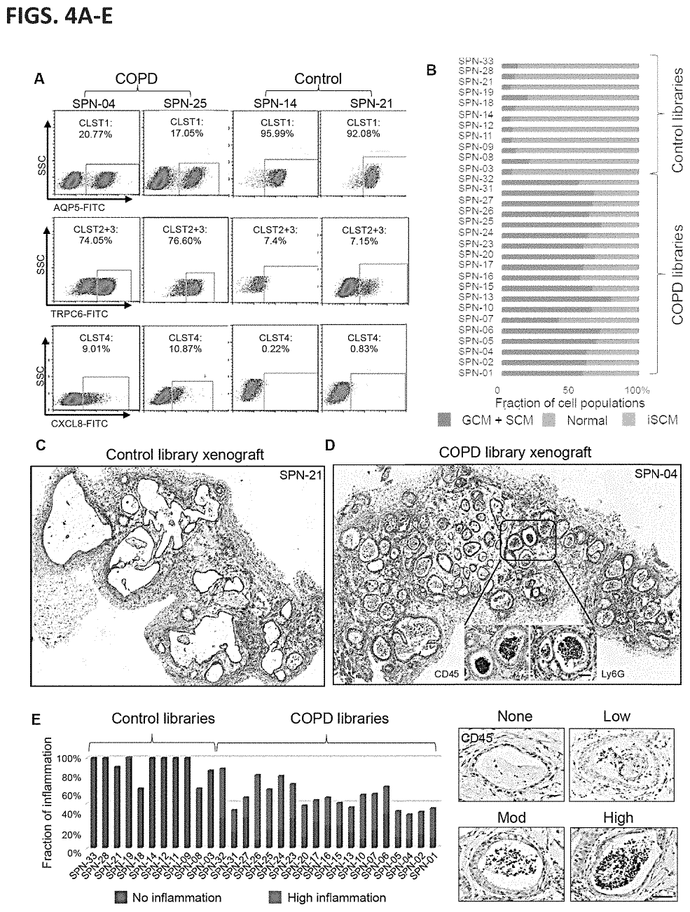

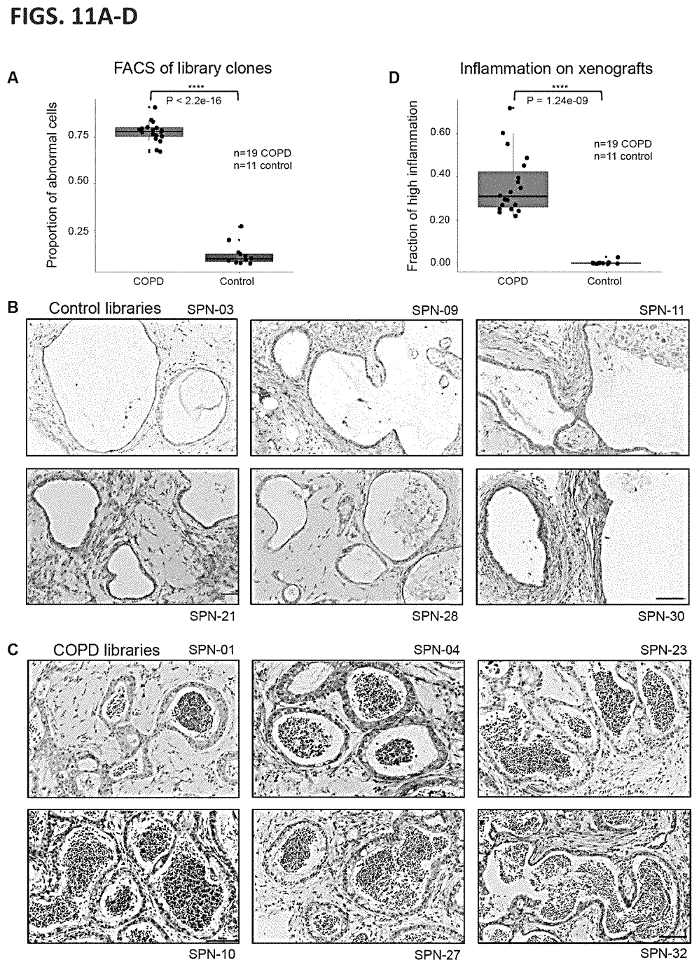

To develop a more simplified means of assessing the heterogeneity of clone libraries from COPD and Control patients, the inventors exploited the consensus markers identified by scRNAseq and RNAseq of representative clones for quantitative flow cytometry (FACS) analysis. Antibodies to AQP5 were specific to Cluster 1 cells, CXCL8 for Cluster 4 cells, and TRPC6 antibodies for both Clusters 2 and 3 (FIG. 4A). This limited set of markers was used to quantify the relative distribution of clone variants across our 19 COPD and 11 Control patients (FIG. 4A, B). Consistent with our analyses of scRNAseq profiles, the quantification of clone types by flow cytometric analyses of markers showed that variants (TRPC6+ for Clusters 2 and 3; CXCL8+ for Cluster 4) comprised 77.7+/−5% of all cells in the 19 COPD clone libraries, while in control specimens, 12.2+/−6% of the p63+ clonogenic cells in the 11 control libraries fell within Clusters 2-4 based on FACS analyses of markers TRPC6 and CXCL8 (P<2.2e−16; FIG. 4B; FIG. 11A).

Xenografts of COPD clone libraries drive neutrophilic inflammation. To assess the pathogenic potential of the COPD epithelial cells, the inventors performed subcutaneous transplants of composite clone libraries of each of the 19 COPD patients and 11 control patients into immunodeficient mice. Over four weeks, clone libraries from control patients yielded epithelial cysts with appropriate basal-apical polarity around largely vacant spaces (FIG. 4C; FIG. 11B). In contrast, clone libraries generated from COPD patients produced cysts occupied by dense arrays of epithelial and nonepithelial cells (FIG. 4D; FIG. 11C). The majority of non-epithelial luminal cells were hematopoietic in origin as evidenced by anti-murine CD45 staining, and the majority of those were positive for antibodies to Ly6G, a marker of neutrophils (FIG. 4D). Similar patterns of leukocyte transepithelial migration (TEM; Brazil and Parkos, 2016) are present in COPD lung (Quint and Wedzicha, 2007; Barnes, 2015; Butler et al., 2018). Quantification of the extent of neutrophil infiltration at the level of individual epithelial cysts revealed that 36.4+/−14% of these cysts scored “severe” (closest neutrophil packing) in the COPD clone library xenografts in contrast to only 0.3+/−0.9% of the cysts in control library xenografts (P=1.2e−09, Student's t-test; FIG. 4E; FIG. 11D).

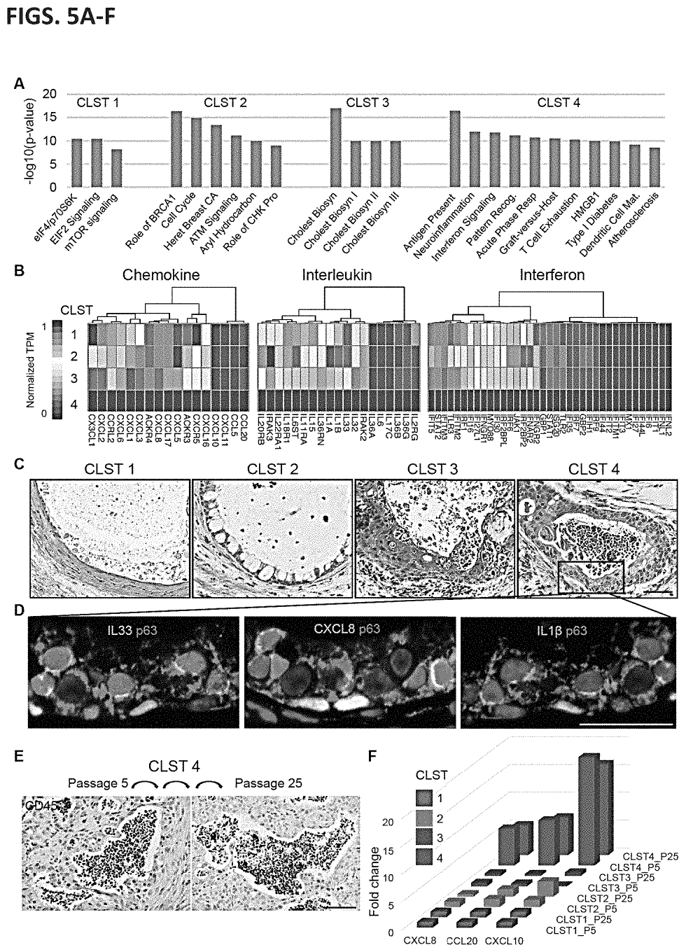

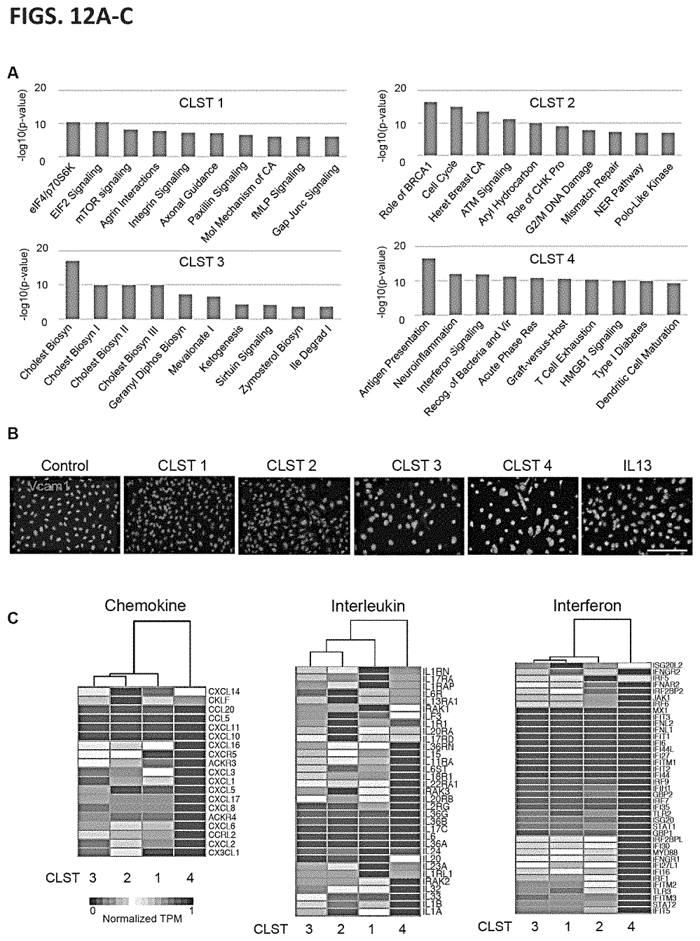

Given the robust inflammatory response triggered by xenografts of COPD clone libraries, the inventors asked if this inflammatory activity was a function of one or more of the constituent clone types. Using pathway enrichment analysis algorithms for the gene expression profiles of the four clone types, the multiple inflammatory pathways highlighted in clones of Cluster 4 was striking including Antigen Presentation, Interferon Signaling, Graft-vs-Host responses, and Dendritic Cell Maturation among others, and their relative absence from clones of Clusters 1, 2, and 3 (FIG. 5A; FIG. 12A). The inventors tested media conditioned by clones of Clusters 1-4 for their ability to trigger phase I and II activation of human airway microvasculature endothelial cells, an essential step in leukocyte recruitment (Filippi, 2016). Endothelial cell activation was assessed by the expression of Vcam1 (CD106), a vascular adhesion protein that binds VLA-1 (alpha4beta1 integrin) on monocytes and lymphocytes, and it was found that Cluster 1 clones from COPD patients showed no induction, whereas IL-13 challenge yielded a 30-fold induction of Vcam1 (FIG. 12B). In this same assay media conditioned by Cluster 2 (GCM) or Cluster 3 (SCM) clones, respectively, yielded 4- and 10-fold inductions of VCAM1, and while media from Cluster 4 (iSCM) clones induced Vcam1 by 60-fold (FIG. 12B). These data provide functional support for the notion that individual clone variants can promote, at least in vitro, an early step in inflammatory responses.

A more detailed expression heatmap of the variant clones revealed that Cluster 4 clones in particular displayed a constitutive expression of a broad array of chemokines, interleukins, and interferon genes compared to clones from Cluster 1, 2, and 3 (FIG. 5B; FIG. 12C). Among the large array of Cluster 4-specific chemokines were key determinants of neutrophil responses including CXCL1, CXCL5, and CXCL8 (Traves et al., 2002; Barnes, 2016; Brazil and Parkos, 2016; Ponce-Gallegos et al., 2017), the lymphocyte and dendritic cell chemoattractant CCL20 (Demedts et al., 2007), and others such as CXCL10 and 11 implicated in Th1 responses (Tokunaga et al., 2018). In addition, Cluster 4 clones expressed a broad array of interleukin genes, including IL1α, IL1β, IL6, IL17C, and IL33, known to interact with multiple cellular mediators of the innate and adaptive immune response (Barnes, 2016; Byers et al., 2013; Holtzman et al., 2014). Lastly, this analysis highlighted Cluster 4 clones as a robust source of Type I and Type II interferon pathway genes typically induced by viral infection (Schneider et al., 2014). In fact Cluster 4 clones showed a constitutively high expression of genes implicated in the entire interferon-γ pathway, including those involved in recognition of pathogen-associated molecular signatures (PAMPs), such as TLR2, TLR3, and MYD88, transcription factors driving the interferon-response genes (IRF1, IRF6, IRF7, and IRF9), as well as genes in the JAK-STAT pathway that cooperate in driving the interferon response such as STAT1, STAT2, and JAK1 (FIG. 5B; FIG. 12C). Together with the patterns of chemokine and interleukin gene expression, these data suggest the potential of Cluster 4 clones to promote an inflammatory response involving diverse cells of the immune system.

To directly test the prediction that Cluster 4 clones were primarily responsible for host neutrophil responses in whole COPD library transplants (cf. FIG. 4D), the inventors performed xenografts of the individual clone types in immunodeficient mice. Significantly, nodules formed by xenografts of Cluster 4 clones showed robust leukocyte infiltration (FIG. 5C). Consistent with their proinflammatory properties in xenografts, the epithelial cysts formed by Cluster 4 clones were unique among the four clone types by showing high protein expression of multiple inflammatory mediators such as IL33, CXCL8, and L1β (FIG. 5D). Lastly, the leukocyte infiltration response to Cluster 4 clones proved stable for eight months of continuous propagation in vitro (FIGS. 5E,F) suggesting that their proinflammatory activity is epigenetically maintained. Together, these data support the conclusion that Cluster 4 clones play a major and likely autonomous role in promoting the observed host leukocyte responses observed in the COPD clone library transplants.

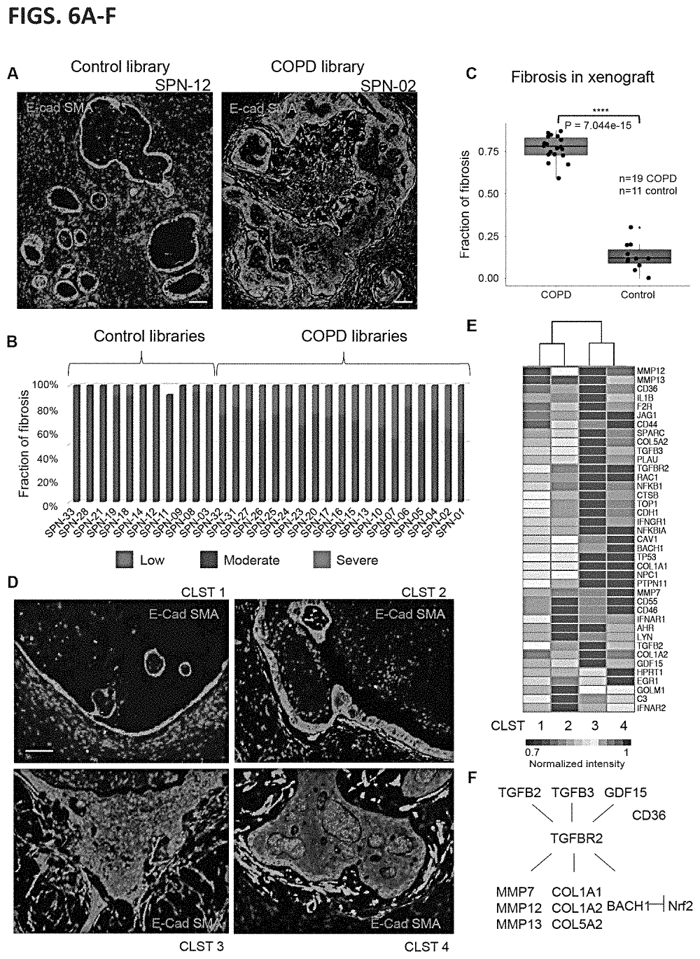

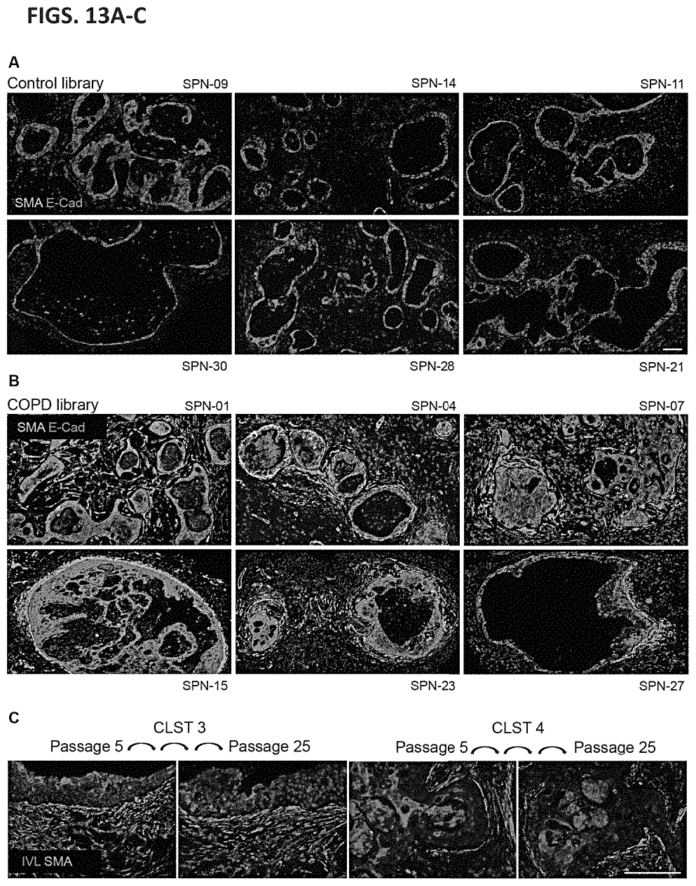

Clonogenic variants drive fibrosis. Fibrosis of small airways and respiratory vasculature is an established feature of COPD (Hogg and Timens, 2009; Barnes, 2016; Araya and Nishimura, 2010; Aschner and Downey, 2016) and has been associated with TGFβ-induced activation of myofibroblasts (Murphy-Ullrich and Suto, 2018; Black et al., 2019). In this regard, the inventors observed that many of the epithelial cysts in xenographs of clone libraries from all 19 COPD patients were surrounded by dense layers of fibroblast-like cells (FIG. 4D). Antibodies to alpha-smooth muscle actin (α-SMA), a key marker of activated myofibroblasts, labeled these submucosal assemblies along the majority (77.1+/−7.2%) of the perimeter of the epithelial cysts in COPD xenografts, whereas only relatively few of these cysts in control xenografts showed these myofibroblast arrays (12.9+/−8.1%; P=7.044e−15, Student's ttest; FIGS. 6A-C; FIGS. 13A,B). Similar accumulations of submucosal myofibroblasts were observed in xenographs of single Cluster 3 or Cluster 4 clones, but not in xenografts of clones of Clusters 1 or 2 (FIG. 6D). These data link the two squamous cell metaplasia produced by Clusters 3 and 4 clones, but not goblet cell metaplasia nor normal airway epithelia, to the accumulation of myofibroblasts. Consistently, gene expression profiling of xenografts derived from clones of the respective Clusters revealed differential expression of multiple, pro-fibrotic genes in xenografts of clones of Cluster 3 and 4 that were not evident in xenografts of Cluster 1 (FIG. 6E). Among this large set of constitutively expressed genes implicated in the regulation of fibrosis were TGFβ3, GDF15, and TGFβR2, downstream genes including collagen isotypes (e.g., COL1A1), matrix metallopeptidases (MMP7, MMP13), and BACH1 (Dhakshinamoorthy et al., 2005), a transcriptional repressor of the Nrf2-activated anti-oxidant pathway known to suppress fibrosis in multiple tissues (FIG. 6F). Finally, like the proinflammatory effect of Cluster 4, the pro-fibrotic effects of clones of Clusters 3 and 4 were stable to long-term propagation in vitro (FIG. 13C).

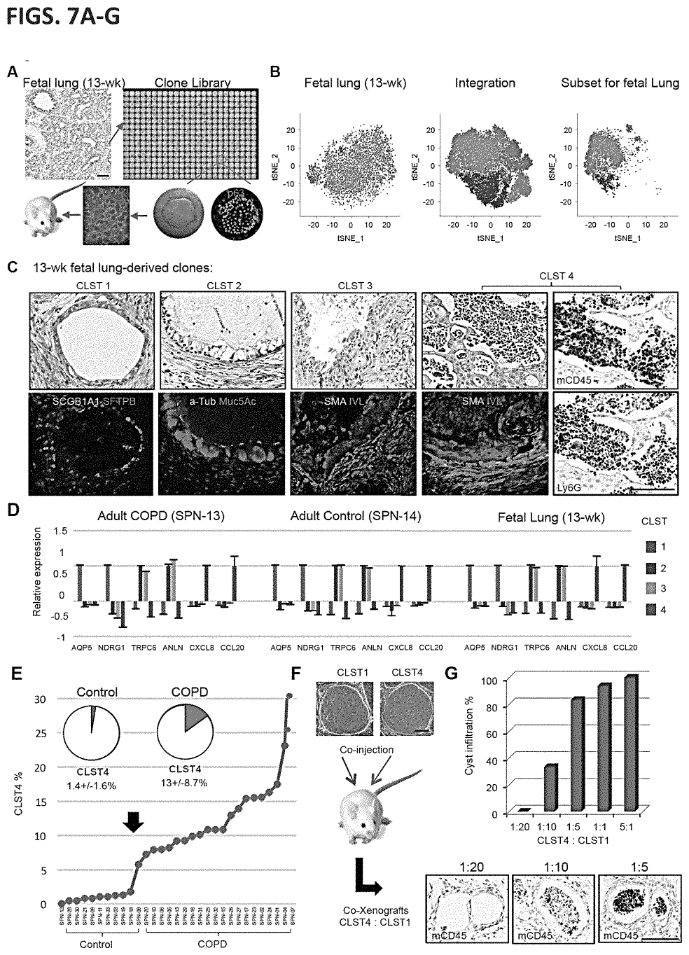

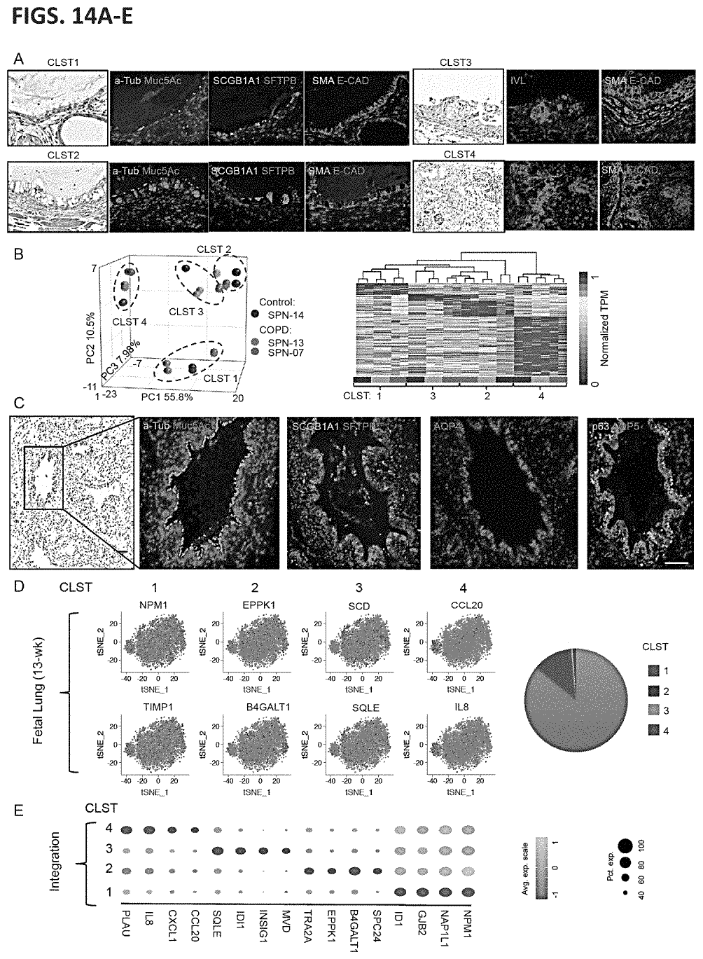

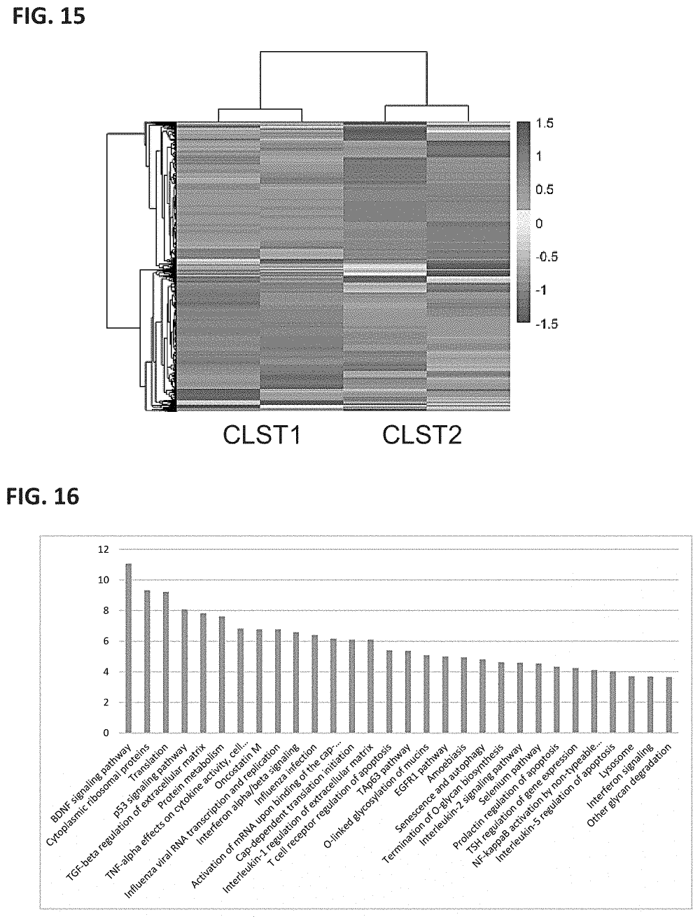

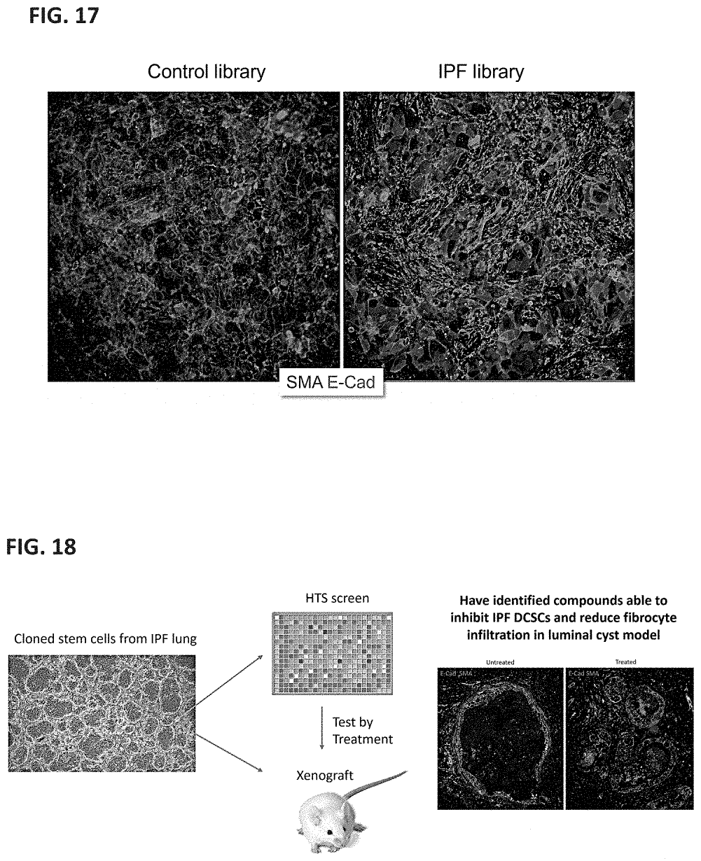

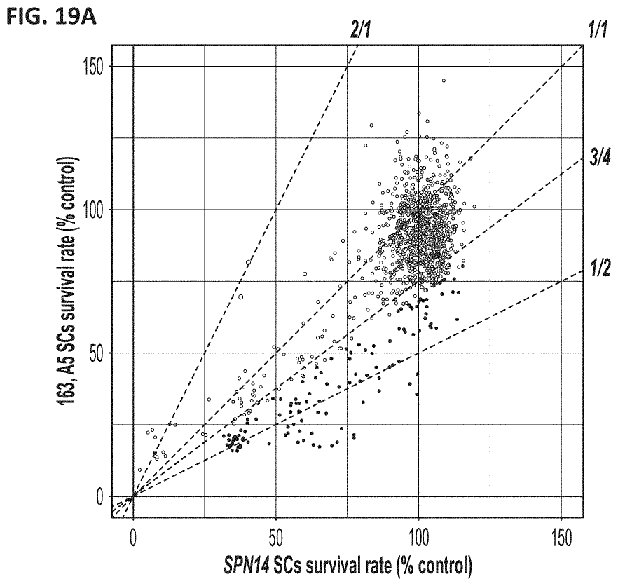

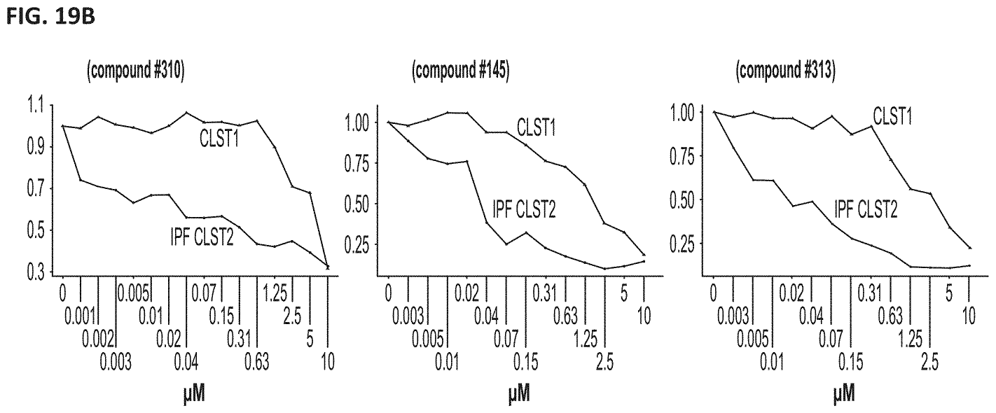

Pre-existence of variant clone types in normal and fetal lungs. If the variant clones that dominate COPD lung indeed contribute to the disease process, their origin becomes a central question. These variant clones, all of which express p63, conceivably arise from an epigenetic ‘reprogramming’ of normal, p63+ distal airway clones. Alternatively, these variant cells might already exist as distinct lineages in the normal distal airway epithelia, albeit at low ratios to the distal airway progenitor. In support of the pre-existence model, both single cell RNA sequencing and FACS profiles of control lungs indicated an aggregate representation of variant clones of approximately 10% (FIG. 3E, FIG. 4B), and direct cloning from control libraries showed that these variants possessed corresponding metaplastic fate commitment and pathological activity in xenografts (FIG. 14A). Consistently, expression data of Cluster 1-4 clones from control case SPN-14 showed significant overlap with those of COPD cases (FIG. 14B; FIG. 10A). However, the inventors could not rule out the possibility that at least some of the control cases had ‘subclinical” disease reflected by these variant clones. To more definitively address this question, they examined the clonal architecture of libraries generated from 13- and 17-week fetal lungs (FIG. 7A, FIG. 14C). Remarkably, scRNAseq profiles of the clone libraries of these pseudoglandular fetal lungs (Schittny, 2017) revealed minor populations of clones similar to those of Cluster 2, 3, and 4 variants (in aggregate, 17-34%) among a majority of normal Cluster 1 clones (FIG. 7B; FIGS. 14D,E). The similarity of these four fetal clone types with those of Clusters 1-4 from COPD cases was confirmed by their respective differentiation fate in vitro and upon xenografting, at the level of gene expression, and finally by their corresponding potential to drive mucus hypersecretion (GCM), fibrosis (SCM and iSCM), and neutrophilic inflammation (iSCM) (FIGS. 7C,D). The preexistence of these variant clone types in both normal adult lung and in developing fetal lung favors some unknown, normal function of these variants in the lung as well as the notion that the evolution of COPD involves a pathological and irreversible expansion of these variant populations that, in turn, drives the disease process.

### B. Discussion

The abnormal clonogenic cell types in COPD lungs offers new insights into the natural history of COPD lung disease. The fates and biological properties of the three major clone types (GCM, SCM, and iSCM) in the COPD lung appear to be autonomously maintained and, in aggregate, dominate the epithelia of moderate and end-stage COPD. There are two implications of this work. One is that these clonogenic variants are stem cells respectively committed to metaplastic lesions that, in turn, promote the fibrotic and inflammatory processes observed in COPD. The second is that these variant cells or the factors they secrete may represent important therapeutic targets in this disease.

The clonal analysis described here was instrumental in defining the heterogeneity of clonogenic epithelial cells in COPD. Unlike the normal clones of Cluster 1 committed to distal bronchiolar fates, the three variant types exhibited distinct pathologic phenotypes, with Cluster 2 clones producing a GCM marked by the excess mucin secretion implicated in airway occlusion (Fahy and Dickey, 2010; Kesimar et al., 2017; Wedzicha, 2017), while SCM (Cluster 3) clones induced an activation of airway endothelial cells in vitro and, in immunodeficient mice, a host myofibroblast response linked to fibrosis. Cluster 4 clones not only activated endothelial cells in vitro but triggered both submucosal myofibroblast recruitment and leukocyte infiltration upon xenografting to immunodeficient mice. Given that clone libraries from all 19 COPD patients induced leukocyte infiltration whereas only 1 of the 11 controls did so, as well as the major role of inflammation in this disease (Barnes, 2016; Brazil and Parkos, 2016; Butler et al., 2018), the inventors predict that Cluster 4 clones will figure prominently in the evolution of COPD.

It should be emphasized that the inflammatory genes expressed in proliferating clones isolated from COPD lung are constitutively activated even in a sterile, in vitro environment (FIG. 5). The inflammatory pathways manifest in these cells are linked to the activation of a broad array of the innate and adaptive immune cell populations associated with COPD, including neutrophils, monocyte-derived macrophages and dendritic cells, as well as a host of T-helper subsets, natural killer cells, and other lineages such as innate lymphoid cells. Many of these immune cell types impact the development of fibrosis (Lee and Kalluri, 2010; Kasembeli et al., 2018). Notably, despite the broad range of the inflammatory pathways expressed in Cluster 4 clones, only neutrophil responses were apparent in xenografts of these cells. At least some of this restricted host response can be attributed to strain of immunodeficient mice (NOD/scid/IL2rγnull) used in these studies, which lacks mature B and T cells, natural killer cells, and shows defects in monocyte-derived macrophages and dendritic cells (Shultz et al., 1995; Shultz at al., 2005; Ito et al., 2012). Therefore, the inventors were unable to assess whether other hematopoietic lineages 5 associated with COPD (Barnes, 2016; Suzuki et al., 2017) might be impacted as well by the variant clones. Xenografts of these clones in immunodeficient strains having a greater complement of hematopoietic lineages may provide a more complete assessment of interactions between these clones and the immune system, as would the identification of homologous variant clones in genetically tractable model organisms that would enable syngeneic studies of interactions across the entire immune system.

This study raises important questions on the origin of the variant clones detected in COPD, how they become hegemonic, and what they mean to the etiology and progression of the disease. Taking just the Cluster 4 clone (iSCM) that promotes host neutrophilic and fibrotic responses as an example, quantitative FACS profiling showed that it represents 13+/−8% of all clonogenic cells across 19 COPD lungs whereas only 1.4+/−1.6% of those in Control lungs (FIG. 7E). These numbers raise the possibility that the COPD phenotype becomes apparent at some percentage threshold of these clones. The inventors tested this hypothesis in a tangential manner by asking what ratios of Cluster 4 to Cluster 1 clones in co-xenografts would elicit a host neutrophil response (FIG. 7F). The resulting xenografts induced a minimal inflammatory response when Cluster 4 clones comprised less than 5% of the total clones, whereas ratios between 10 and 20% of Cluster 4 clones triggered a robust neutrophil response (FIG. 7G). These findings support the notion that a threshold phenomenon, tied to the relative ratios of these variant cells, could be relevant to both the onset of clinical symptoms and the vectored progression of COPD. The inventors anticipate that additional modeling of these variant clones singly or in combination will contribute to understanding their roles in COPD.

Given that similar variant stem cells preexist in control adult lungs as well as in lungs at multiple stages of fetal development, it is likely that these variants play some role in the normal lung perhaps as sentinels for pathogen incursions. Understanding the responses of these variants in normal lung may provide insights into their intrinsic functions, and how these cells might be suborned to disease. In this regard, key questions remain for how these minor variants become major variants in COPD. How do early-life pulmonary events or chronic smoking set in motion their numerical expansion, does the disease process itself further alter these variants during the expansion process, and can this expansion be therapeutically preempted or reversed?

The overall findings here set the stage for more detailed studies of correlations between the abundance of such clones and COPD stage as well as their correlation with the known regional, intrapulmonary variations in COPD pathology (Nambu et al., 2016). The inventors should note here that multiple studies have highlighted the possibility that alterations in epithelial cells in idiopathic pulmonary fibrosis (IPF), chronic rhinosinusitis, and COPD may contribute to the pathology of these conditions (Byers et al., 2013; Xu et al., 2016; Ordovas-Montanes et al., 2018; Habiel et al., 2018; Vieira Braga et al., 2019). While those studies did not examine clonogenic cells per se, their findings support the speculation that many if not all chronic lung conditions involve pathogenic clone variants of the sort described here.

If indeed these clones contribute to COPD, multiple opportunities become available to limit their impact on disease progression. These include neutralizing one or more of the pathogenically relevant chemokines or cytokines secreted by individual variants, targeting particular variants with cell surface-directed antibodies, or through the discovery of small molecules that selectively affect one or more of these clone types. This latter strategy could be predicated on the observation that COPD patients retain normal clones that would potentially compensate for the loss of their pathogenic counterparts.

KEY RESOURCES TABLE

| REAGENT or RESOURCE | SOURCE | IDENTIFIER |
|---|---|---|
| Antibodies | | |
| Rabbit monoclonal Cytokeratin 5 | Thermo Fisher | RRID: A6_2538529 |
| Mouse monoclonal human Cytokeratin 5 | Leica Biosystems | RRID: A6_442073 |
| Mouse monoclonal CC10 | Santa Cruz | RRID: AB_2183388 |
| Rabbit polyclonal AQP4 | Sigma-Aldrich | RRID: AB_1844967 |
| Rabbit polyclonal SFTPB | Sigma-Aldrich | RRID: AB_2674347 |
| Mouse monoclonal acetylated tubulin | Sigma-Aldrich | RRID: A6_609894 |
| Rat monoclonal mouse CD45 | Thermo Fisher | RRID: AB_657749 |
| mouse monoclonal human CD45 | R & D systems | RRID: AB_2174120 |
| Rat monoclonal mouse Ly6G | R & D systems | RRID: AB_2232806 |
| mouse monoclonal alpha smooth muscle Actin | Abcam | RRID: A6_262054 |
| Rabbit polyclonal Involucrin | Sigma-Aldrich | RRID: AB_2682739 |
| Mouse monoclonal Cytokeratin 10 | Bio Legend | RRID: AB_2565038 |
| Rabbit polyclonal Muc56 | Abcam | RRID: AB_10712492 |
| E-Cadherin Antibody | R & D Systems | RRID: AB_355504 |
| Human IL-8/CXCL8 Antibody | R & D Systems | RRID: AB_2249110 |
| Aquaporin 5 antibody [EPR3747 ] | Abcam | RRID: AB_2049171 |
| Anti-TRPC6 Rabbit Polyclonal Antibody | Proteintech | RRID: AB_10859822 |
| Anti-p63 antibody (4a4) | Abcam | RRID: AB _305870 |
| Bacterial and Virus Strains | | |
| N/A | N/A | N/A |
| Biological Samples | | |
| Human Lung samples from COPD | UCONN Health; | |
| and non-COPD patients | UTHSC; UIHC | |
| Chemicals, Peptides, and Recombinant Proteins | | |
| Trizol Reagent | Thermo Fisher | Cat #15596018 |
| Feeder removal beads | Miltenyi Biotec | Cat #130-095-531 |
| Human IL-13 Recombinant Protein | eBioscience ™ | Cat #BMS351 |
| Power SYBR Green PCR master mix | Thermo Fisher | Cat #4368706 |
| TrypLE ™ Express Enzyme (1×), phenol red | Thermo Fisher | Cat #12605036 |
| Corning ™ Matrigel ™ GFR Membrane Matrix | Fisher Scienitific | Cat #CB-40230 |
| COLLAGENASE TYPE IV | Life Technologies | Cat #17104-019 |
| StemECHO ™ PU Ground-State | Nüwa Medical | http://www.stemecho.com |
| Stem Cell Culture System | Systems, Inc. | |
| Critical Commercial Assays | | |
| Fixation/Permeabilization Solution Kit | BD Biosciences | Cat #554714 |
| (Cytofix/Cytoperm) | | |
| Maxima First Strand cDNA Synthesis | Thermo Fisher | Cat #K1671 |
| Kit for RT-qPCR, with dsDNase | | |
| DNeasy Blood & Tissue Kit (250) | Qiagen | Cat #69506 |
| GeneChip ™ Human Exon 1.0 ST Array | Affymetrix | Cat #900650 |
| Deposited Data | | |
| Expression microarray raw data | This paper | GSE118950 |
| Whole genome exome sequencing data | This paper | PRJNA492749 |
| RNAseq data | This paper | PRJNA492749 |
| scRNAseq data | This paper | GSE118950 |
| Statistical analysis raw data | This paper | https://data.mendeley.com/ |
| | | drafts/tf8kyp2ghx |
| Experimental Models: Cell Lines | | |
| HMVEC-L-Human Lung | Lonza | Cat #CC-2527 |
| Microvascular Endothelial Cells | | |
| Experimental Models: Organisms/Strains | | |
| Mouse: NOD.Cg-Prkdc^{scid} 112rg^{tm1Wjl}/SzJ | The Jackson Laboratory | Stock No: 005557 \| NSG |
| Oligonucleotides | | |
| N/A | N/A | N/A |
| Recombinant DNA | | |
| N/A | N/A | N/A |
| Software and Algorithms | | |
| SH800S Cell Sorter Software | SH800S Cell Sorter | https://www.sonybiotech |
| version 2.1 | | nology.com/us/instrume |
| | | nts/sh800s-cell- |
| | | sorter/software/ |
| Trim Galore | | https://www.bioinformatics. |
| | | babraham.ac.uk/projects/ |
| | | trim_galore/ |
| Xenome | Conway et al., 2012 | http://www.nicta.com.au/ |
| | | bioinformatics |
| Salmon | Patro etal., 2017 | https://salmon.readthedocs. |
| | | io/en/latest/salmon.html |
| DEseq2 | Love et al., 2014 | https://bioconductor.org/ |
| | | packages/release/bioc/html/ |
| | | DESeq2.html |
| RUVSeq | Risso et al., 2014 | https://bioconductor.org/ |
| | | packages/release/bioc/html/ |
| | | RUVSeq.html |
| Pheatmap | | https://cransproject.org/ |
| | | web/packages/pheatmap/ |
| | | index.html |
| Enrichr | Chen et al., 2013 | https://amp.pharm.mssm.edu/ |
| | | Enrichr/ |
| Ingenuity Pathway Analysis | Andreas et al., 2014 | https://www.qiagenbioin |
| | | formatics.com/products/ |
| | | features/?gclid=Cj0KCQiAl |
| | | 5zwBRCTARIsAlrukdM9LV |
| | | WhBGtDPLVPTgsVLN1_ |
| | | N-jK9bcU5ZniWNXs- |
| | | UihFNPyaxcOcK8aAl2tEAL |
| | | w_wcB |
| Cellranger toolkit | | https://support.10xgenomics. |
| | | com/single-cell-gene- |
| | | expression/software/pipe |
| | | lines/latest/what-is-cell-ranger |
| Seurat | Satija etal., 2015 | https://satijalab.org/seurat/ |
| Partek Genomics Suite 6.6 | | https://www.partek.com/ |
| | | partek-genomics-suite/ |
| Trimmomatic | Bolger et al., 2014 | http://www.usadellab.org/ |
| | | cms/?page=trimmomatic |
| BWA-mem | Li and Durbin, 2010 | http://bio- |
| | | bwa.sourceforge.net/ |
| GATK | DePristo et al., 2011; | https://software. |
| | Van der | broadinstitute.org/gatk/ |
| | Auwera et al., 2013 | |
| Manta | Chen et al., 2016 | https://github.com/ |
| | | Illumina/manta |
| Strelka | Kim et al., 2018 | https://github.com/ |
| | | target/strelka |
| ANNOVAR | Yang and Wang, 2015 | http://annovar.openbioin |
| | | formatics.org/en/latest/ |
| GnomAD database v2 | Karczewski et al., 2019 | https://gnomad. |
| | | broadinstitute.org/ |
| 1000 Genome database | | https://www. |
| | | internationalgenome.org/ |
| OncoKB database | Chakravarty et al., 2017 | https://www.oncokb.org/ |

### C. Method Details

In vitro culture of clonogenic cells from 19 COPD and 11 control lungs. Minced lung tissue was digested in 2 mg/ml collagenase type IV (Gibco, USA) at 37° C. for 30-60 min with agitation.

Dissociated cells were passed through a 70 μm Nylon mesh (Falcon, USA) to remove masses and then were washed four times in cold F12 media, and seeded onto a feeder layer of lethally irradiated 3T3-J2 cells as described (Kumar et al., 2011; Zuo et al., 2015) and grown in StemECHO™PU culture medium (Nuwa Medical Systems, Inc., Houston, USA). In each case, approximately 2 to 4×10⁶ lung cells were dissociated and 1×10⁶ were finally seeded from 1 cc lung samples after the lysis of erythrocytes. In the control cases, 1,500 to 2,300 colonies were observed at PO after seeding, indicating that 0.15% to 0.23% of all cells were clonogenic. For COPD cases, the inventors obtained 4,500 to 8,000 colonies at P0, indicating that 0.45% to 0.8% of all lung cells were clonogenic. The culture media was changed every two days. Colonies were digested by 0.25% trypsin-EDTA solution (Gibco, USA) for 5-8 min and passaged every 7 to 10 days. Colonies were trypsinized by TrypLE Express solution (Gibco, USA) for 8-15 min at 37° C. and cell suspensions were passed through 30 μm filters (Miltenyi Biotec, Germany). Approximately 20,000 epithelial cells were seeded to each well of 6-well plate. Cloning cylinder (Pyrex, USA) and high vacuum grease (Dow Corning, USA) were used to select single colonies for pedigrees. Gene expression analyses were performed on cells derived from passage 4-10 (P4-P10) cultures.

Histology validation set of five control and 10 Stage-4 COPD lungs. To validate the localization of clone-specific metaplasia in situ, the inventors performed histopathology and immunolocalization studies on serial sections from five control lungs from patients without lung or systemic disease (Okuda et al., 2019) and from 10 patients with Stage 4 COPD. These histological sections were obtained under IRB approval from the University of North Carolina (03-1396), the University of Iowa Carver College of Medicine (199507432), and the University of Texas Health Sciences Center at Houston (HSC-MS-08-0354/HSC-MS-15-1049). Student's t-test was used to determine the statistical difference between groups. P<0.05 was considered statistically significant.

Histology and immunostaining. Histology, hematoxylin and eosin (H&E) staining, immunohistochemistry, and immunofluorescence were performed using standard techniques. For immunofluorescence and immunohistochemistry, 4% paraformaldehyde-fixed, paraffin embedded tissue slides were subjected to antigen retrieval in citrate buffer (pH 6.0, Sigma-Aldrich, USA) at 120° C. for 20 min, and a blocking procedure was performed with 5% bovine serum albumin (BSA, Sigma-Aldrich, USA) and 0.05% Triton X-100 (Sigma-Aldrich, USA) in DPBS(−) (Gibco, USA) at room temperature for 1 hr. The sources of primary antibodies used in this study include: Rabbit monoclonal cytokeratin 5 antibody (RM-2106-S; Thermo Fisher, USA); mouse monoclonal human cytokeratin 5 antibody (NCL-L-CK5; Leica Biosystems, Germany); mouse monoclonal CC10 antibody (sc-365992; Santa Cruz Biotechnology, USA); rabbit polyclonal AQP4 antibody (HPA014784; Sigma-Aldrich, USA); rabbit polyclonal SFTPB antibody (HPA034820; Sigma-Aldrich, USA); mouse monoclonal acetylated tubulin T7451 (Sigma-Aldrich, USA); rat monoclonal mouse CD45 antibody 14-0451-85 (Thermo Fisher, USA); mouse monoclonal human CD45 MAB1430-SP (R&D systems, USA); rat monoclonal mouse Ly6G MAB1037 (R&D systems, USA); mouse monoclonal alpha smooth muscle actin antibody (ab7817; Abcam, UK); rabbit polyclonal Involucrin antibody (HPA055211; Sigma-Aldrich, USA); mouse monoclonal Cytokeratin 10 antibody (904301; BioLegend, USA); rabbit polyclonal Muc5B antibody (ab87376; Abcam, UK); rabbit polyclonal Muc5AC antibody (ab78660; Abcam, UK); goat polyclonal E-Cadherin antibody (AF648; R&D Systems, USA). Secondary antibodies used here are Alexa Fluor-488 or Alexa Fluor-594 Donkey anti-goat/mouse/rabbit IgG antibody (Thermo Fisher, USA). All images were captured by using the Inverted Eclipse Ti-Series (Nikon, Japan) microscope with Lumencor SOLA light engine and Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFilcamera (Nikon, Japan) and NIS-Elements F3.0 software (Nikon, Japan).

In vitro differentiation. Air-liquid interface (ALI) culture of epithelial clones was performed as described (Kumar et al., 2011; Zuo et al., 2015). Briefly, Transwell inserts (Corning, USA) were coated with 20% Matrigel (BD biosciences, USA) and incubated at 37° C. for 30 min to polymerize. 200,000 irradiated 3T3-J2 cells were seeded to each Transwellinsert and incubated at 37° C., 7.5% CO₂ incubator overnight. QuadroMACS Starting Kit (LS) (Miltenyi Biotec, Germany) was used to purify epithelial by removal of feeder cells. 200,000-300,000 cells were seeded into each Transwellinsert and cultured in StemECHO™PU media. At confluency (3-7 days), the apical media on the inserts was removed through careful pipetting and the cultures were continued in differentiation media (PneumaCult-ALI Media, STEMCELL Technologies, Vancouver) for an additional 14-21 days prior to harvesting. The differentiation media was changed every one or two days.

Xenografts in immunodeficient mice. One million epithelial cells were harvested by trypsinization, mixed with 50% Matrigel (Becton Dickinson, Palo Alto) to a volume of 100 ul and injected subcutaneously in NSG (NODscid IL2ra^{null}; Shultz et al., 1995) mice (Jackson Laboratories, Bar Harbor) and harvested two or four weeks later.

RNA sample preparation. For cell colonies, RNA was isolated using PicoPure RNA Isolation Kit (Life Technologies, USA). For ALI and Xenografts structure, RNA was isolated using Trizol RNA Isolation Kit (Life Technologies, USA). RNA quality (RNA integrity number, RIN) was measured by analysis Agilent 2100 Bioanalyzer and Agilent RNA 6000 Nano Kit (Agilent Technologies, USA). RNAs having a RIN>8 were used for microarray analysis.

### Flow Cytometry Analysis

Clonogenic cell libraries from patients with or without COPD were trypsinized and harvested as single cell suspension. Feeders were removed as mentioned above and approximately 300,000 epithelial cells were fixed and permeabilized by using Fixation/Permeabilization Solution Kit (BD biosciences, USA, cat. 554714). After a blocking procedure with Permeabilization solution at 4° C. for 30 min, cells were incubated with primary and Alexa Fluor 488 Secondary antibodies (Thermo Fisher, USA) for 1 hr at 4° C., with five washing events between each step. Primary antibodies used in these experiments include: rabbit monoclonal anti-AQP5 antibody ab92320 (Abcam, UK), rabbit polyclonal TRPC6 antibody (18236-1-AP; Proteintech, USA), mouse monoclonal anti-human IL-8/CXCL8 antibody (MAB208; R&D, USA). Samples were collected and analyzed with on a Sony SH800S Cell Sorter (Sony Biotechnology, USA).

QUANTIFICATION AND STATISTICAL ANALYSIS. Transcriptomic sequencing data analysis. All RNA-seq libraries were sequenced on Illumina NovaSeq 6000 with 150 bp pair-end reads. Raw reads were trimmed to remove low quality bases (phred score <20) and sequencing adapter leftovers using Trim Galore (world wide web at bioinformatics.babraham.ac.uk/projects/trim_galore/). Potential mouse genomic DNA contaminant reads were removed for further analysis using Xenome (Conway et al., 2012). Trimmed RNAseq reads were mapped to the human genome (UCSC hg19) using Salmon (version 0.9.1) with default settings (Patro et al., 2017). Alignment results were then input to DEseq2 (Love et al., 2014) for differential expression analysis with default settings and FDR less than 0.05. Batch effects were estimated and corrected using RUVSeq (Risso et al., 2014). The heatmaps with hierarchical clustering analysis of the global gene expression pattern in different samples were performed using pheatmap package (cran.rproject.org/web/packages/pheatmap/index.html) in R (version 3.5.1). The pathway enrichment analysis was performed using Enrichr (Chen et al., 2013) and Ingenuity Pathway Analysis (IPA) tools (Andreas et al., 2014).

Sequence alignment of single cell RNA sequencing. The single cell mRNA sequencing (scRNA-seq) libraries were established using the 10× Genomics Chromium system (Single Cell 3′ Reagent Kit v2). The scRNA-seq libraries were sequenced on the Illumina HiSeq X Ten with 10K cells for three COPD cases and fetal lung case. For three normal cases, the scRNA-seq libraries were sequenced on the Illumina NextSeq 500 with 2K cells. Demultiplexing, alignment and UMI-collapsing were performed using the Cellranger toolkit (version 2.1.0, 10× Genomics). The raw paired-end reads were trimmed to 26 bps for Read1 and 98 bps for Read2. The trimmed reads were mapped to both the human genome (hg19) and the mouse genome (mm10). The reads uniquely mapped to the human genome were used for downstream analysis.

Single cell RNA sequencing. The scRNA-seq data analyses were performed using the Seurat package (version 2.3.4; Satija et al., 2015). The inventors kept the genes with expression in at least three cells, and excluded cells expressing less than 200 genes. They also excluded the cells with high mitochondrial percentage or with an outlier level of UMI content. The normalization was performed using the global-scaling normalization method, which normalizes the gene expression measurements for each cell by the total expression, and then multiplies by 10,000, and finally log-transforms the result. The variable genes were identified using a function to calculate average expression and dispersion for each gene, divides these genes into bins, and then calculates a z-score for dispersion within each bin (“x.low.cutoff=0.0125”, “x.high.cutoff=3”, and “y.cutoff=0.5”). The inventors scaled the data to regress out the variation of mitochondrial gene expression.

The inventors performed PCA based on the scaled data to identify significant principal components (PCs). They selected the PCs with p-values less than 0.01 as input to perform clustering analysis and visualization by t-SNE. They detected the marker genes in each cell subpopulation using two methods of Wilcoxon rank sum test and DESeq2. For Wilcoxon rank sum test, the inventors used the default parameter. For DEseq2, they kept the marker genes with average log-fold change above 0.1 and adjust p-value fewer than 0.05.

Contaminating 3T3-J2 fibroblast cells were identified by murine reads. In addition, the cells in S stage of cell cycle were identified based on the marker gene of SLBP (Nestorowa et al., 2016). The cells in G2 or M stage of cell cycle were identified based on the marker genes of UBE2C, AURKA, CENPA, CDCl20, HMGB2, CKS2, and CKS1B. The cells in G0 stage of cell cycle were identified based on the marker genes of G0S2. In addition, the ambiguous cells with few marker genes were also removed, which could possibly correspond to sequencing low quality cells. Finally, the inventors integrated the clean data of six cases to perform clustering analysis and visualization by t-SNE.

Expression microarray and bioinformatics. Total RNAs obtained from immature colonies and ALI-differentiated epithelia were used for microarray preparation with WT Pico RNA Amplification System V2 for amplification of DNA and Encore Biotin Module for fragmentation and biotin labeling (NuGEN Technologies, USA). All samples were prepared according to manufacturer's instructions and hybridized onto GeneChip Human Exon 1.0 ST array (Affymetrix, USA). GeneChip operating software was used to process Cel files and calculate probe intensity values. To validate sample quality, quality checks were conducted using Affymetrix Expression Console software. The intensity values were log 2-transformed and imported into the Partek Genomics Suite 6.6 (Partek Incorporated, USA). Exons were summarized to genes and a 1-way ANOVA was performed to identify differentially expressed genes. Unsupervised clustering and heatmap generation were performed with sorted datasets by Euclidean distance based on average linkage by Partek Genomics Suite 6.6. Datasets used in this study have been deposited with the National Center for Biotechnology Information Gene Expression Omnibus (GEO) database (GSE118950).

Whole genome exome sequencing data analysis. For exome capture and high-throughput sequencing, genomic DNA was isolated from single cell-derived pedigrees and 1000 ng of DNA per sample were sheared, end-repaired, A-tailed and adaptor-ligated. Exome capture was conducted using the Agilent SureSelect Human All Exon V6 Kit following the manufacturer's standard protocols. The libraries were sequenced on an Illumina HiSeq X ten platform (150 bp paired-end model). The raw sequencing data were quality controlled by Trimmomatic (Bolger et al., 2014) version 0.36 to trim low-quality bases, filtered by Xenome (Conway et al., 2012) version 1.0.1 to remove mouse sequences, aligned to reference genome (hg19) via BWA-mem (Li and Durbin, 2010) version 0.7.15, realigned through GATK (McKenna et al., 2010) version 3.8.0 in regions near indels (Mills_and_1000G_gold_standard indels), and recalibrated with default settings following the best practices pipeline of GATK (Van der Auwera et al., 2013). Somatic SNVs and Indels were called by Manta (Chen et al., 2016) version 1.3.2 and Strelka (Kim et al., 2018) version 2.9.2 and annotated with ANNOVAR (Yang and Wang, 2015). The inventors require that somatic mutations situated at the targeted capturing regions, pass the Strelka default filters, and have only two genotypes present, a minimum of 5% mutant reads fraction, a minimum 5 mutant reads coverage, and minimum 15 total reads coverage. The corresponding matched normal sample should be homozygous wild type at the mutation sites. Somatic mutations were further filtered to remove possible germline mutations based on a panel of 27 normal samples. Somatic mutations with allele frequencies larger than 0.01 in 1000 Genome database or gnomAD database v2 (Karczewski et al., 2019) were discarded as well. The somatic allelic copy number variants were called by the best practices pipeline of GATK version 4.0.4 (DePristo et al., 2011; Van der Auwera et al., 2013). The modeled copy number segments with less than 7.5 Kbp in length or less than 15 common variants were filtered out. The inventors used default settings in all software. The oncogenes and tumor suppressors were fetched from the OncoKB database (Chakravarty et al., 2017).

Statistical analysis. Unpaired two-tailed student's t-test was used to determine the statistical significance between two groups. Statistical analyses were performed using R (version 3.5.1). The “n” numbers for each experiment are provided in the text and figures. P<0.05 was considered statistically significant. Asterisks denote corresponding statistical significance *p<0.05; **p<0.01; ***p<0.001 and ****p<0.0001.

### D. References for COPD Example

1. Andreas K., Jeff G., Jack P., Jr and Stuart T. (2014) Causal analysis approaches in Ingenuity Pathway Analysis. Bioinformatics 2014; 30: 523-530.
2. Aschner Y, Downey G P. Transforming growth factor-β: master regulator of the respiratory system in health and disease. Am J Respir Cell Mol Biol. 2016; 54: 647-655.
3. Araya J, Cambier S, Markovics J A, Wolters P, Jablons D, Hill A, Finkbeiner W, Jones K, Broaddus V C, Sheppard D, Barzcak A, Xiao Y, Erle D J, Nishimura S L. Squamous metaplasia amplifies pathologic epithelial-mesenchymal interactions in COPD patients. J Clin Invest. 2007; 117: 3551-3562.
4. Araya J, Nishimura S L. Fibrogenic reactions in lung disease. Annu Rev Pathol. 2010; 5: 77-98.
5. Barnes P J, Burney P G, Silverman E K, Celli B R, Vestbo J, Wedzicha J A, Wouters E F. Chronic obstructive pulmonary disease. Nat Rev Dis Primers 2015; 1: 15076.
6. Barnes P J. Inflammatory mechanisms in patients with chronic obstructive pulmonary disease. J Allergy Clin Immunol. 2015; 138: 16-27.
7. Black L M, Lever J M, Agarwal A. (2019). Renal inflammation and fibrosis: a double-edged sword. J Histochem Cytochem. 2019; 67:663-681.
8. Bolger, A. M., Lohse, M., & Usadel, B. Trimmomatic: A flexible trimmer for Illumina Sequence Data. Bioinformatics 2014; btu170.
9. Butler A, Walton G M, Sapey E. (2018). Neutrophilic inflammation in the pathogenesis of chronic obstructive pulmonary disease. COPD 2018; 31: 1-13.
10. Brazil J C, and Parkos C A. Pathobiology of neutrophil-epithelial interactions. Immunol Rev. 2018; 273, 94-111.
11. Boucherat O, Morissette M C, Provencher S, Bonnet S, Maltais F. Bridging lung development with chronic obstructive pulmonary disease. relevance of developmental pathways in chronic obstructive pulmonary disease pathogenesis. Am J Respir Crit Care Med. 2016; 193: 362-375.
12. Busch R, Hobbs B D, Zhou J, Castaldi P J, McGeachie M J, Hardin M E, Hawrylkiewicz I, Sliwinski P, Yim J J, Kim W J, Kim D K, Agusti A, Make B J, Crapo J D, Calverley P M, Donner C F, Lomas D A, Wouters E F, Vestbo J, Tal-Singer R, Bakke P, Gulsvik A, Litonjua A A, Sparrow D, Pare P D, Levy R D, Rennard S I, Beaty T H, Hokanson J, Silverman E K, Cho M H; National Emphysema Treatment Trial Genetics; Evaluation of COPD Longitudinally to Identify Predictive Surrogate End-Points; International COPD Genetics Network; COPDGene Investigators. Genetic association and risk scores in a chronic obstructive pulmonary disease meta-analysis of 16,707 subjects. Am J Respir Cell Mol Biol. 2017; 57: 35-46.
13. Byers D E, Alexander-Brett J, Patel A C, Agapov E, Dang-Vu G, Jin X, Wu K, You Y, Alevy Y, Girard J P, Stappenbeck T S, Patterson G A, Pierce R A, Brody S L, Holtzman M J. Long-term IL-33-producing epithelial progenitor cells in chronic obstructive lung disease. J Clin Invest. 2013; 123:3967-3982.
14. Chakravarty, D., Gao, J., Phillips, S. M., Kundra, R., Zhang, H., Wang, J., Rudolph, J. E., Yaeger, R., Soumerai, T., Nissan, M. H., et al., OncoKB: A Precision Oncology Knowledge Base. JCO Precis Oncol 2017; 2017: 1-16.
15. Chen E Y, Tan C M, Kou Y, Duan Q, Wang Z, Meirelles G V, Clark N R, Ma'ayan A. Enrichr: interactive and collaborative HTML5 gene list enrichment analysis tool. BMC Bioinformatics. 2013; 14:128.
16. Chen, X., Schulz-Trieglaff, O., Shaw, R., Barnes, B., Schlesinger, F., Kallberg, M., Cox, A. J., Kruglyak, S., and Saunders, C. T. Manta: rapid detection of structural variants and indels for germline and cancer sequencing applications. Bioinformatics 2016; 32: 1220-1222.
17. Chilosi M, Poletti V, Murer B, Lestani M, Cancellieri A, Montagna L, Piccoli P, Cangi G, Semenzato G, Doglioni C. (2002). Abnormal re-epithelialization and lung remodeling in idiopathic pulmonary fibrosis: the role of deltaN-p63. Lab Invest. 2002; 82: 1335-1345.
18. Conway T, Wazny J, Bromage A, Tymms M, Sooraj D, Williams E D, and Beresford-Smith B. Xenome—a tool for classifying reads from xenograft samples. Bioinformatics 2012; 28: i172-i178.
19. Duffy S P and Criner G J. Chronic obstructive pulmonary disease: evaluation and management. Med Clin North Am. 2019; 103: 453-461.
20. Demedts I. K., K. R. Bracke, G. Van Pottelberge, D. Testelmans, G. M. Verleden, F. E. Vermassen, G. F. Joos, G. G. Brusselle. Accumulation of dendritic cells and increased CCL20 levels in the airways of patients with chronic obstructive pulmonary disease. Am. J. Respir. Crit. Care Med. 2007; 175: 998-1005.
21. Danahay H, Pessotti A D, Coote J, Montgomery B E, Xia D, Wilson A, Yang H, Wang Z, Bevan L, Thomas C, Petit S, London A, LeMotte P, Doelemeyer A, Vélez-Reyes G L, Bernasconi P, Fryer C J, Edwards M, Capodieci P, Chen A, Hild M, Jaffe A B. Notch2 is required for inflammatory cytokine-driven goblet cell metaplasia in the lung. Cell Rep. 2015; 10: 239-252.
22. Decramer M, Janssens W, Miravitlles M. Chronic obstructive pulmonary disease. Lancet 2012; 379: 1341-51.
23. DePristo, M. A., Banks, E., Poplin, R., Garimella, K. V., Maguire, J. R., Hartl, C., Philippakis, A. A., del Angel, G., Rivas, M. A., Hanna, M., et al., (2011). A framework for variation discovery and genotyping using next-generation DNA sequencing data. Nat Genet 2011; 43: 491-498.
24. Dhakshinamoorthy S, Jain A K, Bloom D A, Jaiswal A K. Bach1 competes with Nrf2 leading to negative regulation of the antioxidant response element (ARE)-mediated NAD(P)H:quinone oxidoreductase 1 gene expression and induction in response to antioxidants. J Biol Chem. 280, 16891-16900 (2005).
25. ENCODE Project Consortium. An integrated encyclopedia of DNA elements in the human genome. Nature 2012; 489: 57-74.
26. Fahy J V, Dickey B F. Airway mucus function and dysfunction. N Engl J Med. 2010; 363, 2233-2247.
27. Filippi M D. Mechanism of diapedesis: Importance of the transcellular route. Adv Immunol. 2016; 129:25-53.
28. Fletcher C, and Peto R. (1977). The natural history of chronic airflow obstruction. Br Med J. 1977; 1: 1645-1648.
29. Gamble E, Grootendorst D C, Hattotuwa K, O'Shaughnessy T, Ram F S, Qiu Y, Zhu J, Vignola A M, Kroegel C, Morell F, Pavord I D, Rabe K F, Jeffery P K, Barnes N C. Airway mucosal inflammation in COPD is similar in smokers and ex-smokers: a pooled analysis. Eur Respir J. 2007; 30: 467-471.
30. Geisler S J, Paro R. Trithorax and Polycomb group-dependent regulation: a tale of opposing activities. Development 2015; 142, 2876-2887.
31. Habiel D M, Espindola M S, Jones I C, Coelho A L, Stripp B, Hogaboam C M. CCR10+ epithelial cells from idiopathic pulmonary fibrosis lungs drive remodeling. JCI Insight. 2018; Aug. 23; 3.
32. Hogg J C, Chu F, Utokaparch S, Woods R, Elliott W M, Buzatu L, Cherniack R M, Rogers R M, Sciurba F C, Coxson H O, Pare P D. The nature of small-airway obstruction in chronic obstructive pulmonary disease. N Engl J Med. 2004; 350: 2645-2653.
33. Hogg J C, Timens W. The pathology of chronic obstructive pulmonary disease. Annu Rev Pathol. 2009; 4: 435-459.
34. Holtzman M J, Byers D E, Brett J A, Patel A C, Agapov E, Jin X, Wu K. Linking acute infection to chronic lung disease. The role of IL-33-expressing epithelial progenitor cells. Ann Am Thorac Soc. 2014; 2:11 Suppl 5:S287-91.
35. Ito R, Katano I, Ida-Tanaka M, Kamisako T, Kawai K, Suemizu H, Aiso S, Ito M. Efficient xenoengraftment in severe immunodeficient NOD/Shi-scid IL2rγnull mice is attributed to a lack of CD11c+B220+CD122+ cells. J Immunol. 2012; 189:4313-4320.
36. Johnson L G, Dickman K G, Moore K L, Mandel U, Boucher R C. Enhanced Na+ transport in an air-liquid interface culture system. Am J Physiol. 1993; 264: L560-5.
37. Karczewski, K. J., Francioli, L. C., Tiao, G., Cummings, B. B., Alföldi, J., Wang, Q., Collins, R. L., Laricchia, K. M., Ganna, A., Birnbaum, D. P., et al., Variation across 141,456 human exomes and genomes reveals the spectrum of loss-of-function intolerance across human protein-coding genes. bioRxiv, 2019; 531210.
38. Kasembeli M M, Bharadwaj U, Robinson P, Tweardy D J. Contribution of STAT3 to inflammatory and fibrotic diseases and prospects for its targeting for treatment. Int J Mol Sci. 2018; Aug. 5; 19(8).
39. Kesimer M, Ford A A, Ceppe A, Radicioni G, Cao R, Davis C W, Doerschuk C M, Alexis N E, Anderson W H, Henderson A G, Barr R G, Bleecker E R, Christenson S A, Cooper C B, Han M K, Hansel N N, Hastie A T, Hoffman E A, Kanner R E, Martinez F, Paine R 3rd, Woodruff P G, O'Neal W K, Boucher R C. Airway mucin concentration as a marker of chronic bronchitis. N Engl J Med. 2017; 377: 911-922.
40. Kim, S., Scheffler, K., Halpern, A. L., Bekritsky, M. A., Noh, E., Kallberg, M., Chen, X., Kim, Y., Beyter, D., Krusche, P., et al., Strelka2: fast and accurate calling of germline and somatic variants. Nat Methods 2018; 15: 591-594.
41. Kumar P A, Hu Y, Yamamoto Y, Hoe N B, Wei T S, Mu D, Sun Y, Joo L S, Dagher R, Zielonka E M, Wang de Y, Lim B, Chow V T, Crum C P, Xian W, McKeon F. Distal airway stem cells yield alveoli in vitro and during lung regeneration following H1N1 influenza infection. Cell 2011; 147, 525-538.
42. Langmead B and Salzberg S L. Fast gapped-read alignment with Bowtie 2. Nat Methods 2012; 9: 357-359.
43. Lapperre T S, Sont J K, van Schadewijk A, Gosman M M, Postma D S, Bajema I M, Timens W, Mauad T, Hiemstra P S; GLUCOLD Study Group. Smoking cessation and bronchial epithelial remodeling in COPD: a cross-sectional study. Respir Res. 2007; 8: 85.
44. Lee S B, and Kalluri R. Mechanistic connection between inflammation and fibrosis. Kidney Int Suppl. 2010; December; (119): S22-6.
45. Lee-Six H, Obro N F, Shepherd M S, Grossmann S, Dawson K, Belmonte M, Osborne R J, Huntly B J P, Martincorena I, Anderson E, O'Neill L, Stratton M R, Laurenti E, Green A R, Kent D G, Campbell P J. Population dynamics of normal human blood inferred from somatic mutations. Nature 2018; 561: 473-478.
46. Li, H., and Durbin, R. Fast and accurate long-read alignment with Burrows-Wheeler transform. Bioinformatics 2010; 26: 589-595.
47. Love M I, Huber W, Anders S. Moderated estimation of fold change and dispersion for RNA-seq data with DESeq2. Genome Biology 2014; 15: 550.
48. Martinez F D. Early-life origins of chronic obstructive pulmonary disease. N Engl J Med. 2016; 375: 871-878.
49. Martinez F J, Han M K, Allinson J P, Barr R G, Boucher R C, Calverley P M A, Celli B R, Christenson S A, Crystal R G, Fagerås M, Freeman C M, Groenke L, Hoffman E A, Kesimer M, Kostikas K, Paine R 3rd, Rafii S, Rennard S I, Segal L N, Shaykhiev R, Stevenson C, Tal-Singer R, Vestbo J, Woodruff P G, Curtis J L, Wedzicha J A. At the root: defining and halting progression of early chronic obstructive pulmonary disease. Am J Respir Crit Care Med. 2018; 197: 1540-1551.
50. McDonough J E, Yuan R, Suzuki M, Seyednejad N, Elliott W M, Sanchez P G, Wright A C, Gefter W B, Litzky L, Coxson H O, Pare P D, Sin D D, Pierce R A, Woods J C, McWilliams A M, Mayo J R, Lam S C, Cooper J D, Hogg J C. Small-airway obstruction and emphysema in chronic obstructive pulmonary disease. N Engl J Med. 2011; 365: 1567-1575.
51. McGeachie M J, Yates K P, Zhou X, Guo F, Sternberg A L, Van Natta M L, Wise R A, Szefler S J, Sharma S, Kho A T, Cho M H, Croteau-Chonka D C, Castaldi P J, Jain G, Sanyal A, Zhan Y, Lajoie B R, Dekker J, Stamatoyannopoulos J, Covar R A, Zeiger R S, Adkinson N F, Williams P V, Kelly H W, Grasemann H, Vonk J M, Koppelman G H, Postma D S, Raby B A, Houston I, Lu Q, Fuhlbrigge A L, Tantisira K G, Silverman E K, Tonascia J, Weiss S T, Strunk R C. Patterns of growth and decline in lung function in persistent childhood asthma. N Engl J Med. 2016; 374: 1842-1852.
52. McKenna, A., Hanna, M., Banks, E., Sivachenko, A., Cibulskis, K., Kernytsky, A., Garimella, K., Altshuler, D., Gabriel, S., Daly, M., et al., The Genome Analysis Toolkit: a MapReduce framework for analyzing next-generation DNA sequencing data. Genome Res 2010; 20: 1297-1303.
53. Miller M, Cho J Y, Pham A, Friedman P J, Ramsdell J, Broide D H. Persistent airway inflammation and emphysema progression on C T scan in ex-smokers observed for 4 years. Chest. 2011; 139: 1380-1387.
54. Murphy-Ullrich J E, Suto M J. Thrombospondin-1 regulation of latent TGF-β activation: A therapeutic target for fibrotic disease. Matrix Biol. 2018; 68-69: 28-43.
55. Nambu A, Zach J, Schroeder J, Jin G, Kim S S, Kim Y I, Schnell C, Bowler R, Lynch D A. Quantitative computed tomography measurements to evaluate airway disease in chronic obstructive pulmonary disease: Relationship to physiological measurements, clinical index and visual assessment of airway disease. Eur J Radiol. 2016; 85: 2144-2151.
56. Nestorowa S, Hamey F K, Pijuan Sala B, Diamanti E, Shepherd M, Laurenti E, Wilson N K, Kent D G, Göttgens B. A single cell resolution map of mouse haematopoietic stem and progenitor cell differentiation running title: single cell map of HSPC differentiation. Blood 2016; 128: 20-32.
57. Okuda K, Chen G, Subramani D B, Wolf M, Gilmore R C, Kato T, Radicioni G, Kesimer M, Chua M, Dang H, Livraghi-Butrico A, Ehre C, Doerschuk C M, Randell S H, Matsui H, Nagase T, O'Neal W K, Boucher R C. Localization of secretory mucins muc5ac and muc5b in normal/healthy human airways. Am J Respir Crit Care Med. 2019; 199: 715-727.
58. Ordovas-Montanes J, Dwyer D F, Nyquist S K, Buchheit K M, Vukovic M, Deb C, Wadsworth M H 2nd, Hughes T K, Kazer S W, Yoshimoto E, Cahill K N, Bhattacharyya N, Katz H R, Berger B, Laidlaw™, Boyce J A, Barrett N A, Shalek A K. (2018). Allergic inflammatory memory in human respiratory epithelial progenitor cells. Nature 2018; 560: 649-654.
59. Patro, R., Duggal, G., Love, M. I., Irizarry, R. A., & Kingsford, C. Salmon provides fast and bias-aware quantification of transcript expression. Nat. Methods 2017; 14: 417-419. 60. Plantier L, Crestani B, Wert S E, Dehoux M, Zweytick B, GuentherA, WhitsettJA. Ectopic respiratory epithelial cell differentiation in bronchiolised distal airspaces in idiopathic pulmonary fibrosis. Thorax 2011; 66: 651-657.
61. Ponce-Gallegos M A, Ramirez-Venegas A, Falfán-Valencia R. Th17 profile in COPD exacerbations. Int J Chron Obstruct Pulmon Dis. 2017; 12: 1857-1865.
62. Quaderi S A, Hurst J R. The unmet global burden of COPD. Glob Health Epidemiol Genom. 2018; Apr. 6; 3:e4.
63. Ramírez F, Ryan D P, Grüning B, Bhardwaj V, Kilpert F, Richter A S, Heyne S, Dündar F, Manke T. deepTools2: a next generation web server for deep-sequencing data analysis. Nucleic Acids Research 2016; Apr. 13:gkw257.
64. Ray S, Chiba N, Yao C, Guan X, McConnell A M, Brockway B, Que L, McQualter J L, Stripp B R. Rare sox2+ airway progenitor cells generate krt5+ cells that repopulate damaged alveolar parenchyma following influenza virus infection. Stem Cell Reports 2016; 7: 817-825.
65. Raju S V, Kim H, Byzek S A, Tang L P, Trombley J E, Jackson P, Rasmussen L, Wells J M, Libby E F, Dohm E, Winter L, Samuel S L, Zinn K R, Blalock J E, Schoeb T R, Dransfield M T, Rowe S M. A ferret model of COPD-related chronic bronchitis. JCI Insight. 2016; 1(15):e87536.
66. Risso D, Ngai J, Speed T, Dudoit S. Normalization of RNA-seq data using factor analysis of control genes or samples. Nature Biotechnology 2014; 32: 896-902.
67. Robinson J T, Thorvaldsdóttir H, Winckler W, Guttman M, Lander E S, Getz G, Mesirov J P. Integrative Genomics Viewer. Nature Biotechnology 2011; 29: 24-26.
68. Scanlon P D, Connett J E, Waller L A, Altose M D, Bailey W C, Buist A S, Tashkin D P; Lung Health Study Research Group. Smoking cessation and lung function in mild-to-moderate chronic obstructive pulmonary disease. The Lung Health Study. Am J Respir Crit Care Med. 2000; 161: 381-390.
69. Schamberger A C, Staab-Weijnitz C A, Mise-Racek N, Eickelberg O. Cigarette smoke alters primary human bronchial epithelial cell differentiation at the air-liquid interface. Sci Rep. 2015; 5:8163.
70. Schneider W M, Chevillotte M D, Rice C M. Interferon-stimulated genes: a complex web of host defenses. Annu Rev Immunol. 2014; 32: 513-545.
71. Schittny J C. Development of the lung. Cell Tissue Res. 2017; 367: 427-444.
72. Seibold M A, Smith R W, Urbanek C, Groshong S D, Cosgrove G P, Brown K K, Schwarz M I, Schwartz D A, Reynolds S D. The idiopathic pulmonary fibrosis honeycomb cyst contains a mucocilary pseudostratified epithelium. PLoS One. 2013; 8(3):e58658.
73. Shultz L D, Schweitzer P A, Christianson S W, Gott B, Schweitzer I B, Tennent B, McKenna S, Mobraaten L, Rajan T V, Greiner D L, et al., Multiple defects in innate and adaptive immunologic function in NOD/LtSz-scid mice. J Immunol. 1995; 154: 180-191.
74. Shultz L D, Lyons B L, Burzenski L M, Gott B, Chen X, Chaleff S, Kotb M, Gillies S D, King M, Mangada J, Greiner D L, Handgretinger R. Human lymphoid and myeloid cell development in NOD/LtSz-scid IL2R gamma null mice engrafted with mobilized human hemopoietic stem cells. J Immunol. 2005; 174: 6477-6489.
75. Siebel C, and Lendahl U. Notch signaling in development, tissue homeostasis, and disease. Physiol Rev. 2017; 97: 1235-1294.
76. Singh D, Agusti A, Anzueto A, Barnes P J, Bourbeau J, Celli B R, Criner G J, Frith P, Halpin D M G, Han M, López Varela M V, Martinez F, Montes de Oca M, Papi A, Pavord I D, Roche N, Sin D D, Stockley R, Vestbo J, Wedzicha J A, Vogelmeier C. Global Strategy for the Diagnosis, Management, and Prevention of Chronic Obstructive Lung Disease: the GOLD science committee report 2019. Eur Respir J. 2019; May 18 53(5).
77. Smirnova N F, Schamberger A C, Nayakanti S, Hatz R, Behr J, Eickelberg O. Detection and quantification of epithelial progenitor cell populations in human healthy and IPF lungs. Respir Res. 2016; 17(1): 83.
78. Sun L, Ren X, Wang I C, Pradhan A, Zhang Y, Flood H M, Han B, Whitsett J A, Kalin T V, Kalinichenko V V. The FOXM1 inhibitor RCM-1 suppresses goblet cell metaplasia and prevents I L-13 and STAT6 signaling in allergen-exposed mice. Sci Signal. 2017; 10(475).
79. Suzuki M, Sze M A, Campbell J D, Brothers J F 2nd, Lenburg M E, McDonough J E, Elliott W M, Cooper J D, Spira A, Hogg J C. (2017). The cellular and molecular determinants of emphysematous destruction in COPD. Sci Rep. 2017; 7: 9562.
80. Tanaka Y, Yamaguchi M, Hirai S, Sumi T, Tada M, Saito A, Chiba H, Kojima T, Watanabe A, Takahashi H, Sakuma Y. Characterization of distal airway stem-like cells expressing N-terminally truncated p63 and thyroid transcription factor-1 in the human lung. Exp Cell Res. 2018; 372: 141-149.
81. Tokunaga R, Zhang W, Naseem M, Puccini A, Berger M D, Soni S, McSkane M, Baba H, Lenz H J. CXCL9, CXCL10, CXCL11/CXCR3 axis for immune activation—A target for novel cancer therapy. Cancer Treat Rev. 2018; 63: 40-47.
82. Traves S L, Culpitt S V, Russell R E, Barnes P J, Donnelly L E. Increased levels of the chemokines GROalpha and MCP-1 in sputum samples from patients with COPD. Thorax. 2002; 57: 590-595.
83. Van der Auwera, G. A., Carneiro, M. O., Hartl, C., Poplin, R., Del Angel, G., Levy-Moonshine, A.,
Jordan, T., Shakir, K., Roazen, D., Thibault, J., et al., From FastQ data to high confidence variant calls: the Genome Analysis Toolkit best practices pipeline. Curr Protoc Bioinformatics 2013; 43: 11 10 11-33.
84. Vaughan A E, Brumwell A N, Xi Y, Gotts J E, Brownfield D G, Treutlein B, Tan K, Tan V, Liu F C, Looney M R, Matthay M A, Rock J R, Chapman H A. (2015). Lineage-negative progenitors mobilize to regenerate lung epithelium after major injury. Nature 2015; 517, 621-625.
85. Vieira Braga F A, Kar G, Berg M, Carpaij O A, Polanski K, Simon L M, Brouwer S, Gomes T, Hesse L, Jiang J, Fasouli E S, Efremova M, Vento-Tormo R, Talavera-L6pez C, Jonker M R, Affleck K, Palit S, Strzelecka P M, Firth H V, Mahbubani K T, Cvejic A, Meyer K B, Saeb-Parsy K, Luinge M, Brandsma C A, Timens W, Angelidis I, Strunz M, Koppelman G H, van Oosterhout A J, Schiller H B, Theis F J, van den Berge M, Nawijn M C, Teichmann S A. A cellular census of human lungs identifies novel cell states in health and in asthma. Nat Med. 2019; 25:1153-1163.
86. Wedzicha J A. (2017). Airway mucins in chronic obstructive pulmonary disease. N Engl J Med. 2017; 377: 986-987.
87. Wen Y, Reid D W, Zhang D, Ward C, Wood-Baker R, Walters E H. Assessment of airway inflammation using sputum, BAL, and endobronchial biopsies in current and ex-smokers with established COPD. Int J Chron Obstruct Pulmon Dis. 2010; 5: 327-334 (2010).
88. Wills-Karp M, Luyimbazi J, Xu X, Schofield B, Neben T Y, Karp C L, Donaldson D D. Interleukin-13: central mediator of allergic asthma. Science 1998; 282: 2258-2261.
89. Xu Y, Mizuno T, Sridharan A, Du Y, Guo M, Tang J, Wikenheiser-Brokamp K A, Perl A T, Funari V A, Gokey J J, Stripp B R, Whitsett J A. Single-cell RNA sequencing identifies diverse roles of epithelial cells in idiopathic pulmonary fibrosis. JCI Insight. 2016; 1:e90558.
90. Yamaguchi T, Hosono Y, Yanagisawa K, Takahashi T. (2013). NKX2-1/TTF-1: an enigmatic oncogene that functions as a double-edged sword for cancer cell survival and progression. Cancer Cell 2013; 23: 718-723.
91. Yang, H., and Wang, K. Genomic variant annotation and prioritization with ANNOVAR and wANNOVAR. Nat Protoc 2015; 10: 1556-1566.
92. Yang Y, Riccio P, Schotsaert M, Mori M, Lu J, Lee D K, Garcia-Sastre A, Xu J, Cardoso W V. Spatial-Temporal Lineage Restrictions of Embryonic p63+ Progenitors Establish Distinct Stem Cell Pools in Adult Airways. Dev Cell 2018; 44: 752-761.
93. Zhou-Suckow Z, Duerr J, Hagner M, Agrawal R, Mall M A. Airway mucus, inflammation and remodeling: emerging links in the pathogenesis of chronic lung diseases. Cell Tissue Res. 2017; 367: 537-550.
94. Zhu L, Gazin C, Lawson N, Pages H, Lin S, Lapointe D and Green M. (2010). ChlPpeakAnno: a Bioconductor package to annotate ChIP-seq and ChIP-chip data. BMC Bioinformatics 2010; 11: 237.
95. Zuo W, Zhang T, Wu D Z, Guan S P, Liew A A, Yamamoto Y, Wang X, Lim S J, Vincent M, Lessard M, Crum C P, Xian W, McKeon F. p63(+)Krt5(+) distal airway stem cells are essential for lung regeneration. Nature 2015; 517: 616-620.

### VIII. Certain Examples—Idiopathic Pulmonary Fibrosis

Idiopathic pulmonary fibrosis (IPF) is a progressive and ultimately fatal interstitial lung disease (ILD) driven by unresolved mechanisms. Epithelial stem cell libraries were generated from lungs of 16 IPF patients and 10 controls without ILD. The inventors assessed the heterogeneity of these libraries using single cell RNA sequencing and performed molecular and functional analyses of single cell clones derived from these libraries. Single cell cloning analyses showed that IPF lungs are uniformly dominated by a stem cell variant (herein “Cluster 2) that drives fibrosis in xenograft models, autonomously converts lung fibroblasts to myofibroblasts in vitro and is distinct from three variants dominating COPD lungs. Differential gene expression indicates that Cluster 2 clones constitutively express gene sets linked to lung fibrosis and inflammation, as well as pathways and risk alleles previously implicated in IPF. In addition to the enhanced ratios of Cluster 2 in IPF lungs compared to control lungs (p=1.455e−11), within IPF lungs Cluster 2 cells are also more prevalent in regions of extensive usual interstitial pneumonia (UIP) histopathology. The presence of Cluster 2 cells at low levels in fetal lung suggests an innate immune function of these cells rather their pathologic conversion from normal lung stem cells. IPF lungs display high levels of a novel stem cell variant which is profibrotic and distinct from the pathogenic variants that dominate COPD lung. Drug screens reveal a differential reliance on signaling pathways by the IPF variant and suggest a route to therapeutics for this condition.

### A. Introduction

The rapid fatal progression of idiopathic pulmonary fibrosis (IPF)¹ underscores the need for understanding the mechanisms that drive this condition. Emerging concepts hold that IPF arises from recurrent, subclinical lung injury that imparts changes to epithelia and stromal cells which in turn compromises lung repair and favors the fibrotic alternative². Epithelial cells in particular have been tied to this condition by mutations in genes expressed by airway cells in familial and sporadic pulmonary fibrosis, by single cell RNA sequencing data, and by genetic risk loci across IPF patients³-8. In addition, a recent analysis of COPD, another lung disease, has linked its pathogenic features to the emergence of three discrete and clonogenic epithelial stem cell variants that separately promote mucin hypersecretion, fibrosis, and neutrophilic inflammation⁹, suggesting a possible role for aberrant stem cells in other prograssive pulmonary conditions characterized by abnormal epithelial cell function. In the present work the inventors detail the spectrum of clonogenic epithelial cells in IPF lungs and identify a single, predominant variant that drives the conversion and recruitment of myofibroblasts from fibroblasts. This epithelial variant in IPF is distinct from the three variants that dominate COPD lungs and may represent a disease-specific target for therapeutic intervention.

### B. Methods

Study Patients. University of Iowa Carver College of Medicine and the University of Texas Health Sciences Center Houston identified and clinically characterized 16 patients in the transplant services with IPF. The diagnosis of IPF was confirmed by histologic examination demonstrating usual interstitial pneumonia (UIP) in eight patients along with high-resolution computed tomography (CT) scanning and the absense of an alternative cause for the patient's progressive fibrotic lung disease¹. In eight patients, histology was not available and the diagnosis was based on clinical and CT scan results. In addition, 10 lungs from individuals without ILD were identified (see Table S1 in Supplementary Appendix). All protocols were approved by the institutional review boards at the University of Iowa, the University of Texas, and the University of Houston, and all subjects or their families provided informed written consent.

Clonogenic Analysis of Lung Stem Cells. Single cell suspensions of lung tissue from IPF and control lungs were plated onto lawns of irradiated 3T3-J2 murine embryonic fibroblasts and grown to stem cell colonies over ten days^{9,10}. The resulting stem cell “libraries” were analyzed by single cell RNA sequencing (scRNAseq)¹ and flow sorted as single cells to 384-well plates to establish discrete clones that were assessed by whole genome RNA sequencing. Stem cell libraries and single cell-derived clones were functionally assessed for pro-inflammatory and pro-fibrotic activity by subcutaneous transplants in highly immunodeficient mice (NOD-scid IL2Rγ^{null}; “NSG”)¹¹ and immunohistological analysis of host immune cell responses and myofibroblast activation in the resulting xenografts using appropriate marker antibodies to epithelia (anti-ECAD, p63), myofibroblasts (anti-αSMA, fibronectin), and neutrophils (anti-CD45, Ly6G). The ability of stem cell libraries or single cell-derived clones to promote the activation of normal lung fibroblasts to myofibroblasts was determined by plating onto lawns of normal lung fibroblasts and monitoring their conversion by antibodies to α-SMA and fibronectin, and quantified by FACS profiling of α-SMA expressing myofibroblasts.

In vitro Air-liquid Interface (ALI) Differentiation. Air-liquid interface (ALI) culture of epithelial clones were performed as described^{9,10}. Briefly, Transweli inserts (Corning, USA) were coated with 20% Matrigel (Becton Dickinson, Palo Alto) and incubated at 37° C. for 30 min to gel. QuadroMACS Starting Kit (LS) (Miltenyi Biotec, Bergisch Gladbach) was used to remove feeder cells from epithelial stem cells. 200,000-300,000 cells were seeded into each Transwell insert and cultured in StemECHO™ PU stem cell growth media. At confluency (3-7 days), the apical media on the inserts was removed through careful pipetting and the cultures were continued in differentiation media (PneumaCult-ALI Media, STEMCELL Technologies, Vancouver) for an additional 14-21 days prior to harvesting. The differentiation media was changed every two days.

Xenografts in immunodeficient mice. One million in vitro cultured stem cells were harvested by trypsinization, mixed with 50% Matrigel (Becton Dickinson, Palo Alto) to a volume of 100 μl and injected subcutaneously in NSG¹¹ (NOD-scid IL2Rγ^{null}) mice (Jackson Laboratories, Bar Harbor) and harvested two weeks later.

Histology and immunostaining. Histology, hematoxylin and eosin (H&E) staining, immunohistochemistry (IHC) and immunofluorescence (IF) staining were performed using standard protocols. For IHC and IF, 4% paraformaldehyde-fixed, paraffin-embedded tissue slides were subjected to antigen retrieval in citrate buffer (pH 6.0, Sigma-Aldrich, USA) at 120° C. for 20 min, and a blocking procedure was performed using 5% bovine serum albumin (BSA, Sigma-Aldrich, USA) and 0.05% Triton X-100 (Sigma-Aldrich, USA) in DPBS (−) (Gibco, USA) at room temperature for 1 hr. The sources of primary antibodies used in this study include: mouse monoclonal human cytokeratin 5 antibody (NCL-L-CK5; Leica Biosystems, Germany); STEM121 mouse monoclonal antibody specific for human cytoplasmic marker (AB-121-U-050; Takara Bio); mouse monoclonal human nucleoli marker antibody (NBP2-32886; Novus Biologicals); rabbit polyclonal AQP4 antibody (HPA014784; Sigma-Aldrich, USA); mouse monoclonal acetylated tubulin (T745126; Sigma-Aldrich, USA); rat monoclonal mouse CD45 antibody (14-0451-85; Thermo Fisher, USA); rat monoclonal mouse Ly6G (MAB1037; R&D systems, USA); rabbit polyclonal Muc5B antibody (ab87376; Abcam, UK); rabbit polyclonal Muc5AC antibody (ab78660; Abcam, UK); rabbit polyclonal anti-p63 antibody (GTX102425; GeneTex); mouse monoclonal anti-p63 antibody (ab735; Abcam); mouse monoclonal anti-CEACAM6 antibody (FAB3934A; R&D Systems); mouse monoclonal Vimentin antibody (MS-129-P1; Thermo Scientific); rabbit polyclonal anti-human GDF15 antibody (MBS9213643; MyBioSource); goat polyclonal E-Cadherin antibody (AF648; R&D Systems, USA); mouse monoclonal alpha smooth muscle actin antibody (ab7817; Abcam, UK); rabbit polyclonal Involucrin antibody (HPA055211; Sigma-Aldrich, USA); goat polyclonal alpha-smooth muscle actin antibody (NB300-978SS; Novus Biologicals). Secondary antibodies used in this study included Alexa Fluor-488, Alexa Fluor-555 or Alexa Fluor-647 donkey anti-goat/rat/mouse/rabbit IgG antibody (Thermo Fisher, USA). All images were captured using the Inverted Eclipse Ti-Series (Nikon, Japan) microscope with a Lumencor SOLA light engine and an Andor Technology Clara Interline CCD camera and NIS-Elements Advanced Research v.4.13 software (Nikon, Japan) or LSM 780 confocal microscope (Carl Zeiss, Germany) with LSM software. Bright field cell culture images were obtained on an Eclipse TS100 microscope (Nikon, Japan) with Digital Sight DSFil camera (Nikon, Japan) and NIS-Elements F3.0 software (Nikon, Japan).

RNA Sample Preparation. For stem cells, ALI, or xenografts derived from IPF or control patients, RNA was isolated using Trizol RNA Isolation Kit (Life Technologies, USA). RNA quality (RNA integrity number, RIN) was measured using the Agilent 2100 Bioanalyzer and Agilent RNA 6000 Nano Kit (Agilent Technologies, USA). RNAs having a RIN>8 were used for RNA-Seq analysis.

Analysis of Fibrosis in Xenografts. Histological sections of xenografts were stained with antibodies to ECAD, an epithelial marker, and αSMA, a marker of myofibroblasts. Fluorescence images of each section were captured and analyzed by measurements of the lengths of cystic epithelia from the transplanted cells and the fraction bounded by uSMA-positive myofibroblasts. The lengths of cystic epithelia and myofibroblast coverage in all fluorescent images were measured, quantified and analyzed by NIS-Elements Advanced Research v.4.13 software (Nikon, Japan). Myofibroblast contact (%) was calculated as length of myofibroblasts/lengths of epithelia×100%.

Flow Cytometry and Sorting. To assess the percentage of Cluster 2 cells in stem cell libraries, the inventors used antibodies to cell surface markers of these cells and flow sorting. In brief, stem cell libraries were trypsinized and harvested as single cell suspensions. 3T3-J2 murine embryonic fibroblast feeder cells were removed using (QuadroMACS™ Separator and Starting Kits; Feeder Removal MicroBeads, mouse; Miltenyi Biotec) and approximately 300,000 stem cells were incubated with anti-CEACAM6 antibody (lug) at 4° C. for 1 hr after a blocking procedure with FACS buffer (PBS+2% FBS+0.05% sodium azide) at 4° C. for 30 min. Cells were then incubated with Alexa Fluor 488 Secondary antibodies (Thermo Fisher, USA) for 1 hr at 4° C., with five times washing between each step. Samples were collected and analyzed on a Sony SH800S Cell Sorter (Sony Biotechnology, USA).

In vitro fibroblast to myofibroblast conversion assay. Human normal lung fibroblasts (HLFs, CC-2512; Lonza) were cultured in StemECHO™ PU stem cell basal media. 50,000 cells were seeded per well in a 24-well plate one day before 25,000 Cluster 1, Cluster 2 or Cluster 1+2 cells were seeded into plates coated with HLFs at 30-50% confluency. Mixtures of stem cells and HLFs were then maintained for 5 days. Media were changed every other day. At Day 6 post co-culture, cells were either fixed with 4% paraformaldehyde for IF staining or trypsinized, harvested as single cell suspensions, then fixed and permeabilized by using Fixation/Permeabilization Solution Kit (BD biosciences, USA, cat. 554714). After a blocking procedure, cells were incubated with mouse monoclonal α-SMA antibody (ab7817; Abcam, UK) and Alexa Fluor 488 Secondary antibodies, with five washings between each step. Samples were collected and analyzed with on a Sony SH800S Cell Sorter (Sony Biotechnology, USA).

Comparison of Upper Lobes and Lower Lobes from IPF patients. Biopsies of upper and lower lobe were bisected and the halves processed for histology and epithelial stem cell library generation. Histological sections were assessed by immunofluorescence with antibodies to GDF15, MUC1, and VIM, and quantified by morphometric analysis. Corresponding stem cell libraries were assessed for CEACAM6+ cells by quantitative FACS, and by xenografting in immunodeficient mice for the association with αSMA+ myofibroblasts.

High Throughput Screening (HTS). Cluster 1 and 2 stem cell clones were seeded on multiple 384-well plates (Griener Bio-One, USA). 1-day post seeding, compounds from arrayed bioactive molecule collections (e.g. Selleck, Prestwick) were added by automation at the High Throughput Research and Screening Center of the Institute of Biosciences and Technology (Houston, Tex.), Texas A&M University. Positive (paclitaxel, 10 uM) and negative control (DMSO) lanes were allocated within each plate. After treatment, plates were sealed with breathable membranes and maintained for 4 days in a 37° C., 7.5% CO₂ incubator. Plates were then fixed, stained, and imaged. In brief, the treated 384-well plates were washed with phosphate buffered saline (Gibco, USA) and fixed with 4% paraformaldehyde at room temperature for 25 minutes. After fixation, plates were then stained with DAPI for 1 hr at room temperature before imaging via a high-content automatic screening system (Thermo Scientific Celllnsight CX7 LED, Thermo Fisher Scientific, Waltham, Mass., USA).

Transcriptomic sequencing data analysis. All RNA-seq libraries were sequenced on Illumina NovaSeq 6000 with 150 bp pair-end reads. Raw reads were trimmed to remove low quality bases (phred score <20) and sequencing adapter leftovers using Trim Galore (world-wide-web at bioinformatics.babraham.ac.uk/projects/trim_galore/). Potential mouse genomic DNA contaminant reads were removed for further analysis using Xenome³². Trimmed RNAseq reads were mapped to the human genome (UCSC hg19) using Salmon (version 0.9.1) with default settings³³. Alignment results were then input to DEseq2³⁴ for differential expression analysis with default settings and FDR less than 0.05. The heatmaps with hierarchical clustering analysis of the global gene expression pattern in different samples were performed using pheatmap package (cran.rproject.org/web/packages/pheatmap/index.html) in R (version 3.5.1). The pathway enrichment analysis was performed using Enrichr³⁵.

Sequence alignment of single cell RNA sequencing. The single cell mRNA sequencing (scRNA-seq) libraries were established using the 10× Genomics Chromium system (Single Cell 3′ Reagent Kit v2). The scRNA-seq libraries were sequenced on an Illumina HiSeq×Ten with 10K cells for IPF, COPD cases and fetal lung case. Demultiplexing, alignment and UMI-collapsing were performed using the Cellranger toolkit (version 2.1.0, 10× Genomics). The raw paired-end reads were trimmed to 26 bps for Read1 and 98 bps for Read2. The trimmed reads were mapped to both the human genome (hg19) and the mouse genome (mm10). The reads uniquely mapped to the human genome were used for downstream analysis.

Single cell RNA sequencing. The scRNA-seq data analyses were performed using the Seurat package (version 2.3.4)³¹. The inventors kept the genes with expression in at least three cells, and excluded cells expressing less than 200 genes. They also excluded the cells with high mitochondrial percentage or with an outlier level of UMI content. The normalization was performed using the global-scaling normalization method, which normalizes the gene expression measurements for each cell by the total expression, and then multiplies by 10,000, and finally log-transforms the result. The variable genes were identified using a function to calculate average expression and dispersion for each gene, divides these genes into bins, and then calculates a z-score for dispersion within each bin (“x.low.cutoff=0.0125”, “x.high.cutoff=3”, and “y.cutoff=0.5”). The inventors scaled the data to regress out the variation of mitochondrial gene expression.

The inventors performed PCA based on the scaled data to identify significant principal components (PCs). They selected the PCs with p-values less than 0.01 as input to perform clustering analysis and visualization by t-SNE. They detected the marker genes in each cell subpopulation using two methods of Wilcoxon rank sum test and DESeq2. For Wilcoxon rank sum tests, they used the default parameter. For DEseq2, they kept the marker genes with average log-fold change above 0.1 and adjust p-value fewer than 0.05.

Contaminating 3T3-J2 fibroblast cells were identified by murine reads. In addition, the cells in S stage of cell cycle were identified based on the marker gene of SLBP³⁶. The cells in G2 or M stage of cell cycle were identified based on the marker genes of UBE2C, AURKA, CENPA, CDCl20, HMGB2, CKS2, and CKS1B. The cells in G0 stage of cell cycle were identified based on the marker genes of GOS2. In addition, the ambiguous cells with few marker genes were also removed, which could possibly correspond to sequencing low quality cells.

High content image analysis and dose-response analysis. HCS Studio Cell Analysis Software version 4.0 (world-wide-web at thermofisher.com/us/en/home/life-science/cell-analysis/cellular-imaging/high-content-screening/hcs-studio-2.html) was used for enumeration of viable cells based on the HTS images and data management. The viable cells labelled with DAPI in each plate were imaged within each stem cell colony. The wells treated with various chemicals as well as controls were assessed based on the fluorescent signal threshold using automated image analysis. The Local Maxima method was used to enumerate cell number per well, which is a kind of peak detection method that identifies spikes in pixel intensity within the spot detection region. An intensity threshold was set by using the Fixed method and any pixel above this threshold intensity was considered an object and used for following analysis. Z-factor of each plate was calculated by HCS Studio Software, which was used as a criterion for assessing image quality of each well. The wells with outliers were ignored. To calculate the survival rate per well, the cell number per well was divided by the median of negative control lanes as a standardization step. To screen potential effective drugs, the survival ratio per well of the plate of Cluster 2 cell lines was divided by the corresponding well of the plate of Cluster 1 cell lines (cut-off <75%). The dose-response curves of drugs were plotted by fitting a four-parameter log-logistic dose-response model to the survival rate data using drc³⁷ v3.0.1 package in R. The ED₅₀ value is estimated by module ED in drc package.

Statistical analysis. Unpaired two-tailed student's t-test was used to determine the statistical significance between two groups. Statistical analyses were performed using R (version 3.5.1). The “n” numbers for each experiment are provided in the text and figures. P<0.05 was considered statistically significant. Asterisks denote corresponding statistical significance *p<0.05; **p<0.01; ***p<0.001 and ****p<0.0001.

### C. Results

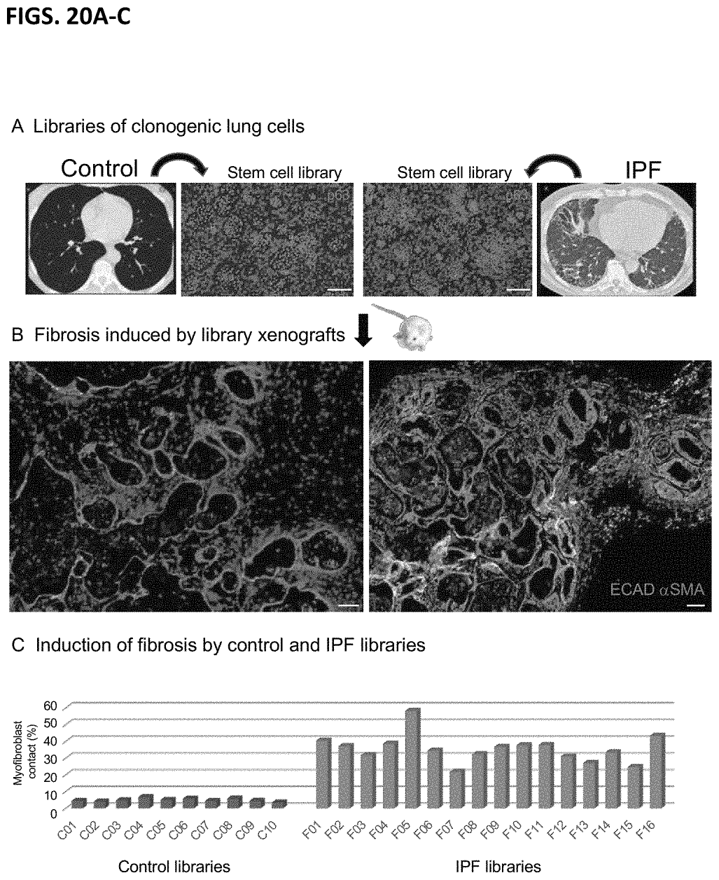

IPF LUNG STEM CELL LIBRARIES ARE PRO-FIBROTIC. Libraries of clonogenic epithelial stem cells were generated from 16 IPF lungs and 10 normal lungs using methods that support the growth of regenerative (p63+/Krt5+) cells but not of differentiated cell types (FIG. 20A)^{9,10}. These clonogenic cells represent approximately 1:1,000 to 1:3,000 of all epithelial cells in the lung tissue sample and display an undifferentiated morphology and unlimited regenerative capacity. To assess potential phenotypic differences between the control and IPF stem cell libraries, the inventors performed subcutaneous transplants to immunodeficient (NODscid IL2Rγ^{null}; “NSG”) mice⁹. Transplanted cells from both control and IPF libraries proliferated and formed polarized, E-cadherin (ECAD)-positive epithelial cysts over a period of two weeks in the murine host⁹ (FIG. 20B). However, unlike those of the control libraries, the IPF library cysts were surrounded by a dense meshwork of (YSMA-expressing myofibroblasts (FIG. 20B), a fibroblast-derived cell that is tightly linked to fibrosis in IPF, COPD, and other lung conditions¹². A morphometric analysis of submucosal myofibroblast association with epithelial cysts in xenografts of 10 control and 16 IPF libraries showed that control xenografts displayed low levels of myofibroblast association (5+/−1.2%), whereas IPF cysts showed an extensive association (35+/−8.3%) with myofibroblasts (p=1.21e−10; FIG. 20C).

Analyses of these xenografts further showed that the myofibroblasts present in the IPF stem cell library xenografts were of host (murine) origin rather than the result of an epithelial-mesenchymal transition (EMT) of the human IPF cells. The inventors also found that, in contrast to the considerable neutrophilic properties of the COPD library transplants versus controls (p=3.07e−09)⁹, the 16 IPF libraries as a group triggered less neutrophils than COPD libraries (p=8.048e−05) but more than controls (p=0.007).

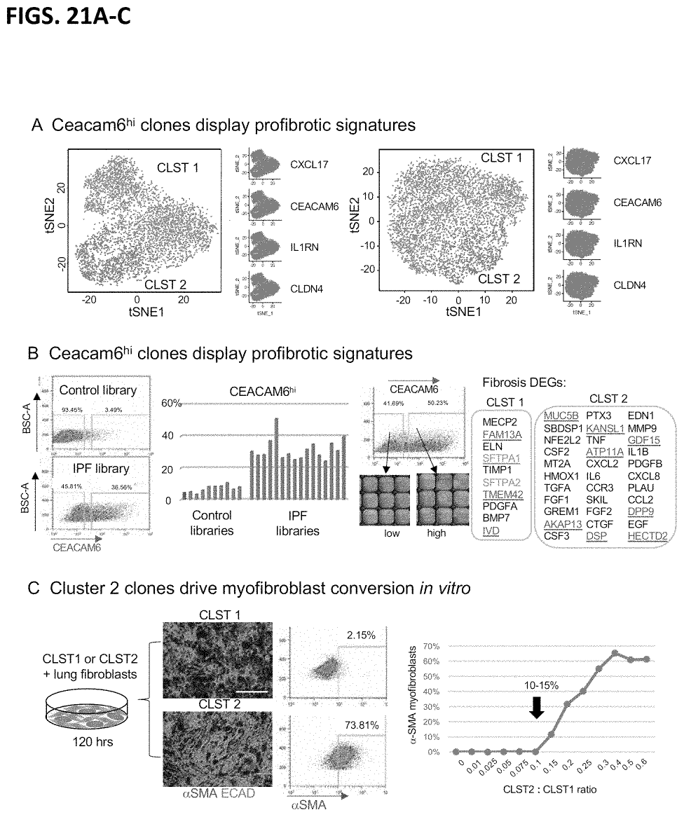

A SINGLE, PRO-FIBROTIC STEM CELL VARIANT IN IPF. scRNA-seq was used to assess the heterogeneity of stem cell libraries of IPF and control patients. IPF stem cell libraries showed two major cell clusters in scRNAseq profiles including normal lung stem cells (Cluster 1)⁹, and a novel population the inventors designated “Cluster 2” that is marked by differential expression of CXCL17, CEACAM6, IL1RN, and CLDN4 (FIG. 21A). Antibodies to CEACAM6 in live cell FACS across all libraries showed that Cluster 2 cells comprised higher percentages in IPF libraries (31.9+/−6.8%) than control libraries (7.1+/−2.3%; p=1.455e−11; FIG. 21B).

To clone stem cell representatives of Cluster 1 and Cluster 2, the inventors sorted single cells from IPF libraries based on CEACAM6 expression to 384-well plates followed by clonal expansion. Xenografts of CEACAM6-negative clones confirmed that they are Cluster 1 cells committed to a terminal bronchiolar epithelium⁹ expressing SCGB1A1, SFTBP, and AQP4, while CEACAM6-positive (Cluster 2) clones differentiated to a cuboidal epithelium marked by expression of genes previously linked to IPF epithelia such as MUC¹³, VIM¹⁴, and GDF15¹⁵. As with the unfractionated IPF libraries, the cloned Cluster 2 cells assembled into polarized epithelial cysts that were surrounded by (YSMA-expressing myofibroblasts. In contrast, the epithelial cysts formed by cloned Cluster 1 cells from IPF libraries were largely devoid of myofibroblast association. Consistent with the high concentration of myofibroblasts about Cluster 2 cysts in xenografts, whole genome expression profiling of Cluster 2 and Cluster 1 cells showed that Cluster 2 cells differentially express (q<0.05) genes linked to lung fibrosis gene sets, including multiple genes implicated in IPF and ILD via molecular genetics and biomarker efforts (FIG. 21B)^{3,5-8,16}.

IPF STEM CELL VARIANT DRIVES MYOFIBROBLAST CONVERSION IN VITRO. Given the robust expression of pro-fibrotic genes expressed by the IPF Cluster 2 variant, the inventors asked if these cells could directly promote the conversion of normal human lung fibroblasts to myofibroblasts. Co-cultures of normal lung fibroblasts and Cluster 2 clones from IPF libraries resulted in a strong induction of the myofibroblast marker αSMA and a quantitative shift in fibroblasts to αSMA+ cells by FACS profiling (FIG. 21C). Challenging these lung fibroblasts with combinations of Cluster 1 and 2 cells at defined ratios showed an inflection point in the activation of myofibroblasts between 10 and 15% Cluster 2 cells (FIG. 21C). Corresponding in vivo studies of defined ratios of Cluster 1 and Cluster 2 in xenografts revealed a similar inflection point between 10 and 20% Cluster 2 cells. Together, these findings indicate that, within the context of experimental design, Cluster 2 cells promote myofibroblast conversion and recruitment when they represent more than 10% of the total epithelial cell population.

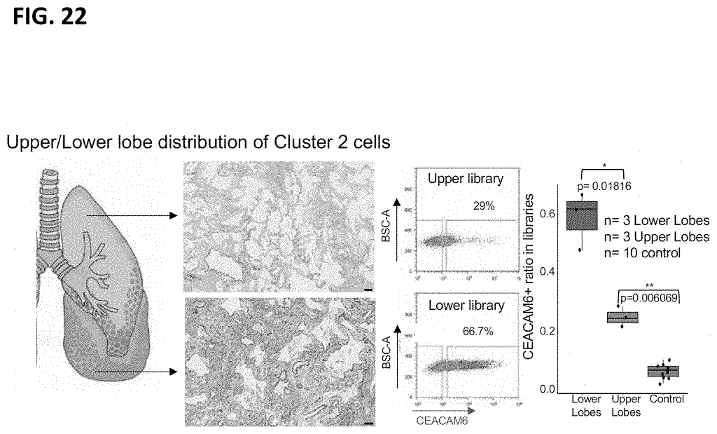

IPF STEM CELL VARIANT CORRELATES WITH UIP HISTOPATHOLOGY. IPF lungs are marked by profound heterogeneity in histopathology with the upper lobes relatively spared and lower lobes marked by UIP histopathology including bilateral and peripheral reticulation and honeycombing^{1,17}. In three IPF lungs, the inventors generated patient-matched libraries from upper lobes marked by moderate pathology and from lower lobes with extensive fibrosis (FIG. 22). CEACAM6 FACS profiling of these stem cell libraries showed that the upper lobe libraries had an average of 25.6+/−3.37% Cluster 2 cells whereas the lower lobes showed an average 59.0%+/−9.66% (p=0.018; FIG. 22). Xenografts of the lower lobe libraries also proved to be more fibrogenic in xenografts than the upper lobe libraries (67.8+/−11.8% vs 29.5+/−6.8%; p=0.016). Consistently, these upper and lower lobes also showed differential labelling with antibodies to GDF15, a noncanonical TGF-beta receptor ligand whose plasma levels have been linked to IPF severity¹⁵ (36.8 vs 73.2%, p=0.0047). Together these data suggest that Cluster 2 stem cells are present at higher ratios relative to Cluster 1 cells in regions of the IPF lung showing more severe UIP histology.

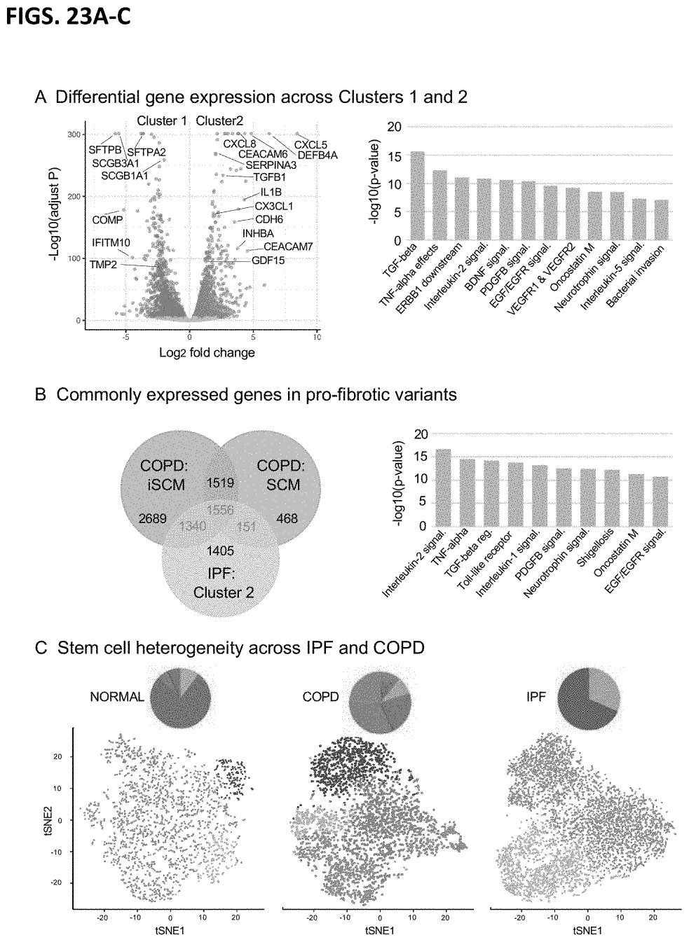

IPF CLUSTER 2 VARIANT IS DISTINCT FROM COPD PRO-FIBROTIC VARIANTS. Given the prominence of fibrosis in both IPF and COPD¹⁸⁻²⁰, and the two pro-fibrotic stem cell variants identified in COPD⁹, the inventors sought to place the IPF Cluster 2 cell in context with the normal and COPD variants. Whole genome RNA sequencing of patient-matched, in vitro differentiated Cluster 1 (normal) and Cluster 2 stem cells showed that the normal terminal bronchiolar stem cells acquired expression of secretoglobulins SCGB3A1 and SCGB1A1 and surfactants SFTPB and SFTPA2 linked to Club and alveolar cells, respectively (FIG. 23A). In contrast, the Cluster 2 cells differentiated to cells expressing GDF15, CEACMA6, TGFB1, IL1B, and other markers linked to IPF. At the gene set level (p<7.6e−08), Cluster 2 cells differentially express genes in associated with signaling by TGF-β, Interleukin-2, BDNF, PDGFB, EGFR, Oncostatin M, and in innate immune responses to pathogens (FIG. 23A).

The two pro-fibrotic stem cell variants that dominate the COPD lung are committed to squamous cell metaplasia⁹, while the IPF Cluster 2 stem cells differentiate to a non-squamous, cuboidal epithelium. Nevertheless, comparison of RNA-seq profiles show an overlap of expressed genes between IPF Cluster 2 and the COPD variants whose gene sets show significant (p<1.9e−11) links to Oncostatin M, TGF-β, Interferons, Toll-like Receptors, and responses to pathogens (FIG. 23B).

Importantly, the scRNAseq profiles of COPD and IPF libraries were also distinct even though both libraries induce myofibroblast activation in xenograft models (FIG. 23C). COPD libraries are dominated by three variants (iSCM, SCM, and GCM), and also show a minor fraction (approximately 10%) of the IPF Cluster 2, which is similar to the composition of the normal lung. Lastly, the IPF Cluster 2 variant, as well as the variants identified in COPD lung, are present at low levels in fetal lung and likely reflect indigious stem cell lineages rather than disease-induced anomolies.

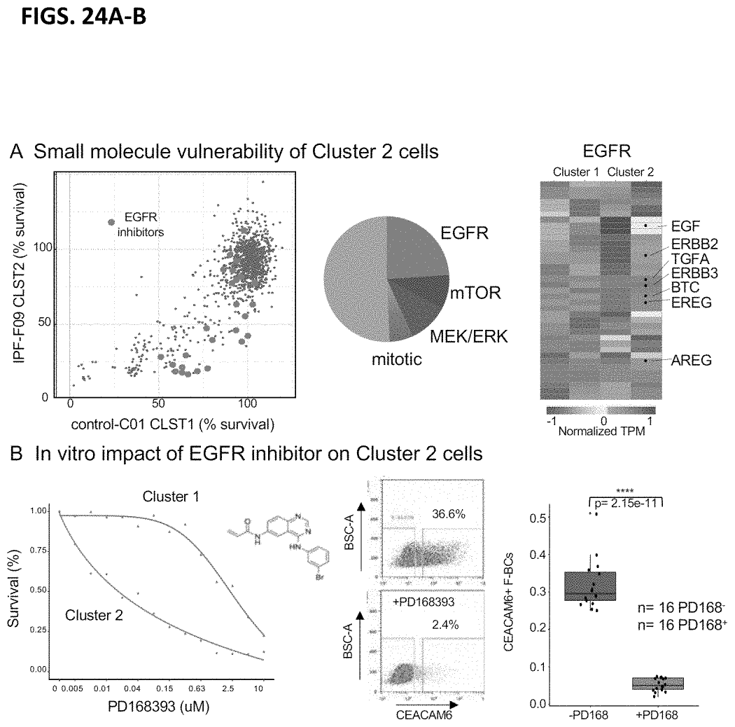

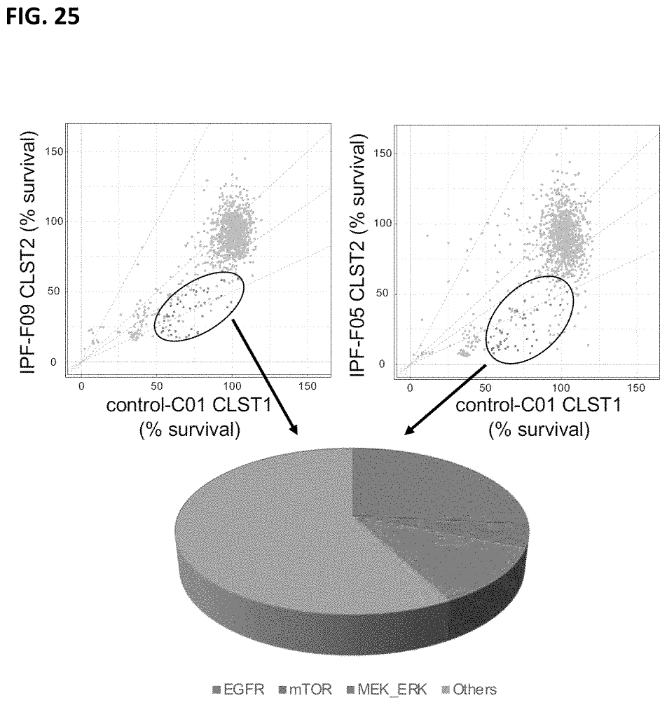

VULNERABILITIES OF IPF STEM CELL VARIANT TO SMALL MOLECULE INHIBITORS. To examine potential vulnerabilities of Cluster 2 cells with respect to the normal lung stem cells of Cluster 1, the inventors adapted these cells to 384-well formats and performed parallel screens of bioactive small molecules containing inhibitors of signal transduction pathways (FIG. 24A; FIG. 25). Whereas the vast majority of these molecules had no effect on either population, of 45 found to differentially affect Cluster 2 versus Cluster 1 cells from two cases (IPF F09 and F03), those targeting certain pathways, such as EGFR signaling, were disproportionally represented (FIG. 24; FIG. 25). Gene expression profiles of Cluster 1 and Cluster 2 cells indicate that Cluster 2 cells differentially express (q<0.05) a large array of EGFR ligands, including EGF, ERBB2, ERBB3, BTC, TGFα, and EREG, and may explain the skewed effects of EGFR inhibitory compounds on Cluster 2 cells (FIG. 24A). Dose-response curves for one of these EGFR inhibitors confirm its in vitro selectivity against Cluster 2 cells across all 16 IPF libraries (FIG. 24B). While aberrant EGFR signaling has been tied to lung fibrosis in mice and epithelial dysfunction in IPF^{21,21}, the extensive use EGFR inhibitors in oncology has not led to reports of IPF remission. Notwithstanding, additional studies into the specific sensitivities of Cluster 2 cells may lead to new and perhaps combination therapeutics for IPF.

### D. Discussion

A clonogenic analysis of lung stem cells across 16 cases of IPF and 10 controls has revealed a major stem cell variant in IPF lung that promotes myofibroblast activation in both in vitro and xenograft models. This pro-fibrotic Cluster 2 variant is marked by constitutive expression of pro-fibrotic and pro-inflammatory genes, and displays lineage commitment and gene expression signatures distinct from the two pro-fibrotic variants that pervade the COPD lung⁹. Importantly, the Cluster 2 stem is present in all control lungs, albeit at levels significantly below those of IPF patients (7.1 versus 32%, p=1.46e−11). Even within single IPF lungs, regions of profound UIP histopathology show higher percentages of Cluster 2 (59%) than upper lobes with less pathology (26%; p=0.018). Aside from these associations, in vitro and in vivo Cluster 2 dose-response experiments reveal a Cluster 2-dependent threshold for myofibroblast conversion with 10⁻²⁰% inflection points midway between levels of Cluster 2 cells in control and IPF lung stem cell libraries. How such a Cluster 2 threshold is surpassed in IPF is unclear, but may be triggered by the recurrent and subclinical events of lung damage thought to precede the onset of IPF and brought about by risk factors such as smoking²³, infection²⁴, gastroesophageal reflux²⁵, and genetics^{3,5-8}.

Consistent with the notion that innate immune processes underly the pathogenesis of IPFU, IPF Cluster 2 cells show a constitutive expression of genes tied to the response to bacteria, viruses, and protozoans, including cytokines (TGFB1, CXCL8), pathogen detection (TLR3, ATP11A, IL1RN), and epithelial barriers (DSP, DPP9) genetically linked to the risk of IPF^{6-8,16,26}. The presence of the Cluster 2 variant in control and even fetal lung, coupled with its expression of fibrotic and inflammatory pathways, supports the concept that Cluster 2 cells comprise one element of a network of minor epithelial variants, which coordinate the response to pathogens. Understanding how these minor variants are differentially amplified in COPD, IPF, and perhaps other lung conditions will refine the specific risk factors for these diseases. Conversely, deciphering why these variants come to dominate the lung could aid in the resolution of these conditions. In this regard, the inventors note that SFTPA1 and SFTPA2, two surfactant genes in which non-synonymous mutations are linked to familial IPF^{3,26-30}, are highly expressed in the normal Cluster 1 cell. The specific activation of unfolded protein responses^{3,26-30} in Cluster 1 cells may provide a competitive advantage to Cluster 2 cells and contribute to the sway of this pro-fibrotic variant.

Lastly, there is a critical need for therapeutics that arrest or reverse the rapid evolution of IPF. If the Cluster 2 variant figures in the pathogenesis of IPF, these cells represent targets of prospective therapies. Bioactive compound screening of patient-matched Cluster 1 and Cluster 2 can identify specific vulnerabilities of Cluster 2 cells to inhibition of EGFR and mTOR signaling pathways, among others, and may guide therapeutic strategies for IPF.

### E. References for IPF Example

1. Lederer D J and Martinez F J. Idiopathic Pulmonary Fibrosis. N Engl J Med. 2018 378:1811-1823.
2. Wolters P J, Blackwell T S, Eickelberg O et al. Time for a change: is idiopathic pulmonary fibrosis still idiopathic and only fibrotic? Lancet Respir Med. 2018 6:154-160.
3. Whitsett J A, Wert S E, and Weaver T E. Diseases of pulmonary surfactant homeostasis.
Annu Rev Pathol. 2015 10:371-93.
4. Xu Y, Mizuno T, Sridharan A et al. Single-cell RNA sequencing identifies diverse roles of epithelial cells in idiopathic pulmonary fibrosis. JCI Insight. 2016 1:e90558.
5. Seibold M A, Wise A L, Speer M C et al. A common MUC5B promoter polymorphism and pulmonary fibrosis. N Engl J Med. 2011 364:1503-12.
6. Fingerlin T E, Murphy E, Zhang W et al. Genome-wide association study identifies multiple susceptibility loci for pulmonary fibrosis. Nat Genet. 2013 45:613-20.
7. Moore C, Blumhagen R Z, Yang I V et al. Resequencing study confirms that host defense and cell senescence gene variants contribute to the risk of idiopathic pulmonary fibrosis. Am J Respir Crit Care Med. 2019 200:199-208.
8. Allen R J, Guillen-Guio B, Oldham J M et al., Genome-Wide Association Study of Susceptibility to Idiopathic Pulmonary Fibrosis. Am J Respir Crit Care Med. 2020 201:564-574.
9. Rao W, Wang S, Duleba M et al. Regenerative metaplastic clones in COPD lung drive inflammation and fibrosis. Cell. 2020 181:848-864.
10. Rao W, Niroula S, Wang S et al. Protocol for cloning epithelial stem cell variants from human lung. STAR Protoc. 2020 1:100063.
11. Walsh N C, Kenney L L, Jangalwe S et al., Humanized mouse models of clinical disease. Annu Rev Pathol. 2017 12:187-215.
12. Duffield J S, Lupher M, Thannickal V J et al. Host responses in tissue repair and fibrosis. Annu Rev Pathol. 2013 8:241-76.
13. Milara J, Ballester B, Montero P et al. MUC1 intracellular bioactivation mediates lung fibrosis. Thorax. 2020 75:132-142.
14. Marmai C, Sutherland R E, Kim K K et al. Alveolar epithelial cells express mesenchymal proteins in patients with idiopathic pulmonary fibrosis. Am J Physiol Lung Cell Mol Physiol. 2011 301:L71-8.
15. Zhang Y, Jiang M, Nouraie M et al., GDF15 is an epithelial-derived biomarker of idiopathic pulmonary fibrosis. Am J Physiol Lung Cell Mol Physiol. 2019 317:L510-L521.
16. Konigsberg I R, Borie R, Walts A D et al. Molecular signatures of idiopathic pulmonary fibrosis. Am J Respir Cell Mol Biol. 2021 May 26. doi: 10.1165/rcmb.2020-05460C. PMID:34038697
17. Bjoraker J A, Ryu J H, Edwin M K et al. Prognostic significance of histopathologic subsets in idiopathic pulmonary fibrosis. Am J Respir Crit Care Med. 1998 157:199-203.
18. Hogg J C. Pathophysiology of airflow limitation in chronic obstructive pulmonary disease. Lancet. 2004 364:709-21.
19. McDonough J E, Yuan R, Suzuki M et al. Small airway obstruction and emphysema in chronic obstructive pulmonary disease. N Engl J Med. 2011 365:1567-75.
20. Barnes P J. Small airway fibrosis in COPD. Int J Biochem Cell Biol. 2019 116:105598.
21. Korfhagen T R, Swantz R J, Wert S E et al. Respiratory epithelial cell expression of human transforming growth factor-alpha induces lung fibrosis in transgenic mice. J Clin Invest. 1994 April; 93(4):1691-1699.
22. Stancil, I T, Michalski, J E, David-Hall, D et al. Pulmonary fibrosis distal airway epithelia are dynamically and structurally dysfunctional. Nat. Comm. (in press).
23. Baumgartner K B, Samet J M, Stidley C A et al. Cigarette smoking: a risk factor for idiopathic pulmonary fibrosis. Am J Respir Crit Care Med. 1997 155:242-8.
24. Moore B B, and Moore T A. Viruses in Idiopathic Pulmonary Fibrosis. Etiology and Exacerbation. Ann Am Thorac Soc. 2015 Suppl 2:S186-92.
25. Raghu G, Freudenberger T D, Yang S et al. High prevalence of abnormal acid gastro-oesophageal reflux in idiopathic pulmonary fibrosis. Eur Respir J. 2006 27:136-42.
26. Michalski J E and Schwartz D A. Genetic Risk Factors for Idiopathic Pulmonary Fibrosis: Insights into Immunopathogenesis. J Inflamm Res. 202113:1305-1318.
27. Selman, M., Lin, H. M., Montano, M et al. (2003). Surfactant protein A and B genetic variants predispose to idiopathic pulmonary fibrosis. Human Genetics, 113: 542-550.
28. Wang, Y., Kuan, P. J., Xing, C. et al. Genetic defects in surfactant protein A2 are associated with pulmonary fibrosis and lung cancer. American Journal of Human Genetics 2009 84:52-59.
29. Lawson, W. E., Crossno, P. F., Polosukhin, V. V. et al. Endoplasmic reticulum stress in alveolar epithelial cells is prominent in IPF: Association with altered surfactant protein processing and herpesvirus infection. American Journal of Physiology. Lung Cellular and Molecular Physiology 2008 294:L1119-1126.
30. Burman A, Tanjore H, and Blackwell T S. Endoplasmic reticulum stress in pulmonary fibrosis. Matrix Biol. 2018 68-69:355-365.
31. Satija R, Farrell J A, Gennert D, Schier A F, Regev A. Spatial reconstruction of single-cell gene expression data. Nat Biotechnol 2015 33:495-502.
32. Conway T, Wazny J, Bromage A, et al. Xenome—a tool for classifying reads from xenograft samples. Bioinformatics 2012 28:i172-178.
33. Patro R, Duggal G, Love M I, Irizarry R A, Kingsford C. Salmon provides fast and bias-aware quantification of transcript expression. Nat Methods 2017 14:417-419.
34. Love M I, Huber W, Anders S. Moderated estimation of fold change and dispersion for RNA-seq data with DESeq2. Genome Biol 2014 15:550.
35. Chen E Y, Tan C M, Kou Y, et al. Enrichr: interactive and collaborative HTML5 gene list enrichment analysis tool. BMC Bioinformatics 2013 14:128.
36. Nestorowa S, Hamey F K, Pijuan Sala B, et al. A single-cell resolution map of mouse hematopoietic stem and progenitor cell differentiation. Blood 2016 128:e20-31.
37. Ritz C, Baty F, Streibig J C, et al. Dose-response analysis using R. PLoS One 2015 10:e0146021.

1. A method for treating a patient presenting with one or more of an inflammatory disease, metaplasia, dysplasia or cancer of pulmonary tissues which method comprises administering to the patient an agent that selectively kills or inhibits the proliferation or induces differentiation of pathogenic lung epithelial stem cells (PLESCs) relative to normal pulmonary stem cells.

2. A method of reducing proliferation, survival, migration, or colony formation ability of pathogenic lung epithelial stem cells (PLESCs) in a subject in need thereof comprising contacting the cell with a therapeutically effective amount of an agent that selectively kills or inhibits the proliferation or induces differentiation of PLESC relative to normal pulmonary stem cells.

3. A pharmaceutical preparation for treating one or more of an inflammatory disease, metaplasia, dysplasia or cancer of pulmonary tissues, the preparation comprises an agent that selectively kills or inhibits the proliferation or induces differentiation of pathogenic lung epithelial stem cells (PLESCs) relative to normal pulmonary stem cells.

4. A pulmonary delivery device for treating one or more of an inflammatory disease, metaplasia, dysplasia or cancer of pulmonary tissues, which device comprises an inhalation formulation of an agent that selectively kills or inhibits the proliferation or induces differentiation of pathogenic lung epithelial stem cells (PLESCs) relative to normal pulmonary stem cells, which device delivers a dose of the agent to the afflicted lung tissue; wherein the pulmonary delivery device is optionally a metered dose inhaler, a nebulizer or a dry powder inhaler.

5. The method of claim 1, for the treatment of an inflammatory lung disease, such as COPD, bronchopulmonary dysplasia, chronic bronchitis, emphysema, idiopathic pulmonary fibrosis, Asthma, chronic rhinosinusitis or a combination thereof.

6. The method of claim 1, for the treatment of COPD or IPF.

7. The method of claim 1, wherein the agent is administered as part of a therapy including administration of one or more anti-inflammatory agents.

8. The method of claim 1, wherein the agent is administered by inhalation.

9. The preparation of claim 3, wherein the agent is formulated for inhalation delivery.

10. The method of claim 8, wherein the agent is formulated for and provided as part of a metered dose inhaler, a nebulizer or a dry powder inhaler.

11. The method of claim 8, wherein the agent is formulated as part of a drug-eluting particle, drug eluting matrix or drug-eluting gel.

12. The method of claim 8, wherein the agent is formulated for oral delivery.

13. The method of claim 1, wherein the agent selectively inhibits the proliferation or induces differentiation of PLESCs, or selectively kills PLESCs, with an IC₅₀ that is ⅕^{th} or less the IC₅₀ for normal pulmonary stem cells, more preferably 1/10^{th}, 1/20^{th}, 1/50^{th}, 1/100^{th}, 1/250^{th}, 1/500^{th} or even 1/1000^{th}.

14. The method of claim 1, wherein the agent inhibits the proliferation or induces differentiation of PLESCs, or kills PLESCs, with an IC₅₀ of 10⁻⁶ M or less, more preferably 10⁻⁷ M or less, 10⁻⁸ M or less or 10⁻⁹M or less.

15. The method of claim 1, wherein the agent is an mTOR inhibitor.

16. The method of claim 15, wherein the mTOR inhibitor is a PI3K/mTOR inhibitor.

17. The method of claim 1, wherein the agent is a CDK inhibitor.

18. The method of claim 17, wherein the CK inhibitor is a CDK2 inhibitor.

19. The method of claim 1, wherein the agent is an HDAC inhibitor.

20. The method of claim 1, wherein the agent is an AKT inhibitor.

21. The method of claim 1, wherein the agent is an an RAR antagonist.

22. The method of claim 1, wherein the agent is a proteasome inhibitor.

23. The method, preparation or device of any of claims of claim 22, wherein the proteasome inhibitor, is an immunoproteasome inhibitor.

24. The method of claim 1, wherein the agent is an Aryl Hydrocarbon Receptor antagonist, an EGFR Inhibitor, an IAP inhibitor or a multiple ion channel blocker.

25. The method of claim 1, wherein the agent is an HSP90 inhibitor, an HSP70 inhibitor or a dual HSP90/HSP70 inhibitor.

26. The method of claim 1, further comprises combining the agent with a second drug agent that selectively promotes proliferation of normal pulmonary stem cells with an EC₅₀ at least 5 times more potent than for PLESCs, more preferably with an EC₅₀ 10 times, 50 times, 100 times or even 1000 times more potent than for PLESCs.

27. The method of claim 26, wherein the second drug agent promotes proliferation of normal pulmonary stem cells with an EC₅₀ of 10⁻⁶ M or less, more preferably 10⁻⁷ M or less, 10⁻⁸ M or less or 10⁻⁹ M or less.

28. The method of claim 26, wherein the second drug agent is a BACE inhibitor, such as a BACE1 inhibitor.

29. The method of claim 26, wherein the second drug agent is a BCR-ABL kinase Inhibitor or FLT3 Inhibitor.

30. The method of claim 26, wherein the second drug agent is an FAK Inhibitor.

31. The method of claim 26, wherein the second drug agent is a VEGFR inhibitor.

32. The method of claim 26, wherein the second drug agent is an AKT inhibitor.

33. The method of claim 26, wherein the agent and the second agent are administered to the patient as separate formulations.

34. The method of claim 26, wherein the agent and the second agent are co-formulated together.

35. The method of claim 1, wherein the patient is a human patient.
